(19) United States
(12) Patent Application Publication
(10) Pub. No.: US 2025/0152743 A1
(43) Pub. Date: May 15, 2025

(54) SYNPOPTOSIS CIRCUITS FOR PROGRAMMABLE CELL DEATH CONTROL

(71) Applicant: California Institute of Technology, Pasadena, CA (US)

(72) Inventors: Michael B. Elowitz, Pasadena, CA (US); Shiyu Xia, Pasadena, CA (US); Andrew C. Lu, Pasadena, CA (US); Lukas Moeller, Pasadena, CA (US)

(21) Appl. No.: 18/945,412

(22) Filed: Nov. 12, 2024

Related U.S. Application Data
(60) Provisional application No. 63/548,335, filed on Nov. 13, 2023.

(51) Int. Cl. A61K 48/00; C07K 7/06; C12N 9/50

(52) U.S. Cl. CPC A61K 48/0058; C07K 7/06; C12N 9/50; C12Y 304/22056; C07K 2319/50

(57) ABSTRACT

Disclosed herein include methods, compositions, and kits suitable for use in programmable cell death control. Compositions (e.g., nucleic acid compositions, synthetic protein circuits) provided herein can comprise one or more apoptosis polypeptides; and/or one or more pyroptosis polypeptides; and/or one or more input polypeptides. In some embodiments, expression of a synthetic protein circuit of the disclosure can induce, e.g., apoptosis and/or pyroptosis in a cell.

### RELATED APPLICATIONS

This application claims the benefit under 35 U.S.C. § 119(e) of U.S. Provisional Patent Application Ser. No. 63/548,335, filed Nov. 13, 2023, the content of this related application is incorporated herein by reference in its entirety for all purposes.

### STATEMENT REGARDING FEDERALLY SPONSORED R&D

This invention was made with government support under Grant No. EB030015 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-810001-US_SequenceListing, created Nov. 12, 2024, which is 52,231 bytes in size. The information in the electronic format of the Sequence Listing is incorporated herein by reference in its entirety.

### BACKGROUND

### Field

The present disclosure relates generally to the field of synthetic biology.

### Description of the Related Art

Mammalian systems use distinct cell death programs to eliminate harmful cells and shape immunity. Apoptosis is immunologically silent or “cold”. By contrast, pyroptosis is immunologically “hot” and involves substantial release of damage-associated molecular patterns (DAMPs).

Apoptosis and pyroptosis can each be advantageous depending on immunological context. The immunostimulatory nature of pyroptosis can promote cell killing. For example, inducing pyroptosis in a 15% minority of cells was sufficient to clear an entire tumor by boosting anti-tumor immunity. Consistently, expression of the gasdermin (GSDM) family of pore-forming proteins, the executioners of pyroptosis, positively correlates with cancer patient survival, and cytotoxic lymphocytes upregulate GSDM expression in cancer cells. To escape pyroptosis, cancer cells generate loss-of-function GSDM mutations, silence GSDM expression, and express non-pyroptotic GSDM variants. Although beneficial in some contexts, pyroptosis can lead to pathological inflammation if triggered excessively. Therefore, it would be desirable to controllably induce apoptosis or pyroptosis and tune their relative frequencies.

Existing cell-killing approaches cannot fully direct the mode of cell death. Cytotoxic drugs are often limited to triggering apoptosis in cold tumors. CAR-T cells can effectively target cells expressing either a single antigen or multiple antigens. However, to kill target cells, CAR-T cells typically use granzymes, which may induce either apoptosis or pyroptosis. Granzyme-independent approaches have also been attempted, including engineered TRAIL-presenting cells and synthetic circuits that regulate caspases, BID, or BAX. However, these approaches are similarly restricted to induction of apoptosis.

To enable tailored control of cell death, there is a need for a set of synthetic circuits with the following features. First, the circuits should allow activation and repression of both apoptosis and pyroptosis. Second, they should steer the mode of cell death in various cell contexts. Third, they should allow the integration and computation of multiple input signals. Fourth, they should be able to selectively kill target cells. Fifth, they should support cell-cell transmission, offering the potential to engineer synthetic killer cells that use designed death programs to eliminate other cells.

### SUMMARY

Disclosed herein include synthetic protein circuits comprising: a first apoptosis polypeptide comprising a large subunit of an apoptotic effector protein and a small subunit of an apoptotic effector protein separated by a first heterologous protease cleavage site, wherein two first apoptosis polypeptides are capable of associating with each other to constitute a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, wherein the first apoptotic protein complex in the first apoptotic protein complex active state is capable of inducing apoptosis in a cell. In some embodiments, the synthetic protein circuit further comprises a first input polypeptide comprising the first heterologous protease.

Disclosed herein include synthetic protein circuits comprising: a first apoptosis polypeptide comprising a large subunit of an apoptotic effector protein and a first partner domain; and a second apoptosis polypeptide comprising a small subunit of an apoptotic effector protein, a second partner domain capable of binding the first partner domain, a first heterologous protease cleavage site, and a first degron, wherein a first heterologous protease in a first heterologous protease active state is capable of cutting the first heterologous protease cleavage site of the second apoptosis polypeptide and thereby releasing the first degron, and wherein the first heterologous protease cleavage site of the second apoptosis polypeptide being cut changes the second apoptosis polypeptide from a second apoptosis polypeptide destabilized state to a second apoptosis polypeptide stabilized state, wherein the first apoptosis polypeptide and the second apoptosis polypeptide in the second apoptosis polypeptide stabilized state are capable of associating via binding of the first partner domain and the second partner domain to form a subunit, and wherein two subunits are capable of associating with each other to constitute a first apoptotic protein complex capable of being in a first apoptotic protein complex active state, wherein the first apoptotic protein complex in the first apoptotic protein complex active state is capable of inducing apoptosis in a cell. In some embodiments, the synthetic protein circuit further comprises a first input polypeptide comprising the first heterologous protease.

Disclosed herein include synthetic protein circuits comprising: a first apoptosis polypeptide comprising a large subunit of an apoptotic effector protein, a first partner domain, a first degron, and a first heterologous protease cleavage site; and a second apoptosis polypeptide comprising a small subunit of an apoptotic effector protein and a second partner domain capable of binding the first partner domain, wherein the first apoptosis polypeptide and the second apoptosis polypeptide are capable of associating via binding of the first partner domain and the second partner domain to form a subunit, wherein two subunits are capable of associating with each other to constitute a first apoptotic protein complex capable of being in a first apoptotic protein complex active state, wherein the first apoptotic protein complex in the first apoptotic protein complex active state is capable of inducing apoptosis in a cell, wherein a first heterologous protease in a first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state. In some embodiments, the synthetic protein circuit further comprises a first input polypeptide comprising the first heterologous protease.

In some embodiments, the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof. In some embodiments, the first heterologous protease comprises or is derived from one or more of a prokaryotic protease, bacterial protease, archaeal protease, eukaryotic protease, fungal protease, plant protease, mammalian protease, and human protease. In some embodiments, the first heterologous protease is engineered. In some embodiments, the first heterologous protease comprises tobacco etch virus (TEV) protease, tobacco vein mottling virus (TVMV) protease, hepatitis C virus protease (HCVP), derivatives thereof, or any combination thereof. In some embodiments, the first heterologous protease cleavage site comprises the sequence of SEQ ID NO: 1 or SEQ ID NO: 2. In some embodiments, the first heterologous protease cleavage site is natural or engineered, e.g., the cleavage sequence of a human apoptotic effector protease, e.g., a caspase cleavage sequence. In some embodiments, the first heterologous protease cleavage site is configured to modulate an activation threshold of the synthetic protein circuit. In some embodiments, said configuring comprises introducing one or more amino acid insertions, substitutions, or deletions relative to a wild type first heterologous protease cleavage site. In some embodiments, the first heterologous protease cleavage site comprises the sequence of any one of SEQ ID NOs: 3-7. In some embodiments, the first apoptotic protein complex in the first apoptotic protein complex active state is capable of being inhibited by a small molecule inhibitor of apoptosis, e.g., Quinoline-Val-Asp-Difluorophenoxymethylketone (Q-VD-OPh), carbobenzoxy-valyl-alanyl-aspartyl-[O-methyl]-fluoromethylketone (Z-VAD-FMK), and/or emricasan.

In some embodiments, the first apoptosis polypeptide and/or the second apoptosis polypeptide comprise one or more third partner domains and one or more fourth partner domains capable of binding the third partner domain, wherein binding of the third partner domain and the fourth partner domain is capable of inhibiting change of the first apoptotic protein complex from the first apoptotic protein complex inactive state to the first apoptotic protein complex active state in the absence of the first heterologous protease in the first heterologous protease active state. In some embodiments, said third partner domains and fourth partner domains are capable of multimerization. In some embodiments, the first partner domain and the second partner domain are homodimers, and/or wherein the third partner domain and the fourth partner domain are homodimers. In some embodiments, the first partner domain and the second partner domain are heterodimers and/or wherein the third partner domain and the fourth partner domain are heterodimers. In some embodiments, the binding between the first partner domain and the second partner domain is reversible and/or wherein the binding between the third partner domain and the fourth partner domain is reversible.

In some embodiments, the first partner domain, the second partner domain, the third partner domain, and/or the fourth partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37 BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the first partner domain, the second partner domain, the third partner domain, and/or the fourth partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the first partner domain and the second partner domain and/or the third partner domain and the fourth partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the first partner domain, the second partner domain, the third partner domain, and/or the fourth partner domain comprise CZp, NZp, or any combination thereof. In some embodiments, the first partner domain, the second partner domain, the third partner domain, and/or the fourth partner domain comprise nHalo, cHalo, or any combination thereof.

Disclosed herein include synthetic protein circuits comprising: a first pyroptosis polypeptide comprising a pyroptosis effector domain and a first inhibitory domain separated by a first heterologous protease cleavage site, wherein the first inhibitory domain is capable of inhibiting activity of the pyroptosis effector domain, thereby the first pyroptosis polypeptide is in a first pyroptosis polypeptide inactive state, wherein a first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in a cell. In some embodiments, the synthetic protein circuit further comprises a first input polypeptide comprising the first heterologous protease.

In some embodiments, the first inhibitory domain comprises a protein domain configured to reduce or abrogate the activity of the pyroptosis effector domain, e.g., a bulky domain. In some embodiments, the first inhibitory domain comprises an auto-inhibitory domain of a gasdermin family of pore-forming proteins or maltose-binding protein. In some embodiments, the cell does not express an endogenous protein comprising a pyroptosis effector domain.

Disclosed herein include synthetic protein circuits comprising: a first pyroptosis polypeptide comprising a pyroptosis effector domain and a first degron separated by a first heterologous protease cleavage site, wherein a first heterologous protease in a first heterologous protease active state is capable of cutting the first heterologous protease cleavage site of the first pyroptosis polypeptide and thereby releasing the first degron, and wherein the first heterologous protease cleavage site of the first pyroptosis polypeptide being cut changes the first pyroptosis polypeptide from a first pyroptosis polypeptide destabilized state to a first pyroptosis polypeptide stabilized state, wherein the first pyroptosis polypeptide in the first pyroptosis polypeptide stabilized state is capable of being in a first pyroptosis polypeptide active state, wherein the first pyroptosis polypeptide in the first pyroptosis polypeptide active state is capable of inducing pyroptosis in a cell. In some embodiments, the synthetic protein circuit further comprises a first input polypeptide comprising the first heterologous protease.

Disclosed herein include synthetic protein circuits comprising: a first pyroptosis polypeptide comprising a pyroptosis effector domain, a first degron, and a first heterologous protease cleavage site, wherein the first pyroptosis polypeptide is capable of being in a first pyroptosis polypeptide active state, wherein the first pyroptosis polypeptide in the first pyroptosis polypeptide active state is capable of inducing pyroptosis in a cell, and wherein a first heterologous protease in a first heterologous protease active state is capable of cutting the first heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state. In some embodiments, the synthetic protein circuit further comprises a first input polypeptide comprising the first heterologous protease.

In some embodiments, the pyroptosis effector domain comprises an N-terminal domain of a gasdermin (GSDM) protein. In some embodiments, the GSDM protein is from the gasdermin (GSDM) family, including GSDMA, GSDMB, GSDMC, GSDMD, GSDME, or DFNB59. In some embodiments, the first heterologous protease comprises or is derived from one or more of a prokaryotic protease, bacterial protease, archaeal protease, eukaryotic protease, fungal protease, plant protease, mammalian protease, and human protease. In some embodiments, the first heterologous protease is engineered. In some embodiments, the first heterologous protease comprises tobacco etch virus (TEV) protease, tobacco vein mottling virus (TVMV) protease, hepatitis C virus protease (HCVP), derivatives thereof, or any combination thereof. In some embodiments, the first heterologous protease cleavage site comprises the sequence of SEQ ID NO: 1 or SEQ ID NO: 2. In some embodiments, the first heterologous protease cleavage site is natural or engineered, e.g., the cleavage sequence of a human apoptotic effector protease, e.g., a caspase cleavage sequence. In some embodiments, the first heterologous protease cleavage site is configured to modulate an activation threshold of the synthetic protein circuit. In some embodiments, said configuring comprises introducing one or more amino acid insertions, substitutions, or deletions relative to a wild type first heterologous protease cleavage site. In some embodiments, the first heterologous protease cleavage site comprises the sequence of any one of SEQ ID NOs: 3-7.

In some embodiments, the first pyroptosis polypeptide comprises one or more third partner domains and one or more fourth partner domains capable of binding the third partner domain, wherein binding of the third partner domain and the fourth partner domain is capable of inhibiting change of the first pyroptosis polypeptide from the first pyroptosis polypeptide inactive state to the first pyroptosis polypeptide active state in the absence of the first heterologous protease in the first heterologous protease active state. In some embodiments, said third partner domains and fourth partner domains are capable of multimerization. In some embodiments, the third partner domain and the fourth partner domain are homodimers. In some embodiments, the third partner domain and the fourth partner domain are heterodimers. In some embodiments, the binding between the third partner domain and the fourth partner domain is reversible.

In some embodiments, the third partner domain and/or the fourth partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13 XAAA heterodimer a, DHD13 XAXA heterodimer a, DHD13 XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34 XAAXA heterodimer a, DHD34 XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37 AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the third partner domain and/or the fourth partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the third partner domain and the fourth partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the third partner domain and/or the fourth partner domain comprise CZp, NZp, or any combination thereof. In some embodiments, the third partner domain and/or the fourth partner domain comprise nHalo, cHalo, or any combination thereof. In some embodiments, inducing pyroptosis in the cell causes the cell to induce key signatures of pyroptosis: chromatin condensation and DNA fragmentation, pore formation, cell swelling, and osmotic lysis, followed by release of one or more inflammatory cytokines. In some embodiments, the one or more inflammatory cytokines comprise IL-18, IL-1β, IL-6, IL-8, interferon gamma (IFN-γ), and/or tumor necrosis factor-alpha (TNF-α).

Disclosed herein include synthetic protein circuits comprising: one or more apoptosis polypeptides; and/or one or more pyroptosis polypeptides; and/or one or more input polypeptides.

Disclosed herein include synthetic protein circuits comprising: (i) one or more apoptosis polypeptides or one or more pyroptosis polypeptides and (ii) one or more input polypeptides, configured to form one or more logic gates selected from the group comprising an OR logic gate, AND logic gate, NOR logic gate, NAND logic gate, IMPLY logic gate, NIMPLY logic gate, XOR logic gate, and an XNOR logic gate.

The synthetic protein circuit can comprise: (i) a first apoptosis polypeptide comprising a large subunit of an apoptotic effector protein and a small subunit of an apoptotic effector protein separated by a first heterologous protease cleavage site, a second heterologous protease cleavage site, and a first degron, wherein the first apoptosis polypeptide is capable of being in a first apoptosis polypeptide destabilized state; (ii) a first input polypeptide comprising a first heterologous protease; and/or (iii) a second input polypeptide comprising a second heterologous protease, wherein the second heterologous protease in a second heterologous protease active state is capable of cutting the second heterologous protease cleavage site of the first apoptosis polypeptide, wherein the second heterologous protease cleavage site of the first apoptosis polypeptide being cut changes the first apoptosis polypeptide from the first apoptosis polypeptide destabilized state to a first apoptosis polypeptide stabilized state; wherein two of the first apoptosis polypeptides in the first apoptosis polypeptide stabilized state are capable of forming a first apoptotic protein complex in a first apoptotic protein complex inactive state, and wherein the first heterologous protease in a first heterologous protease active state is capable of cutting the first heterologous protease cleavage site of the first apoptosis polypeptide, wherein the first heterologous protease cleavage site of the first apoptosis polypeptide being cut changes the first apoptotic protein complex from the first apoptotic protein complex inactive state to a first apoptotic protein complex active state, and wherein the first apoptotic protein complex in the first apoptotic protein complex active state is capable of inducing apoptosis in a cell.

The synthetic protein circuit can comprise: (i) a first apoptosis polypeptide comprising a large subunit of an apoptotic effector protein and a small subunit of an apoptotic effector protein separated by a first heterologous protease cleavage site and a second heterologous protease cleavage site, wherein two of the first apoptosis polypeptides are capable of forming a first apoptotic protein complex in a first apoptotic protein complex inactive state; (ii) a first input polypeptide comprising a first heterologous protease; and/or (iii) a second input polypeptide comprising a second heterologous protease, wherein: (I) the first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state; (II) the second heterologous protease in a second heterologous protease active state is capable of cutting the second heterologous protease cleavage site of the first apoptosis polypeptide, and wherein the second heterologous protease cleavage site of the first apoptosis polypeptide being cut changes the first apoptotic protein complex from the first apoptotic protein complex inactive state to the first apoptotic protein complex active state; or (III) the first heterologous protease in the first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to the first apoptotic protein complex active state, and/or the second heterologous protease in the second heterologous protease active state is capable of cutting the second heterologous protease cleavage site of the first apoptosis polypeptide, and wherein the second heterologous protease cleavage site of the first apoptosis polypeptide being cut changes the first apoptotic protein complex from the first apoptotic protein complex inactive state to the first apoptotic protein complex active state, wherein the first apoptotic protein complex in the first apoptotic protein complex active state is capable of inducing apoptosis in a cell.

The synthetic protein circuit can comprise: (i) a first apoptosis polypeptide comprising a large subunit of an apoptotic effector protein, a first partner domain, a first degron, and a first heterologous protease cleavage site; (ii) a second apoptosis polypeptide comprising a small subunit of an apoptotic effector protein, a second partner domain capable of binding the first partner domain, a second heterologous protease cleavage site, and a second degron, (iii) a first input polypeptide comprising a first heterologous protease; and/or (iv) a second input polypeptide comprising a second heterologous protease, wherein: (I) the first heterologous protease in a first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state; (II) the second heterologous protease in a second heterologous protease active state is capable of cutting the second heterologous protease cleavage site of the second apoptosis polypeptide and thereby releasing the second degron, and wherein the second heterologous protease cleavage site of the second apoptosis polypeptide being cut changes the second apoptosis polypeptide from a second apoptosis polypeptide destabilized state to a second apoptosis polypeptide stabilized state, wherein the first apoptosis polypeptide and the second apoptosis polypeptide in the second apoptosis polypeptide stabilized state are capable of associating via binding of the first partner domain and the second partner domain to form a subunit, and wherein two subunits are capable of associating with each other to constitute a first apoptotic protein complex capable of being in a first apoptotic protein complex active state, wherein the first apoptotic protein complex in the first apoptotic protein complex active state is capable of inducing apoptosis in a cell; or (III) wherein the first heterologous protease in the first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from the first apoptosis polypeptide stabilized state to the first apoptosis polypeptide destabilized state, and the second heterologous protease in the second heterologous protease active state is capable of cutting the second heterologous protease cleavage site of the second apoptosis polypeptide and thereby releasing the second degron, and wherein the second heterologous protease cleavage site of the second apoptosis polypeptide being cut changes the second apoptosis polypeptide from the second apoptosis polypeptide destabilized state to the second apoptosis polypeptide stabilized state.

The synthetic protein circuit can comprise: (i) a first apoptosis polypeptide comprising a large subunit of an apoptotic effector protein and a small subunit of an apoptotic effector protein separated by a first heterologous protease cleavage site, a second heterologous protease cleavage site, and a first degron; (ii) a second apoptosis polypeptide comprising the large subunit of the apoptotic effector protein and the small subunit of the apoptotic effector protein separated by the second heterologous protease cleavage site, the first heterologous protease cleavage site, and a second degron, wherein two of the first apoptosis polypeptides are capable forming a first apoptotic protein complex in a first apoptotic protein complex inactive state, two of the second apoptosis polypeptides are capable forming a second apoptotic protein complex in a second apoptotic protein complex inactive state, and/or one of the first apoptosis polypeptide and one of the second apoptosis polypeptide are capable forming a third apoptotic protein complex in a third apoptotic protein complex inactive state; (iii) a first input polypeptide comprising a first heterologous protease; and/or (iv) a second input polypeptide comprising a second heterologous protease, wherein: (I) the first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, and the first heterologous protease in the first heterologous protease active state is capable of cutting the first heterologous protease cleavage site of the second apoptosis polypeptide to expose the second degron, and wherein the second degron of the second apoptosis polypeptide being exposed changes the second apoptosis polypeptide from a second apoptosis polypeptide stabilized state to a second apoptosis polypeptide destabilized state, wherein the first apoptotic protein complex in the first apoptotic protein complex active state is capable of inducing apoptosis in a cell; (II) wherein the second heterologous protease in a second heterologous protease active state is capable of cutting the second heterologous protease cleavage site of the second apoptosis polypeptide, and wherein the second heterologous protease cleavage site of the second apoptosis polypeptide being cut changes the second apoptotic protein complex from the second apoptotic protein complex inactive state to a second apoptotic protein complex active state, and wherein the second heterologous protease in the second heterologous protease active state is capable of cutting the second heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state, wherein the second apoptotic protein complex in the second apoptotic protein complex active state is capable of inducing apoptosis in the cell; or (III) wherein the first heterologous protease in the first heterologous protease active state is capable of cutting the first heterologous protease cleavage site of the second apoptosis polypeptide to expose the first degron, and wherein the first degron of the second apoptosis polypeptide being exposed changes the second apoptosis polypeptide from the second apoptosis polypeptide stabilized state to the second apoptosis polypeptide destabilized state; and wherein the second heterologous protease in the second heterologous protease active state is capable of cutting the second heterologous protease cleavage site of the first apoptosis polypeptide to expose the first degron, and wherein the first degron of the first apoptosis polypeptide being exposed changes the first apoptosis polypeptide from the first apoptosis polypeptide stabilized state to the first apoptosis polypeptide destabilized state.

The synthetic protein circuit can comprise: (i) a first apoptosis polypeptide comprising a large subunit of an apoptotic effector protein, and a first partner domain and a first degron separated by a first heterologous protease cleavage site, and a second heterologous protease cleavage site; (ii) a second apoptosis polypeptide comprising a small subunit of an apoptotic effector protein and a second partner domain capable of binding the first partner domain, wherein the first apoptosis polypeptide and the second apoptosis polypeptide are capable of associating via binding of the first partner domain and the second partner domain to form a subunit; wherein two subunits are capable of associating with each other to constitute a first apoptotic protein complex capable of being in a first apoptotic protein complex active state, wherein the first apoptotic protein complex in the first apoptotic protein complex active state is capable of inducing apoptosis in a cell; (iii) a first input polypeptide comprising a first heterologous protease; and/or (iv) a second input polypeptide comprising a second heterologous protease, wherein: (I) the first heterologous protease in a first heterologous protease active state is capable of cutting the first heterologous protease cleavage site of the first apoptosis polypeptide, thereby releasing the first degron; (II) the second heterologous protease in a second heterologous protease active state is capable of cutting the second heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state.

The synthetic protein circuit can comprise: (i) a first apoptosis polypeptide comprising a large subunit of an apoptotic effector protein, a first partner domain, a first degron, and a first heterologous protease cleavage site; (ii) a second apoptosis polypeptide comprising the large subunit of the apoptotic effector protein, the first partner domain, a second degron, and a second heterologous protease cleavage site; (iii) a third apoptosis polypeptide comprising a small subunit of an apoptotic effector protein and a second partner domain capable of binding the first partner domain, wherein the first apoptosis polypeptide and the third apoptosis polypeptide are capable of associating via binding of the first partner domain and the second partner domain to form a first subunit, and/or the second apoptosis polypeptide and the third apoptosis polypeptide are capable of associating via binding of the first partner domain and the second partner domain to form a second subunit, wherein two first subunits, two second subunits, and/or one first subunit and one second subunit are capable of associating with each other to constitute a first apoptotic protein complex capable of being in a first apoptotic protein complex active state, wherein the first apoptotic protein complex in the first apoptotic protein complex active state is capable of inducing apoptosis in a cell; (iv) a first input polypeptide comprising a first heterologous protease; and/or (v) a second input polypeptide comprising a second heterologous protease, wherein: (I) the first heterologous protease in a first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state; (II) the second heterologous protease in a second heterologous protease active state is capable of cutting the second heterologous protease cleavage site of the second apoptosis polypeptide to expose the second degron, and wherein the second degron of the second apoptosis polypeptide being exposed changes the second apoptosis polypeptide from a second apoptosis polypeptide stabilized state to a second apoptosis polypeptide destabilized state; or (III) the first heterologous protease in the first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from the first apoptosis polypeptide stabilized state to the first apoptosis polypeptide destabilized state, and the second heterologous protease in the second heterologous protease active state is capable of cutting the second heterologous protease cleavage site of the second apoptosis polypeptide to expose the second degron, and wherein the second degron of the second apoptosis polypeptide being exposed changes the second apoptosis polypeptide from the second apoptosis polypeptide stabilized state to the second apoptosis polypeptide destabilized state.

In some embodiments, the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof. In some embodiments, the first and/or second heterologous protease comprises or is derived from one or more of a prokaryotic protease, bacterial protease, archaeal protease, eukaryotic protease, fungal protease, plant protease, mammalian protease, and human protease. In some embodiments, the first and/or second heterologous protease is engineered. In some embodiments, wherein the first and/or second heterologous protease comprises tobacco etch virus (TEV) protease, tobacco vein mottling virus (TVMV) protease, hepatitis C virus protease (HCVP), derivatives thereof, or any combination thereof. In some embodiments, the first and/or second heterologous protease cleavage site comprises the sequence of SEQ ID NO: 1 or SEQ ID NO: 2. In some embodiments, the first and/or second heterologous protease cleavage site is natural or engineered, e.g., a cleavage sequence of a human apoptotic effector protease, e.g., a caspase cleavage sequence. In some embodiments, the first and/or the second heterologous protease cleavage site is configured to modulate an activation threshold of the synthetic protein circuit. In some embodiments, said configuring comprises introducing one or more amino acid insertions, substitutions, or deletions relative to a wild type first and/or second heterologous protease cleavage site. In some embodiments, the first and/or second heterologous protease cleavage site comprises the sequence of any one of SEQ ID NOs: 3-7. In some embodiments, the first and second heterologous protease are different from each other. In some embodiments, the first, second, and/or third apoptotic protein complex in the first, second, and/or third apoptotic protein complex active state is capable of being inhibited by a small molecule inhibitor of apoptosis, e.g., Quinoline-Val-Asp-Difluorophenoxymethylketone (Q-VD-OPh), carbobenzoxy-valyl-alanyl-aspartyl-[O-methyl]-fluoromethylketone (Z-VAD-FMK), and/or emricasan

In some embodiments, the first apoptosis polypeptide, the second apoptosis polypeptide and/or the third apoptosis polypeptide comprises one or more third partner domains and one or more fourth partner domains capable of binding the third partner domain, wherein binding of the third partner domain and the fourth partner domain is capable of inhibiting change of the first, second, and/or third apoptotic protein complex in the first, second, and/or third apoptotic protein complex inactive state to the first, second, and/or third apoptotic protein complex active state in the absence of the first heterologous protease in the first heterologous protease active state and/or the second heterologous protease in the second heterologous protease in the second heterologous protease active state. In some embodiments, said third partner domains and fourth partner domains are capable of multimerization. In some embodiments, the first partner domain and the second partner domain are homodimers and/or wherein the third partner domain and the fourth partner domain are homodimers. In some embodiments, the first partner domain and the second partner domain are heterodimers and/or wherein the third partner domain and the fourth partner domain are heterodimers. In some embodiments, the binding between the first partner domain and the second partner domain is reversible and/or wherein the binding between the third partner domain and the fourth partner domain is reversible.

In some embodiments, the first partner domain, the second partner domain, the third partner domain, and/or the fourth partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34 XAAXA heterodimer a, DHD34 XAXXA heterodimer a, DHD34 XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13 XAAA heterodimer b, DHD13 XAXA heterodimer b, DHD13 XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the first partner domain, the second partner domain, the third partner domain, and/or the fourth partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the first partner domain and the second partner domain and/or the third partner domain and the fourth partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the first partner domain, the second partner domain, the third partner domain, and/or the fourth partner domain comprise CZp, NZp, or any combination thereof. In some embodiments, the first partner domain, the second partner domain, the third partner domain, and/or the fourth partner domain comprise nHalo, cHalo, or any combination thereof.

The synthetic protein circuit can comprise: (i) a first pyroptosis polypeptide comprising a pyroptosis effector domain, a first inhibitory domain, and a second inhibitory domain, wherein the pyroptosis effector domain and the first inhibitory domain are separated by a first heterologous protease cleavage site, and the pyroptosis effector domain and the second inhibitory domain are separated by a second heterologous cleavage site, wherein the first and/or second inhibitory domain is capable of inhibiting activity of the pyroptosis effector domain, thereby the first pyroptosis polypeptide is in a first pyroptosis polypeptide inactive state; (ii) a first input polypeptide comprising a first heterologous protease; and/or (iii) a second input polypeptide comprising a second heterologous protease, wherein: (I) the first heterologous protease in a first heterologous protease active state is capable of cutting the first heterologous protease cleavage site of the first pyroptosis polypeptide, and wherein the first heterologous protease cleavage site of the first pyroptosis polypeptide being cut releases the first inhibitory domain from the first pyroptosis polypeptide, thereby the second inhibitory domain is capable of inhibiting the activity of the pyroptosis effector domain, thereby the first pyroptosis polypeptide is in the first pyroptosis polypeptide inactive state; (II) the second heterologous protease in a second heterologous protease active state is capable of cutting the second heterologous protease cleavage site of the first pyroptosis polypeptide, and wherein the second heterologous protease cleavage site of the first pyroptosis polypeptide being cut releases the second inhibitory domain from the first pyroptosis polypeptide, thereby the first inhibitory domain is capable of inhibiting the activity of the pyroptosis effector domain, thereby the first pyroptosis polypeptide is in the first pyroptosis polypeptide inactive state; or (III) wherein the first heterologous protease in the first heterologous protease active state is capable of cutting the first heterologous protease cleavage site of the first pyroptosis polypeptide, and wherein the first heterologous protease cleavage site of the first pyroptosis polypeptide being cut releases the first inhibitory domain from the first pyroptosis polypeptide, and the second heterologous protease in the second heterologous protease active state is capable of cutting the second heterologous protease cleavage site of the first pyroptosis polypeptide, and wherein the second heterologous protease cleavage site of the first pyroptosis polypeptide being cut releases the second inhibitory domain from the first pyroptosis polypeptide, wherein the first heterologous protease cleavage site of the first pyroptosis polypeptide being cut and the second heterologous protease cleavage site of the first pyroptosis polypeptide being cut changes the first pyroptosis polypeptide from the first pyroptosis polypeptide inactive state to a first pyroptosis polypeptide active state, and wherein the first pyroptosis polypeptide in the first pyroptosis polypeptide active state is capable of inducing pyroptosis in a cell.

In some embodiments, the first and/or second inhibitory domain comprises a protein domain configured to reduce or abrogate the activity of the pyroptosis effector domain, e.g., a bulky domain. In some embodiments, the first and/or second inhibitory domain comprises an auto-inhibitory domain of a gasdermin family of pore-forming proteins or maltose-binding protein. In some embodiments, the cell does not express an endogenous protein comprising a pyroptosis effector domain.

The synthetic protein circuit can comprise: (i) a first pyroptosis polypeptide comprising a pyroptosis effector domain and a first inhibitory domain separated by a first heterologous protease cleavage site and a second heterologous protease cleavage site, wherein the first inhibitory domain is capable of inhibiting activity of the pyroptosis effector domain, thereby the first pyroptosis polypeptide is in a first pyroptosis polypeptide inactive state; (ii) a first input polypeptide comprising a first heterologous protease; and/or (iii) a second input polypeptide comprising a second heterologous protease, wherein: (I) the first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in a cell; (II) the second heterologous protease in a second heterologous protease active state is capable of cutting the second heterologous protease cleavage site of the first pyroptosis polypeptide, and wherein the second heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in a cell; or (III) the first heterologous protease in the first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, and/or the second heterologous protease in the second heterologous protease active state is capable of cutting the second heterologous protease cleavage site of the first pyroptosis polypeptide, and wherein the second heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in the cell.

In some embodiments, the first inhibitory domain comprises a protein domain configured to reduce or abrogate the activity of the pyroptosis effector domain, e.g., a bulky domain. In some embodiments, the first inhibitory domain comprises an auto-inhibitory domain of a gasdermin family of pore-forming proteins or maltose-binding protein. In some embodiments, the cell does not express an endogenous protein comprising a pyroptosis effector domain.

The synthetic protein circuit can comprise: (i) a first pyroptosis polypeptide comprising a pyroptosis effector domain and a first degron separated by a first heterologous protease cleavage site, a second degron, and a second heterologous protease cleavage site, wherein the first pyroptosis polypeptide is capable of being in a first pyroptosis polypeptide destabilized state; (ii) a first input polypeptide comprising a first heterologous protease; and/or (iii) a second input polypeptide comprising a second heterologous protease, wherein: (I) the first heterologous protease in a first heterologous protease active state is capable of cutting the first heterologous protease cleavage site of the first pyroptosis polypeptide and thereby releasing the first degron, and wherein the first heterologous protease cleavage site of the first pyroptosis polypeptide being cut changes the first pyroptosis polypeptide from the first pyroptosis polypeptide destabilized state to a first pyroptosis polypeptide stabilized state, wherein the first pyroptosis polypeptide in the first pyroptosis polypeptide stabilized state is capable of being in a first pyroptosis polypeptide active state, wherein the first pyroptosis polypeptide in the first pyroptosis polypeptide active state is capable of inducing pyroptosis in a cell; or (II) the first heterologous protease in the first heterologous protease active state is capable of cutting the first heterologous protease cleavage site of the first pyroptosis polypeptide and thereby releasing the first degron, and wherein the first heterologous protease cleavage site of the first pyroptosis polypeptide being cut changes the first pyroptosis polypeptide from the first pyroptosis polypeptide destabilized state to the first pyroptosis polypeptide stabilized state, and the second heterologous protease in the second heterologous protease active state is capable of cutting the second heterologous protease cleavage site of the first pyroptosis polypeptide in the first pyroptosis polypeptide destabilized state to expose the second degron, and wherein the second degron of the first pyroptosis polypeptide in the first pyroptosis polypeptide stabilized state being exposed changes the first pyroptosis polypeptide from the first pyroptosis polypeptide stabilized state to the first pyroptosis polypeptide destabilized state.

The synthetic protein circuit can comprise: (i) a first pyroptosis polypeptide comprising a pyroptosis effector domain and a first inhibitory domain separated by a first heterologous protease cleavage site, a second heterologous cleavage site, and a first degron, wherein the first inhibitory domain is capable of inhibiting activity of the pyroptosis effector domain of the first pyroptosis polypeptide, thereby the first pyroptosis polypeptide is in a first pyroptosis polypeptide inactive state; (ii) a second pyroptosis polypeptide comprising a pyroptosis effector domain and a second inhibitory domain separated by the second heterologous protease cleavage site, the first heterologous cleavage site, and a second degron, wherein the second inhibitory domain is capable of inhibiting activity of the pyroptosis effector domain of the second pyroptosis polypeptide, thereby the second pyroptosis polypeptide is in a second pyroptosis polypeptide inactive state; (iii) a first input polypeptide comprising a first heterologous protease; and/or (iv) a second input polypeptide comprising a second heterologous protease, wherein: (I) the first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in a cell, and wherein the first heterologous protease in the first heterologous protease active state is capable of cutting the first heterologous protease cleavage site of the second pyroptosis polypeptide to expose the second degron, and wherein the second degron of the second pyroptosis polypeptide being exposed changes the second pyroptosis polypeptide from a second pyroptosis polypeptide stabilized state to a second pyroptosis polypeptide destabilized state; (II) the second heterologous protease in a second heterologous protease active state is capable of cutting the second heterologous protease cleavage site of the second pyroptosis polypeptide, and wherein the second heterologous protease cleavage site of the second pyroptosis polypeptide being cut changes the second pyroptosis polypeptide from the second pyroptosis polypeptide inactive state to a second pyroptosis polypeptide active state, wherein the second pyroptosis polypeptide in the second pyroptosis polypeptide active state is capable of inducing pyroptosis in the cell, and wherein the second heterologous protease in the second heterologous protease active state is capable of cutting the second heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state; or (III) the first heterologous protease in the first heterologous protease active state is capable of cutting the first heterologous protease cleavage site of the second pyroptosis polypeptide to expose the second degron, and wherein the second degron of the second pyroptosis polypeptide being exposed changes the second pyroptosis polypeptide from the second pyroptosis polypeptide stabilized state to the second pyroptosis polypeptide destabilized state, and wherein the second heterologous protease in the second heterologous protease active state is capable of cutting the second heterologous protease cleavage site of the first pyroptosis polypeptide to expose the first degron, and wherein the first degron of the first pyroptosis polypeptide being exposed changes the first pyroptosis polypeptide from the first pyroptosis polypeptide stabilized state to the first pyroptosis polypeptide destabilized state.

In some embodiments, the first and/or second inhibitory domain comprises a protein domain configured to reduce or abrogate the activity of the pyroptosis effector domain, e.g., a bulky domain. In some embodiments, the first and/or second inhibitory domain comprises an auto-inhibitory domain of a gasdermin family of pore-forming proteins or maltose-binding protein. In some embodiments, the cell does not express an endogenous protein comprising a pyroptosis effector domain.

The synthetic protein circuit can comprise: (i) a first pyroptosis polypeptide comprising a pyroptosis effector domain and a first degron separated by a first heterologous protease cleavage site, and a second heterologous protease cleavage site, wherein the first pyroptosis polypeptide is capable of being in a first pyroptosis polypeptide active state, wherein the first pyroptosis polypeptide in the first pyroptosis polypeptide active state is capable of inducing pyroptosis in a cell; (ii) a first input polypeptide comprising a first heterologous protease; and/or (iii) a second input polypeptide comprising a second heterologous protease, wherein: (I) the first heterologous protease in a first heterologous protease active state is capable of cutting the first heterologous protease cleavage site of the first pyroptosis polypeptide and thereby releasing the first degron from the first pyroptosis polypeptide; or (II) the second heterologous protease in a second heterologous protease active state is capable of cutting the second heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state.

The synthetic protein circuit can comprise: (i) a first pyroptosis polypeptide comprising a pyroptosis effector domain, a first degron, and a first heterologous protease cleavage site, wherein the first pyroptosis polypeptide is capable of being in a first pyroptosis polypeptide active state, wherein the first pyroptosis polypeptide in the first pyroptosis polypeptide active state is capable of inducing pyroptosis in a cell; (ii) a second pyroptosis polypeptide comprising a pyroptosis effector domain, a second degron, and a second heterologous protease cleavage site, wherein the second pyroptosis polypeptide is capable of being in a second pyroptosis polypeptide active state, wherein the second pyroptosis polypeptide in the second pyroptosis polypeptide active state is capable of inducing pyroptosis in the cell; (iii) a first input polypeptide comprising a first heterologous protease; and/or (iv) a second input polypeptide comprising a second heterologous protease, wherein: (I) the first heterologous protease in a first heterologous protease active state is capable of cutting the first heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state; (II) the second heterologous protease in a second heterologous protease active state is capable of cutting the second heterologous protease cleavage site of the second pyroptosis polypeptide to expose the second degron, and wherein the second degron of the second pyroptosis polypeptide being exposed changes the second pyroptosis polypeptide from a second pyroptosis polypeptide stabilized state to a second pyroptosis polypeptide destabilized state; or (III) the first heterologous protease in the first heterologous protease active state is capable of cutting the first heterologous protease cleavage site of the first pyroptosis polypeptide to expose the first degron, and wherein the first degron of the first pyroptosis polypeptide being exposed changes the first pyroptosis polypeptide from the first pyroptosis polypeptide stabilized state to the first pyroptosis polypeptide destabilized state, and the second heterologous protease in the second heterologous protease active state is capable of cutting the second heterologous protease cleavage site of the second pyroptosis polypeptide to expose the second degron, and wherein the second degron of the second pyroptosis polypeptide being exposed changes the second pyroptosis polypeptide from the second pyroptosis polypeptide stabilized state to the second pyroptosis polypeptide destabilized state.

In some embodiments, the pyroptosis effector domain comprises an N-terminal domain of a gasdermin (GSDM) protein. In some embodiments, the GSDM protein is from the gasdermin (GSDM) family, including GSDMA, GSDMB, GSDMC, GSDMD, GSDME, or DFNB59. In some embodiments, the first and/or second heterologous protease comprises or is derived from one or more of a prokaryotic protease, bacterial protease, archaeal protease, eukaryotic protease, fungal protease, plant protease, mammalian protease, and human protease. In some embodiments, the first and/or second heterologous protease is engineered. In some embodiments, the first and/or second heterologous protease comprises tobacco etch virus (TEV) protease, tobacco vein mottling virus (TVMV) protease, hepatitis C virus protease (HCVP), derivatives thereof, or any combination thereof. In some embodiments, the first and/or second heterologous protease cleavage site comprises the sequence of SEQ ID NO: 1 or SEQ ID NO: 2. In some embodiments, the first and/or second heterologous protease cleavage site is natural or engineered, e.g., the cleavage sequence of a human apoptotic effector protease, e.g., a caspase cleavage sequence. In some embodiments, the first and/or second heterologous protease cleavage site is configured to modulate an activation threshold of the synthetic protein circuit. In some embodiments, said configuring comprises introducing one or more amino acid insertions, substitutions, or deletions relative to a wild type first and/or second heterologous protease cleavage site. In some embodiments, the first and/or second heterologous protease cleavage site comprises the sequence of any one of SEQ ID NOs: 3-7. In some embodiments, the first and/or second heterologous protease are different from each other.

In some embodiments, the first pyroptosis polypeptide and/or the second pyroptosis polypeptide comprise one or more third partner domains and one or more fourth partner domains capable of binding the third partner domain, wherein binding of the third partner domain and the fourth partner domain is capable of, inhibiting change of the first and/or second pyroptosis polypeptide from the first and/or second pyroptosis polypeptide inactive state to the first and/or second pyroptosis polypeptide active state, in the absence of the first and/or second heterologous protease in the first and/or second heterologous protease active state. In some embodiments, said third partner domains and fourth partner domains are capable of multimerization. In some embodiments, the third partner domain and the fourth partner domain are homodimers. In some embodiments, the third partner domain and the fourth partner domain are heterodimers. In some embodiments, the binding between the third partner domain and the fourth partner domain is reversible.

In some embodiments, the third partner domain and/or the fourth partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37 AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37 BBBB heterodimer b, DHD37 XBXB heterodimer b, DHD37 AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the third partner domain and/or the fourth partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the third partner domain and the fourth partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the third partner domain and/or the fourth partner domain comprise CZp, NZp, or any combination thereof. In some embodiments, the third partner domain and/or the fourth partner domain comprise nHalo, cHalo, or any combination thereof. In some embodiments, inducing pyroptosis in the cell causes the cell to release one or more inflammatory cytokines. In some embodiments, the one or more inflammatory cytokines comprise IL-18, IL-1β, IL-6, IL-8, interferon gamma (IFN-γ), and/or tumor necrosis factor-alpha (TNF-α).

In some embodiments, the first apoptosis polypeptide, the second apoptosis polypeptide, the third apoptosis polypeptide, the first pyroptosis polypeptide, the second pyroptosis polypeptide, the first input polypeptide, and/or the second input polypeptide comprise one or more linkers. In some embodiments, the linker: is a flexible linker, a rigid linker, or a hybrid linker; is hydrophilic or hydrophobic; is between 1 and 250 amino acids; comprises one or more flexible amino acid residues, e.g., about 1 to about 18 flexible amino acid residues. In some embodiments, the flexible amino acid residues comprise glycine, serine, or a combination thereof; and/or comprises 2 repeating amino acid subunits or more.

In some embodiments: (i) the first, second, and/or third apoptosis polypeptide; (ii) the first and/or second pyroptosis polypeptide; and/or (iii) the first and/or second input polypeptide, are configured to be in a first localized state. In some embodiments, the first localized state comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first, second, and/or third apoptosis polypeptide, the first and/or second pyroptosis polypeptide, and/or the first and/or second input polypeptide is: (i) tethered to an intracellular organelle and/or membrane; or (ii) fused to a polypeptide that recruits to said localized state. In some embodiments: (i) the first, second, and/or third apoptosis polypeptide; (ii) the first and/or second pyroptosis polypeptide; and/or (iii) the first and/or second input polypeptide, are configured to be in second localized state(s), wherein the first localized state and the second localized state(s) are different. In some embodiments, the second localized state(s) comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first, second, and/or third apoptosis polypeptide, the first and/or second pyroptosis polypeptide, and/or the first and/or second input polypeptide is: (i) tethered to an intracellular organelle and/or membrane; or (ii) fused to a polypeptide that recruits to said localized state. In some embodiments: (i) the first, second, and/or third apoptosis polypeptide; (ii) the first and/or second pyroptosis polypeptide; and/or (iii) the first and/or second input polypeptide comprise a first localization signal. In some embodiments, the first localization signal is adjacent to a third degron and/or a third heterologous cleavage site. In some embodiments: (i) the first, second, and/or third apoptosis polypeptide; (ii) the first and/or second pyroptosis polypeptide; and/or (iii) the first and/or second input polypeptide comprise second localization signal(s). In some embodiments, the second localization signal is adjacent to a third degron and/or a third heterologous protease cleavage site. In some embodiments, the presence of the third degron and/or wherein the third heterologous cleavage site being cut changes: (i) the first, second, and/or third apoptosis polypeptide from a non-localized state or the first localized state to the second localized state(s) or to a non-localized state; (ii) the first and/or second pyroptosis polypeptide from a non-localized state or the first localized state to the second localized state(s) or to a non-localized state; and/or (iii) the first and/or second input polypeptide from a non-localized state or the first localized state to the second localized state(s) or to a non-localized state. In some embodiments, the synthetic protein circuit further comprises a third input polypeptide comprising a third heterologous protease capable of cutting the third heterologous protease cleavage site. In some embodiments, the third heterologous protease comprises or is derived from one or more of a prokaryotic protease, bacterial protease, archaeal protease, eukaryotic protease, fungal protease, plant protease, mammalian protease, and human protease. In some embodiments, the third heterologous protease is engineered. In some embodiments, the third heterologous protease comprises tobacco etch virus (TEV) protease, tobacco vein mottling virus (TVMV) protease, hepatitis C virus protease (HCVP), derivatives thereof, or any combination thereof. In some embodiments, the third heterologous protease cleavage site comprises the sequence of SEQ ID NO: 1 or SEQ ID NO: 2. In some embodiments, the third heterologous protease cleavage site is natural or engineered, e.g., the cleavage sequence of a human apoptotic effector protease, e.g., a caspase cleavage sequence. In some embodiments, the third heterologous protease cleavage site is configured to modulate an activation threshold of the synthetic protein circuit. In some embodiments, said configuring comprises introducing one or more amino acid insertions, substitutions, or deletions relative to a wild type third heterologous protease cleavage site. In some embodiments, the third heterologous protease cleavage site comprises the sequence of any one of SEQ ID NOs: 3-7. In some embodiments, the first localization signal and/or second localization signal(s) is selected from the group comprising a nuclear localization signal (NLS), a nuclear export signal (NES), a peroxisomal targeting signal (PTS), a mitochondrial targeting sequence (MTS), a ER signal peptide, CAAX box, a peroxisomal targeting signal (PTS), PTS1, PTS2, a dileucine motif, YxxΦ motif, a palmitoylation motif, a GPI anchor signal, myristoylation signal, HDEL, KDEL, KKXX motif, RXR motif, SV40 NLS, SV40 NES, CAAX membrane tether, ER recruitment, portions thereof, derivatives thereof, or any combination thereof. In some embodiments: (a) the first, second, and/or third apoptosis polypeptide in the first localized state; (b) the first, second, and/or third apoptosis polypeptide in the second localized state(s); (c) the first and/or second pyroptosis in the first localized state; (d) the first and/or second pyroptosis polypeptide in the second localized state(s); (e) the first and/or second input polypeptide in the first localized state; and/or (f) the first and/or second input polypeptide in the second localized state(s); is capable of modulating an activation threshold and/or sensitivity of the synthetic protein circuit.

In some embodiments, the synthetic protein circuit is present in a cell. In some embodiments, the cell is: a cell of a subject, e.g., a subject suffering from a disease or disorder. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor; and/or an in vivo cell, an ex vivo cell, an in vitro cell or an in situ cell. In some embodiments, the cell is a eukaryotic cell, e.g., a mammalian cell. In some embodiments, the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

Disclosed herein include synthetic protein circuits comprising: one or more first, second, or third apoptosis polypeptides; one or more first or second pyroptosis polypeptides; one or more input polypeptides; one or more pyroptosis effector proteins; and/or one or more mutant pyroptosis effector proteins, wherein the synthetic protein circuit is capable of inducing in a cell: apoptosis via a first apoptotic protein complex in first apoptotic protein complex active state; and/or pyroptosis via a first and/or second pyroptosis polypeptide in a first and/or second pyroptosis polypeptide active state or a pyroptosis effector protein in a pyroptosis effector protein active state.

Disclosed herein include synthetic protein circuits comprising: (i) a first apoptosis polypeptide comprising a large subunit of an apoptotic effector protein and a small subunit of an apoptotic effector protein separated by a first heterologous protease cleavage site, wherein two of the first apoptosis polypeptides are capable of forming a first apoptotic protein complex in a first apoptotic protein complex inactive state, and wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic complex inactive state to a first apoptotic protein complex active state, wherein the first apoptotic protein complex in the first apoptotic protein complex active state is capable of inducing apoptosis in a cell; and (ii) a first pyroptosis polypeptide comprising a pyroptosis effector domain and a first inhibitory domain separated by the first heterologous protease cleavage site, wherein the first inhibitory domain is capable of inhibiting activity of the pyroptosis effector domain, thereby the first pyroptosis polypeptide is capable of being in a first pyroptosis polypeptide inactive state, wherein the first heterologous protease in the first heterologous protease active state is capable of cutting the first heterologous protease cleavage site of the first of the first pyroptosis polypeptide, wherein the first pyroptosis polypeptide being cut changes the first pyroptosis polypeptide from the first pyroptosis polypeptide inactive state to a first pyroptosis polypeptide active state, and wherein the first pyroptosis polypeptide in the first pyroptosis polypeptide active state is capable of (a) inducing pyroptosis in the cell; and (b) inhibiting the induction of apoptosis in the cell by the first apoptotic protein complex in the first apoptotic protein complex active state. In some embodiments, said inhibition is at least 10%. In some embodiments, the synthetic protein circuit further comprises a first input polypeptide comprising the first heterologous protease.

In some embodiments, the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof. In some embodiments, the pyroptosis effector domain comprises an N-terminal domain of a gasdermin (GSDM) protein. In some embodiments, the GSDM protein is GSDMA, GSDMB, GSDMC, GSDMD, GSDME, or DFNB59. In some embodiments, the first heterologous protease comprises or is derived from one or more of a prokaryotic protease, bacterial protease, archaeal protease, eukaryotic protease, fungal protease, plant protease, mammalian protease, and human protease. In some embodiments, the first heterologous protease is engineered. In some embodiments, the first heterologous protease comprises tobacco etch virus (TEV) protease, tobacco vein mottling virus (TVMV) protease, hepatitis C virus protease (HCVP), derivatives thereof, or any combination thereof. In some embodiments, the first heterologous protease cleavage site comprises the sequence of SEQ ID NO: 1 or SEQ ID NO: 2. In some embodiments, the first heterologous protease cleavage site is natural or engineered, e.g., the cleavage sequence of a human apoptotic effector protease, e.g., a caspase cleavage sequence. In some embodiments, the first heterologous protease cleavage site is configured to modulate an activation threshold of the synthetic protein circuit. In some embodiments, said configuring comprises introducing one or more amino acid insertions, substitutions, or deletions relative to a wild type first heterologous protease cleavage site. In some embodiments, the first heterologous protease cleavage site comprises the sequence of any one of SEQ ID NOs: 3-7.

In some embodiments, the first apoptosis polypeptide comprises one or more third partner domains and one or more fourth partner domains capable of binding the third partner domain, wherein binding of the third partner domain and the fourth partner domain is capable of inhibiting change of the first apoptotic protein complex from the first apoptotic protein complex inactive state to the first apoptotic protein complex active state in the absence of the first heterologous protease in the first heterologous protease active state. In some embodiments, said third partner domains and fourth partner domains are capable of multimerization. In some embodiments, the first pyroptosis polypeptide comprises one or more third partner domains and one or more fourth partner domains capable of binding the third partner domain, wherein binding of the third partner domain and the fourth partner domain is capable of inhibiting change of the first pyroptosis polypeptide from the first pyroptosis polypeptide inactive state to the first pyroptosis polypeptide active state in the absence of the first heterologous protease in the first heterologous protease active state. In some embodiments, said third partner domains and fourth partner domains are capable of multimerization. In some embodiments, the third partner domain and the fourth partner domain are homodimers. In some embodiments, the third partner domain and the fourth partner domain are heterodimers. In some embodiments, the binding between the third partner domain and the fourth partner domain is reversible.

In some embodiments, the third partner domain and/or the fourth partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the third partner domain and/or the fourth partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the third partner domain and the fourth partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the third partner domain and/or the fourth partner domain comprise CZp, NZp, or any combination thereof. In some embodiments, the third partner domain and/or the fourth partner domain comprise nHalo, cHalo, or any combination thereof.

In some embodiments, the first inhibitory domain comprises a protein domain configured to reduce or abrogate the activity of the pyroptosis effector domain, e.g., a bulky domain In some embodiments, the first inhibitory domain comprises an auto-inhibitory domain of a gasdermin family of pore-forming proteins or maltose-binding protein. In some embodiments, the cell does not express an endogenous protein comprising a pyroptosis effector domain.

Disclosed herein include synthetic protein circuits comprising: a first apoptosis polypeptide comprising a large subunit of an apoptotic effector protein and a small subunit of an apoptotic effector protein, separated by a first heterologous protease cleavage site, wherein two of the first apoptosis polypeptides are capable of forming a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptosis protease complex inactive state to a first apoptotic protein complex active state, wherein the first apoptotic protein complex in the first apoptotic protein complex active state is capable of: (a) inducing apoptosis in a cell expressing a pyroptosis effector protein; and/or (b) cutting the pyroptosis effector protein, wherein the pyroptosis effector protein being cut changes the pyroptosis effector protein from a pyroptosis effector protein inactive state to a pyroptosis effector protein active state, wherein the pyroptosis effector protein in the pyroptosis effector protein active state is capable of: (c) inducing pyroptosis in the cell; and (d) inhibiting the induction of apoptosis in the cell by the first apoptotic protein complex in the first apoptotic protein complex active state. In some embodiments, the synthetic protein circuit further comprises a first input polypeptide comprising the first heterologous protease.

Disclosed herein include synthetic protein circuits comprising: (i) a first apoptosis polypeptide comprising a large subunit of an apoptotic effector protein and a small subunit of an apoptotic effector protein, separated by a first heterologous protease cleavage site, wherein two of the first apoptosis polypeptides are capable of forming a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, wherein the first apoptotic protein complex in the first apoptotic protein complex active state is capable of: (a) inducing apoptosis in a cell expressing a pyroptosis effector protein; and (b) cutting the pyroptosis effector protein, wherein the pyroptosis effector protein being cut changes the pyroptosis effector protein from a pyroptosis effector protein inactive state to a pyroptosis effector protein active state, wherein the pyroptosis effector protein in the pyroptosis effector protein active state is capable of: (c) inducing pyroptosis in the cell; and (d) inhibiting the induction of apoptosis in the cell by the apoptotic protein complex in the first apoptotic protein complex active state; and (ii) a first pyroptosis polypeptide comprising a mutant pyroptosis effector domain comprising a mutation, capable of inhibiting the pyroptosis effector protein in the pyroptosis effector protein active state, thereby changing the pyroptosis effector protein from the pyroptosis effector protein active state to a pyroptosis effector protein inactive state; thereby the first apoptotic protein complex in the first apoptotic protein complex active state is capable of inducing apoptosis in the cell. In some embodiments, the synthetic protein circuit comprises a first input polypeptide comprising the first heterologous protease. In some embodiments, the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof.

In some embodiments, the first apoptosis polypeptide comprises one or more third partner domains and one or more fourth partner domains capable of binding the third partner domain, wherein binding of the third partner domain and the fourth partner domain is capable of inhibiting change of the first apoptotic protein complex from the first apoptotic protein complex inactive state to the first apoptotic protein complex active state, in the absence of the first heterologous protease in the first heterologous protease active state. In some embodiments, said third partner domains and fourth partner domains are capable of multimerization. In some embodiments, the third partner domain and the fourth partner domain are homodimers. In some embodiments, the third partner domain and the fourth partner domain are heterodimers. In some embodiments, the binding between the third partner domain and the fourth partner domain is reversible.

In some embodiments, the third partner domain and/or the fourth partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the third partner domain and/or the fourth partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the third partner domain and the fourth partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the third partner domain and/or the fourth partner domain comprise CZp, NZp, or any combination thereof. In some embodiments, the third partner domain and/or the fourth partner domain comprise nHalo, cHalo, or any combination thereof.

In some embodiments, the pyroptosis effector protein comprises a gasdermin (GSDM) protein. In some embodiments, the GSDM protein is GSDMA, GSDMB, GSDMC, GSDMD, GSDME, or DFNB59.

In some embodiments, the first heterologous protease comprises or is derived from one or more of a prokaryotic protease, bacterial protease, archaeal protease, eukaryotic protease, fungal protease, plant protease, mammalian protease, and human protease. In some embodiments, the first heterologous protease is engineered. In some embodiments, the first heterologous protease comprises tobacco etch virus (TEV) protease, tobacco vein mottling virus (TVMV) protease, hepatitis C virus protease (HCVP), derivatives thereof, or any combination thereof. In some embodiments, the first heterologous protease cleavage site comprises the sequence of SEQ ID NO: 1 or SEQ ID NO: 2. In some embodiments, the first heterologous protease cleavage site is natural or engineered, e.g., the cleavage sequence of a human apoptotic effector protease, e.g., a caspase cleavage sequence. In some embodiments, the first heterologous protease cleavage site is configured to modulate an activation threshold of the synthetic protein circuit. In some embodiments, said configuring comprises introducing one or more amino acid insertions, substitutions, or deletions relative to a wild type first heterologous protease cleavage site. In some embodiments, the first heterologous protease cleavage site comprises the sequence of any one of SEQ ID NOs: 3-7.

In some embodiments, the mutant pyroptosis effector domain is derived from an N-terminal domain of a gasdermin (GSDM) protein. In some embodiments, the GSDM protein is GSDMA, GSDMB, GSDMC, GSDMD, GSDME, or DFNB59. In some embodiments, the one or more mutations comprises a V99N, L101N, L103N, V193E, A195E, G199E, and/or I217N mutation in GSDME. In some embodiments, the mutation is an I217N mutation in GSDME.

In some embodiments, inducing pyroptosis in the cell causes the cell to release one or more inflammatory cytokines. In some embodiments, the one or more inflammatory cytokines comprise IL-18, IL-1β, IL-6, IL-8, interferon gamma (IFN-γ), and/or tumor necrosis factor-alpha (TNF-α). In some embodiments, inhibition of the pyroptosis effector protein by the first pyroptosis polypeptide is dose-dependent, thereby the induction of apoptosis in the cell is dose-dependent. In some embodiments, a concentration of the first pyroptosis peptide is at least two-folder higher than a concentration of the pyroptosis effector protein in the cell, thereby the inhibition of the pyroptosis effector protein by the first pyroptosis polypeptide is increased relative to a cell wherein the concentration of the first pyroptosis polypeptide is not at least two-fold higher than the concentration of the pyroptosis effector protein.

In some embodiments, the first apoptosis polypeptide, the first pyroptosis polypeptide, and/or the first input polypeptide comprise one or more linkers. In some embodiments, the linker: is a flexible linker, a rigid linker, or a hybrid linker; is hydrophilic or hydrophobic; is between 1 and 250 amino acids; comprises one or more flexible amino acid residues, e.g., about 1 to about 18 flexible amino acid residues. In some embodiments, the flexible amino acid residues comprise glycine, serine, or a combination thereof; and/or comprises 2 repeating amino acid subunits or more.

In some embodiments: (i) the first apoptosis polypeptide; (ii) the first pyroptosis polypeptide, and/or (iii) the first input polypeptide, are configured to be in a first localized state. In some embodiments, the first localized state comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first apoptosis polypeptide, the first pyroptosis polypeptide, and/or the first input polypeptide is: (i) tethered to an intracellular organelle and/or membrane; or (ii) fused to a polypeptide that recruits to said localized state. In some embodiments: (i) the first apoptosis polypeptide; (ii) the first pyroptosis polypeptide, and/or (iii) the first input polypeptide, are configured to be in second localized state(s), wherein the first localized state and the second localized state(s) are different. In some embodiments, the second localized state(s) comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first apoptosis polypeptide, the first pyroptosis polypeptide, and/or the first input polypeptide is: (i) tethered to an intracellular organelle and/or membrane; or (ii) fused to a polypeptide that recruits to said localized state. In some embodiments: (i) the first apoptosis polypeptide; (ii) the first pyroptosis polypeptide, and/or (iii) the first input polypeptide comprise a first localization signal. In some embodiments, the first localization signal is adjacent to a third degron and/or a third heterologous cleavage site. In some embodiments: (i) the first apoptosis polypeptide; (ii) the first pyroptosis polypeptide, and/or (iii) the first input polypeptide comprise second localization signal(s). In some embodiments, the second localization signal is adjacent to a third degron and/or a third heterologous protease cleavage site. In some embodiments, the presence of the third degron and/or wherein the third heterologous cleavage site being cut changes: (i) the first apoptosis polypeptide from a non-localized state or the first localized state to the second localized state(s) or a non-localized state; (ii) the first pyroptosis polypeptide from a non-localized state or the first localized state to the second localized state(s) or a non-localized state; and/or (iii) the first input polypeptide from a non-localized state or the first localized state to the second localized state(s) or a non-localized state. In some embodiments, the synthetic protein circuit further comprises a third input polypeptide comprising a third heterologous protease capable of cutting the third heterologous protease cleavage site. In some embodiments, the third heterologous protease comprises or is derived from one or more of a prokaryotic protease, bacterial protease, archaeal protease, eukaryotic protease, fungal protease, plant protease, mammalian protease, and human protease. In some embodiments, the third heterologous protease is engineered. In some embodiments, the third heterologous protease comprises tobacco etch virus (TEV) protease, tobacco vein mottling virus (TVMV) protease, hepatitis C virus protease (HCVP), derivatives thereof, or any combination thereof. In some embodiments, the third heterologous protease cleavage site comprises the sequence of SEQ ID NO: 1 or SEQ ID NO: 2. In some embodiments, the third heterologous protease cleavage site is natural or engineered, e.g., the cleavage sequence of a human apoptotic effector protease, e.g., a caspase cleavage sequence. In some embodiments, the third heterologous protease cleavage site is configured to modulate an activation threshold of the synthetic protein circuit. In some embodiments, said configuring comprises introducing one or more amino acid insertions, substitutions, or deletions relative to a wild type third heterologous protease cleavage site. In some embodiments, the third heterologous protease cleavage site comprises the sequence of any one of SEQ ID NOs: 3-7. In some embodiments: (a) the first apoptosis polypeptide in the first localized state; (b) the first apoptosis polypeptide in the second localized state(s); (c) the first pyroptosis in the first localized state; (d) the first pyroptosis polypeptide in the second localized state(s); (e) the first input polypeptide in the first localized state; and/or (f) the first second input polypeptide in the second localized state(s), is capable of modulating an activation threshold and/or sensitivity of the synthetic protein circuit. In some embodiments, the first localization signal and/or second localization signal(s) is selected from the group comprising a nuclear localization signal (NLS), a nuclear export signal (NES), a peroxisomal targeting signal (PTS), a mitochondrial targeting sequence (MTS), a ER signal peptide, CAAX box, a peroxisomal targeting signal (PTS), PTS1, PTS2, a dileucine motif, YxxΦ motif, a palmitoylation motif, a GPI anchor signal, myristoylation signal, HDEL, KDEL, KKXX motif, RXR motif, SV40 NLS, SV40 NES, CAAX membrane tether, ER recruitment, portions thereof, derivatives thereof, or any combination thereof.

In some embodiments, the synthetic protein circuit is present in a cell. In some embodiments, the cell is: a cell of a subject, e.g., a subject suffering from a disease or disorder. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor; and/or an in vivo cell, an ex vivo cell, an in vitro cell or an in situ cell. In some embodiments, the cell is a eukaryotic cell, e.g., a mammalian cell. In some embodiments, the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

Disclosed herein include synthetic protein circuits comprising: a first polypeptide comprising a first signal transducer binding domain and a first part of a first cell death executioner, wherein the first signal transducer binding domain is capable of binding a first signal transducer to form a first signal transducer-bound polypeptide; a second polypeptide comprising a second signal transducer binding domain and a second part of the first cell death executioner, wherein the second signal transducer binding domain is capable of binding a second signal transducer to form a second signal transducer-bound polypeptide, and wherein the first part of the first cell death executioner and the second part of the first cell death executioner are capable of associating with each other to constitute a first cell death executioner capable of being in a first cell death executioner active state when the first signal transducer and the second signal transducer are in close proximity at an association location; and wherein the first cell death executioner in the first cell death executioner active state is capable of inducing apoptosis or pyroptosis in a cell.

In some embodiments, the first signal transducer binding domain of the first polypeptide and the second signal transducer binding domain of the second polypeptide are identical. In some embodiments, the first transducer and the second transducer are identical and/or are the same protein. In some embodiments, the first cell death executioner comprises an apoptosis effector protein or a pyroptosis effector protein. In some embodiments, the first part of the first cell death executioner comprises a large subunit of the apoptotic effector protein and/or wherein the second part of second first cell death executioner comprises a small subunit of the apoptotic effector protein. In some embodiments, the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof. In some embodiments, the pyroptosis effector protein comprises a gasdermin (GSDM) protein. In some embodiments, the GSDM protein is GSDMA, GSDMB, GSDMC, GSDMD, GSDME, or DFNB59.

In some embodiments, the first signal transducer, the second signal transducer, or both, are capable of being localized at the association location. In some embodiments, the first signal transducer when in a first signal transducer active state, the second signal transducer when in a second signal transducer active state, or both, are capable of being localized at the association location. In some embodiments, the first signal transducer when in a first inactive state, the second signal transducer when in a second inactive state, or both, are capable of being localized at the association location. In some embodiments, the first signal transducer binding domain of the first polypeptide is capable of binding to the first signal transducer, wherein the second signal transducer binding domain of the second polypeptide is capable of binding to the second signal transducer, or both. In some embodiments, the first signal transducer binding domain of the first polypeptide is capable of binding to the first signal transducer in a first signal transducer active state, wherein the second signal transducer binding domain of the second polypeptide is capable of binding to the second signal transducer in a second signal transducer active state, or both. In some embodiments, the first signal transducer binding domain of the first polypeptide is capable of binding to the first signal transducer in a first inactive state, wherein the second signal transducer binding domain of the second polypeptide is capable of binding to the second signal transducer in a second inactive state, or both. In some embodiments, the signal transducer binding domain of the first polypeptide is capable of binding the first signal transducer to form the first signal transducer-bound polypeptide at the association location, wherein the signal transducer binding domain of the first polypeptide is capable of binding the first signal transducer to form the first signal transducer-bound polypeptide at the association location, or both. In some embodiments, the signal transducer binding domain of the first polypeptide is capable of binding the first signal transducer to form the first signal transducer-bound polypeptide at a first cellular location other than the association location, wherein the signal transducer binding domain of the first polypeptide is capable of binding the first signal transducer to form the first signal transducer-bound polypeptide at a second cellular location other than the association location, or both. In some embodiments, the first cellular location, the second cellular location, or both comprise one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the association location comprises one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof.

In some embodiments, a first concentration of the first signal transducer-bound polypeptide is at least two-fold higher at the association location as compared to a first cellular location other than the association location when the first signal transducer is in a first signal transducer active state, and/or wherein a second concentration of the second signal transducer-bound polypeptide is at least two-fold higher at the association location as compared to a second cellular location other than the association location when the second signal transducer is in a second signal transducer active state. In some embodiments, a first concentration of the first cell death executioner in the first cell death executioner active state is at least two-fold higher at the association location as compared to a cellular location other than the association location when the first signal transducer is in a first signal transducer active state and/or when the second signal transducer is in a second signal transducer active state. In some embodiments, the first part of the first cell death executioner and the second part of the first cell death executioner have the weak association affinity when the first signal transducer is in a first signal transducer inactive state and/or the second signal transducer is in a second signal transducer inactive state. In some embodiments, the first part of the first cell death executioner and the second part of the first cell death executioner are incapable of associating to form the first cell death executioner in the first cell death executioner active state when the first signal transducer is in a first signal transducer inactive state and/or the second signal transducer is in a second signal transducer inactive state. In some embodiments, a first concentration of the first signal transducer-bound polypeptide and a second concentration of the second signal transducer-bound polypeptide at the association location are insufficient for the first part of the first cell death executioner and the second part of the first cell death executioner to form an active first cell death executioner when the first signal transducer is in a first signal transducer inactive state and/or the second signal transducer is in a second signal transducer inactive state. In some embodiments, a first concentration of the first signal transducer-bound polypeptide at the association location is comparable to a first cellular location other than the association location when the first signal transducer is in a first signal transducer inactive state, and/or wherein a second concentration of the second signal transducer-bound polypeptide at the association location is comparable to a second cellular location other than the association location when the second signal transducer is in a second signal transducer inactive state. In some embodiments, the first part of the first cell death executioner and the second part of the first cell death executioner are capable of associating with each other to form the first cell death executioner in the first cell death executioner active state at a threshold first polypeptide concentration and a threshold second polypeptide concentration at the association location. In some embodiments, the threshold first polypeptide concentration and the threshold second polypeptide concentration at the association location are reached at a threshold signal transducer activation level of the signal transducer.

In some embodiments, the first signal transducer binding domain and/or the second signal transducer binding domain are identical. In some embodiments, the first signal transducer binding domain and/or the second signal transducer binding domain are different. In some embodiments, the first signal transducer binding domain and/or the second signal transducer binding domain each is capable of binding molecules of the first signal transducer and/or the second signal transducer. In some embodiments, the first signal transducer and/or the second signal transducer belong to a signal transduction pathway. In some embodiments, the first signal transducer binding domain and/or the second signal transducer binding domain comprise a RAS binding domain (RBD) and/or RAS association domain (RAD). In some embodiments, the RAS binding domain comprises or is derived from a RAS interacting protein, optionally selected from the group comprising AGO2, APBB1IP, APPL1, ARAF, ARL1, ARL2, ARRB1, ARRB2, BAIAP2, BCL2, BCL2L1, BRAF, BRAP, BSG, CALM1, CALM3, CALML3, CALML4, CALML5, CALML6, CNKSR1, CNKSR2, CSK, DAB2IP, EGFR, ERBIN, FGA, FGB, FGG, FN1, GRB2, HK1, IFNGR1, IL6, IQGAP1, ITGA2B, ITGB3, KSR1, KSR2, LGALS3, LYN, LZTR1, MAP2K1, MAP2K2, MAPK1, MAPK14, MAPK3, MAPKAP1, MARK2, MARK3, MBP, MSI2, MTOR, NCBP2AS2, NF1, NIBAN2, PDE4DIP, PDE6D, PDPK1, PEBP1, PIK3CA, PIK3CB, PIK3CD, PIK3R1, PIK3R2, PIP5K1A, PLCE1, PPIA, PRKCZ, PTGS2, RAF1, RALB, RALGDS, RAP1A, RAP1B, RAP1GDS1, RASA1, RASA2, RASA3, RASA4, RASAL1, RASAL2, RASAL3, RASSF1, RASSF2, RASSF5, RGL1, RGL3, RIN1, SHOC2, SOS1, SOS2, SPRED1, SPRED2, SPRED3, SRC, SYNGAP1, TIAM1, TLN1, VCL, VWF, YWHAB, or any combination thereof. In some embodiments, the first signal transducer binding domain and/or the second signal transducer binding domain comprises a lipid binding domain. In some embodiments, the lipid binding domain comprises a Pleckstrin homology (PH) domain. In some embodiments, the first signal transducer binding domain and/or the second signal transducer binding domain comprises an antibody, an antibody fragment, a binding domain derived from a natural protein, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the first signal transducer is capable of binding the first signal transducer binding domain and/or the second signal transducer is capable of binding the second signal transducer binding domain following a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof. In some embodiments, the first signal transducer, the second signal transducer, or both are endogenous proteins. In some embodiments, the first signal transducer, the second signal transducer, or both comprise AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCy, PLCy, NF-kB, FAK, CREB, αIIIβ3, FcεRI, BAD, p70S6K, STAT1, STAT2, STAT3, STAT5, STAT6, or any combination thereof. In some embodiments, the first signal transducer and/or the second signal transducer are capable of regulating cell survival, cell growth, cell proliferation, cell adhesion, cell migration, cell metabolism, cell morphology, cell differentiation, apoptosis, or any combination thereof. In some embodiments, the first signal transducer, the second signal transducer, or both comprise a RAS protein. In some embodiments, the RAS protein is KRAS, NRAS, HRAS, or any combination thereof. In some embodiments, the first signal transducer, the second signal transducer, or both are exogenous proteins. In some embodiments, the synthetic protein circuit comprises the first signal transducer, the second signal transducer, or both. In some embodiments, the first signal transducer, the second signal transducer, or both comprise a lipid. In some embodiments, the lipid comprises a phospholipid. In some embodiments, the phospholipid is phosphatidylinositol 3-phosphate.

In some embodiments, the synthetic protein circuit is capable of detecting an activity of the first signal transducer and an activity of the second signal transducer. In some embodiments, an activity of the first cell death executioner correlates with an activity of the first signal transducer and/or an activity of the second signal transducer. In some embodiments, the synthetic protein circuit is capable of detecting activities of the first signal transducer and activities of the second signal transducer over a period of time. In some embodiments, activities of the first cell death executioner correlate with activities of the first signal transducer and activities of the second signal transducer over a period of time. In some embodiments, the synthetic protein circuit is capable of detecting an aberrant signaling. In some embodiments, aberrant signaling involves an active signal transducer. In some embodiments, the aberrant signaling involves an overactive signal transducer. In some embodiments, the aberrant signaling involves a constitutively active signal transducer over a period of time. In some embodiments, the synthetic protein circuit is capable of detecting an activity of a signal transducer activator and/or an activity of a signal transducer repressor. In some embodiments, the aberrant signaling involves an active signal transducer repressor and an active signal transducer. In some embodiments, the aberrant signaling involves an inactive signal transducer activator and an active signal transducer. In some embodiments, the aberrant signaling involves an inactive signal transducer. In some embodiments, the aberrant signaling involves an underactive signal transducer. In some embodiments, the aberrant signaling involves a constitutively inactive signal transducer over a period of time. In some embodiments, the aberrant signaling involves an inactive signal transducer repressor and an inactive signal transducer. In some embodiments, the aberrant signaling involves an active signal transducer activator and an inactive signal transducer. In some embodiments, the aberrant signaling involves an active signal transducer, and wherein the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof. In some embodiments, the synthetic protein circuit is capable of directly or indirectly inducing cell death in the presence of the aberrant signaling. In some embodiments, the first cell death executioner is capable of directly or indirectly inducing cell death in the presence of aberrant signaling. In some embodiments, the synthetic protein circuit is capable of directly or indirectly inducing cell death when a first level of activation of the first signal transducer is above a first signal transducer activation threshold and/or a second level of activation of the second signal transducer is below a second signal transducer activation threshold. In some embodiments, the effector protein is capable of directly or indirectly inducing cell death when a first level of activation of the first signal transducer is above a first signal transducer activation threshold and/or a second level of activation of the second signal transducer is below a second signal transducer activation threshold.

In some embodiments, the synthetic protein circuit is present in a cell. In some embodiments, the cell is: a cell of a subject, e.g., a subject suffering from a disease or disorder. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor; and/or an in vivo cell, an ex vivo cell, an in vitro cell or an in situ cell. In some embodiments, the cell is a eukaryotic cell, e.g., a mammalian cell. In some embodiments, the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

Disclosed herein include nucleic acid compositions. In some embodiments, the composition comprises: one or more polynucleotides encoding any of the synthetic protein circuits of the disclosure. In some embodiments, the one or more polynucleotides comprise: one or more first polynucleotides encoding a first apoptosis polypeptide, a first pyroptosis polypeptide, or a first polypeptide; one or more second polynucleotides encoding a second apoptosis polypeptide, a second pyroptosis polypeptide, or a second polypeptide; and/or one or more third polynucleotides encoding a third apoptosis polypeptide. In some embodiments, the nucleic acid composition comprises one or more polynucleotides encoding a first and/or second input polypeptide.

In some embodiments, at least two of the one or more polynucleotides are operably linked to a tandem gene expression element. In some embodiments, the one or more polynucleotides comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the one or more polynucleotides are operably connected to a promoter selected from the group comprising: a minimal promoter, e.g., TATA, miniCMV, and/or miniPromo; a tissue-specific promoter and/or a lineage-specific promoter; and/or a ubiquitous promoter, e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, β-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

In some embodiments, the nucleic acid composition is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines. In some embodiments, the modified nucleoside is selected from pseudouridine (ψ), N 1-methyl-pseudouridine (m 1ψ), and 5-methyl-uridine (m5U).

In some embodiments, the nucleic acid composition is complexed or associated with one or more lipids or lipid-based carriers, thereby forming liposomes, lipid nanoparticles (LNPs), lipoplexes, and/or nanoliposomes, e.g., encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), a bacterial cell, a bacteriophage, or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the one or more polynucleotides are comprised in the one or more vectors. In some embodiments, the one or more polynucleotides are comprised in the same vector and/or different vectors. In some embodiments, the one or more polynucleotides are situated on the same nucleic acid and/or different nucleic acids. In some embodiments, the nucleic acid composition comprises circular mRNA, circular DNA, self-amplifying RNA, self-amplifying RNA, and/or mRNA.

In some embodiments, the nucleic acid composition is configured to achieve relative levels of the first apoptotic polypeptide, the second apoptotic polypeptide, the third apoptotic polypeptide, the first input polypeptide, the second input polypeptide, the first polypeptide and/or the second polypeptide desired by a user. In some embodiments, the nucleic acid composition is configured to achieve relative levels of the first pyroptotic polypeptide, the second pyroptotic polypeptide, the first input polypeptide, the second input polypeptide, the first polypeptide and/or the second polypeptide desired by a user. In some embodiments, the expression of one or more of the first apoptotic polypeptide, the second apoptotic polypeptide, the third apoptotic polypeptide, first pyroptotic polypeptide, the second pyroptotic polypeptide, the first input polypeptide, and/or the second polypeptide is configured to be dosage invariant and/or robust to tissue tropism and stochastic expression. In some embodiments, the induction of apoptosis or the induction of pyroptosis can be tuned by adjusting the relative levels of the first apoptotic polypeptide, the second apoptotic polypeptide, the third apoptotic polypeptide, first pyroptotic polypeptide, the second pyroptotic polypeptide, the first input polypeptide, and/or the second polypeptide.

Disclosed herein include compositions. In some embodiments, the composition comprises: a first population of sender cells comprising: (i) one or more first polynucleotide(s) encoding a first apoptosis polypeptide comprising a large subunit of an apoptotic effector protein and a small subunit of an apoptotic effector protein separated by a first heterologous protease cleavage site, wherein two of the first apoptosis polypeptides are capable of forming a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, and wherein the first apoptotic protein complex in the first apoptotic protein complex active state is capable of inducing apoptosis in a cell; and (ii) a first vector; and a second population of sender cells comprising: (i) one or more second polynucleotide(s) encoding a first input polypeptide; and (ii) a second vector.

In some embodiments, the first vector of the first population of sender cells and the second vector of the second population of sender cells are capable of delivering the one or more first polynucleotide(s) and the one or more second polynucleotide(s) to receiver cells. In some embodiments, the first apoptosis polypeptide and the first input polypeptide are expressed in the receiver cells, thereby the first apoptotic protein complex in the first apoptotic protein complex active state is capable of inducing apoptosis in the receiver cells. In some embodiments, the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof. In some embodiments, the first heterologous protease comprises or is derived from one or more of a prokaryotic protease, bacterial protease, archaeal protease, eukaryotic protease, fungal protease, plant protease, mammalian protease, and human protease. In some embodiments, the first heterologous protease is engineered. In some embodiments, the first heterologous protease comprises tobacco etch virus (TEV) protease, tobacco vein mottling virus (TVMV) protease, hepatitis C virus protease (HCVP), derivatives thereof, or any combination thereof. In some embodiments, the first heterologous protease cleavage site comprises the sequence of SEQ ID NO: 1 or SEQ ID NO: 2. In some embodiments, the first heterologous protease cleavage site is natural or engineered, e.g., the cleavage sequence of a human apoptotic effector protease, e.g., a caspase cleavage sequence. In some embodiments, the first heterologous protease cleavage site is configured to modulate an activation threshold of the synthetic protein circuit. In some embodiments, said configuring comprises introducing one or more amino acid insertions, substitutions, or deletions relative to a wild type first heterologous protease cleavage site. In some embodiments, the first heterologous protease cleavage site comprises the sequence of any one of SEQ ID NOs: 3-7. In some embodiments, the first apoptosis polypeptide, the first input polypeptide, or both comprise a membrane-localization domain. In some embodiments, the membrane-localization domain comprises a CAAX domain.

Disclosed herein include compositions. In some embodiments, the composition comprises: a first population of sender cells comprising: (i) one or more first polynucleotide(s) encoding a first pyroptosis polypeptide comprising a pyroptosis effector domain and a first inhibitory domain separated by a first heterologous protease cleavage site, wherein the first inhibitory domain is capable of inhibiting activity of the pyroptosis effector domain, thereby the first pyroptosis polypeptide is in a first pyroptosis polypeptide inactive state, wherein a first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in a cell, and (ii) a first vector; and a second population of sender cells comprising: (i) one or more second polynucleotide(s) encoding a first input polypeptide comprising the first heterologous protease; and (ii) a second vector.

In some embodiments, the first vector of the first population of sender cells and the second vector of the second population of sender cells are capable of delivering the one or more first polynucleotide(s) and the one or more second polynucleotide(s) to receiver cells. In some embodiments, the first pyroptosis polypeptide and the first input polypeptide are expressed in the receiver cells, thereby the first pyroptosis polypeptide in the first pyroptosis polypeptide active state is capable of inducing pyroptosis in the receiver cells. In some embodiments, the pyroptosis effector domain comprises an N-terminal domain of a gasdermin (GSDM) protein. In some embodiments, the GSDM protein is GSDMA, GSDMB, GSDMC, GSDMD, GSDME, or DFNB59. In some embodiments, the first heterologous protease comprises or is derived from one or more of a prokaryotic protease, bacterial protease, archaeal protease, eukaryotic protease, fungal protease, plant protease, mammalian protease, and human protease. In some embodiments, the first heterologous protease is engineered. In some embodiments, wherein the first heterologous protease comprises tobacco etch virus (TEV) protease, tobacco vein mottling virus (TVMV) protease, hepatitis C virus protease (HCVP), derivatives thereof, or any combination thereof. In some embodiments, the first heterologous protease cleavage site comprises the sequence of SEQ ID NO: 1 or SEQ ID NO: 2. In some embodiments, the first heterologous protease cleavage site is natural or engineered, wherein the first heterologous protease cleavage site is configured to modulate an activation threshold of the synthetic protein circuit. In some embodiments, said configuring comprises introducing one or more amino acid insertions, substitutions, or deletions relative to a wild type first heterologous protease cleavage site. In some embodiments, the first heterologous protease cleavage site comprises the sequence of any one of SEQ ID NOs: 3-7.

In some embodiments, the first inhibitory domain comprises a protein domain configured to reduce or abrogate the activity of the pyroptosis effector domain, e.g., a bulky domain. In some embodiments, the first inhibitory domain comprises an auto-inhibitory domain of a gasdermin family of pore-forming proteins or maltose-binding protein. In some embodiments, the cell does not express an endogenous protein comprising a pyroptosis effector domain.

In some embodiments, the first apoptosis polypeptide or the first pyroptosis polypeptide comprise one or more third partner domains and one or more fourth partner domains capable of binding the third partner domain, wherein binding of the third partner domain and the fourth partner domain of the first apoptosis polypeptide is capable of inhibiting change of the first apoptotic protein complex from the first apoptotic protein complex inactive state to the first apoptotic protein complex active state in the absence of the first heterologous protease in the first heterologous protease active state, or wherein binding of the third partner domain and the fourth partner domain of the first pyroptosis polypeptide is capable of inhibiting change of the first pyroptosis polypeptide from the first pyroptosis polypeptide inactive state to the first pyroptosis polypeptide active state in the absence of the first heterologous protease in the first heterologous protease active state. In some embodiments, said third partner domains and fourth partner domains are capable of multimerization. In some embodiments, the third partner domain and the fourth partner domain are homodimers. In some embodiments, the third partner domain and the fourth partner domain are heterodimers. In some embodiments, the binding between the third partner domain and the fourth partner domain is reversible.

In some embodiments, the third partner domain and/or the fourth partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the third partner domain and/or the fourth partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the third partner domain and the fourth partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the third partner domain and/or the fourth partner domain comprise CZp, NZp, or any combination thereof. In some embodiments, the third partner domain and/or the fourth partner domain comprise nHalo, cHalo, or any combination thereof.

Disclosed herein include compositions. In some embodiments, the composition comprises: a first population of sender cells comprising: (i) one or more first polynucleotide(s) encoding a first pyroptosis polypeptide comprising a pyroptosis effector domain and and a first partner domain; and (ii) a first vector, wherein the first population of sender cells express a silencer polypeptide comprising a first inhibitory domain and a second partner domain capable of binding the first partner domain, and wherein the inhibitor domain of the silencer polypeptide is capable of inhibiting the first pyroptosis polypeptide when the first pyroptosis polypeptide associates with the silencer polypeptide via binding of the first partner domain and the second partner domain, thereby the first pyroptosis polypeptide is in a first pyroptosis polypeptide inactive state.

In some embodiments, the first vector is capable of delivering the one or more first polynucleotide(s) to receiver cells. In some embodiments, the first pyroptosis polypeptide is expressed in the receiver cells, wherein the first pyroptosis polypeptide is capable of being in a first pyroptosis polypeptide active state, wherein the first pyroptosis polypeptide in the first pyroptosis polypeptide active state is capable of inducing pyroptosis in the receiver cells. In some embodiments, the receiver cells do not express the silencer polypeptide. In some embodiments, the first inhibitory domain comprises a protein domain configured to reduce or abrogate the activity of the pyroptosis effector domain, e.g., a bulky domain. In some embodiments, the first inhibitory domain comprises an auto-inhibitory domain of a gasdermin family of pore-forming proteins or maltose-binding protein. In some embodiments, the cell does not express an endogenous protein comprising a pyroptosis effector domain. In some embodiments, the pyroptosis effector domain comprises an N-terminal domain of a gasdermin (GSDM) protein. In some embodiments, the GSDM protein is GSDMA, GSDMB, GSDMC, GSDMD, GSDME, or DFNB59.

In some embodiments, the first partner domain and the second partner domain are homodimers. In some embodiments, the first partner domain and the second partner domain are heterodimers. In some embodiments, the binding between the first partner domain and the second partner domain is reversible.

In some embodiments, the first partner domain and/or the second partner domain comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37 XBXB heterodimer b, DHD37 AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, P1 peptide, P2 peptide, P3 peptide, P4 peptide, P5 peptide, P6 peptide, P7 peptide, P8 peptide, P9 peptide, P10 peptide, P11 peptide, P12 peptide, N5 heterodimer, P5A heterodimer, N6 heterodimer, P6A heterodimer, N7 heterodimer, P7A heterodimer, N7 heterodimer, P7A heterodimer, N8 heterodimer, P8A heterodimer, an S1h peptide, an S2h peptide, an S3h peptide, an S4h peptide, a P5f peptide, a P6f peptide, a P13f peptide, a P14f peptide, portions thereof, derivatives thereof, or any combination thereof.

In some embodiments, the first partner domain and/or the second partner domain comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the first partner domain and the second partner domain are a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. In some embodiments, the first partner domain and/or the second partner domain comprise CZp, NZp, or any combination thereof. In some embodiments, inducing pyroptosis in a receiver cell causes the receiver cell to release one or more inflammatory cytokines. In some embodiments, the one or more inflammatory cytokines comprise IL-18, IL-1β, IL-6, IL-8, interferon gamma (IFN-γ), and/or tumor necrosis factor-alpha (TNF-α).

In some embodiments, the first apoptosis polypeptide, the first pyroptosis polypeptide, the first input polypeptide, and/or the silencer polypeptide comprises one or more linkers. In some embodiments, the linker: is a flexible linker, a rigid linker, or a hybrid linker; is hydrophilic or hydrophobic; is between 1 and 250 amino acids; comprises one or more flexible amino acid residues, e.g., about 1 to about 18 flexible amino acid residues. In some embodiments, the flexible amino acid residues comprise glycine, serine, or a combination thereof; and/or comprises 2 repeating amino acid subunits or more. In some embodiments: (i) the first apoptosis polypeptide; (ii) the first pyroptosis polypeptide, and/or (iii) the first input polypeptide, are configured to be in a first localized state. In some embodiments, the first localized state comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, first apoptosis polypeptide, the first pyroptosis polypeptide, and/or the first input polypeptide is: (i) tethered to an intracellular organelle and/or membrane; or (ii) fused to a polypeptide that recruits to said localized state. In some embodiments: (i) the first apoptosis polypeptide; (ii) the first pyroptosis polypeptide, and/or (iii) the first input polypeptide, are configured to be in second localized state(s), wherein the first localized state and the second localized state(s) are different. In some embodiments, the second localized state(s) comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first apoptosis polypeptide, the first pyroptosis polypeptide, and/or the first input polypeptide is: (i) tethered to an intracellular organelle and/or membrane; or (ii) fused to a polypeptide that recruits to said localized state. In some embodiments: (i) the first apoptosis polypeptide; (ii) the first pyroptosis polypeptide, and/or (iii) the first input polypeptide comprise a first localization signal. In some embodiments, the first localization signal is adjacent to a third degron and/or a third heterologous cleavage site. In some embodiments: (i) the first apoptosis polypeptide; (ii) the first pyroptosis polypeptide, and/or (iii) the first input polypeptide comprise second localization signal(s). In some embodiments, the second localization signal is adjacent to a third degron and/or a third heterologous protease cleavage site. In some embodiments, the presence of the third degron and/or wherein the third heterologous cleavage site being cut changes: (i) the first apoptosis polypeptide from a non-localized state or the first localized state to the second localized state(s) or a non-localized state; (ii) the first pyroptosis polypeptide from a non-localized state or the first localized state to the second localized state(s) or a non-localized state; and/or (iii) the first input polypeptide from a non-localized state or the first localized state to the second localized state(s) or a non-localized state. In some embodiments, the synthetic protein circuit further comprises a third input polypeptide comprising a third heterologous protease capable of cutting the third heterologous protease cleavage site. In some embodiments, the third heterologous protease comprises or is derived from one or more of a prokaryotic protease, bacterial protease, archaeal protease, eukaryotic protease, fungal protease, plant protease, mammalian protease, and human protease. In some embodiments, the third heterologous protease is engineered. In some embodiments, the third heterologous protease comprises tobacco etch virus (TEV) protease, tobacco vein mottling virus (TVMV) protease, hepatitis C virus protease (HCVP), derivatives thereof, or any combination thereof. In some embodiments, the third heterologous protease cleavage site comprises the sequence of SEQ ID NO: 1 or SEQ ID NO: 2. In some embodiments, the third heterologous protease cleavage site is natural or engineered, e.g., the cleavage sequence of a human apoptotic effector protease, e.g., a caspase cleavage sequence. In some embodiments, the third heterologous protease cleavage site is configured to modulate an activation threshold of the synthetic protein circuit. In some embodiments, said configuring comprises introducing one or more amino acid insertions, substitutions, or deletions relative to a wild type third heterologous protease cleavage site. In some embodiments, the third heterologous protease cleavage site comprises the sequence of any one of SEQ ID NOs: 3-7. In some embodiments: (a) the first apoptosis polypeptide in the first localized state; (b) the first apoptosis polypeptide in the second localized state(s); (c) the first pyroptosis in the first localized state; (d) the first pyroptosis polypeptide in the second localized state(s); (e) the first input polypeptide in the first localized state; and/or (f) the first second input polypeptide in the second localized state(s), is capable of modulating an activation threshold and/or sensitivity of the synthetic protein circuit. In some embodiments, the first localization signal and/or second localization signal(s) is selected from the group comprising a nuclear localization signal (NLS), a nuclear export signal (NES), a peroxisomal targeting signal (PTS), a mitochondrial targeting sequence (MTS), a ER signal peptide, CAAX box, a peroxisomal targeting signal (PTS), PTS1, PTS2, a dileucine motif, YxxΦ motif, a palmitoylation motif, a GPI anchor signal, myristoylation signal, HDEL, KDEL, KKXX motif, RXR motif, SV40 NLS, SV40 NES, CAAX membrane tether, ER recruitment, portions thereof, derivatives thereof, or any combination thereof.

In some embodiments, the first and/or second vector is a viral vector, a plasmid, a naked DNA vector, a naked RNA vector, a lipid nanoparticle, or any combination thereof. In some embodiments, the viral vector is an AAV vector, a lentivirus vector, a retrovirus vector, an integration-deficient lentivirus (IDLV) vector. In some embodiments, the AAV vector comprises single-stranded AAV (ssAAV) vector or a self-complementary AAV (scAAV) vector. In some embodiments, the receiver cell or the sender cell is: a cell of a subject, e.g., a subject suffering from a disease or disorder. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor; and/or an in vivo cell, an ex vivo cell, or an in situ cell.

In some embodiments, the first and/or the second vector is capable of delivering the one or more first polynucleotides and/or the one or more second polynucleotides to one or more tissues of a subject. In some embodiments, the receiver cells are situated within one or more tissues of a subject; and/or the one or more tissues comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof.

In some embodiments, the sender cell and/or the receiver cell is a eukaryotic cell, e.g., a mammalian cell. In some embodiments, the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the receiver cell comprises a unique cell type and/or a unique cell state. In some embodiments, the unique cell type and/or the unique cell state comprises a unique gene expression pattern. In some embodiments, the unique cell type and/or unique cell state comprises (i) one or more mutations of a protein, (ii) structural variants and/or copy-number alternations of one or more protein-coding genes, (iii) epigenetic signature(s), and/or (iv) a unique anatomic location. In some embodiments, the unique cell type and/or the unique cell state comprises anatomically locally unique gene expression; wherein the unique cell type and/or the unique cell state is caused by hereditable, environmental, and/or idiopathic factors; wherein the unique cell type and/or the cell in the unique cell state (i) causes and/or aggravates a disease or disorder and/or (ii) is associated with the pathology of a disease or disorder; and/or wherein the unique cell state comprises a senescent cell state induced by a tumor microenvironment. In some embodiments, the senescent cell state induced by a tumor microenvironment comprises expression of CD57, KRLG1, TIGIT, p21, p53, phospho-p53, DEC1, PPP1A, γH2AX, 53BPI, Rad17, ATR, ATM, MDC1, TIF, IL-6, IL-8, CXCR2, IGF2, IGFBP3, IGFBP5, IGFBP7, STC1, GDF15, SERPIN, ICAM-1, DEP1, B2MG, NOTCH3, DcR2, or any combination thereof. In some embodiments, the unique cell state and/or unique cell type is characterized by one or more of: aberrant signaling of one or more signal transducer(s); cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment; acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression; and nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion. In some embodiments, the unique cell state comprises: a physiological state, e.g., a cell cycle state, a differentiation state, a development state a metabolic state, or a combination thereof; and/or a pathological state, e.g., a disease state, a human disease state, a diabetic state, an immune disorder state, a neurodegenerative disorder state, an oncogenic state, or a combination thereof. In some embodiments, the unique cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof. In some embodiments, the receiver cell is characterized by aberrant signaling of one or more signal transducers, and wherein the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. In some embodiments, the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof. In some embodiments, the signal transduscer(s) is AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCy, PLCy, NF-kB, FAK, CREB, αIIIβ3, FcεRI, BAD, p70S6K, STAT1, STAT2, STAT3, STAT5, STAT6, or any combination thereof.

Provided herein are methods of selectively killing a target cell. In some embodiments, the method comprises: expressing any of the synthetic protein circuits or any of the nucleic acid compositions of the disclosure in the target cell, wherein the synthetic protein circuit is configured to be responsive to a unique cell type and/or unique cell state of the target cell. In some embodiments, the first and/or second heterologous protease is configured to be in the first and/or second heterologous protease active state in response to the unique cell type and/or unique cell state of the target cell.

In some embodiments, the unique cell type and/or the unique cell state comprises a unique gene expression pattern. In some embodiments, the unique cell type and/or unique cell state comprises: (i) one or more mutations of a protein, (ii) structural variants and/or copy-number alternations of one or more protein-coding genes, (iii) epigenetic signature(s), and/or (iv) a unique anatomic location. In some embodiments, the unique cell type and/or the unique cell state comprises anatomically locally unique gene expression. In some embodiments, the unique cell type and/or the unique cell state is caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. In some embodiments, the unique cell state comprises a senescent cell state induced by a tumor microenvironment. In some embodiments, the senescent cell state induced by a tumor microenvironment comprises expression of CD57, KRLG1, TIGIT, p21, p53, phospho-p53, DEC1, PPP1A, γH2AX, 53BPI, Rad17, ATR, ATM, MDC1, TIF, IL-6, IL-8, CXCR2, IGF2, IGFBP3, IGFBP5, IGFBP7, STC1, GDF15, SERPIN, ICAM-1, DEP1, B2MG, NOTCH3, DcR2, or any combination thereof. In some embodiments, the unique cell state and/or unique cell type is characterized by aberrant signaling of one or more signal transducer(s). In some embodiments, the unique cell state comprises: a physiological state, e.g., a cell cycle state, a differentiation state, a development state a metabolic state, or a combination thereof; and/or a pathological state, e.g., a disease state, a human disease state, a diabetic state, an immune disorder state, a neurodegenerative disorder state, an oncogenic state, or a combination thereof. In some embodiments, the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment. In some embodiments, the unique cell state and/or unique cell type is characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression. In some embodiments, the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion. In some embodiments, the unique cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof. In some embodiments, the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. In some embodiments, the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof. In some embodiments, the signal transducer(s) is AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STAT3, STAT5, STAT6.

In some embodiments, configuring the first and/or the second heterologous protease to be in the first and/or the second heterologous protease active state in response to the unique cell type and/or the unique cell state of the target cell comprises: expressing a second synthetic protein circuit in the target cell, wherein the second synthetic protein circuit comprises: a first polypeptide comprising a first signal transducer binding domain and a first part of a first protease domain of the first or second heterologous protease, wherein the first signal transducer binding domain is capable of binding a first signal transducer to form a first signal transducer-bound polypeptide; a second polypeptide comprising a second signal transducer binding domain and a second part of the first protease domain of the first or second heterologous protease, wherein the second signal transducer binding domain is capable of binding a second signal transducer to form a second signal transducer-bound polypeptide, wherein the first part of the first protease domain and the second part of the first protease domain have weak association affinity, and wherein the first part of the first protease domain and the second part of the first protease domain are capable of associating with each other to constitute the first or second heterologous protease capable in a first or second heterologous protease active state capable of cutting: (i) the first, second, or third apoptosis polypeptide at the first or second heterologous protease cleavage site when the first signal transducer and the second signal transducer are in close proximity at an association location; or (ii) the first or second pyroptosis polypeptide at the first or second heterologous protease cleavage site when the first signal transducer and the second signal transducer are in close proximity at an association location.

In some embodiments, the first signal transducer binding domain of the first polypeptide and the second signal transducer binding domain of the second polypeptide are identical. In some embodiments, the first signal transducer and the second signal transducer are identical and/or are the same protein. In some embodiments, the first signal transducer, the second signal transducer, or both, are capable of being localized at the association location. In some embodiments, the first signal transducer when in a first signal transducer active state, the second signal transducer when in a second signal transducer active state, or both, are capable of being localized at the association location. In some embodiments, the first signal transducer when in a first inactive state, the second signal transducer when in a second inactive state, or both, are capable of being localized at the association location. In some embodiments, the first signal transducer binding domain of the first polypeptide is capable of binding to the first signal transducer, wherein the second signal transducer binding domain of the second polypeptide is capable of binding to the second signal transducer, or both. In some embodiments, the first signal transducer binding domain of the first polypeptide is capable of binding to the first signal transducer in a first signal transducer active state, wherein the second signal transducer binding domain of the second polypeptide is capable of binding to the second signal transducer in a second signal transducer active state, or both. In some embodiments, the first signal transducer binding domain of the first polypeptide is capable of binding to the first signal transducer in a first inactive state, wherein the second signal transducer binding domain of the second polypeptide is capable of binding to the second signal transducer in a second inactive state, or both. In some embodiments, the first signal transducer binding domain of the first polypeptide is capable of binding the first signal transducer to form the first signal transducer-bound polypeptide at the association location, wherein the signal transducer binding domain of the first polypeptide is capable of binding the first signal transducer to form the first signal transducer-bound polypeptide at the association location, or both. In some embodiments, the signal transducer binding domain of the first polypeptide is capable of binding the first signal transducer to form the first signal transducer-bound polypeptide at a first cellular location other than the association location, wherein the signal transducer binding domain of the first polypeptide is capable of binding the first signal transducer to form the first signal transducer-bound polypeptide at a second cellular location other than the association location, or both.

In some embodiments, the first cellular location, the second cellular location, or both comprise one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the association location comprises one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof.

In some embodiments, the first signal transducer binding domain and the second signal transducer binding domain are different. In some embodiments, the first signal transducer binding domain and/or the second signal transducer binding domain each is capable of binding molecules of the first signal transducer and/or the second signal transducer. In some embodiments, the first signal transducer and/or the second signal transducer belong to a signal transduction pathway. In some embodiments, the first signal transducer binding domain and/or the second signal transducer binding domain comprise a RAS binding domain (RBD) and/or RAS association domain (RAD). In some embodiments, the RAS binding domain comprises or is derived from a RAS interacting protein, optionally selected from the group comprising AGO2, APBB1IP, APPL1, ARAF, ARL1, ARL2, ARRB1, ARRB2, BAIAP2, BCL2, BCL2L1, BRAF, BRAP, BSG, CALM1, CALM3, CALML3, CALML4, CALML5, CALML6, CNKSR1, CNKSR2, CSK, DAB2IP, EGFR, ERBIN, FGA, FGB, FGG, FN1, GRB2, HK1, IFNGR1, IL6, IQGAP1, ITGA2B, ITGB3, KSR1, KSR2, LGALS3, LYN, LZTR1, MAP2K1, MAP2K2, MAPK1, MAPK14, MAPK3, MAPKAP1, MARK2, MARK3, MBP, MSI2, MTOR, NCBP2AS2, NF1, NIBAN2, PDE4DIP, PDE6D, PDPK1, PEBP1, PIK3CA, PIK3CB, PIK3CD, PIK3R1, PIK3R2, PIP5K1A, PLCE1, PPIA, PRKCZ, PTGS2, RAF1, RALB, RALGDS, RAP1A, RAP1B, RAP1GDS1, RASA1, RASA2, RASA3, RASA4, RASAL1, RASAL2, RASAL3, RASSF1, RASSF2, RASSF5, RGL1, RGL3, RIN1, SHOC2, SOS1, SOS2, SPRED1, SPRED2, SPRED3, SRC, SYNGAP1, TIAM1, TLN1, VCL, VWF, YWHAB, or any combination thereof. In some embodiments, the first signal transducer binding domain and/or the second signal transducer binding domain comprises a lipid binding domain. In some embodiments, the lipid binding domain comprises a Pleckstrin homology (PH) domain. In some embodiments, the first signal transducer binding domain and/or the second signal transducer binding domain comprises an antibody, an antibody fragment, a binding domain derived from a natural protein, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the first signal transducer is capable of binding the first signal transducer binding domain and/or the second signal transducer is capable of binding the second signal transducer binding domain following a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof.

In some embodiments, the first signal transducer, the second signal transducer, or both are endogenous proteins. In some embodiments, the first signal transducer, the second signal transducer, or both comprise AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STAT3, STAT5, STAT6, or any combination thereof. In some embodiments, the first signal transducer and/or the second signal transducer are capable of regulating cell survival, cell growth, cell proliferation, cell adhesion, cell migration, cell metabolism, cell morphology, cell differentiation, apoptosis, or any combination thereof. In some embodiments, the first signal transducer, the second signal transducer, or both comprise a RAS protein, a CTNNB1 protein, or a TP53 protein. In some embodiments, the RAS protein is KRAS, NHAS, HRAS, or any combination thereof. In some embodiments, the RAS protein comprises a G12 mutation, G13 mutation, a Q61 mutation, and/or an A146 mutation. In some embodiments, the G12 mutation is selected from the group comprising G12A, G12C, G12D, G12R, G12S, G12V, and any combination thereof. In some embodiments, the G13 mutation is selected from the group comprising G13C, G13D, G13dup, G13R, G13S, G13V, and any combination thereof. In some embodiments, the Q61 mutation is selected from the group comprising Q61H, Q61K, Q61L, Q61R, Q61E, Q61P, Q61*, and any combination thereof. In some embodiments, the A146 mutation is selected from the group comprising A146P, A146T, A146V, and any combination thereof. In some embodiments, the signature detected by the input polypeptide(s) is correlated with any other cellular signature, protein state, cell type, and/or cell state capable of being read out as a biomarker. In some embodiments, the signal transducer is CTNNB1, and wherein the cell state is defined by CTNNB1 mutation(s) and/or localization and/or concentration and/or protein turnover and/or multimerization and/or PTM(s). In some embodiments, the signal transducer is TP53, and wherein the cell state is defined by TP53 mutation(s) and/or elevated TP53 concentration and/or altered TP53 oligomerization/multimerization state and/or TP53 localization pattern and/or PTM(s) and/or turnover. In some embodiments, the signal transducer(s) are associated with disease, e.g., cancer. In some embodiments, the first signal transducer, the second signal transducer, or both are exogenous proteins. In some embodiments, the second synthetic protein circuit comprises the first signal transducer, the second signal transducer, or both. In some embodiments, the first signal transducer, the second signal transducer, or both comprise a lipid. In some embodiments, the lipid comprises a phospholipid. In some embodiments, the phospholipid is phosphatidylinositol 3-phosphate.

Disclosed herein include methods of treating or preventing a disease or disorder in a subject in need thereof. In some embodiments, the method comprises: expressing any of the synthetic protein circuit, the synthetic protein circuit and/or the second synthetic protein circuit of the disclosure, in a cell of the subject.

In some embodiments, the method comprises: administering to the subject an effective amount of a nucleic acid composition or a composition disclosed herein, thereby treating or preventing the disease or disorder in the subject. In some embodiments, administering comprises: (i) isolating one or more cells from the subject; (ii) contacting said one or more cells with any of the nucleic acid compositions disclosed herein, thereby generating engineered cells, optionally the contacting comprises transfection; and (iii) administering the one or more engineered cells into a subject after the contacting step.

In some embodiments, the disease or disorder is a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof, e.g., a solid tumor.

In some embodiments, the disease or disorder is an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru(EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

In some embodiments, the disease is associated with expression of a tumor-associated antigen. In some embodiments, the disease associated with expression of a tumor antigen-associated is selected from the group consisting of a proliferative disease, a precancerous condition, a cancer, and a non-cancer related indication associated with expression of the tumor antigen.

In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is non-resectable.

In some embodiments, the cancer comprises one or more mutations in KRAS, NRAS, HRAS, or any combination thereof, e.g., a G12 mutation, G13 mutation, a Q61 mutation, and/or an A146 mutation. In some embodiments, the G12 mutation is selected from the group comprising G12A, G12C, G12D, G12R, G12S, G12V, and any combination thereof. In some embodiments, the G13 mutation is selected from the group comprising G13C, G13D, G13dup, G13R, G13S, G13V, and any combination thereof. In some embodiments, the Q61 mutation is selected from the group comprising Q61H, Q61K, Q61L, Q61R, Q61E, Q61P, Q61*, and any combination thereof. In some embodiments, the A146 mutation is selected from the group comprising A146P, A146T, A146V, and any combination thereof

In some embodiments, the subject was previously treated with approved medication(s) and/or surgery. In some embodiments, the cancer comprises metastasis to liver and/or lung, e.g., (i) metastasis of colorectal cancer to liver and/or lung or (ii) metastasis of pancreatic cancer to liver and/or lung. In some embodiments, said metastasis is non-resectable. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, administering comprises one or more administrations, e.g., via aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, the administering comprises continuous administration. In some embodiments, the administering comprises a first administration and a second administration. In some embodiments, separated by a period of time of at least one hour, at least one day, at least one week, or at least one month. In some embodiments, administering comprises one or more repeated administrations. In some embodiments, repeated hourly, every 2 hours, every 4 hours, every 6 hours, every 8 hours, every 10 hours, every 12 hours, every 16 hours, every 20 hours, every 24 hours, every 2 days, every 3 days, every 4 days, every 5 days, every 6 days, every 7 days, every 10 days, or every 14 days.

Any of the synthetic protein circuits, nucleic acid compositions, or methods can comprise a supplementary protein circuit. In some embodiments, the supplementary protein circuit comprises: a first polypeptide comprising an optional first supplementary domain and a first part of a first protease domain of a supplementary heterologous protease; a second polypeptide comprising a second supplementary domain and a second part of the first protease domain of the supplementary protease, wherein the first part of the first protease domain and the second part of the first protease domain have weak association affinity, and wherein the first part of the first protease domain and the second part of the first protease domain are capable of associating with each other to constitute the supplementary heterologous protease, optionally the first and/or second supplementary domain is a signal transducer binding domain, optionally the supplementary heterologous protease in a supplementary heterologous protease active state is capable of cutting (i) the first, second, or third apoptosis polypeptide at the first or second heterologous protease cleavage site, and/or (ii) the first or second pyroptosis polypeptide at the first or second heterologous protease cleavage site, further optionally when a first signal transducer and a second signal transducer are in close proximity at an association location the supplementary heterologous protease is the first or second heterologous protease.

In some embodiments, the induction of apoptosis and/or pyroptosis is dependent on the dose of one or more synthetic protein circuit components. In some embodiments, cell death is triggered when a threshold amount of one or more of the following is reached: (i) the first, second, and/or third apoptotic protein complex in the first, second, and/or third apoptotic protein complex active state; (ii) the first and/or second pyroptosis polypeptide in a first and/or second pyroptosis polypeptide active state; and/or (iii) a pyroptosis effector protein in a pyroptosis effector protein active state.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1E display non-limiting exemplary data showing synpoptosis circuits control user-selectable cell death programs. The schematic in FIG. 1A shows synthetic cell death “synpoptosis” circuits steer the mode of cell death by operating orthogonally to cell-intrinsic death programs. Shown in FIG. 1B are schematics of exemplary molecular building blocks of synpoptosis circuits of the disclosure, include caspase-3 subunits, gasdermin domains, viral proteases such as TEVP, degrons, maltose-binding protein, and leucine zippers. As shown in FIG. 1C, synthetic apoptosis modules use viral proteases, such as TEVP, to activate or repress engineered variants of caspase-3. HEK cells were transiently transfected with plasmid DNA encoding the synthetic modules and then cell death was quantified by staining and flow cytometry. Throughout the figure, the gray window indicates the fractions established by negative and positive transient DNA transfection controls; dots represent biological replicates (distinct culture wells). As shown in FIG. 1D, synthetic pyroptosis modules similarly use TEVP to regulate engineered GSDMA. FIG. 1E displays exemplary data showing apoptosis and pyroptosis exhibit different staining patterns with Annexin and Sytox. Annexin stains both apoptotic and pyroptotic cells, while Sytox primarily stains pyroptotic cells. We calculated the fraction of cells that stained positive for each dye after gating on the transfected cells based on fluorescence of a co-transfected marker. Data represent three independent experiments.

FIG. 2A-FIG. 2H display non-limiting exemplary data showing synpoptosis circuits lead to canonical features of cell death. FIG. 2A displays data showing transient transfection of HEK cells with plasmid DNA encoding the TEVP-activated caspase-3 circuit triggered asynchronous apoptosis, shown by flow cytometry. Between 16 and 24 hours after transfection (gray window), Sytox remains low in apoptotic cells and therefore can reliably distinguish between apoptotic and pyroptotic cells. Throughout the disclosure, dots represent biological replicates (distinct culture wells). Similarly, the TEVP-activated GSDMA circuits triggered asynchronous pyroptosis (FIG. 2B). FIG. 2C displays graphs showing Q-VD-OPh suppressed circuit-induced apoptosis but not pyroptosis, shown by flow cytometry. The gray window indicates the fractions established by negative and positive transient DNA transfection controls. As shown in FIG. 2D, TO-PRO-3 stains cells killed by the apoptosis and pyroptosis circuits, shown by flow cytometry. FIG. 2E displays exemplary data showing the apoptosis and pyroptosis circuits differently triggered the release of IL-1β and IL-18 from engineered HEK cells that stably express these cytokines. As shown in FIG. 2F, transient transfection using in vitro transcribed mRNA transcripts of the synpoptosis circuits led to loss of ATP-based cell viability. FIG. 2G displays exemplary data showing the mRNA version of the pyroptosis circuit triggered more LDH release than the apoptosis circuit. The mRNA version of the pyroptosis circuit triggered more ATP release than the apoptosis circuit. The same wells were repeatedly captured across a time course (FIG. 2H).

FIG. 3A-FIG. 3F display non-limiting exemplary data showing synpoptosis circuits direct the mode of cell death. FIG. 3A displays exemplary data showing transient transfection of plasmid DNA encoding TEVP and engineered TEVP-activatable caspase-3 induced apoptosis in HEK cells, shown by flow cytometry. TEVP-activatable GSDMA overrode the apoptotic program, leading to pyroptosis. Throughout the figure, horizontal lines indicate the fractions established by negative and positive transient DNA transfection controls, established separately for Annexin and Sytox; dots represent biological replicates (distinct culture wells). As shown in FIG. 3B, cells transfected with plasmid DNA encoding wildtype GSDME, a natural substrate of caspase-3, underwent pyroptosis in response to TEVP-mediated caspase-3 activation. GSDME N(mut) overcame the tendency of wildtype GSDME-expressing cells to undergo pyroptosis downstream of caspase-3 activation and promoted apoptosis (FIG. 3C). FIG. 3D displays exemplary data showing the ratio between the two forms of cell death is tunable by adjusting the plasmid DNA amount of GSDME N(mut) relative to wildtype GSDME (low, 1:1; medium, 2:1; high, 4:1). The heights of the stacked bars indicate means of three biological replicates (distinct culture wells) measured by flow cytometry. FIG. 3E displays exemplary data showing in vitro transcribed mRNA versions of synpoptosis circuits drove apoptosis and pyroptosis in Jurkat and THP-1 cells, which have more sophisticated endogenous cell death circuitry than HEK cells. As shown in FIG. 3F, in response to natural death inducers, cells execute their default death programs. GSDM-negative and GSDM-positive cells tend to die by apoptosis and pyroptosis, respectively. Described herein are synpoptosis circuits that dictate death mode by overriding these cell-intrinsic preferences.

FIG. 4A-FIG. 4F display non-limiting exemplary data showing synpoptosis circuits perform combinatorial computation. As shown in FIG. 4A, synpoptosis circuits enabled context-dependent apoptosis by responding to logical combinations of protease inputs (TEVP as Input 1 and TVMVP as Input 2). In the first demonstration, transient transfection of plasmid DNA encoding both protease inputs must be present to activate the engineered caspase-3 and cause apoptosis, shown by flow cytometry. In the second demonstration, either input is sufficient to trigger apoptosis. In the third demonstration, apoptosis occurs only when the first input is present and the second input is absent. Throughout the figure, the gray window indicates the fractions established by negative and positive transient DNA transfection controls; dots represent biological replicates (distinct culture wells). Similar design principles were applied to engineer the same gating functions for pyroptosis (FIG. 4B). FIG. 4C displays exemplary data showing that using these synpoptosis gates, one can eliminate specific cells within mixed populations that exhibit distinct intracellular states defined by the expression profiles of input proteases and their fluorescence reporters, shown by flow cytometry. The cell states were established by stable protein expression using lentiviral transduction. Data represent three independent experiments. Quantification of Annexin staining revealed the efficacy of the synpoptosis gates (FIG. 4D). In FIG. 4D, each quadrant is shown from left to right for the indicated circuit. Horizontal lines indicate quadrant-specific negative and positive controls of the Annexin signal. Bars indicate means of three biological replicates (distinct culture wells). FIG. 4E displays a schematic showing synpoptosis circuits can achieve context-dependent killing by responding to logical combinations of protease inputs. As shown in FIG. 4F, using these synpoptosis gates, we can eliminate specific cells within mixed populations that exhibit distinct intracellular states.

FIG. 5A-FIG. 5D display non-limiting exemplary data showing synpoptosis circuits selectively eliminate target cells. As shown in FIG. 5A, synpoptosis circuits can selectively kill target cells by incorporating a sensor module that identifies the target cells. A synthetic sensor of active Ras was used. The sensor is a split TEVP with each half—nTEVP and cTEVP—tethered to a Ras-binding domain (RBD). In wildtype (WT) HEK cells with inactive Ras, the sensor is catalytically inactive. In cells stably expressing active Ras (RasGC cells), TEVP is reconstituted by proximity. FIG. 5B displays exemplary data showing that transient DNA transfection of a caspase-3-based synpoptosis circuit including the synthetic Ras sensor module enabled selective apoptosis of RasGC cells, but not WT cells. Throughout the figure, horizontal lines in the bar plots indicate the fractions established by negative and positive transient DNA transfection controls, separately for each dye and each cell line; dots represent biological replicates (distinct culture wells); bars indicate means; histograms represent three independent experiments. Similarly, a Ras-sensing synpoptosis circuit using GSDMA as the output triggered selective pyroptosis of RasGC cells (FIG. 5C). As shown in FIG. 5D, synpoptosis circuits can selectively kill target cells by incorporating a sensor module that identifies the targets. This feature is desired if we need to restrict synpoptosis to harmful cells within a mixture among healthy cells.

FIG. 6A-FIG. 6H display non-limiting exemplary data showing that synpoptosis circuits support intercellular operations. FIG. 6A displays an exemplary schematic showing synpoptosis circuits can be transmitted intercellularly by virus-like particles (VLPs). To address the key challenge of sender cell death, a simple strategy is to use a split-sender system, in which an inactive executioner and an activating protease are packaged separately. As shown in FIG. 6B, a model VLP transiently delivered nucleic acid encoding Cherry from sender HEK cells to receiver HEK cells, where the cargo was expressed. Experiments were performed by supernatant transfer from sender to receiver cells after transient DNA transfection of sender cells. Line segments connect means at different time points. Dots represent biological replicates (distinct culture wells). In FIG. 6C, two model cargoes, Cherry and Citrine, were separately packaged by two sender populations and co-delivered to the same receiver population, supporting the split-sender system. Scatter plots represent three independent experiments. FIG. 6D displays exemplary data showing a synthetic pyroptosis circuit, consisting of inactive GSDMA and the activating protease TEVP, was delivered by VLP using the split-sender system. Throughout the figure, the gray window indicates the fractions established by negative and positive transient DNA transfection controls; bars indicate means; dots in the two-dimensional sender death-receiver death plots indicate means of three biological replicates (distinct culture wells). Analogously, the split-sender system enabled VLP delivery of a synthetic apoptosis circuit, by separately packaging inactive caspase-3 and the activating protease TEVP (FIG. 6E). Appending CAAX tails to both circuit components enhanced apoptosis. A more elegant, compact, single-sender system directly delivers an active executioner. This strategy requires sender-specific silencing of the active executioner (FIG. 6F). As shown in FIG. 6G, a synthetic protein-level silencer (GSDMA Z-C) potently inhibited an engineered active executioner (GSDMA N-Z), likely by using leucine zippers (Z) to mask a loop critical for the executioner's activity, shown by AlphaFold models. The inhibitory curve was obtained by transient transfection of HEK cells with plasmid DNA encoding the active executioner and the silencer at indicated plasmid mass ratios. Senders transfected with plasmid DNA encoding the silencer, but not wildtype senders, could directly package VLPs that express the active executioner (FIG. 6H).

FIG. 7A-FIG. 7E display non-limiting exemplary data showing synpoptosis circuits orthogonally control cell death. FIG. 7A displays a graph showing that HEK cells express negligible levels of endogenous GSDMs. Expression data were obtained from the Human Protein Atlas. As shown in FIG. 7B, the majority of experiments in instant disclosure were performed using the transient transfection method, as shown in the schematic with mock data. The cells were co-transfected with plasmid DNA encoding the synthetic circuits and a fluorescent protein marker. After 16-24 hours post-transfection, both floating and attached cells were collected for staining and flow cytometry. As shown in FIG. 7C, there are several possible sources of cells that stain low for the fluorescent co-transfection marker (Cherry) and high for the death dye (Annexin). First, the transfection reagent itself causes some toxicity; second, the cell collection procedure involving pelleting and resuspending kills some cells; and third, when the circuits kill the cells, they also reduce the level of the fluorescent protein in the cells, making some transfected cells appear Cherry-low. Given the uncertain origins, Cherry-high cells were focused on by gating, which allowed definitive attribution of observed cell death to the transfected synthetic circuits. Data represent three independent experiments. FIG. 7D displays exemplary data showing three viral proteases, TEVP, TVMVP, and HCVP, orthogonally activated their cognate engineered GSDMs containing the cleavage sites (tev, tvmv, and hcv, respectively). Data represent three independent experiments. Colors indicate means. As shown in FIG. 7E, naturally occurring GSDMD inactivation by caspase-3 prompted us to insert a TEVP cleavage site (tev) at an equivalent location in GSDMA. The GSDMD structure was from the Protein Data Bank (PDB 6N9O). The engineered GSDMA model was predicted by AlphaFold.

FIG. 8A-FIG. 8D display non-limiting exemplary data related to synpoptosis circuits show typical features of natural death programs. As shown in FIG. 8A, in transient DNA transfection experiments, engineered auto-inhibited caspase-3 or GSDMA induced modest cell death when they were highly expressed, without the activating TEVP. Within each bin on the x-axis, dots represent biological replicates (distinct culture wells). FIG. 8B displays exemplary data showing titrating the DNA amounts of the circuit plasmids enabled dose-dependent control over the fraction of cell killing. Throughout the figure, the gray window indicates the y-ranges established by positive and negative transient DNA transfection controls; dots represent biological replicates (distinct culture wells). As shown in FIG. 8C, at a constant amount of the plasmid DNA, the penetrance of synpoptosis circuits could be tuned by modulating protein expression at the mRNA level, using a synthetic miRNA-based incoherent feedforward loop (IFFL). The strength of mRNA inhibition by the miRNA is tunable by adjusting base complementarity between mRNA and miRNA. In the GFP panel, dots mark median fluorescence values. FIG. 8D displays exemplary images showing cells killed by synpoptosis circuits displayed canonical morphological characteristics of apoptosis and pyroptosis. Images are representative of three independent experiments and on the same brightness and color scales. Scale bars: 200 μm (left) and 20 μm (right).

FIG. 9A-FIG. 9D display non-limiting exemplary data showing synpoptosis circuits modulate cell death. As shown in FIG. 9A, GSDME mutants displayed various pyroptotic activity compared to wildtype GSDME N-domain (N), shown by transient DNA transfection and flow cytometry. The last mutant was defective and later referred to as GSDME N(mut). GSDME N(mut) inhibited wildtype GSDME N-induced pyroptosis. Ratio of mutant to wildtype GSDME (mass of DNA plasmids) from left to right: 1-to-1 (low), 2-to-1 (medium), and 4-to-1 (high). The gray window indicates the fractions established by negative and positive transient DNA transfection controls. Throughout the figure, dots represent biological replicates (distinct culture wells). Time-course flow cytometry experiments suggested that GSDME N(mut) promoted apoptosis to various degrees, shown by different Sytox levels and similar Annexin levels, in cells expressing wildtype GSDME (FIG. 9B). The gray window indicates that Sytox remains low in apoptotic cells between 16 and 24 hours post DNA transfection. Using GSDMD-KO THP-1 cells, we demonstrated that the mRNA-encoded synpoptosis circuits trigger the expected mode of cell death, independently of endogenous death circuitry (FIG. 9C). Horizontal lines indicate the fractions established by negative and positive transient mRNA transfection controls. Shown in FIG. 9D is a schematic of natural mechanism the cell uses to attenuate pyroptosis is the trans inhibition of pyroptotic GSDMB (variants 3 and 4) by non-pyroptotic GSDMB (variants 1, 2, and 5). This architecture inspired the design of synpoptosis circuits that force apoptosis in GSDM-positive cells.

FIG. 10A-FIG. 10E display non-limiting exemplary data showing synpoptosis circuits enable complex functions. As shown in FIG. 10A, more gates were built that synthetically control apoptosis based on different protease input combinations, shown by transient DNA transfection and flow cytometry experiments. Throughout the figure, the gray window indicates the fractions established by negative and positive transient DNA transfection controls; dots represent biological replicates (distinct culture wells). The corresponding logic gates for synthetic control of pyroptosis were constructed similarly (FIG. 10B). As shown in FIG. 10C, cell death programs naturally respond to logical input combinations. GSDMD is proteolytically activated by either caspase-1 or caspase-11, like an OR gate. On the other hand, cleavage and lipidation of GSDMD by the zDHHC enzymes are both required for GSDMD activity, like an AND gate. Besides AND, OR, and NIMPLY, we built the other five binary logic gating functions for synthetic apoptosis (FIG. 10D). Similar principles were transferable to the design of synthetic pyroptosis gates (FIG. 10E).

FIG. 11A-FIG. 11C display non-limiting exemplary data showing synpoptosis circuits exhibit target cell selectivity. FIG. 11A shows a schematic of Proximity-reconstituted TEVP in RasGC cells should protect a degron-tagged Citrine reporter (Citrine-tev-Deg) from degradation, enhancing Citrine fluorescence. As shown in FIG. 11B, the synthetic Ras sensor enabled selective targeting of RasGC cells, shown by RasGC-specific enhancement of Citrine fluorescence, measured by flow cytometry after transient DNA transfection. Light and dark gray colors indicate WT and RasGC cells, respectively. The Citrine-tev-Deg reporter was stably integrated into RasGC and WT cells by lentiviral transduction. Data represent three independent experiments. FIG. 11C displays exemplary data showing the combination of nTEVP-RBD and cTEVP-RBD enhanced Citrine fluorescence in RasGC cells, shown on both linear and log scales. Horizontal lines indicate the median fluorescence values established by negative and positive transient DNA transfection controls, established separately for each cell line; bars indicate the means of median fluorescence values; dots represent biological replicates (distinct culture wells).

FIG. 12A-FIG. 12E display non-limiting exemplary data showing synpoptosis circuits allow cell-cell transmission. As shown in FIG. 12A, VLPs expressing a circuit component could complement the missing component in the cell, shown by flow cytometry after supernatant transfer. The gray window indicates the fractions established by negative and positive transient DNA transfection controls. Throughout the figure, dots represent biological replicates (distinct culture wells), and bars indicate means. FIG. 12B displays a graph showing inhibition of GSDMA N by GSDMA C requires a zipper (Z) on each of the two molecules, shown by transient DNA transfection and flow cytometry experiments. The gray window indicates the fractions established by negative and positive transient DNA transfection controls. FIG. 12C displays a graph of data showing senders expressing GSDMA Z-C (the silencer) after transient DNA transfection were protected from pyroptosis when treated with supernatants containing VLPs that express GSDMA N-Z (the engineered active executioner). The gray window indicates the fractions established by wildtype senders treated with empty VLPs and GSDMA N-Z VLPs, respectively. FIG. 12D displays exemplary images showing GSDMA Z-C (the silencer) suppressed cytotoxicity caused by GSDMA N-Z (the engineered active executioner). Images are representative of three independent experiments and on the same brightness and color scales. Scale bar: 200 μm. FIG. 12E displays exemplary data related to the single-sender system functions in a co-culture context. Sender cells were transiently transfected with DNA to express the silencer and package GSDMA N-Z VLPs or empty VLPs as a control. The senders, but not receivers, were stained with Vybrant DiD. To show that Vybrant DiD persists in dead cells HEK cells pre-stained by Vybrant DiD were transfected with plasmid DNA encoding GSDMA N-Z, followed by Sytox staining and flow cytometry.

FIG. 13 displays a schematic of the exemplary methods and compositions of the disclosure.

FIG. 14 displays a diagrams of the motivation for fine-tuning of cell death executioner activation thresholds.

FIG. 15A-FIG. 15B show non-limiting exemplary data related to the fine-tuning of cell death executioner activation thresholds. Graphs in FIG. 15A show mutation of protease cut site or addition of protease-cleavable degrons enables background suppression (cf. N.C. panel) and fine-tuning of the caspase activation threshold. Cell death quantified with Annexin V (APC channel). Caspase co-transfected with GFP to quantify caspase dosage. N.C.=nTEVP+cTEVP (no active TEVP reconstitution); P.C.=P3-nTEVP+P4-cTEVP (P3, P4 zipper domains trigger TEVP reconstitution); NeoR=no TEVP transfected. As shown in FIG. 15B, fine-tuning with select protease cleavage site modifications does not affect maximal killing. Cell death quantified with Annexin V.

FIG. 16 displays non-limiting exemplary data showing caspase localization can boost input sensitivity or reduce background killing. On the left is shown a schematic of design strategy. As shown in the graph on the right, when membrane TEVP is co-localized with membrane Casp3, the amount of induced cell death is higher than that of membrane TEVP with cytoplasmic Casp3.

FIG. 17 displays exemplary data showing gasdermin localization can boost input sensitivity or reduce background killing.

FIG. 18 shows data related to rational protein design can reduce background and boost sensitivity.

FIG. 19A-FIG. 19B display non-limiting exemplary data showing that split caspases can sense Ras and conditionally trigger cell death without requiring protease module. FIG. 19A depicts an exemplary schematic of a split-caspase circuit of the disclosure. FIG. 19B shows a graph of apoptosis induction in cells using an exemplary split caspase circuit.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “vector” refers to a polynucleotide construct, typically a plasmid or a virus, used to transmit genetic material to a host cell (e.g., a target cell). Vectors can be, for example, viruses, plasmids, cosmids, or phage. A vector can be a viral vector. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, 2A sequences or elements refer to small peptides introduced as a linker between two proteins, allowing autonomous intraribosomal self-processing of polyproteins (See e.g., de Felipe. Genetic Vaccines and Ther. 2: 13 (2004); deFelipe et al. Traffic 5:616-626 (2004)). These short peptides allow co-expression of multiple proteins from a single vector. Many 2A elements are known in the art. Examples of 2A sequences that can be used in the methods and system disclosed herein, without limitation, include 2A sequences from the foot-and-mouth disease virus (F2A), equine rhinitis A virus (E2A), Thosea asigna virus (T2A), and porcine tescho virus-1 (P2A).

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “variant” refers to a polynucleotide (or polypeptide) having a sequence substantially similar to a reference polynucleotide (or polypeptide). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, the term “effective amount” refers to an amount sufficient to effect beneficial or desirable biological and/or clinical results.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may reduce the level of RAS signaling in the subject, thereby to reduce, alleviate, or eradicate the symptom(s) of the disease(s). As used herein, the term “prevention” refers to any activity that reduces the burden of the individual later expressing those RAS-related disease symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, ammo acids, carbohydrates including glucose, mannose, or dextrins, chelating agents such as EDTA, sugar alcohols such as mannitol or sorbitol, salt-forming counterions such as sodium, and nonionic surfactants such as Tween, polyethylene glycol (PEG), and Pluronics. Auxiliary, stabilizer, emulsifier, lubricant, binder, pH adjuster controller, isotonic agent and other conventional additives may also be added to the carriers.

Described herein include synthetic protein-level cell death circuits, which can be collectively termed “synpoptosis” circuits. To engineer these circuits, inspiration was taken from natural cell death pathways that use regulated proteolysis along with protein-level caging and degradation mechanisms. Synpoptosis circuits and methods provide for rationally designed, programmable control of mammalian cell death. This disclosure presents a set of naturally inspired synthetic protein-level circuits, collectively termed “synpoptosis” circuits, that programmably control user-selectable death programs in target mammalian cells.

The ability to kill the right cells in the right way can address a variety of therapeutic challenges, such as cancer, fibrosis, senescence, and infection. Synpoptosis circuits provide a foundation for rationally designed, programmable control of mammalian cell death, and should enable new and broad therapeutic strategies. Described below are non-limiting, exemplary specific use cases.

In some embodiments, synpoptosis circuits can be delivered as mRNA-encoded therapeutics encapsulated in lipid nanoparticles (LNPs) to kill target cells. Circuit-based therapeutics only recently became possible thanks to development of LNP technologies that facilitate mRNA delivery. LNPs containing mRNA-encoded synpoptosis circuits can serve as targeted therapeutics that selectively eliminate diseased cells using user-specified death programs.

In some embodiments, synpoptosis circuits can be delivered by engineered cells via virus-like particles (VLPs) to kill target cells. A nascent therapeutic paradigm involves cell-based delivery systems, in which engineered sender cells secrete VLPs containing cargo that can be taken up by non-engineered receiver cells. Synpoptosis circuits can be used as the cargo in VLPs to achieve intercellular transmission and non-cell-autonomous killing.

In some embodiments, synpoptosis circuits can serve as outputs for a variety of existing synthetic circuits. Previous synthetic circuits, including oncogene-sensing circuits and neural network-based classification circuits, have focused on input signal processing while lacking functional, biologically relevant outputs. Synpoptosis circuits can interface with these circuits to form large synthetic networks that conditionally trigger cell death programs.

The circuits, compositions, and methods described herein represent several improvements. Many previous efforts have focused on creating systems that ectopically induce cell death. While these approaches generally enable induction of cell death, they have lacked capabilities that are essential for control, specificity, and delivery: They cannot control the mode of cell death, and thereby modulate the immune system. They have limited ability to interface with endogenous protein signals within the cell. And, they have not supported cell-to-cell transmission, which would enable engineering of synthetic killer cells that can kill other cells without killing themselves.

Provided herein are a set of engineered synpoptosis circuits capable of programmably controlling user-defined death programs in target mammalian cells. Synpoptosis circuits have the following key features. First, they allow activation and repression of different forms of death in target mammalian cells, overriding cell-intrinsic default death preferences. Second, they conditionally and combinatorially respond to intracellular protein signals, allowing target cell selectivity. Third, they can be transmitted intercellulary via virus-like particles, enabling engineering of synthetic killer cells that induce specific modes of cell death in target cells.

Synpoptosis circuits dictate the death mode in target cells regardless of cell-intrinsic death preferences. Motivated by natural proteolysis mechanisms, we created a set of synpoptosis circuits that use synthetic proteolysis to potently activate or repress cell death programs, in both immunologically cold and hot forms. These circuits operate orthogonally to endogenous pathways, override the cell-intrinsic death defaults, and can even induce two death modes simultaneously at a tunable ratio.

Synpoptosis circuits selectively kill target cells by performing molecular computation in response to input signals. Synpoptosis circuits can respond to cellular inputs such as elevated Ras signaling, and combinatorially integrate inputs, to enable selective elimination of target cells within mixed cell populations.

Synpoptosis circuits support intercellular killing operations through cell-cell circuit transmission. Using VLPs, a newly emerging cell-based delivery paradigm, engineered sender cells can deliver synpoptosis circuits to non-engineered receiver cells. This allows design of synthetic killer cells that activate user-specified death programs in target cells. These synthetic pathways are independent of natural cytotoxic mechanisms such as granzyme delivery. To enable this, a system was designed that prevents synthetic killer cells from killing themselves

Natural cell death pathways such as apoptosis and pyroptosis play dual roles: they eliminate harmful cells and modulate the immune system by dampening or stimulating inflammation. Synthetic protein circuits capable of triggering specific death programs in target cells may similarly remove harmful cells while appropriately modulating immune responses. However, cells actively influence their death modes in response to natural signals, making it challenging to control death modes. Described herein are naturally inspired synthetic circuits (e.g., “synpoptosis”) circuits that proteolytically regulate engineered executioner proteins and mammalian cell death. These circuits direct cell death modes, respond to combinations of protease inputs, and selectively eliminate target cells. Furthermore, synpoptosis circuits can be transmitted intercellularly, offering methods for engineering synthetic killer cells that induce desired death programs in target cells without self-destruction.

Synthetic biology allows for rational design of circuits that confer new functions in living cells. For example, CHOMP (circuits of hacked orthogonal modular proteases) enables design of composable protein circuit components. Many natural cellular functions can be implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a protein, e.g., a polynucleotide of the disclosure at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No.2018/0142307 and 2020/0172968, the contents of which are incorporated herein by reference in their entirety. The systems, methods, compositions, and kits provided herein can, in some embodiments, be employed in concert with the systems, methods, compositions, and kits for described in U.S. Patent Publication No. 2023/0076395, entitled, “CELL-TO-CELL DELIVERY OF RNA CIRCUITS,” and in U.S. Patent Publication No. 2023/0071834, entitled, “EXPORTED RNA REPORTERS FOR LIVE-CELL MEASUREMENT,” the contents of which are incorporated herein by reference in their entirety. The systems, methods, compositions, and kits provided herein can, in some embodiments, be employed in concert with the systems, methods, compositions, and kits described in PCT Application No. PCT/US23/69663, entitled, “A SYNTHETIC PROTEIN-LEVEL NEURAL NETWORK IN MAMMALIAN CELLS,” filed Jul. 5, 2023, the content of which is incorporated herein by reference in its entirety. The systems, methods, compositions, and kits provided herein can, in some embodiments, be employed in concert with the systems, methods, compositions, and kits described in PCT Application Publication No. WO2020117713A1, entitled, “In situ readout of dna barcodes,” the content of which is incorporated herein by reference in its entirety. The systems, methods, compositions, and kits provided herein can, in some embodiments, be employed in concert with the systems, methods, compositions, and kits described in U.S. patent application Ser. Nos. 17/820,232, 17/820,235, and 18/757,460, the contents of which are incorporated herein by reference in their entireties. The systems, methods, compositions, and kits provided herein can, in some embodiments, be employed in concert with the systems, methods, compositions, and kits described in PCT Application Publication No. WO2024081912A1, entitled, “PROTEIN-BASED SIGNAL AMPLIFICATION,” the content of which is incorporated herein by reference in its entirety. The systems, methods, compositions, and kits provided herein can, in some embodiments, be employed in concert with the systems, methods, compositions, and kits described in U.S. patent application Ser. No. 18/799,870, entitled, “MOLECULAR RECORDING METHODS AND SYSTEMS TO CAPTURE LINEAGE RELATIONSHIPS IN DIFFERENTIATING STEM CELLS,” filed Aug. 9, 2024, the content of which is incorporated herein by reference in its entirety. The systems, methods, compositions, and kits provided herein can, in some embodiments, be employed in concert with the systems, methods, compositions, and kits described in PCT Patent Application No. PCT/US24/48247, entitled, “MIRNA CIRCUITS FOR CONTROLLED GENE EXPRESSION,” filed Sep. 24, 2024, the content of which is incorporated herein by reference in its entirety. The systems, methods, compositions, and kits provided herein can, in some embodiments, be employed in concert with the systems, methods, compositions, and kits described in Vlahos A E, Kang J, Aldrete C A, Zhu R, Chong L S, Elowitz M B, Gao X J. Protease-controlled secretion and display of intercellular signals. Nat Commun. 2022 Feb. 17; 13(1):912. doi: 10.1038/s41467-022-28623-y. PMID: 35177637; PMCID: PMC8854555, the content of which is hereby incorporated by reference in its entirety. The systems, methods, compositions, and kits provided herein can, in some embodiments, be employed in concert with the systems, methods, compositions, and kits described in Xia et al. (“Synthetic protein circuits for programmable control of mammalian cell death.” Cell (2024): 2785-2800), the content of which, including its star methods, supplementary materials and supplemental data, is incorporated herein by reference in its entirety.

### Synpoptosis Circuits

Disclosed herein are synthetic protein circuits (e.g., synpoptosis circuits). Disclosed herein include synthetic protein circuits comprising: one or more apoptosis polypeptides; and/or one or more pyroptosis polypeptides; and/or one or more input polypeptides.

Disclosed herein include synthetic protein circuits comprising: a first apoptosis polypeptide comprising a large subunit of an apoptotic effector protein and a small subunit of an apoptotic effector protein separated by a first heterologous protease cleavage site, wherein two first apoptosis polypeptides are capable of associating with each other to constitute a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, wherein the first apoptotic protein complex in the first apoptotic protein complex active state is capable of inducing apoptosis in a cell. In some embodiments, the synthetic protein circuit further comprises a first input polypeptide comprising the first heterologous protease.

Disclosed herein include synthetic protein circuits comprising: a first apoptosis polypeptide comprising a large subunit of an apoptotic effector protein and a first partner domain; and a second apoptosis polypeptide comprising a small subunit of an apoptotic effector protein, a second partner domain capable of binding the first partner domain, a first heterologous protease cleavage site, and a first degron, wherein a first heterologous protease in a first heterologous protease active state is capable of cutting the first heterologous protease cleavage site of the second apoptosis polypeptide and thereby releasing the first degron, and wherein the first heterologous protease cleavage site of the second apoptosis polypeptide being cut changes the second apoptosis polypeptide from a second apoptosis polypeptide destabilized state to a second apoptosis polypeptide stabilized state, wherein the first apoptosis polypeptide and the second apoptosis polypeptide in the second apoptosis polypeptide stabilized state are capable of associating via binding of the first partner domain and the second partner domain to form a subunit, and wherein two subunits are capable of associating with each other to constitute a first apoptotic protein complex capable of being in a first apoptotic protein complex active state, wherein the first apoptotic protein complex in the first apoptotic protein complex active state is capable of inducing apoptosis in a cell. In some embodiments, the synthetic protein circuit further comprises a first input polypeptide comprising the first heterologous protease.

Disclosed herein include synthetic protein circuits comprising: a first apoptosis polypeptide comprising a large subunit of an apoptotic effector protein, a first partner domain, a first degron, and a first heterologous protease cleavage site; and a second apoptosis polypeptide comprising a small subunit of an apoptotic effector protein and a second partner domain capable of binding the first partner domain, wherein the first apoptosis polypeptide and the second apoptosis polypeptide are capable of associating via binding of the first partner domain and the second partner domain to form a subunit, wherein two subunits are capable of associating with each other to constitute a first apoptotic protein complex capable of being in a first apoptotic protein complex active state, wherein the first apoptotic protein complex in the first apoptotic protein complex active state is capable of inducing apoptosis in a cell, wherein a first heterologous protease in a first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state. In some embodiments, the synthetic protein circuit further comprises a first input polypeptide comprising the first heterologous protease.

Disclosed herein include synthetic protein circuits comprising: a first pyroptosis polypeptide comprising a pyroptosis effector domain and a first inhibitory domain separated by a first heterologous protease cleavage site, wherein the first inhibitory domain is capable of inhibiting activity of the pyroptosis effector domain, thereby the first pyroptosis polypeptide is in a first pyroptosis polypeptide inactive state, wherein a first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in a cell. In some embodiments, the synthetic protein circuit further comprises a first input polypeptide comprising the first heterologous protease.

Disclosed herein include synthetic protein circuits comprising: a first pyroptosis polypeptide comprising a pyroptosis effector domain and a first degron separated by a first heterologous protease cleavage site, wherein a first heterologous protease in a first heterologous protease active state is capable of cutting the first heterologous protease cleavage site of the first pyroptosis polypeptide and thereby releasing the first degron, and wherein the first heterologous protease cleavage site of the first pyroptosis polypeptide being cut changes the first pyroptosis polypeptide from a first pyroptosis polypeptide destabilized state to a first pyroptosis polypeptide stabilized state, wherein the first pyroptosis polypeptide in the first pyroptosis polypeptide stabilized state is capable of being in a first pyroptosis polypeptide active state, wherein the first pyroptosis polypeptide in the first pyroptosis polypeptide active state is capable of inducing pyroptosis in a cell. In some embodiments, the synthetic protein circuit further comprises a first input polypeptide comprising the first heterologous protease.

Disclosed herein include synthetic protein circuits comprising: a first pyroptosis polypeptide comprising a pyroptosis effector domain, a first degron, and a first heterologous protease cleavage site, wherein the first pyroptosis polypeptide is capable of being in a first pyroptosis polypeptide active state, wherein the first pyroptosis polypeptide in the first pyroptosis polypeptide active state is capable of inducing pyroptosis in a cell, and wherein a first heterologous protease in a first heterologous protease active state is capable of cutting the first heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state. In some embodiments, the synthetic protein circuit further comprises a first input polypeptide comprising the first heterologous protease.

Any of the protein circuits disclosed herein can be configured into a logic gate. Disclosed herein include synthetic protein circuits comprising: (i) one or more apoptosis polypeptides or one or more pyroptosis polypeptides and (ii) one or more input polypeptides, configured to form one or more logic gates selected from the group comprising an OR logic gate, AND logic gate, NOR logic gate, NAND logic gate, IMPLY logic gate, NIMPLY logic gate, XOR logic gate, and an XNOR logic gate. In some embodiments, two, three, four or more inputs (e.g., heterologous protease cleavage sites and proteases) can be incorporated into a synthetic protein circuit to configure the logic gate.

Disclosed herein include synthetic protein circuits comprising: one or more first, second, or third apoptosis polypeptides; one or more first or second pyroptosis polypeptides; one or more input polypeptides; one or more pyroptosis effector proteins; and/or one or more mutant pyroptosis effector proteins, wherein the synthetic protein circuit is capable of inducing in a cell: apoptosis via a first apoptotic protein complex in first apoptotic protein complex active state; and/or pyroptosis via a first and/or second pyroptosis polypeptide in a first and/or second pyroptosis polypeptide active state or a pyroptosis effector protein in a pyroptosis effector protein active state.

Disclosed herein include synthetic protein circuits comprising: (i) a first apoptosis polypeptide comprising a large subunit of an apoptotic effector protein and a small subunit of an apoptotic effector protein separated by a first heterologous protease cleavage site, wherein two of the first apoptosis polypeptides are capable of forming a first apoptotic protein complex in a first apoptotic protein complex inactive state, and wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic complex inactive state to a first apoptotic protein complex active state, wherein the first apoptotic protein complex in the first apoptotic protein complex active state is capable of inducing apoptosis in a cell; and (ii) a first pyroptosis polypeptide comprising a pyroptosis effector domain and a first inhibitory domain separated by the first heterologous protease cleavage site, wherein the first inhibitory domain is capable of inhibiting activity of the pyroptosis effector domain, thereby the first pyroptosis polypeptide is capable of being in a first pyroptosis polypeptide inactive state, wherein the first heterologous protease in the first heterologous protease active state is capable of cutting the first heterologous protease cleavage site of the first of the first pyroptosis polypeptide, wherein the first pyroptosis polypeptide being cut changes the first pyroptosis polypeptide from the first pyroptosis polypeptide inactive state to a first pyroptosis polypeptide active state, and wherein the first pyroptosis polypeptide in the first pyroptosis polypeptide active state is capable of (a) inducing pyroptosis in the cell; and (b) inhibiting the induction of apoptosis in the cell by the first apoptotic protein complex in the first apoptotic protein complex active state. In some embodiments, said inhibition is at least 10%. In some embodiments, the synthetic protein circuit further comprises a first input polypeptide comprising the first heterologous protease.

Disclosed herein include synthetic protein circuits comprising: a first apoptosis polypeptide comprising a large subunit of an apoptotic effector protein and a small subunit of an apoptotic effector protein, separated by a first heterologous protease cleavage site, wherein two of the first apoptosis polypeptides are capable of forming a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptosis protease complex inactive state to a first apoptotic protein complex active state, wherein the first apoptotic protein complex in the first apoptotic protein complex active state is capable of (a) inducing apoptosis in a cell expressing a pyroptosis effector protein; and/or (b) cutting the pyroptosis effector protein, wherein the pyroptosis effector protein being cut changes the pyroptosis effector protein from a pyroptosis effector protein inactive state to a pyroptosis effector protein active state, wherein the pyroptosis effector protein in the pyroptosis effector protein active state is capable of: (c) inducing pyroptosis in the cell; and (d) inhibiting the induction of apoptosis in the cell by the first apoptotic protein complex in the first apoptotic protein complex active state. In some embodiments, the synthetic protein circuit further comprises a first input polypeptide comprising the first heterologous protease.

Disclosed herein include synthetic protein circuits comprising: (i) a first apoptosis polypeptide comprising a large subunit of an apoptotic effector protein and a small subunit of an apoptotic effector protein, separated by a first heterologous protease cleavage site, wherein two of the first apoptosis polypeptides are capable of forming a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, wherein the first apoptotic protein complex in the first apoptotic protein complex active state is capable of: (a) inducing apoptosis in a cell expressing a pyroptosis effector protein; and (b) cutting the pyroptosis effector protein, wherein the pyroptosis effector protein being cut changes the pyroptosis effector protein from a pyroptosis effector protein inactive state to a pyroptosis effector protein active state, wherein the pyroptosis effector protein in the pyroptosis effector protein active state is capable of: (c) inducing pyroptosis in the cell; and (d) inhibiting the induction of apoptosis in the cell by the apoptotic protein complex in the first apoptotic protein complex active state; and (ii) a first pyroptosis polypeptide comprising a mutant pyroptosis effector domain comprising a mutation, capable of inhibiting the pyroptosis effector protein in the pyroptosis effector protein active state, thereby changing the pyroptosis effector protein from the pyroptosis effector protein active state to a pyroptosis effector protein inactive state; thereby the first apoptotic protein complex in the first apoptotic protein complex active state is capable of inducing apoptosis in the cell. In some embodiments, the synthetic protein circuit comprises a first input polypeptide comprising the first heterologous protease.

Also disclosed herein are, e.g., split cell death execution systems. Disclosed herein include synthetic protein circuits comprising: a first polypeptide comprising a first signal transducer binding domain and a first part of a first cell death executioner, wherein the first signal transducer binding domain is capable of binding a first signal transducer to form a first signal transducer-bound polypeptide; a second polypeptide comprising a second signal transducer binding domain and a second part of the first cell death executioner, wherein the second signal transducer binding domain is capable of binding a second signal transducer to form a second signal transducer-bound polypeptide, and wherein the first part of the first cell death executioner and the second part of the first cell death executioner are capable of associating with each other to constitute a first cell death executioner capable of being in a first cell death executioner active state when the first signal transducer and the second signal transducer are in close proximity at an association location; and wherein the first cell death executioner in the first cell death executioner active state is capable of inducing apoptosis or pyroptosis in a cell.

### Apoptosis and Pyroptosis Polypeptides and Effector Proteins

Disclosed herein include synthetic protein circuits comprising: one or more apoptosis polypeptides; and/or one or more pyroptosis polypeptides; and/or one or more input polypeptides.

The synthetic protein circuits provided herein can comprise one or more polypeptides comprising cell death executioners or any portions or derivatives thereof. A cell death executioner can comprise any protein capable of inducing cell death, e.g., an apoptotic effector protein, a pyroptotic effector protein, or any variants or portions thereof.

The synthetic protein circuits described herein can comprise apoptosis effector protein(s) and/or pyroptosis effector protein(s), or any variants or portions thereof. In some embodiments, apoptosis effector proteins are capable of promoting apoptosis in a cell. Apoptosis is a form of programmed cell death that occurs in multicellular organisms and in some eukaryotic, single-celled microorganisms such as yeast. Biochemical events lead to characteristic cell changes (morphology) and death. These changes include blebbing, cell shrinkage, nuclear fragmentation, chromatin condensation, DNA fragmentation, and mRNA decay.

Apoptosis is a highly regulated and controlled process that confers advantages during an organism's life cycle. Because apoptosis cannot stop once it has begun, it is a highly regulated process. Apoptosis can be initiated through one of two pathways. In the intrinsic pathway the cell kills itself because it senses cell stress, while in the extrinsic pathway the cell kills itself because of signals from other cells. Weak external signals may also activate the intrinsic pathway of apoptosis. Both pathways induce cell death by activating caspases, which are proteases, or enzymes that degrade proteins. The two pathways both activate initiator caspases, which then activate executioner (e.g., effector) caspases, which then kill the cell by degrading proteins indiscriminately. In addition to its importance as a biological phenomenon, defective apoptotic processes have been implicated in a wide variety of diseases. Excessive apoptosis causes atrophy, whereas an insufficient amount results in uncontrolled cell proliferation, such as cancer. Some factors like Fas receptors and caspases promote apoptosis, while some members of the Bcl-2 family of proteins inhibit apoptosis.

In some embodiments, pyroptosis effector proteins are capable of promoting pyroptosis in a cell. Pyroptosis is a highly inflammatory form of lytic programmed cell death that occurs most frequently upon infection with intracellular pathogens and is likely to form part of the antimicrobial response. This process promotes the rapid clearance of various bacterial, viral, fungal and protozoan infections by removing intracellular replication niches and enhancing the host's defensive responses. Pyroptosis can take place in immune cells and is also reported to occur in keratinocytes and some epithelial cells.

The process is initiated by formation of a large supramolecular complex termed the inflammasome (also known as a pyroptosome) upon intracellular danger signals. The inflammasome activates a different set of caspases as compared to apoptosis, for example, caspase-1/4/5 in humans and caspase-11 in mice. These caspases contribute to the maturation and activation of the pro-inflammatory cytokines IL-1β and IL-18, as well as the pore-forming protein gasdermin D. Formation of pores causes cell membrane rupture and release of cytokines, as well as various damage-associated molecular pattern (DAMP) molecules such as HMGB-1, ATP and DNA, out of the cell. These molecules recruit more immune cells and further perpetuate the inflammatory cascade in the tissue.

In some embodiments, an apoptotic effector protein comprises caspase-1, caspase-2, caspase-3, caspase-4, caspase-5, caspase-6, caspase-7, caspase-8, caspase-9, caspase-10, caspase-14, or any variant, portion, or derivative thereof. In some embodiments, a pyroptosis effector comprises a gasdermin (GSDM) protein or any variant, portion, or derivative thereof. In some embodiments, the GSDM protein is GSDMA, GSDMB, GSDMC, GSDMD, GSDME, or DFNB59.

An apoptosis or pyroptosis polypeptide can comprise any portion of an apoptotic effector protein or a pyroptotic effector protein. For example, in some embodiments, a first apoptosis polypeptide can comprise a large subunit of an apoptotic effector protein; and a second apoptosis polypeptide can comprise a small subunit of an apoptotic effector protein. In other embodiments, a first apoptosis polypeptide can comprise a small subunit of an apoptotic effector protein; and a second apoptosis polypeptide comprise a large subunit of an apoptotic effector protein. Any pyroptosis polypeptide (e.g., a first and/or second pyroptosis polypeptide) can comprise inhibitory domain(s). In some embodiments, an inhibitory domain (e.g., the first inhibitory domain) comprises a protein domain configured to reduce or abrogate the activity of the pyroptosis effector domain, e.g., a bulky domain. The first inhibitory domain can comprise an auto-inhibitory domain of a gasdermin family of pore-forming proteins or maltose-binding protein.

Polypeptides comprising one or more components of a synthetic protein circuit are provided herein. First, second, and third apoptosis or pyroptosis polypeptides are provided herein. In some embodiments, a polypeptide (e.g., a first, second, and/or third apoptosis or pyroptosis polypeptide) can comprise apoptosis effector protein(s) and/or pyroptosis effector protein(s), or any variants or portions thereof. In some embodiments, a component of a synthetic protein circuit described herein (e.g., the first, second, and/or third apoptosis or pyroptosis polypeptide) can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to any one of SEQ ID NOs: 10-14, 16-26, and 28-45. In some embodiments, a component of a synthetic protein circuit described herein (e.g., the first, second, and/or third apoptosis or pyroptosis polypeptide) can comprise or consist of an amino acid sequence of any one of SEQ ID NOs: 10-14, 16-26, and 28-45.

### Degrons

Any of the polypeptides disclosed herein can comprise a degron. A degron is a portion of a protein that is important in regulation of protein degradation rates. Known degrons include short amino acid sequences, structural motifs, and exposed amino acids (often Lysine or Arginine) located anywhere in the protein. In fact, some proteins can even contain multiple degrons. Degrons are present in a variety of organisms, from the N-degrons (see N-end Rule) first characterized in yeast to the PEST sequence of mouse omithine decarboxylase. Degrons have been identified in prokaryotes as well as eukaryotes. While there are many types of different degrons, and a high degree of variability even within these groups, degrons are all similar for their involvement in regulating the rate of a protein's degradation. Much like protein degradation (see proteolysis) mechanisms are categorized by their dependence or lack thereof on Ubiquitin, a small protein involved in proteasomal protein degradation, degrons are also be referred to as “Ubiquitin-dependent” or “Ubiquitin-independent.

In some embodiments, the degron comprises an N-degron, a dihydrofolate reductase (DHFR) degron, a FKB protein (FKBP) degron, derivatives thereof, or any combination thereof. The degron can comprise an N-degron. Some degrons are ubiquitin-dependent or ubiquitin-independent. A degron can comprise DHFR degron, an N-degron, a phospho degron, a heat inducible degron, a photosensitive degron, an oxygen dependent degron, omithine decarboxylase degron, estrogen receptor domain degrons, a ecDHFR degron, an FKBP degron, a UnaG degron, or any combination thereof. As a non-limiting example, the degron may be an omithine decarboxylase degron. The degron can comprise a ecDHFR degron. The presence of a degron sequence in a polypeptide can result in the polypeptide being targeted for degradation, e.g., be in “a destabilized state.”

### Proteases and Protease Cleavage Sites

In some embodiments, a polypeptide (e.g., protein) disclosed herein can comprise a heterologous protease cleavage site. There are provided, in some embodiments, polypeptides (e.g., proteins) comprising a heterologous protease (or a portion thereof). “Heterologous”, as used herein, shall be given its ordinary meaning, and shall also refer to two or more polynucleotides (e.g., RNA or DNA sequences) or two or more polypeptides (e.g., proteins or protein domains) that are not found in the same relationship to each other in nature. For example, while wild-type gasdermins can comprise sequences capable of being cleaved by native proteases such as active caspase-3, in some embodiments provided herein, the cleavage site capable of being cut by, e.g., caspase-3, has been engineered to generate a heterologous protease cleavage site recognized by a natural (e.g., human, viral, etc.) protease or engineered protease which does not cleave gasdermin in nature. In some embodiments, a polypeptide of the disclosure can comprise a heterologous protease cleavage site, e.g., a tobacco etch virus (TEV) cleavage site. In some embodiments, the heterologous protease cleavage site is a wild-type or engineered cleavage site of a mammalian (e.g., human) protease. Synthetic protein circuits provided herein can comprise polypeptides (e.g., proteins) wherein a natural or engineered protease (or portion thereof) is an element of a protein or protein complex not found in nature—and hence heterologous.

Provided herein are input polypeptides (e.g., first and/or second input polypeptides) comprising a heterologous protease (e.g., a first and/or second heterologous protease). The protease capable of cutting a heterologous protease cleavage site (e.g., a heterologous protease) can be any protein capable of cutting the heterologous protease cleavage site. The heterologous protease can be an endogenous protease or exogenous protease. In some embodiments, the protease is a viral protease. In some embodiments, the protease is a mammalian (e.g., human protease). The protease (e.g., a heterologous protease) can comprise tobacco etch virus (TEV) protease, tobacco vein mottling virus (TVMV) protease, hepatitis C virus protease (HCVP), derivatives thereof, or any combination thereof. The protease can comprise TEVP. The cut site can comprise an amino acid sequence at least about 25% (e.g., 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) homologous to the canonical cut of site of a protease.

In some embodiments, the first heterologous protease comprises or is derived from one or more of a prokaryotic protease, bacterial protease, archaeal protease, eukaryotic protease, fungal protease, plant protease, mammalian protease, and human protease. In some embodiments, the first heterologous protease is engineered. In some embodiments, the first heterologous protease comprises tobacco etch virus (TEV) protease, tobacco vein mottling virus (TVMV) protease, hepatitis C virus protease (HCVP), derivatives thereof, or any combination thereof. In some embodiments, the first heterologous protease cleavage site comprises the sequence of SEQ ID NO: 1 or SEQ ID NO: 2. In some embodiments, the first heterologous protease cleavage site is natural or engineered, e.g., the cleavage sequence of a human apoptotic effector protease, e.g., a caspase cleavage sequence. In some embodiments, the first heterologous protease cleavage site is configured to modulate an activation threshold of the synthetic protein circuit. In some embodiments, said configuring comprises introducing one or more amino acid insertions, substitutions, or deletions relative to a wild type first heterologous protease cleavage site. In some embodiments, the first heterologous protease cleavage site comprises the sequence of any one of SEQ ID NOs: 3-7.

The heterologous protease cleavage site can comprise a sequence of SEQ ID NOs: 1-7 or a sequence having one, two, or three mismatches relative to any of SEQ ID NOs: 1-7. In some embodiments, the protease comprises a sequence comprising the sequence of SEQ ID NO: 15 or 27, or a sequence 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 15 or 27. Any of the polypeptides provided herein (e.g., an apoptosis polypeptide or a pyroptosis polypeptide) can comprise one or more heterologous protease cleavage sites. For example, a polypeptide can comprise one, two, three, four, five or more heterologous protease cleavage sites. A heterologous protease cleavage site can be at any position within a polypeptide (e.g., N-terminal, C-terminal, or internal). Similarly, the synthetic protein circuit can comprise one or more proteases (e.g., one or more heterologous proteases). For example, the synthetic protein circuit can comprise one, two, three, four, five or more proteases (e.g., heterologous proteases). In some embodiments, any two of the one or more proteases (e.g., heterologous proteases) are different from each other, and/or are each capable of cutting different heterologous protease cleavage sites. In other embodiments, any two of the one or more proteases (e.g., heterologous proteases) are the same.

### Partner Domains

Any of the polynucleotides of the disclosure can comprise one or more partner domains (e.g., a first, a second, a third, and/or a fourth partner domain). The one or more partner domains can, e.g., facilitate intramolecular binding to stabilize an apoptotic protein complex or a pyroptotic polypeptide in an apoptotic protein complex inactive state or a pyroptosis polypeptide inactive state. The one or more partner domains can, e.g., facilitate intermolecular binding of different polypeptides (e.g., apoptosis and/or pyroptosis polypeptides).

A partner domain (e.g., the first partner domain, second partner domain, third partner domain, and/or fourth partner domain) can comprise an intein or a fragment thereof. The first partner domain, second partner domain, third, and/or fourth partner can comprise a split intein or a fragment thereof. An “intein” is a segment of a protein that is able to excise itself and join the remaining portions (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein. For example, in cyanobacteria, DnaE, the catalytic subunit of DNA polymerase III, is encoded by two separate genes, dnaE-n and dnaE-c. The intein encoded by the dnaE-n gene is herein referred as “intein-N.” The intein encoded by the dnaE-c gene is herein referred as “intein-C.” Other intein systems may also be used. For example, a synthetic intein based on the dnaE intein, the Cfa-N and Cfa-C intein pair, has been described (e.g., in Stevens et al., J Am Chem Soc. 2016 Feb. 24; 138(7):2162-5, incorporated herein by reference). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

The first partner domain, second partner domain, third partner domain, and/or fourth partner domain can comprise an intein-N, an intein-C, a fragment thereof, or any combination thereof. For example, a partner domain can comprise CfaN, CfaC, NpuC, NpuN, gp41-1, gp41-2, gp41-3, gp41-4, gp41-5, gp41-6, gp41-7, gp41-8, IMPDH-1, NrdA-1, NrdA-2, NrdA-4, NrdA-5, NrdA-6, NrdJ-1, NrdJ-2 a fragment thereof, or any combination thereof.

Any of the partner domains of the disclosure can comprise at least one mutation, e.g., an amino acid modification. The first partner domain can comprise at least one amino acid modification, such as a deletion, substitution, addition, or a set of amino acid modifications, that affects binding to the second partner domain. The second partner domain can comprise at least one amino acid modification, such as a deletion, substitution, addition, or a set of amino acid modifications, that affects binding to the first partner domain. The third partner domain can comprise at least one amino acid modification, such as a deletion, substitution, addition, or a set of amino acid modifications, that affects binding to the fourth partner domain. The fourth partner domain can comprise at least one amino acid modification, such as a deletion, substitution, addition, or a set of amino acid modifications, that affects binding to the third partner domain. The first partner domain and second partner domain can be homodimers or heterodimers. The third partner domain and fourth partner domain can be homodimers or heterodimers.

The binding between the first partner domain and the second partner domain and/or the third partner domain and the fourth partner domain can be reversible. The binding between the first partner domain and the second partner domain and/or the third partner domain and the fourth partner domain can be irreversible.

A partner domain (e.g., a first, second, third, and/or fourth partner domain) can comprise SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, or any combination thereof. A partner domain (e.g., a first, second, third, and/or fourth partner domain) can comprise a PDZ domain and/or a PDZ domain ligand. A partner domain (e.g., a first, second, third, and/or fourth partner domain) can comprise an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. The (i) first partner domain and second partner domain and/or (ii) third partner domain and fourth partner domain can be a pair of constitutive protein partner domains selected from the group consisting of (a) cognate leucine zipper domains, (b) cognate PSD95-Dlgl-Zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide. A partner domain (e.g., a first, second, third, and/or fourth partner domain) can comprise CZp, NZp, or any combination thereof. A partner domain (e.g., a first, second, third, and/or fourth partner domain) can comprise a SYNZIP domain, a I- and N-series CC-Di-AB dimerization domain, a Designed Heterodimerization Domain (DHD), a National Institute of Chemistry (NICP) Set domain, a domain from the CCNG1 & CCmax Libraries, a Designed Heterotrimerization Domains (DHT), a Phosphorylation-responsive Coiled-coil heterotetramer, a DHD13_XAAA domain, a 4-helix bundle domain, and/or a Designed Binders of Repeating Peptides. In some embodiments, a partner domain (e.g., the first partner domain, the second partner domain, the third partner domain, and/or the fourth partner domain) comprises nHalo, cHalo, or any combination thereof.

Dimerization domains that may be used are also described in the following publications, the contents of which are hereby incorporated by reference in their entireties: Reinke, A. W.; Grant, R. A.; Keating, A. E. A Synthetic Coiled-Coil Interactome Provides Heterospecific Modules for Molecular Engineering. J. Am. Chem. Soc. 2010, 132 (17), 6025-6031; Thompson, K. E.; Bashor, C. J.; Lim, W. A.; Keating, A. E. SYNZIP Protein Interaction Toolbox: In Vitro and in Vivo Specifications of Heterospecific Coiled-Coil Interaction Domains. ACS Synth. Biol. 2012, 1 (4), 118-129; Thomas, F.; Boyle, A. L.; Burton, A. J.; Woolfson, D. N. A Set of de Novo Designed Parallel Heterodimeric Coiled Coils with Quantified Dissociation Constants in the Micromolar to Sub-Nanomolar Regime. J. Am. Chem. Soc. 2013, 135 (13), 5161-5166; Baryshev, A.*; La Fleur, A.*; Groves, B.; Michel, C.; Baker, D.; Ljubetič, A.; Seelig, G. Massively Parallel Measurement of Protein-Protein Interactions by Sequencing Using MP3-Seq. Nat. Chem. Biol. 2024, 1-10; Kurgan, K. W.; Martin, F. J. O.; Dawson, W. M.; Brunnock, T.; Orr-Ewing, A. J.; Woolfson, D. N. Exchange, Promiscuity, and Orthogonality in de Novo Designed Coiled-Coil Peptide Assemblies. bioRxiv 2024; Chen, Z.; Boyken, S. E.; Jia, M.; Busch, F.; Flores-Solis, D.; Bick, M. J.; Lu, P.; VanAemum, Z. L.; Sahasrabuddhe, A.; Langan, R. A.; Bermeo, S.; Brunette, T. J.; Mulligan, V. K.; Carter, L. P.; DiMaio, F.; Sgourakis, N. G.; Wysocki, V. H.; Baker, D. Programmable Design of Orthogonal Protein Heterodimers. Nature 2019, 565 (7737), 106-111; Nguyen, T. H.*; Dods, G.*; Gómez-Schiavon, M.; Wu, M.; Chen, Z.; Kibler, R.; Baker, D.; El-Samad, H.; Ng, A. H. Competitive Displacement of De Novo Designed HeteroDimers Can Reversibly Control Protein-Protein Interactions and Implement Feedback in Synthetic Circuits. GEN Biotechnol. 2022, 1 (1), 91-100; Baryshev, A.*; La Fleur, A.*; Groves, B.; Michel, C.; Baker, D.; Ljubetič, A.; Seelig, G. Massively Parallel Protein-Protein Interaction Measurement by Sequencing (MP3-Seq) Enables Rapid Screening of Protein Heterodimers. bioRxiv Feb. 9, 2023, p 2023.02.08.527770; Lebar, T.; Lainšček, D.; Merljak, E.; Aupič, J.; Jerala, R. A Tunable Orthogonal Coiled-Coil Interaction Toolbox for Engineering Mammalian Cells. Nat. Chem. Biol. 2020, 16 (5), 513-519; Plaper, T.; Aupič, J.; Dekleva, P.; Lapenta, F.; Keber, M. M.; Jerala, R.; Benčina, M. Coiled-Coil Heterodimers with Increased Stability for Cellular Regulation and Sensing SARS-CoV-2 Spike Protein-Mediated Cell Fusion. Sci. Rep. 2021, 11 (1), 9136; Baryshev, A.*; La Fleur, A.*; Groves, B.; Michel, C.; Baker, D.; Ljubetič, A.; Seelig, G. Massively Parallel Protein-Protein Interaction Measurement by Sequencing (MP3-Seq) Enables Rapid Screening of Protein Heterodimers. bioRxiv Feb. 9, 2023, p 2023.02.08.527770; Boldridge, W. C.*; Ljubetič, A.*; Kim, H.; Lubock, N.; Szilágyi, D.; Lee, J.; Brodnik, A.; Jerala, R.; Kosuri, S. A Multiplexed Bacterial Two-Hybrid for Rapid Characterization of Protein-Protein Interactions and Iterative Protein Design. Nat. Commun. 2023, 14 (1), 4636; Sahtoe, D. D.*; Praetorius, F.*; Courbet, A.; Hsia, Y.; Wicky, B. I. M.; Edman, N. I.; Miller, L. M.; Timmermans, B. J. R.; Decarreau, J.; Morris, H. M.; Kang, A.; Bera, A. K.; Baker, D. Reconfigurable Asymmetric Protein Assemblies through Implicit Negative Design. Science 2022, 375 (6578), eabj7662; Bermeo, S.*; Favor, A.*; Chang, Y.-T.*; Norris, A.; Boyken, S. E.; Hsia, Y.; Haddox, H. K.; Xu, C.; Brunette, T. J.; Wysocki, V. H.; Bhabha, G.; Ekiert, D. C.; Baker, D. De Novo Design of Obligate ABC-Type Heterotrimeric Proteins. Nat. Struct. Mol. Biol. 2022, 29 (12), 1266-1276; Thompson, H. F.; Beesley, J. L.; Langlands, H. D.; Edgell, C. L.; Savery, N. J.; Woolfson, D. N. Rational Design of Phosphorylation-Responsive Coiled Coil-Peptide Assemblies. ACS Synth. Biol. 2023; Merljak, E.; Malovrh, B.; Jerala, R. Segmentation Strategy of de Novo Designed Four-Helical Bundles Expands Protein Oligomerization Modalities for Cell Regulation. Nat. Commun. 2023, 14 (1), 1995; Smith, A. J.*; Naudin, E. A.*; Edgell, C. L.; Baker, E. G.; Mylemans, B.; FitzPatrick, L.; Herman, A.; Rice, H. M.; Andrews, D. M.; Tigue, N.; Woolfson, D. N.; Savery, N. J. Design and Selection of Heterodimerizing Helical Hairpins for Synthetic Biology. ACS Synth. Biol. 2023, 12 (6), 1845-1858; Chubb, J. J.*; Albanese, K. I.*; Rodger, A.; Woolfson, D. N. De Novo Design of Parallel and Antiparallel A3B3 Heterohexameric Alpha-Helical Barrels. bioRxiv 2024; Wu, K.*; Bai, H.*; Chang, Y.-T.; Redler, R.; McNally, K. E.; Sheffler, W.; Brunette, T. J.; Hicks, D. R.; Morgan, T. E.; Stevens, T. J.; Broerman, A.; Goreshnik, I.; DeWitt, M.; Chow, C. M.; Shen, Y.; Stewart, L.; Derivery, E.; Silva, D. A.; Bhabha, G.; Ekiert, D. C.; Baker, D. De Novo Design of Modular Peptide-Binding Proteins by Superhelical Matching. Nature 2023, 1-9.

### Additional Components

The polypeptides provided herein can comprise one or more additional components.

For example, any of the polypeptides provided herein can comprise one or more linkers. The linker can be at any location within, e.g., an apoptosis, pyroptosis, or input polypeptide.

The first apoptosis polypeptide, the second apoptosis polypeptide, the third apoptosis polypeptide, the first pyroptosis polypeptide, the second pyroptosis polypeptide, the first input polypeptide, and/or the second input polypeptide can comprise one or more linkers. In some embodiments, the linker: is a flexible linker, a rigid linker, or a hybrid linker; is hydrophilic or hydrophobic; is between 1 and 250 amino acids; comprises one or more flexible amino acid residues, e.g., about 1 to about 18 flexible amino acid residues. In some embodiments, the flexible amino acid residues comprise glycine, serine, or a combination thereof; and/or comprises 2 repeating amino acid subunits or more.

Any polypeptide of the disclosure can be configured to be localized at a particular location in a cell. In some embodiments: (i) the first, second, and/or third apoptosis polypeptide; (ii) the first and/or second pyroptosis polypeptide; and/or (iii) the first and/or second input polypeptide, are configured to be in a first localized state. In some embodiments, the first localized state comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first, second, and/or third apoptosis polypeptide, the first and/or second pyroptosis polypeptide, and/or the first and/or second input polypeptide is: (i) tethered to an intracellular organelle and/or membrane; or (ii) fused to a polypeptide that recruits to said localized state.

In some embodiments: (i) the first, second, and/or third apoptosis polypeptide; (ii) the first and/or second pyroptosis polypeptide; and/or (iii) the first and/or second input polypeptide, are configured to be in second localized state(s), wherein the first localized state and the second localized state(s) are different. In some embodiments, the second localized state(s) comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first, second, and/or third apoptosis polypeptide, the first and/or second pyroptosis polypeptide, and/or the first and/or second input polypeptide is: (i) tethered to an intracellular organelle and/or membrane; or (ii) fused to a polypeptide that recruits to said localized state.

In some embodiments: (i) the first, second, and/or third apoptosis polypeptide; (ii) the first and/or second pyroptosis polypeptide; and/or (iii) the first and/or second input polypeptide comprise a first localization signal. In some embodiments, the first localization signal is adjacent to a third degron and/or a third heterologous cleavage site. In some embodiments: (i) the first, second, and/or third apoptosis polypeptide; (ii) the first and/or second pyroptosis polypeptide; and/or (iii) the first and/or second input polypeptide comprise second localization signal(s). In some embodiments, the second localization signal is adjacent to a third degron and/or a third heterologous protease cleavage site.

The synthetic protein circuit can comprise a third input polypeptide comprising a third heterologous protease capable of cutting the third heterologous protease cleavage site. In some embodiments, the third heterologous protease comprises or is derived from one or more of a prokaryotic protease, bacterial protease, archaeal protease, eukaryotic protease, fungal protease, plant protease, mammalian protease, and human protease. In some embodiments, the third heterologous protease is engineered. In some embodiments, the third heterologous protease comprises tobacco etch virus (TEV) protease, tobacco vein mottling virus (TVMV) protease, hepatitis C virus protease (HCVP), derivatives thereof, or any combination thereof. In some embodiments, the third heterologous protease cleavage site comprises the sequence of SEQ ID NO: 1 or SEQ ID NO: 2.

In some embodiments, the third heterologous protease cleavage site is natural or engineered, e.g., the cleavage sequence of a human apoptotic effector protease, e.g., a caspase cleavage sequence. In some embodiments, the third heterologous protease cleavage site is configured to modulate an activation threshold of the synthetic protein circuit. In some embodiments, said configuring comprises introducing one or more amino acid insertions, substitutions, or deletions relative to a wild type third heterologous protease cleavage site. In some embodiments, the third heterologous protease cleavage site comprises the sequence of any one of SEQ ID NOs: 3-7.

In some embodiments, the first localization signal and/or second localization signal(s) is selected from the group comprising a nuclear localization signal (NLS), a nuclear export signal (NES), a peroxisomal targeting signal (PTS), a mitochondrial targeting sequence (MTS), a ER signal peptide, CAAX box, a peroxisomal targeting signal (PTS), PTS1, PTS2, a dileucine motif, YxxΦ motif, a palmitoylation motif, a GPI anchor signal, myristoylation signal, HDEL, KDEL, KKXX motif, RXR motif, SV40 NLS, SV40 NES, CAAX membrane tether, ER recruitment, portions thereof, derivatives thereof, or any combination thereof.

Any of the apoptosis polypeptides of the disclosure can comprise, in any order, a large subunit of an apoptotic effector protein and/or a large subunit of an apoptotic effector protein (or any portion or variant thereof), one or more heterologous protease cleavage sites, one or more degrons, one or more linkers, and/or one or more localization signals. Any of the pyroptosis polypeptides of the disclosure can comprise, in any order, a pyroptosis effector domain and/or an inhibitory domain (or any portion or variant thereof), one or more heterologous protease cleavage sites, one or more degrons, one or more linkers, and/or one or more localization signals. Any of the apoptosis and/or pyroptosis polypeptides can comprise one or more partner domains (e.g., first, second, third and/or fourth partner domains).

### Signal Transducers

A variety of signal transducers are contemplated herein. The first signal transducer can be capable of binding the first signal transducer binding domain and/or the second signal transducer can be capable of binding the second signal transducer binding domain following a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof. The first signal transducer, the second signal transducer, or both can be endogenous proteins. The first signal transducer, the second signal transducer, or both comprise AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCy, PLCy, NF-kB, FAK, CREB, αIIIβ3, FcεRI, BAD, p70S6K, STAT1, STAT2, STAT3, STAT5, STAT6, or any combination thereof.

The first signal transducer and/or the second signal transducer can be capable of regulating cell survival, cell growth, cell proliferation, cell adhesion, cell migration, cell metabolism, cell morphology, cell differentiation, apoptosis, or any combination thereof. The first signal transducer, the second signal transducer, or both can comprise a RAS protein (e.g., KRAS, NRAS, HRAS). The first signal transducer, the second signal transducer, or both can be exogenous proteins. In some embodiments, the synthetic protein circuit comprises the first signal transducer, the second signal transducer, or both. In some embodiments, the first signal transducer, the second signal transducer, or both comprise a lipid (e.g., a phospholipid, phosphatidylinositol 3-phosphate). The RAS protein can comprise one or more mutations. For example, the RAS protein (e.g., KRAS, NRAS, HRAS) can comprise a mutation at one or more of positions G12, G13, Q61, A146, in the RAS protein. In some embodiments, the mutation is G12A, G12C, G12D, G12R, G12R, G12S, G12V, G13C, G13D, G13dup, G13R, G13S, G13V, Q61H, Q61K, Q61L, Q61R, Q61E, Q61P, Q61*, A146P, A146T, and/or A146V.

Signal transducers can be can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 1-3.

TABLE 1
DISEASES AND DISORDERS OF INTEREST

| Diseases/Disorders | Genes |
|---|---|
| Neoplasia | PTEN; ATM; ATR; EGFR; ERBB2; ERBB3; ERBB4; Notch1; |
| | Notch2; Notch3; Notch4; AKT; AKT2; AKT3; HIF; HIF1a; HIF3a; |
| | Met; HRG; Bcl2; PPAR alpha; PPAR gamma; WT1 (Wilms Tumor); |
| | FGF Receptor Family members (5 members: 1, 2, 3, 4, 5); CDKN2a; |
| | APC; RB (retinoblastoma); MEN1; VHL; BRCA1; BRCA2; AR |
| | (Androgen Receptor); TSG101; IGF; IGF Receptor; Igf1 (4 variants); |
| | Igf2 (3 variants); Igf 1 Receptor; Igf 2 Receptor; Bax; Bcl2; caspases |
| | family (9 members: 1, 2, 3, 4, 6, 7, 8, 9, 12); Kras; Apc |
| Age-related Macular | Abcr; Ccl2; Cc2; cp (ceruloplasmin); Timp3; cathepsinD; Vldlr; Ccr2 |
| Degeneration | |
| Schizophrenia | Neuregulin1 (Nrg1); Erb4 (receptor for Neuregulin); Complexin1 |
| | (Cplx1); Tph1 Tryptophan hydroxylase; Tph2 Tryptophan hydroxylase |
| | 2; Neurexin 1; GSK3; GSK3a; GSK3b |
| Disorders | 5-HTT (Slc6a4); COMT; DRD (Drd1a); SLC6A3; DAOA; DTNBP1; |
| | Dao (Dao1) |
| Trinucleotide Repeat | HTT (Huntington's Dx); SBMA/SMAX1/AR (Kennedy's Dx); |
| Disorders | FXN/X25 (Friedrich's Ataxia); ATX3 (Machado- Joseph's Dx); |
| | ATXN1 and ATXN2 (spinocerebellar ataxias); DMPK (myotonic |
| | dystrophy); Atrophin-1 and Atn1 (DRPLA Dx); CBP (Creb-BP - |
| | global instability); VLDLR (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin (Ncstn); PEN-2 |
| Disorders | |
| Others | Nos1; Parp1; Nat1; Nat2 |
| Prion-related | Prp |
| disorders | |
| ALS | SOD1; ALS2; STEX; FUS; TARDBP; VEGF (VEGF-a; VEGF-b; |
| | VEGF-c) |
| Drug addiction | Prkce (alcohol); Drd2; Drd4; ABAT (alcohol); GRIA2; Grm5; Grin1; |
| | Htr1b; Grin2a; Drd3; Pdyn; Gria1 (alcohol) |
| Autism | Mecp2; BZRAP1; MDGA2; Sema5A; Neurexin 1; Fragile X (FMR2 |
| | (AFF2); FXR1; FXR2; Mglur5) |
| Alzheimer's Disease | E1; CHIP; UCH; UBB; Tau; LRP; PICALM; Clusterin; PS1; SORL1; |
| | CR1; Vldlr; Uba1; Uba3; CHIP28 (Aqp1, Aquaporin 1); Uchl1; Uchl3; |
| | APP |
| Inflammation | IL-10; IL-1 (IL-1a; IL-1b); IL-13; IL-17 (IL-17a (CTLA8); IL- 17b; |
| | IL-17c; IL-17d; IL-17f); II-23; Cx3cr1; ptpn22; TNFa; |
| | NOD2/CARD15 for IBD; IL-6; IL-12 (IL-12a; IL-12b); CTLA4; |
| | Cx3cl1 |
| Parkinson's Disease | x-Synuclein; DJ-1; LRRK2; Parkin; PINK1 |

TABLE 2
SIGNAL TRANSDUCERS

| | |
|---|---|
| Blood and | Anemia (CDAN1, CDA1, RPS19, DBA, PKLR, PK1, NT5C3, UMPH1, |
| coagulation | PSN1, RHAG, RH50A, NRAMP2, SPTB, ALAS2, ANH1, ASB, ABCB7, |
| diseases and | ABC7, ASAT); Bare lymphocyte syndrome (TAPBP, TPSN, TAP2, |
| disorders | ABCB3, PSF2, RING11, MHC2TA, C2TA, RFX5, RFXAP, RFX5); Bleeding |
| | disorders (TBXA2R, P2RX1, P2X1); Factor H and factor H-like 1 (HF1, |
| | CFH, HUS); Factor V and factor VIII (MCFD2); Factor VII deficiency |
| | (F7); Factor X deficiency (F10); Factor XI deficiency (F11); Factor XII |
| | deficiency (F12, HAF); Factor XIIIA deficiency (F13A1, F13A); Factor |
| | XIIIB deficiency (F13B); Fanconi anemia (FANCA, FACA, FA1, FA, FAA, |
| | FAAP95, FAAP90, FLJ34064, FANCB, FANCC, FACC, BRCA2, FANCD1, |
| | FANCD2, FANCD, FACD, FAD, FANCE, FACE, FANCF, XRCC9, |
| | FANCG, BRIP1, BACH1, FANCJ, PHF9, FANCL, FANCM, KIAA1596); |
| | Hemophagocytic lymphohistiocytosis disorders (PRF1, HPLH2, UNC13D, |
| | MUNC13-4, HPLH3, HLH3, FHL3); Hemophilia A (F8, F8C, HEMA); |
| | Hemophilia B (F9, HEMB), Hemorrhagic disorders (PI, ATT, F5); |
| | Leukocyde deficiencies and disorders (ITGB2, CD18, LCAMB, LAD, |
| | EIF2B1, EIF2BA, EIF2B2, EIF2B3, EIF2B5, LVWM, CACH, CLE, EIF2B4); |
| | Sickle cell anemia (HBB); Thalassemia (HBA2, HBB, HBD, LCRB, HBA1). |
| Cell | B-cell non-Hodgkin lymphoma (BCL7A, BCL7); Leukemia (TAL1 TCL5, |
| dysregulation | SCL, TAL2, FLT3, NBS1, NBS, ZNFN1A1, IK1, LYF1, HOXD4, HOX4B, |
| and oncology | BCR, CML, PHL, ALL, ARNT, KRAS2, RASK2, GMPS, AF10, |
| diseases and | ARHGEF12, LARG, KIAA0382, CALM, CLTH, CEBPA, CEBP, CHIC2, |
| disorders | BTL, FLT3, KIT, PBT, LPP, NPM1, NUP214, D9S46E, CAN, CAIN, |
| | RUNX1, CBFA2, AML1, WHSC1L1, NSD3, FLT3, AF1Q, NPM1, NUMA1, |
| | ZNF145, PLZF, PML, MYL, STAT5B, AF10, CALM, CLTH, ARL11, |
| | ARLTS1, P2RX7, P2X7, BCR, CML, PHL, ALL, GRAF, NF1, VRNF, WSS, |
| | NFNS, PTPN11, PTP2C, SHP2, NS1, BCL2, CCND1, PRAD1, BCL1, |
| | TCRA, GATA1, GF1, ERYF1, NFE1, ABL1, NQO1, DIA4, NMOR1, |
| | NUP214, D9S46E, CAN, CAIN). |
| Inflammation | AIDS (KIR3DL1, NKAT3, NKB1, AMB11, KIR3DS1, IFNG, CXCL12, |
| and immune | SDF1); Autoimmune lymphoproliferative syndrome (TNFRSF6, APT1, |
| related | FAS, CD95, ALPS1A); Combined immunodeficiency, (IL2RG, SCIDX1, |
| diseases and | SCIDX, IMD4); HIV-1 (CCL5, SCYA5, D17S136E, TCP228), HIV |
| disorders | susceptibility or infection (IL10, CSIF, CMKBR2, CCR2, CMKBR5, |
| | CCCKR5 (CCR5)); Immunodeficiencies (CD3E, CD3G, AICDA, AID, |
| | HIGM2, TNFRSF5, CD40, UNG, DGU, HIGM4, TNFSF5, CD40LG, |
| | HIGM1, IGM, FOXP3, IPEX, AIID, XPID, PIDX, TNFRSF14B, TACI); |
| | Inflammation (IL-10, IL-1 (IL-1a, IL-1b), IL-13, IL-17 (IL-17a (CTLA8), IL- |
| | 17b, IL-17c, IL-17d, IL-17f), II-23, Cx3cr1, ptpn22, TNFa, NOD2/CARD15 |
| | for IBD, IL-6, IL-12 (IL-12a, IL-12b), CTLA4, Cx3cl1); Severe combined |
| | immunodeficiencies (SCIDs)(JAK3, JAKL, DCLRE1C, ARTEMIS, SCIDA, |
| | RAG1, RAG2, ADA, PTPRC, CD45, LCA, IL7R, CD3D, T3D, IL2RG, |
| | SCIDX1, SCIDX, IMD4). |
| Metabolic, | Amyloid neuropathy (TTR, PALB); Amyloidosis (APOA1, APP, AAA, |
| liver, kidney | CVAP, AD1, GSN, FGA, LYZ, TTR, PALB); Cirrhosis (KRT18, KRT8, |
| and protein | CIRH1A, NAIC, TEX292, KIAA1988); Cystic fibrosis (CFTR, ABCC7, CF, |
| diseases and | MRP7); Glycogen storage diseases (SLC2A2, GLUT2, G6PC, G6PT, |
| disorders | G6PT1, GAA, LAMP2, LAMPB, AGL, GDE, GBE1, GYS2, PYGL, PFKM); |
| | Hepatic adenoma (TCF1, HNF1A, MODY3), Hepatic failure, early onset, |
| | and neurologic disorder (SCOD1, SCO1), Hepatic lipase deficiency (LIPC), |
| | Hepatoblastoma, cancer and carcinomas (CTNNB1, PDGFRL, PDGRL, |
| | PRLTS, AXIN1, AXIN, CTNNB1, TP53, P53, LFS1, IGF2R, MPRI, MET, |
| | CASP8, MCH5); Medullary cystic kidney disease (UMOD, HNFJ, FJHN, |
| | MCKD2, ADMCKD2); Phenylketonuria (PAH, PKU1, QDPR, DHPR, PTS); |
| | Polycystic kidney and hepatic disease (FCYT, PKHD1, ARPKD, PKD1, |
| | PKD2, PKD4, PKDTS, PRKCSH, G19P1, PCLD, SEC63). |
| Muscular/ | Becker muscular dystrophy (DMD, BMD, MYF6), Duchenne Muscular |
| Skeletal | Dystrophy (DMD, BMD); Emery-Dreifuss muscular dystrophy (LMNA, |
| diseases and | LMN1, EMD2, FPLD, CMD1A, HGPS, LGMD1B, LMNA, LMN1, EMD2, |
| disorders | FPLD, CMD1A); Facioscapulohumeral muscular dystrophy (FSHMD1A, |
| | FSHD1A); Muscular dystrophy (FKRP, MDC1C, LGMD2I, LAMA2, |
| | LAMM, LARGE, KIAA0609, MDC1D, FCMD, TTID, MYOT, CAPN3, |
| | CANP3, DYSF, LGMD2B, SGCG, LGMD2C, DMDA1, SCG3, SGCA, ADL, |
| | DAG2, LGMD2D, DMDA2, SGCB, LGMD2E, SGCD, SGD, LGMD2F, |
| | CMD1L, TCAP, LGMD2G, CMD1N, TRIM32, HT2A, LGMD2H, FKRP, |
| | MDC1C, LGMD2I, TTN, CMD1G, TMD, LGMD2J, POMT1, CAV3, |
| | LGMD1C, SEPN1, SELN, RSMD1, PLEC1, PLTN, EBS1); Osteopetrosis |
| | (LRP5, BMND1, LRP7, LR3, OPPG, VBCH2, CLCN7, CLC7, OPTA2, |
| | OSTM1, GL, TCIRG1, TIRC7, OC116, OPTB1); Muscular atrophy (VAPB, |
| | VAPC, ALS8, SMN1, SMA1, SMA2, SMA3, SMA4, BSCL2, SPG17, GARS, |
| | SMAD1, CMT2D, HEXB, IGHMBP2, SMUBP2, CATF1, SMARD1). |
| Neurological | ALS (SOD1, ALS2, STEX, FUS, TARDBP, VEGF (VEGF-a, VEGF-b, |
| and neuronal | VEGF-c); Alzheimer disease (APP, AAA, CVAP, AD1, APOE, AD2, |
| diseases and | PSEN2, AD4, STM2, APBB2, FE65L1, NOS3, PLAU, URK, ACE, DCP1, |
| disorders | ACE1, MPO, PACIP1, PAXIP1L, PTIP, A2M, BLMH, BMH, PSEN1, AD3); |
| | Autism (Mecp2, BZRAP1, MDGA2, Sema5A, Neurexin 1, GLO1, MECP2, |
| | RTT, PPMX, MRX16, MRX79, NLGN3, NLGN4, KIAA1260, AUTSX2); |
| | Fragile X Syndrome (FMR2, FXR1, FXR2, mGLUR5); Huntington's |
| | disease and disease like disorders (HD, IT15, PRNP, PRIP, JPH3, JP3, |
| | HDL2, TBP, SCA17); Parkinson disease (NR4A2, NURR1, NOT, TINUR, |
| | SNCAIP, TBP, SCA17, SNCA, NACP, PARK1, PARK4, DJ1, PARK7, |
| | LRRK2, PARK8, PINK1, PARK6, UCHL1, PARK5, SNCA, NACP, PARK1, |
| | PARK4, PRKN, PARK2, PDJ, DBH, NDUFV2); Rett syndrome (MECP2, |
| | RTT, PPMX, MRX16, MRX79, CDKL5, STK9, MECP2, RTT, PPMX, |
| | MRX16, MRX79, x-Synuclein, DJ-1); Schizophrenia (Neuregulin1 (Nrg1), |
| | Erb4 (receptor for Neuregulin), Complexin1 (Cplx1), Tph1 Tryptophan |
| | hydroxylase, Tph2, Tryptophan hydroxylase 2, Neurexin 1, GSK3, GSK3a, |
| | GSK3b, 5-HTT (Slc6a4), COMT, DRD (Drd1a), SLC6A3, DAOA, DTNBP1, |
| | Dao (Dao1)); Secretase Related Disorders (APH-1 (alpha and beta), |
| | Presenilin (Psen1), nicastrin, (Ncstn), PEN-2, Nos1, Parp1, Nat1, Nat2); |
| | Trinucleotide Repeat Disorders (HTT (Huntington's Dx), |
| | SBMA/SMAX1/AR (Kennedy's Dx), FXN/X25 (Friedrich's Ataxia), ATX3 |
| | (Machado- Joseph's Dx), ATXN1 and ATXN2 (spinocerebellar ataxias), |
| | DMPK (myotonic dystrophy), Atrophin-1 and Atn1 (DRPLA Dx), CBP (Creb- |
| | BP - global instability), VLDLR (Alzheimer's), Atxn7, Atxn10). |
| Occular | Age-related macular degeneration (Abcr, Ccl2, Cc2, cp (ceruloplasmin), |
| diseases and | Timp3, cathepsinD, Vldlr, Ccr2); Cataract (CRYAA, CRYA1, CRYBB2, |
| disorders | CRYB2, PITX3, BFSP2, CP49, CP47, CRYAA, CRYA1, PAX6, AN2, |
| | MGDA, CRYBA1, CRYB1, CRYGC, CRYG3, CCL, LIM2, MP19, CRYGD, |
| | CRYG4, BFSP2, CP49, CP47, HSF4, CTM, HSF4, CTM, MIP, AQP0, |
| | CRYAB, CRYA2, CTPP2, CRYBB1, CRYGD, CRYG4, CRYBB2, CRYB2, |
| | CRYGC, CRYG3, CCL, CRYAA, CRYA1, GJA8, CX50, CAE1, GJA3, |
| | CX46, CZP3, CAE3, CCM1, CAM, KRIT1); Corneal clouding and |
| | dystrophy (APOA1, TGFBI, CSD2, CDGG1, CSD, BIGH3, CDG2, |
| | TACSTD2, TROP2, M1S1, VSX1, RINX, PPCD, PPD, KTCN, COL8A2, |
| | FECD, PPCD2, PIP5K3, CFD); Cornea plana congenital (KERA, CNA2); |
| | Glaucoma (MYOC, TIGR, GLC1A, JOAG, GPOA, OPTN, GLC1E, FIP2, |
| | HYPL, NRP, CYP1B1, GLC3A, OPA1, NTG, NPG, CYP1B1, GLC3A); |
| | Leber congenital amaurosis (CRB1, RP12, CRX, CORD2, CRD, RPGRIP1, |
| | LCA6, CORD9, RPE65, RP20, AIPL1, LCA4, GUCY2D, GUC2D, LCA1, |
| | CORD6, RDH12, LCA3); Macular dystrophy (ELOVL4, ADMD, STGD2, |
| | STGD3, RDS, RP7, PRPH2, PRPH, AVMD, AOFMD, VMD2). |

TABLE 3
SIGNAL TRANSDUCTION PATHWAYS

| Pathway | Genes |
|---|---|
| PI3K/AKT Signaling | PRKCE; ITGAM; ITGA5; IRAK1; PRKAA2; EIF2AK2; PTEN; |
| | EIF4E; PRKCZ; GRK6; MAPK1; TSC1; PLK1; AKT2; IKBKB; |
| | PIK3CA; CDK8; CDKN1B; NFKB2; BCL2; PIK3CB; PPP2R1A; |
| | MAPK8; BCL2L1; MAPK3; TSC2; ITGA1; KRAS; EIF4EBP1; |
| | RELA; PRKCD; NOS3; PRKAA1; MAPK9; CDK2; PPP2CA; |
| | PIM1; ITGB7; YWHAZ; ILK, TP53; RAF1; IKBKG; RELB; |
| | DYRK1A; CDKN1A; ITGB1; MAP2K2; JAK1; AKT1; JAK2; |
| | PIK3R1; CHUK; PDPK1; PPP2R5C; CTNNB1; MAP2K1; |
| | NFKB1; PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; EIF2AK2; |
| | RAC1; RAP1A; TLN1; EIF4E; ELK1; GRK6; MAPK1; RAC2; |
| | PLK1; AKT2; PIK3CA; CDK8; CREB1; PRKCI; PTK2; FOS; |
| | RPS6KA4; PIK3CB; PPP2R1A; PIK3C3; MAPK8; MAPK3; |
| | ITGA1; ETS1; KRAS; MYCN; EIF4EBP1; PPARG; PRKCD; |
| | PRKAA1; MAPK9; SRC; CDK2; PPP2CA, PIM1; PIK3C2A; |
| | ITGB7; YWHAZ; PPP1CC; KSR1; PXN; RAF1; FYN; DYRK1A; |
| | ITGB1; MAP2K2; PAK4; PIK3R1; STAT3; PPP2R5C; MAP2K1; |
| | PAK3; ITGB3; ESR1; ITGA2; MYC; TTK; CSNK1A1; CRKL; |
| | BRAF; ATF4; PRKCA; SRF; STAT1; SGK |
| Glucocorticoid | RAC1; TAF4B; EP300; SMAD2; TRAF6; PCAF; ELK1; |
| Receptor | MAPK1; SMAD3; AKT2; IKBKB; NCOR2; UBE2I; PIK3CA; |
| Signaling | CREB1; FOS; HSPA5; NFKB2; BCL2; MAP3K14; STAT5B; |
| | PIK3CB; PIK3C3; MAPK8; BCL2L1; MAPK3; TSC22D3; |
| | MAPK10; NRIP1; KRAS; MAPK13; RELA; STAT5A; MAPK9; |
| | NOS2A; PBX1; NR3C1; PIK3C2A; CDKN1C; TRAF2; |
| | SERPINE1; NCOA3; MAPK14; TNF; RAF1; IKBKG; MAP3K7; |
| | CREBBP; CDKN1A; MAP2K2; JAK1; IL8; NCOA2; AKT1; |
| | JAK2; PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; IGF1; |
| Signaling | RAC1; RAP1A; E1F4E; PRKCZ; NRP1; NTRK2; ARHGEF7; |
| | SMO; ROCK2; MAPK1; PGF; RAC2; PTPN11; GNAS; AKT2; |
| | PIK3CA; ERBB2; PRKCI; PTK2; CFL1; GNAQ; PIK3CB; |
| | CXCL12; PIK3C3; WNT11; PRKD1; GNB2L1; ABL1; MAPK3; |
| | ITGA1, KRAS; RHOA; PRKCD; PIK3C2A; ITGB7; GLI2; PXN; |
| | VASP; RAF1; FYN; ITGB1; MAP2K2; PAK4; ADAM17; AKT1; |
| | PIK3R1; GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor | PRKCE; ITGAM; ROCK1; ITGA5; CXCR4; IRAK1; PRKAA2; |
| Signaling | EIF2AK2; RAC1; RAP1A; GRK6; ROCK2; MAPK1; PGF; |
| | RAC2; PTPN11; GNAS; PLK1; AKT2; DOK1; CDK8; CREB1; |
| | PTK2; CFL1; GNAQ; MAP3K14; CXCL12; MAPK8; GNB2L1; |
| | ABL1; MAPK3; ITGA1; KRAS; RHOA; PRKCD; PRKAA1; |
| | MAPK9; SRC; CDK2; PIM1; ITGB7; PXN; RAF1; FYN; |
| | DYRK1A; ITGB1; MAP2K2; PAK4, AKT1; JAK2; STAT3; |
| | ADAM10; MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; PRKAA2; |
| Signaling | EIF2AK2; RAC1; INS; ARHGEF7; GRK6; ROCK2; MAPK1; |
| | RAC2; PLK1; AKT2; PIK3CA; CDK8; PTK2; CFL1; PIK3CB; |
| | MYH9; DIAPH1; PIK3C3; MAPK8; F2R; MAPK3; SLC9A1; |
| | ITGA1; KRAS; RHOA; PRKCD; PRKAA1; MAPK9; CDK2; |
| | PIM1; PIK3C2A; ITGB7; PPP1CC; PXN; VIL2; RAF1; GSN; |
| | DYRK1A; ITGB1; MAP2K2; PAK4; PIP5K1A; PIK3R1; |
| | MAP2K1; PAK3; ITGB3; CDC42; APC; ITGA2; TTK; |
| | CSNK1A1; CRKL; BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKC1; HSPA5; REST; GNAQ; |
| | PIK3CB; PIK3C3; MAPK8; IGF1R; PRKD1; GNB2L1; BCL2L1; |
| | CAPN1; MAPK3; CASP8; HDAC2; HDAC7A; PRKCD; |
| | HDAC11; MAPK9; HDAC9; PIK3C2A; HDAC3; TP53; CASP9; |
| | CREBBP; AKT1; PIK3R1; PDPK1; CASP1; APAF1; FRAP1; |
| | CASP2; JUN; BAX; ATF4; AKT3; PRKCA; CLTC; SGK; |
| | HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; PRKCD; |
| | PRKAA1; MAPK9; CDK2; PIM1; TP53; TNF; RAF1; IKBKG; |
| | RELB; CASP9; DYRK1A; MAP2K2; CHUK; APAF1; MAP2K1; |
| | NFKB1; PAK3; LMNA; CASP2; BIRC2; TTK; CSNK1A1; |
| | BRAF; BAX; PRKCA; SGK; CASP3; BIRC3; PARP1 |
| B Cell Receptor | RAC1; PTEN; LYN; ELK1; MAPK1; RAC2; PTPN11; AKT2; |
| Signaling | IKBKB; PIK3CA; CREB1; SYK; NFKB2; CAMK2A; MAP3K14; |
| | PIK3CB; PIK3C3; MAPK8; BCL2L1; ABL1; MAPK3; ETS1; |
| | KRAS; MAPK13; RELA; PTPN6; MAPK9; EGR1; PIK3C2A; |
| | BTK; MAPK14; RAF1; IKBKG; RELB; MAP3K7; MAP2K2; |
| | AKT1; PIK3R1; CHUK; MAP2K1; NFKB1; CDC42; GSK3A; |
| | FRAP1; BCL6; BCL10; JUN; GSK3B; ATF4; AKT3; VAV3; |
| | RPS6KB1 |
| Leukocyte | ACTN4; CD44; PRKCE; ITGAM; ROCK1; CXCR4; CYBA; |
| Extravasation | RAC1; RAP1A; PRKCZ; ROCK2; RAC2; PTPN11; MMP14; |
| Signaling | PIK3CA; PRKCI; PTK2; PIK3CB; CXCL12; PIK3C3; MAPK8; |
| | PRKD1; ABL1; MAPK10; CYBB; MAPK13; RHOA; PRKCD; |
| | MAPK9; SRC; PIK3C2A; BTK; MAPK14; NOX1; PXN; VIL2; |
| | VASP; ITGB1; MAP2K2; CTNND1; PIK3R1; CTNNB1; |
| | CLDN1; CDC42; F11R; ITK; CRKL; VAV3; CTTN; PRKCA; |
| | MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; CAPN2; |
| | PIK3CA; PTK2; PIK3CB; PIK3C3; MAPK8; CAV1; CAPN1; |
| | ABL1; MAPK3; ITGA1; KRAS; RHOA; SRC; PIK3C2A; ITGB7; |
| | PPP1CC; ILK; PXN; VASP; RAF1; FYN; ITGB1; MAP2K2; |
| | PAK4; AKT1; PIK3R1; TNK2; MAP2K1; PAK3; ITGB3; |
| | CDC42; RND3; ITGA2; CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; PIK3CB; |
| | MAPK8; RIPK1; MAPK3; IL6ST; KRAS; MAPK13; IL6R; |
| | RELA; SOCS1; MAPK9; FTL; NR3C1; TRAF2; SERPINE1; |
| | MAPK14; TNF; RAF1; PDK1; IKBKG; RELB; MAP3K7; |
| | MAP2K2; AKT1; JAK2; PIK3R1; CHUK; STAT3; MAP2K1; |
| | NFKB1; FRAP1; CEBPB; JUN; AKT3; IL1R1; IL6 |
| PTEN Signaling | ITGAM; ITGA5; RAC1; PTEN; PRKCZ; BCL2L11; MAPK1; |
| | RAC2; AKT2; EGFR; IKBKB; CBL; PIK3CA; CDKN1B; PTK2; |
| | NFKB2; BCL2; PIK3CB; BCL2L1; MAPK3; ITGA1; KRAS; |
| | ITGB7; ILK; PDGFRB; INSR; RAF1; IKBKG; CASP9; |
| | CDKN1A; ITGB1; MAP2K2; AKT1; PIK3R1; CHUK; PDGFRA; |
| | PDPK1; MAP2K1; NFKB1; ITGB3; CDC42; CCND1; GSK3A; |
| | ITGA2; GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; PIK3CB; |
| | PIK3C3; MAPK8; THBS1; ATR; BCL2L1; E2F1; PMAIP1; |
| | CHEK2; TNFRSF10B; TP73; RB1; HDAC9; CDK2; PIK3C2A; |
| | MAPK14; TP53; LRDD; CDKN1A; HIPK2; AKT1; PIK3R1; |
| | RRM2B; APAF1; CTNNB1; SIRT1; CCND1; PRKDC; ATM; |
| | SFN; CDKN2A; JUN; SNAI2; GSK3B; BAX; AKT3 |
| Aryl Hydrocarbon | HSPB1; EP300; FASN; TGM2; RXRA; MAPK1; NQO1; |
| Receptor Signaling | NCOR2; SP1; ARNT; CDKN1B; FOS; CHEK1; SMARCA4; |
| | NFKB2; MAPK8; ALDH1A1; ATR; E2F1; MAPK3; NRIP1; |
| | CHEK2; RELA; TP73; GSTP1; RB1; SRC; CDK2; AHR; |
| | NFE2L2; NCOA3; TP53; TNF; CDKN1A; NCOA2; APAF1; |
| | NFKB1; CCND1; ATM; ESR1; CDKN2A; MYC; JUN; ESR2; |
| | BAX; IL6; CYP1B1; HSP90AA1 |
| Xenobiotic Metabolism | PRKCE; EP300; PRKCZ; RXRA; MAPK1; NQO1; NCOR2; |
| Signaling | PIK3CA; ARNT; PRKCI; NFKB2; CAMK2A; PIK3CB; |
| | PPP2R1A; PIK3C3; MAPK8; PRKD1; ALDH1A1; MAPK3; |
| | NRIP1; KRAS; MAPK13; PRKCD; GSTP1; MAPK9; NOS2A; |
| | ABCB1; AHR; PPP2CA; FTL; NFE2L2; PIK3C2A; PPARGC1A; |
| | MAPK14; TNF; RAF1; CREBBP; MAP2K2; PIK3R1; PPP2R5C; |
| | MAP2K1; NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; GRK6; |
| | MAPK1; GADD45A; RAC2; PLK1; AKT2; PIK3CA; FADD; |
| | CDK8; PIK3CB; PIK3C3; MAPK8; RIPK1; GNB2L1; IRS1; |
| | MAPK3; MAPK10; DAXX; KRAS; PRKCD; PRKAA1; MAPK9; |
| | CDK2; PIM1; PIK3C2A; TRAF2; TP53; LCK; MAP3K7; |
| | DYRK1A; MAP2K2; PIK3R1; MAP2K1; PAK3; CDC42; JUN; |
| | TTK; CSNK1A1; CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; ABCA1; |
| | GNAQ; NFKB2; MAP3K14; STAT5B; MAPK8; IRS1; MAPK3; |
| | KRAS; RELA; PRKAA1; PPARGC1A; NCOA3; MAPK14; |
| | INSR; RAF1; IKBKG; RELB; MAP3K7; CREBBP; MAP2K2; |
| | JAK2; CHUK; MAP2K1; NFKB1; TGFBR1; SMAD4; JUN; |
| | IL1R1; PRKCA; IL6; HSP90AA1; ADIPOQ |
| NF-KB Signaling | IRAK1; EIF2AK2; EP300; INS; MYD88; PRKCZ: TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; KRAS; |
| | RELA; PIK3C2A; TRAF2; TLR4: PDGFRB; TNF; INSR; LCK; |
| | IKBKG; RELB; MAP3K7; CREBBP; AKT1; PIK3R1; CHUK; |
| | PDGFRA; NFKB1; TLR2; BCL10; GSK3B; AKT3; TNFAIP3; |
| | IL1R1 |
| Neuregulin Signaling | ERBB4; PRKCE; ITGAM; ITGA5: PTEN; PRKCZ; ELK1; |
| | MAPK1; PTPN11; AKT2; EGFR; ERBB2; PRKCI; CDKN1B; |
| | STAT5B; PRKD1; MAPK3; ITGA1; KRAS; PRKCD; STAT5A; |
| | SRC; ITGB7; RAF1; ITGB1; MAP2K2; ADAM17; AKT1; |
| | PIK3R1; PDPK1; MAP2K1; ITGB3; EREG; FRAP1; PSEN1; |
| | ITGA2; MYC; NRG1; CRKL; AKT3; PRKCA; HSP90AA1; |
| | RPS6KB1 |
| Wnt & Beta catenin | CD44; EP300; LRP6; DVL3; CSNK1E; GJA1; SMO; AKT2; |
| Signaling | PIN1; CDH1; BTRC; GNAQ; MARK2; PPP2R1A; WNT11; SRC; |
| | DKK1; PPP2CA; SOX6; SFRP2; ILK; LEF1; SOX9; TP53; |
| | MAP3K7; CREBBP; TCF7L2; AKT1; PPP2R5C; WNT5A; |
| | LRP5; CTNNB1; TGFBR1; CCND1; GSK3A; DVL1; APC; |
| | CDKN2A; MYC; CSNK1A1; GSK3B; AKT3; SOX2 |
| Insulin Receptor | PTEN; INS; EIF4E; PTPN1; PRKCZ; MAPK1; TSC1; PTPN11; |
| Signaling | AKT2; CBL; PIK3CA; PRKCI; PIK3CB; PIK3C3; MAPK8; |
| | IRS1; MAPK3; TSC2; KRAS; EIF4EBP1; SLC2A4; PIK3C2A; |
| | PPP1CC; INSR; RAF1; FYN; MAP2K2; JAK1; AKT1; JAK2; |
| | PIK3R1; PDPK1; MAP2K1; GSK3A; FRAP1; CRKL; GSK3B; |
| | AKT3; FOXO1; SGK; RPS6KB1 |
| IL-6 Signaling | HSPB1; TRAF6; MAPKAPK2; ELK1; MAPK1; PTPN11; |
| | IKBKB; FOS; NFKB2: MAP3K14; MAPK8; MAPK3; MAPK10; |
| | IL6ST; KRAS; MAPK13; IL6R; RELA; SOCS1; MAPK9; |
| | ABCB1; TRAF2; MAPK14; TNF; RAF1; IKBKG; RELB; |
| | MAP3K7; MAP2K2; IL8; JAK2; CHUK; STAT3; MAP2K1; |
| | NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; PRKD1; |
| | MAPK10; RELA; PRKCD; MAPK9; ABCB1; TRAF2; TLR4; |
| | TNF; INSR; IKBKG; RELB; MAP3K7; IL8; CHUK; NR1H2; |
| | TJP2; NFKB1; ESR1; SREBF1; FGFR4; JUN; IL1R1; PRKCA; |
| | IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; YWHAZ, |
| | PXN; RAF1; CASP9; MAP2K2; AKT1; PIK3R1; PDPK1; |
| | MAP2K1; IGFBP2; SFN; JUN; CYR61; AKT3; FOXO1; SRF; |
| | CTGF; RPS6KB1 |
| NRF2-mediated | PRKCE; EP300; SOD2; PRKCZ; MAPK1; SQSTM1; NQO1; |
| Oxidative Stress | PIK3CA; PRKCI; FOS; PIK3CB; PIK3C3; MAPK8; PRKD1; |
| Response | MAPK3; KRAS; PRKCD; GSTP1; MAPK9; FTL; NFE2L2; |
| | PIK3C2A; MAPK14; RAF1; MAP3K7; CREBBP; MAP2K2; |
| | AKT1; PIK3R1; MAP2K1; PPIB; JUN; KEAP1; GSK3B; ATF4; |
| | PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; IGF1R; |
| | IL6R; RELA; TLR4; PDGFRB; TNF; RELB; IL8; PDGFRA; |
| | NFKB1; TGFBR1; SMAD4; VEGFA; BAX; IL1R1; CCL2; HGF; |
| | MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; NRIP1; |
| | KRAS; PPARG; RELA; STAT5A; TRAF2; PPARGC1A; |
| | PDGFRB; TNF; INSR; RAF1; IKBKG; RELB; MAP3K7; |
| | CREBBP; MAP2K2; CHUK; PDGFRA; MAP2K1; NFKB1; JUN; |
| | IL1R1; HSP90AA1 |
| Fc Epsilon RI Signaling | PRKCE; RAC1; PRKCZ; LYN; MAPK1; RAC2; PTPN11; AKT2; |
| | PIK3CA; SYK; PRKCI; PIK3CB; PIK3C3; MAPK8; PRKD1; |
| | MAPK3; MAPK10; KRAS; MAPK13; PRKCD; MAPK9; |
| | PIK3C2A; BTK; MAPK14; TNF; RAF1; FYN; MAP2K2; AKT1; |
| | PIK3R1; PDPK1; MAP2K1; AKT3; VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA, SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; MAPK1; |
| Metabolism | PLK1; AKT2; PIK3CA; CDK8; PIK3CB; PIK3C3; MAPK8; |
| | MAPK3; PRKCD; PRKAA1; MAPK9; CDK2; PIM1; PIK3C2A; |
| | DYRK1A; MAP2K2; PIP5K1A; PIK3R1; MAP2K1; PAK3; |
| | ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; PIK3R1; |
| | PDGFRA; STAT3; SPHK1; MAP2K1; MYC; JUN; CRKL; |
| | PRKCA; SRF; STAT1; SPHK2 |
| VEGF Signaling | ACTN4; ROCK1; KDR; FLT1; ROCK2; MAPK1; PGF; AKT2; |
| | PIK3CA; ARNT; PTK2; BCL2; PIK3CB; PIK3C3; BCL2L1; |
| | MAPK3; KRAS; HIF1A; NOS3; PIK3C2A; PXN; RAF1; |
| | MAP2K2; ELAVL1; AKT1; PIK3R1; MAP2K1; SFN; VEGFA; |
| | AKT3; FOXO1; PRKCA |
| Natural Killer Cell | PRKCE; RAC1; PRKCZ; MAPK1; RAC2; PTPN11; KIR2DL3; |
| Signaling | AKT2; PIK3CA; SYK; PRKCI; PIK3CB; PIK3C3; PRKD1; |
| | MAPK3; KRAS; PRKCD; PTPN6; PIK3C2A; LCK; RAF1; FYN; |
| | MAP2K2; PAK4; AKT1; PIK3R1; MAP2K1; PAK3; AKT3; |
| | VAV3; PRKCA |
| Cell Cycle: G1/S | HDAC4; SMAD3; SUV39H1; HDAC5; CDKN1B; BTRC; ATR; |
| Checkpoint Regulation | ABL1; E2F1; HDAC2; HDAC7A; RB1; HDAC11; HDAC9; |
| | CDK2; E2F2; HDAC3; TP53; CDKN1A; CCND1; E2F4; ATM; |
| | RBL2; SMAD4; CDKN2A; MYC; NRG1; GSK3B; RBL1; |
| | HDAC6 |
| T Cell Receptor | RAC1; ELK1; MAPK1; IKBKB; CBL; PIK3CA; FOS; NFKB2; |
| Signaling | PIK3CB; PIK3C3; MAPK8; MAPK3; KRAS; RELA, PIK3C2A; |
| | BTK; LCK; RAF1; IKBKG; RELB, FYN; MAP2K2; PIK3R1; |
| | CHUK; MAP2K1; NFKB1; ITK; BCL10; JUN; VAV3 |
| Death Receptor | CRADD; HSPB1; BID; BIRC4; TBK1; IKBKB; FADD; FAS; |
| Signaling | NFKB2; BCL2; MAP3K14; MAPK8; RIPK1; CASP8; DAXX; |
| | TNFRSF10B; RELA; TRAF2; TNF; IKBKG; RELB; CASP9; |
| | CHUK; APAF1; NFKB1; CASP2; BIRC2; CASP3; BIRC3 |
| FGF Signaling | RAC1; FGFR1; MET; MAPKAPK2; MAPK1; PTPN11; AKT2; |
| | PIK3CA; CREB1; PIK3CB; PIK3C3; MAPK8; MAPK3; |
| | MAPK13; PTPN6; PIK3C2A; MAPK14; RAF1; AKT1; PIK3R1; |
| | STAT3; MAP2K1; FGFR4; CRKL; ATF4; AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; ETS1; |
| | KRAS; RUNX1; PIM1; PIK3C2A; RAF1; MAP2K2; AKT1; |
| | JAK2; PIK3R1; STAT3; MAP2K1; CCND1; AKT3; STAT1 |
| Amyotrophic Lateral | BID; IGF1; RAC1; BIRC4; PGF; CAPNS1; CAPN2; PIK3CA; |
| Sclerosis Signaling | BCL2; PIK3CB; PIK3C3; BCL2L1; CAPN1; PIK3C2A; TP53; |
| | CASP9; PIK3R1; RAB5A; CASP1; APAF1; VEGFA; BIRC2; |
| | BAX; AKT3; CASP3; BIRC3 |
| JAK/Stat Signaling | PTPN1; MAPK1; PTPN11; AKT2; PIK3CA; STAT5B; PIK3CB; |
| | PIK3C3; MAPK3; KRAS; SOCS1; STAT5A; PTPN6; PIK3C2A; |
| | RAF1; CDKN1A; MAP2K2; JAK1; AKT1; JAK2; PIK3R1; |
| | STAT3; MAP2K1; FRAP1; AKT3; STAT1 |
| Nicotinate and | PRKCE; IRAK1; PRKAA2; EIF2AK2; GRK6; MAPK1; PLK1; |
| Nicotinamide | AKT2; CDK8; MAPK8; MAPK3; PRKCD; PRKAA1; PBEF1; |
| Metabolism | MAPK9; CDK2; PIM1; DYRK1A; MAP2K2; MAP2K1; PAK3; |
| | NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; STAT5B; |
| | PIK3CB; PIK3C3; MAPK8; MAPK3; KRAS; SOCS1; STAT5A; |
| | PIK3C2A; LCK; RAF1; MAP2K2; JAK1; AKT1; PIK3R1; |
| | MAP2K1; JUN; AKT3 |
| Synaptic Long Term | PRKCE; IGF1; PRKCZ; PRDX6; LYN; MAPK1; GNAS; PRKCI; |
| Depression | GNAQ; PPP2R1A; IGF1R; PRKD1; MAPK3; KRAS; GRN; |
| | PRKCD; NOS3; NOS2A; PPP2CA; YWHAZ; RAF1; MAP2K2; |
| | PPP2R5C; MAP2K1; PRKCA |
| Estrogen Receptor | TAF4B; EP300; CARM1; PCAF; MAPK1; NCOR2; SMARCA4; |
| Signaling | MAPK3; NRIP1; KRAS; SRC; NR3C1; HDAC3; PPARGC1A; |
| | RBM9; NCOA3; RAF1; CREBBP; MAP2K2; NCOA2; MAP2K1; |
| | PRKDC; ESR1; ESR2 |
| Protein Ubiquitination | TRAF6; SMURF1; BIRC4; BRCA1; UCHL1; NEDD4; CBL; |
| Pathway | UBE2I; BTRC; HSPA5; USP7; USP10; FBXW7; USP9X; |
| | STUB1; USP22; B2M; BIRC2; PARK2; USP8; USP1; VHL; |
| | HSP90AA1; BIRC3 |
| IL-10 Signaling | TRAF6; CCR1; ELK1; IKBKB; SP1; FOS; NFKB2; MAP3K14; |
| | MAPK8; MAPK13; RELA; MAPK14; TNF; IKBKG; RELB; |
| | MAP3K7; JAK1; CHUK; STAT3; NFKB1; JUN; IL1R1; IL6 |
| VDR/RXR Activation | PRKCE; EP300; PRKCZ; RXRA; GADD45A; HES1; NCOR2; |
| | SP1; PRKCI; CDKN1B; PRKD1; PRKCD; RUNX2; KLF4; YY1; |
| | NCOA3; CDKN1A; NCOA2; SPP1; LRP5; CEBPB; FOXO1; |
| | PRKCA |
| TGF-beta Signaling | EP300; SMAD2; SMURF1; MAPK1; SMAD3; SMAD1; FOS; |
| | MAPK8; MAPK3; KRAS; MAPK9; RUNX2; SERPINE1; RAF1; |
| | MAP3K7; CREBBP; MAP2K2; MAP2K1; TGFBR1; SMAD4; |
| | JUN; SMAD5 |
| Toll-like Receptor | IRAK1; EIF2AK2; MYD88; TRAF6; PPARA; ELK1; IKBKB; |
| Signaling | FOS; NFKB2; MAP3K14; MAPK8; MAPK13; RELA; TLR4; |
| | MAPK14; IKBKG; RELB; MAP3K7; CHUK; NFKB1; TLR2; |
| | JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; SRF; |
| | STAT1 |
| Neurotrophin/TRK | NTRK2; MAPK1; PTPN11; PIK3CA; CREB1; FOS; PIK3CB; |
| Signaling | PIK3C3; MAPK8; MAPK3; KRAS, PIK3C2A; RAF1; MAP2K2; |
| | AKT1; PIK3R1; PDPK1; MAP2K1; CDC42; JUN; ATF4 |
| FXR/RXR Activation | INS; PPARA; FASN; RXRA; AKT2; SDC1; MAPK8; APOB; |
| | MAPK10; PPARG; MTTP; MAPK9; PPARGC1A; TNF; |
| | CREBBP; AKT1; SREBF1; FGFR4; AKT3; FOXO1 |
| Synaptic Long Term | PRKCE; RAP1A; EP300; PRKCZ; MAPK1; CREB1; PRKCI; |
| Potentiation | GNAQ; CAMK2A; PRKD1; MAPK3; KRAS; PRKCD; PPP1CC; |
| | RAF1; CREBBP; MAP2K2; MAP2K1; ATF4; PRKCA |
| Calcium Signaling | RAP1A; EP300; HDAC4; MAPK1; HDAC5; CREB1; CAMK2A; |
| | MYH9; MAPK3; HDAC2; HDAC7A; HDAC11; HDAC9; |
| | HDAC3; CREBBP; CALR; CAMKK2; ATF4; HDAC6 |
| EGF Signaling | ELK1; MAPK1; EGFR; PIK3CA; FOS; PIK3CB; PIK3C3; |
| | MAPK8; MAPK3; PIK3C2A; RAF1, JAK1; PIK3R1; STAT3; |
| | MAP2K1; JUN; PRKCA; SRF; STAT1 |
| Hypoxia Signaling in the | EDN1; PTEN; EP300; NQO1; UBE2I; CREB1; ARNT; HIF1A; |
| Cardiovascular System | SLC2A4; NOS3; TP53; LDHA; AKT1; ATM; VEGFA; JUN; |
| | ATF4; VHL; HSP90AA1 |
| LPS/IL-1 Mediated | IRAK1; MYD88; TRAF6; PPARA; RXRA; ABCA1, MAPK8; |
| Inhibition of RXR | ALDH1A1; GSTP1; MAPK9; ABCB1; TRAF2; TLR4; TNF; |
| Function | MAP3K7; NR1H2; SREBF1; JUN; IL1R1 |
| LXR/RXR Activation | FASN; RXRA; NCOR2; ABCA1; NFKB2; IRF3; RELA; NOS2A; |
| | TLR4; TNF; RELB; LDLR; NR1H2; NFKB1; SREBF1; IL1R1; |
| | CCL2; IL6; MMP9 |
| Amyloid Processing | PRKCE; CSNK1E; MAPK1; CAPNS1; AKT2; CAPN2; CAPN1; |
| | MAPK3; MAPK13; MAPT; MAPK14; AKT1; PSEN1; |
| | CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; CHEK1; |
| Damage Checkpoint | ATR; CHEK2; YWHAZ; TP53; CDKN1A; PRKDC; ATM; SFN; |
| Regulation | CDKN2A |
| Nitric Oxide Signaling | KDR; FLT1; PGF; AKT2; PIK3CA; PIK3CB; PIK3C3; CAV1; |
| in the Cardiovascular | PRKCD; NOS3; PIK3C2A; AKT1; PIK3R1; VEGFA; AKT3; |
| System | HSP90AA1 |
| Purine Metabolism | NME2; SMARCA4; MYH9; RRM2; ADAR; EIF2AK4; PKM2; |
| | ENTPD1; RAD51; RRM2B; TJP2; RAD51C; NT5E; POLD1; |
| | NME1 |
| cAMP-mediated | RAP1A; MAPK1; GNAS; CREB1; CAMK2A; MAPK3; SRC; |
| Signaling | RAF1; MAP2K2; STAT3; MAP2K1; BRAF; ATF4 |
| Mitochondrial | SOD2; MAPK8; CASP8; MAPK10; MAPK9; CASP9; PARK7; |
| Dysfunction | PSEN1; PARK2; APP; CASP3 |
| Notch Signaling | HES1; JAG1; NUMB; NOTCH4; ADAM17; NOTCH2; PSEN1; |
| | NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; NT5E; |
| | POLD1; NME1 |
| Parkinson's Signaling | UCHL1; MAPK8; MAPK13; MAPK14; CASP9; PARK7; |
| | PARK2; CASP3 |
| Cardiac & Beta | GNAS; GNAQ; PPP2R1A; GNB2L1; PPP2CA; PPP1CC; |
| Adrenergic Signaling | PPP2R5C |
| Glycolysis/ | HK2; GCK; GPI; ALDH1A1; PKM2; LDHA; HK1 |
| Gluconeogenesis | |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRKIB |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid | PRDX6; GRN; YWHAZ; CYP1B1 |
| Metabolism | |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics by | |
| Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative Stress | |
| Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; Trpa1; |
| | Pomc; Cgrp; Crf; Pka; Era; Nr2b; TRPM5; Prkaca; Prkacb; |
| | Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; Wnt2b; |
| Neurology | Wnt3a; Wnt4; Wnt5a; Wnt6; Wnt7b; Wnt8b; Wnt9a; Wnt9b; |
| | Wnt10a; Wnt10b; Wnt16); beta-catenin; Dkk-1; Frizzled related |
| | proteins; Otx-2; Gbx2; FGF-8; Reelin; Dab1; unc-86 (Pou4fl or |
| | Brn3a); Numb; Reln |

### Signal Transducer Binding Domains

There are provided, in some embodiments, first and/or second signal transducer binding domains. The first and/or second signal transducer binding domains can be identical. The first and/or second signal transducer binding domains can be different. The first and/or second signal transducer binding domains each can be capable of binding molecules of the first signal transducer and/or the second signal transducer. In some embodiments, the first signal transducer and/or the second signal transducer belong to a signal transduction pathway.

The first and/or second signal transducer binding domain can comprise a RAS binding domain (RBD) and/or RAS association domain (RAD). In some embodiments the first and/or second signal transducer binding domain can comprise or be derived from a protein capable of binding RAS. Examples of proteins capable of binding RAS include, but are not limited to: AGO2, APBB1IP, APPL1, ARAF, ARL1, ARL2, ARRB1, ARRB2, BAIAP2, BCL2, BCL2L1, BRAF, BRAP, BSG, CALM1, CALM3, CALML3, CALML4, CALML5, CALML6, CNKSR1, CNKSR2, CSK, DAB2IP, EGFR, ERBIN, FGA, FGB, FGG, FN1, GRB2, HK1, IFNGR1, IL6, IQGAP1, ITGA2B, ITGB3, KSR1, KSR2, LGALS3, LYN, LZTR1, MAP2K1, MAP2K2, MAPK1, MAPK14, MAPK3, MAPKAP1, MARK2, MARK3, MBP, MSI2, MTOR, NCBP2AS2, NF1, NIBAN2, PDE4DIP, PDE6D, PDPK1, PEBP1, PIK3CA, PIK3CB, PIK3CD, PIK3R1, PIK3R2, PIP5K1A, PLCE1, PPIA, PRKCZ, PTGS2, RAF1, RALB, RALGDS, RAP1A, RAP1B, RAP1GDS1, RASA1, RASA2, RASA3, RASA4, RASAL1, RASAL2, RASAL3, RASSF1, RASSF2, RASSF5, RGL1, RGL3, RIN1, SHOC2, SOS1, SOS2, SPRED1, SPRED2, SPRED3, SRC, SYNGAP1, TIAM1, TLN1, VCL, VWF, or YWHAB.

In some embodiments, the first and/or second signal transducer binding domain comprises a lipid binding domain (e.g., a Pleckstrin homology (PH) domain). The first and/or second signal transducer binding domain can comprise a nanobody, a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), single-domain antibody (sdAb), or any combination thereof.

There are provided, in some embodiments, antigen-binding moieties (e.g., monobodies). In some embodiments, the first and/or second signal transducer binding domain comprise an antigen binding moiety. The antigen-binding moiety can be configured to bind any of the signal transducers contemplated herein, such as those listed in Tables 1-3. The antigen-binding moiety can be configured to bind a signal transducer in an active and/or inactive state as described herein.

Antigen-binding moieties can comprise antibodies, antibody fragments, and variants. In some embodiments, antibody fragments and variants may comprise antigen binding regions from intact antibodies. Examples of antibody fragments and variants may include, but are not limited to Fab, Fab′, F(ab′)2, and Fv fragments; diabodies; linear antibodies; single-chain antibody molecules such as single chain variable fragment (scFv); and multi specific antibodies formed from antibody fragments.

For the purposes herein, an “antibody” may comprise a heavy and light variable domain as well as an Fc region. As used herein, the term “native antibody” usually refers to a heterotetrameric glycoprotein of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end: the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FVV) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments based on comparisons with other antibodies can also be used. Determining residues that make up CDRs may include the use of numbering schemes including, but not limited to, those taught by Kabai, Chothia, and Honegger.

H and VL domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs has favored canonical structures with the exception of the CDR-H3, which comprises amino acid sequences that may be highly variable in sequence and length between antibodies resulting in a variety of three-dimensional structures in antigen-binding domains. In some cases, CDR-H3s may be analyzed among a panel of related antibodies to assess antibody diversity. Various methods of determining CDR sequences are known in the art and may be applied to known antibody sequences.

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a fl exible linker between the light chain variable domain and the heavy chain variable domain (to form a single chain Fv (scFv)) or through the introduction of a disulfide bridge between heavy and light chain variable domains.

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen. Using molecular genetics, two scFvs can be engineered in tandem into a single polypeptide, separated by a linker domain, called a “tandem scFv” (tascFv). Construction of a tascFv with genes for two different scFvs yields a “bispecific single-chain variable fragments” (bis-scFvs).

As used herein, the term “bispecific antibody” refers to an antibody capable of binding two different antigens. Such antibodies typically comprise regions from at least two different antibodies. As used herein, the term “diabody” refers to a small antibody fragment with two antigen-binding sites. Diabodies are functional bispecific single-chain antibodies (bscAb). Diabodies comprise a heavy chain variable domain VH connected to a light chain variable domain VL in the same polypeptide chain. By using a linker that is too short to allow pairing between the two domains on the same chain, the domains are forced to pair with the complementary domains of another chain and create two antigen-binding sites.

The term “intrabody” can refer to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling and cell division. In some embodiments, methods provided herein may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

The modifier “monoclonal” can indicate the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity. In one embodiment, the antibody may be a humanized full-length antibody.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multi-specific antibody variants (e.g., bispecific variants), and antibody fragments.

In some embodiments, the antigen-binding moieties provided herein comprise antibody mimetics (e.g., monobodies). As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affiiins, affitins, anticalins, avimers, Centyrins, DARPINSTM, Fynomers and Kunitz and domain peptides. In other embodiments, antibody mimetics may include one or more non-peptide regions.

In some embodiments, the antigen-binding moieties provided herein comprise multispecific antibodies that bind more than one epitope. As used herein, the terms “multibody” or “multispecific antibody” refer to an antibody wherein two or more variable regions bind to different epitopes. The epitopes may be on the same or different targets. In some embodiments, a multi-specific antibody is a “bispecific antibody” which recognizes two different epitopes on the same or different antigens. In one aspect, bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

In some embodiments, the antigen-binding moieties provided herein comprise antibodies comprising a single antigen-binding domain (e.g., nanobodies). These molecules are extremely small, with molecular weights approximately one-tenth of those observed for full-sized mAbs. Further antibodies may include “nanobodies” derived from the antigen-binding variable heavy chain regions (VHHs) of heavy chain antibodies found m camels and llamas, which lack light chains (Nelson, A. L., MAbs.2010. January-February; 2(1):77-83).

In some embodiments, the antibody may be “miniaturized”. Among the best examples of mAb miniaturization are the small modular immunopharmaceuticals (SMIPs) from Trubion Pharmaceuticals. These molecules, which can be monovalent or bivalent, are recombinant single-chain molecules containing one VL, one VH antigen-binding domain, and one or two constant “effector” domains, all connected by linker domains. One example of miniaturized antibodies is called “unibody” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo.

In some embodiments, the antigen-binding moieties provided herein comprise single-domain antibodies (sdAbs, or nanobodies) which are antibody fragment consisting of a single monomelic variable antibody domain. In some embodiments, it is able to bind selectively to a specific antigen (e.g., like a whole antibody). In one aspect, a sdAb may be a “Camel Ig or “camelid VHH”. As used herein, the term “camel Ig” refers to the smallest known antigen-binding unit of a heavy chain antibody (Koch-No lte, et al, FASEB J., 2007, 21: 3490-3498). A “heavy chain antibody” or a “camelid antibody” refers to an antibody that contains two VH domains and no light chains (Riechmann L. et al, J. Immunol. Methods, 1999, 231: 25-38; international patent publication NOs. WO 1994/04678 and WO 1994/025591; and U.S. Pat. No. 6,005,079). In another aspect, a sdAb may be a “immunoglobulin new antigen receptor” (IgNAR). As used herein, the term “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

In some embodiments, the antigen-binding moieties provided herein comprise intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. Sequences from donor antibodies may be used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form, a single chain antibody comprising the variable domains of the heavy and light chains joined by a flexible linker polypeptide, intrabodies typically lack disulfide bonds and are capable of modulating the expression or activity of target genes through their specific binding activity. Single chain intrabodies are often expressed from a recombinant nucleic acid molecule and engineered to be retained intracellulariy (e.g., retained in the cytoplasm, endoplasmic reticulum, or periplasm).

### Nucleic Acids

Disclosed herein include nucleic acid compositions. In some embodiments, the composition comprises: one or more polynucleotides encoding any of the synthetic protein circuits of the disclosure. In some embodiments, the one or more polynucleotides comprise: one or more first polynucleotides encoding a first apoptosis polypeptide, a first pyroptosis polypeptide, or a first polypeptide; one or more second polynucleotides encoding a second apoptosis polypeptide, a second pyroptosis polypeptide, or a second polypeptide; and/or one or more third polynucleotides encoding a third apoptosis polypeptide. In some embodiments, the nucleic acid composition comprises one or more polynucleotides encoding a first and/or second input polypeptide.

The nucleic acid can comprise at least one regulatory element for expression of the synthetic protein circuit. The nucleic acid can comprise a vector. In some embodiments, the vector can comprise a adenovirus vector, an adeno-associated virus vector, an Epstein-Barr virus vector, a Herpes virus vector, an attenuated HIV vector, a retroviral vector, a vaccinia virus vector, or any combination thereof. In some embodiments, the vector can comprise an RNA viral vector. In some embodiments, the vector can be derived from one or more negative-strand RNA viruses of the order Mononegavirales. In some embodiments, the vector can be a rabies viral vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Retroviral vectors can be “defective”, i.e. unable to produce viral proteins required for productive infection. Replication of the vector can require growth in the packaging cell line. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a synthetic protein circuit component) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

In some embodiments, the vectors can include a regulatory sequence that allows, for example, the translation of multiple proteins from a single mRNA. Non-limiting examples of such regulatory sequences include internal ribosome entry site (IRES) and 2A self-processing sequence. In some embodiments, the 2A sequence is a 2A peptide site from foot-and-mouth disease virus (F2A sequence). In some embodiments, the F2A sequence has a standard furin cleavage site. In some embodiments, the vector can also comprise regulatory control elements known to one of skill in the art to influence the expression of the RNA and/or protein products encoded by the polynucleotide within desired cells of the subject. In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of said intron. Another example of a regulatory element is a transcription termination signal and/or a polyadenylation sequences.

Expression control elements and promoters include those active in a particular tissue or cell type, referred to herein as a “tissue-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

In some embodiments, the nucleic acid comprises a promoter operably linked to a polynucleotide of the disclosure. In some embodiments, the promoter is capable of inducing the transcription of the polynucleotide. In some embodiments, the nucleic acid comprises one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). In some embodiments, the polynucleotide further comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the promoter comprises a ubiquitous promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof. In some embodiments, the promoter is an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter. In some embodiments, the promoter comprises a tissue-specific promoter and/or a lineage-specific promoter. In some embodiments, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. In some embodiments, the tissue specific promoter is a neuron-specific promoter. In some embodiments, the neuron-specific promoter comprises a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. In some embodiments, the tissue specific promoter is a muscle-specific promoter. In some embodiments, the muscle-specific promoter comprises a creatine kinase (MCK) promoter. In some embodiments, the promoter comprises an intronic sequence. In some embodiments, the promoter comprises a bidirectional promoter and/or an enhancer. In some embodiments, the enhancer is a CMV enhancer.

Expression control elements also can confer expression in a manner that is regulatable, that is, a signal or stimuli increases or decreases expression of the operably linked polynucleotide. A regulatable element that increases expression of the operably linked polynucleotide m response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

Any apoptosis polypeptide, pyroptosis polypeptide, input polypeptide, or other synthetic protein circuit component disclosed herein can be encoded on a single open reading frame, and wherein two or more of, e.g., a first apoptotis can be separated by one or more self-cleaving peptides. Any apoptosis polypeptide, pyroptosis polypeptide, input polypeptide, or other synthetic protein circuit component (e.g., a first apoptosis polypeptide and a first input polypeptide) disclosed herein can be encoded on a single transcript. In one, non-limiting example, wherein translations of, e.g., the first apoptosis polypeptide and the first input polypeptide can be each driven by a separate internal ribosome entry site. The sequences of the internal ribosome entry sites can be identical or different.

In some embodiments, the nucleic acid composition is configured to achieve relative levels of the first apoptotic polypeptide, the second apoptotic polypeptide, the third apoptotic polypeptide, the first input polypeptide, the second input polypeptide, the first polypeptide and/or the second polypeptide desired by a user. In some embodiments, the nucleic acid composition is configured to achieve relative levels of the first pyroptotic polypeptide, the second pyroptotic polypeptide, the first input polypeptide, the second input polypeptide, the first polypeptide and/or the second polypeptide desired by a user. In some embodiments, the expression of one or more of the first apoptotic polypeptide, the second apoptotic polypeptide, the third apoptotic polypeptide, first pyroptotic polypeptide, the second pyroptotic polypeptide, the first input polypeptide, and/or the second polypeptide is configured to be dosage invariant and/or robust to tissue tropism and stochastic expression. In some embodiments, the induction of apoptosis or the induction of pyroptosis can be tuned by adjusting the relative levels of the first apoptotic polypeptide, the second apoptotic polypeptide, the third apoptotic polypeptide, first pyroptotic polypeptide, the second pyroptotic polypeptide, the first input polypeptide, and/or the second polypeptide.

### Intercellular Operations

Also disclosed herein are methods and compositions for delivery of a synthetic protein circuit of the disclosure to a cell (e.g., a receiver cell). Disclosed herein include compositions. In some embodiments, the composition comprises: a first population of sender cells comprising: (i) one or more first polynucleotide(s) encoding a first apoptosis polypeptide comprising a large subunit of an apoptotic effector protein and a small subunit of an apoptotic effector protein separated by a first heterologous protease cleavage site, wherein two of the first apoptosis polypeptides are capable of forming a first apoptotic protein complex in a first apoptotic protein complex inactive state, wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, and wherein the first apoptotic protein complex in the first apoptotic protein complex active state is capable of inducing apoptosis in a cell; and (ii) a first vector; and a second population of sender cells comprising: (i) one or more second polynucleotide(s) encoding a first input polypeptide; and (ii) a second vector. In some embodiments, the first vector of the first population of sender cells and the second vector of the second population of sender cells are capable of delivering the one or more first polynucleotide(s) and the one or more second polynucleotide(s) to receiver cells. In some embodiments, the first apoptosis polypeptide and the first input polypeptide are expressed in the receiver cells, thereby the first apoptotic protein complex in the first apoptotic protein complex active state is capable of inducing apoptosis in the receiver cells.

Disclosed herein include compositions. In some embodiments, the composition comprises: a first population of sender cells comprising: (i) one or more first polynucleotide(s) encoding a first pyroptosis polypeptide comprising a pyroptosis effector domain and a first inhibitory domain separated by a first heterologous protease cleavage site, wherein the first inhibitory domain is capable of inhibiting activity of the pyroptosis effector domain, thereby the first pyroptosis polypeptide is in a first pyroptosis polypeptide inactive state, wherein a first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, wherein the first pyroptosis polypeptide in the first pyroptosis polypeptide active state is capable of inducing pyroptosis in a cell, and (ii) a first vector; and a second population of sender cells comprising: (i) one or more second polynucleotide(s) encoding a first input polypeptide comprising the first heterologous protease; and (ii) a second vector. In some embodiments, the first vector of the first population of sender cells and the second vector of the second population of sender cells are capable of delivering the one or more first polynucleotide(s) and the one or more second polynucleotide(s) to receiver cells. In some embodiments, the first pyroptosis polypeptide and the first input polypeptide are expressed in the receiver cells, thereby the first pyroptosis polypeptide in the first pyroptosis polypeptide active state is capable of inducing pyroptosis in the receiver cells.

Disclosed herein include compositions. In some embodiments, the composition comprises: a first population of sender cells comprising: (i) one or more first polynucleotide(s) encoding a first pyroptosis polypeptide comprising a pyroptosis effector domain and a first partner domain; and (ii) a first vector, wherein the first population of sender cells express a silencer polypeptide comprising a first inhibitory domain and a second partner domain capable of binding the first partner domain, and wherein the inhibitor domain of the silencer polypeptide is capable of inhibiting the first pyroptosis polypeptide when the first pyroptosis polypeptide associates with the silencer polypeptide via binding of the first partner domain and the second partner domain, thereby the first pyroptosis polypeptide is in a first pyroptosis polypeptide inactive state. In some embodiments, the first vector is capable of delivering the one or more first polynucleotide(s) to receiver cells. In some embodiments, the first pyroptosis polypeptide is expressed in the receiver cells, wherein the first pyroptosis polypeptide is capable of being in a first pyroptosis polypeptide active state, wherein the first pyroptosis polypeptide in the first pyroptosis polypeptide active state is capable of inducing pyroptosis in the receiver cells. In some embodiments, the receiver cells do not express the silencer polypeptide.

### Methods for Selective Cell Killing

Provided herein are methods of selectively killing a target cell. In some embodiments, the method comprises: expressing any of the synthetic protein circuits or any of the nucleic acid compositions of the disclosure in the target cell, wherein the synthetic protein circuit is configured to be responsive to a unique cell type and/or unique cell state of the target cell. In some embodiments, the first and/or second heterologous protease is configured to be in the first and/or second heterologous protease active state in response to the unique cell type and/or unique cell state of the target cell.

### Methods of Treating

Disclosed herein include methods of treating or preventing a disease or disorder in a subject in need thereof. In some embodiments, the method comprises: expressing any of the synthetic protein circuit, the synthetic protein circuit and/or the second synthetic protein circuit of the disclosure, in a cell of the subject.

In some embodiments, the method comprises: administering to the subject an effective amount of a nucleic acid composition or a composition disclosed herein, thereby treating or preventing the disease or disorder in the subject. In some embodiments, administering comprises: (i) isolating one or more cells from the subject; (ii) contacting said one or more cells with any of the nucleic acid compositions disclosed herein, thereby generating engineered cells, optionally the contacting comprises transfection; and (iii) administering the one or more engineered cells into a subject after the contacting step.

Any of the synthetic protein circuits, nucleic acid compositions, or methods can comprise a supplementary protein circuit. In some embodiments, the supplementary protein circuit comprises: a first polypeptide comprising an optional first supplementary domain and a first part of a first protease domain of a supplementary heterologous protease; a second polypeptide comprising a second supplementary domain and a second part of the first protease domain of the supplementary protease, wherein the first part of the first protease domain and the second part of the first protease domain have weak association affinity, and wherein the first part of the first protease domain and the second part of the first protease domain are capable of associating with each other to constitute the supplementary heterologous protease, optionally the first and/or second supplementary domain is a signal transducer binding domain, optionally the supplementary heterologous protease in a supplementary heterologous protease active state is capable of cutting (i) the first, second, or third apoptosis polypeptide at the first or second heterologous protease cleavage site, and/or (ii) the first or second pyroptosis polypeptide at the first or second heterologous protease cleavage site, further optionally when a first signal transducer and a second signal transducer are in close proximity at an association location the supplementary heterologous protease is the first or second heterologous protease.

### Pharmaceutical Compositions and Methods of Administration

Also disclosed herein are pharmaceutical compositions comprising one or more of the nucleic acids, vectors, and/or compositions provided herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioners.

The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Titers of vectors to be administered will vary depending, for example, on the particular viral vector, the mode of administration, the treatment goal, the individual, and the cell type(s) being targeted, and can be determined by methods standard in the art.

As will be readily apparent to one skilled in the art, the useful in vivo dosage of the nucleic acids, vectors, and/or compositions to be administered and the particular mode of administration will vary depending upon the age, weight, the severity of the affliction, and animal species treated, the particular IFFL that is used, and the specific use for which the IFFL is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. Dosages of nucleic acids, vectors, and/or compositions provided can depend primarily on factors such as the condition being treated, the age, weight and health of the patient, and may thus vary among patients. For example, a therapeutically effective human dosage of the viral vector is generally in the range of from about 0.1 ml to about 100 ml of solution containing concentrations of from about 1×10⁹ to 1×10¹⁶ genomes virus viral. A preferred human dosage can be about 1×10¹³ to 1×10¹⁶ viral vector genomes. The dosage of a nucleic acid can be, e.g., 0.01-5 mg/kg total nucleic acid per administration, including 0.01 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, or 5 mg/kg, or a number or a range between any two of these values. The dosage will be adjusted to balance the therapeutic benefit against any side effects and such dosages may vary depending upon the therapeutic application for which the recombinant vector is employed. The levels of expression of a nucleic acid can be monitored to determine the amount and/or frequency of dosage resulting from the viral vector in some embodiments.

Nucleic acids, vectors, and/or compositions disclosed herein can be administered to a subject (e.g., a human) in need thereof. The route of the administration is not particularly limited. For example, a therapeutically effective amount of nucleic acids, vectors, and/or compositions can be administered to the subject by via routes standard in the art. Route(s) of administration can be readily determined by one skilled in the art taking into account the infection and/or disease state being treated and the target cells/tissue(s) that are to express the a synthetic protein circuit component.

The administering can comprise systemic administration (e.g., intravenous, intramuscular, intraperitoneal, or intraarticular). Administering can comprise intrathecal administration, intracranial injection, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal

Nucleic acids, vectors, and/or compositions to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of nucleic acids, vectors, and/or compositions expressing the therapeutic protein is administered to a host in need of such treatment. The use of the nucleic acids, vectors, and/or compositions provided herein in the manufacture of a medicament for inducing immunity in, or providing gene therapy to, a host is within the scope of the present application.

A therapeutically effective amount of the nucleic acids, vectors, and/or compositions provided herein can be administered to a subject at various points of time. For example, the nucleic acids, vectors, and/or compositions provided herein can be administered to the subject prior to, during, or after the subject has developed a disease, disorder, and/or infection. The nucleic acids, vectors, and/or compositions provided herein can also be administered to the subject prior to, during, or after the occurrence of a disease, disorder, and/or infection. In some embodiments, the nucleic acids, vectors, and/or compositions provided herein are administered to the subject during remission of the disease or disorder. In some embodiments, the nucleic acids, vectors, and/or compositions provided herein are administered prior to the onset of the disease or disorder in the subject. In some embodiments, nucleic acids, vectors, and/or compositions provided herein are administered to a subject at a risk of developing the disease or disorder.

The dosing frequency of the nucleic acids, vectors, and/or compositions provided herein can vary. For example, nucleic acids, vectors, and/or compositions provided herein can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the nucleic acids, vectors, and/or compositions provided herein are administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

### EXAMPLES

Some aspects of the embodiments discussed above are disclosed in further detail in the following examples, which are not in any way intended to limit the scope of the present disclosure.

### Example 1

### Synpoptosis Circuits for Programmable Control of Mammalian Cell Death

Provided herein are synthetic protein-level cell death circuits, collectively termed “synpoptosis” circuits. To engineer these circuits, inspiration was taken from natural cell death pathways that use regulated proteolysis along with protein-level caging and degradation mechanisms. Synpoptosis circuits provide a foundation for rationally designed, programmable control of mammalian cell death.

### Synpoptosis Circuits Control User-Selectable Cell Death Programs

Due to the inherent propensity of cells towards apoptosis or pyroptosis, natural inducers do not completely direct the mode of cell death. For example, when cytotoxic lymphocytes deliver granzymes into target cells, target cells lacking GSDMs undergo apoptosis, whereas those expressing functional GSDMs undergo pyroptosis. This lack of control allows target cells to activate undesirable death programs that favor their own survival or trigger systemic toxicity.

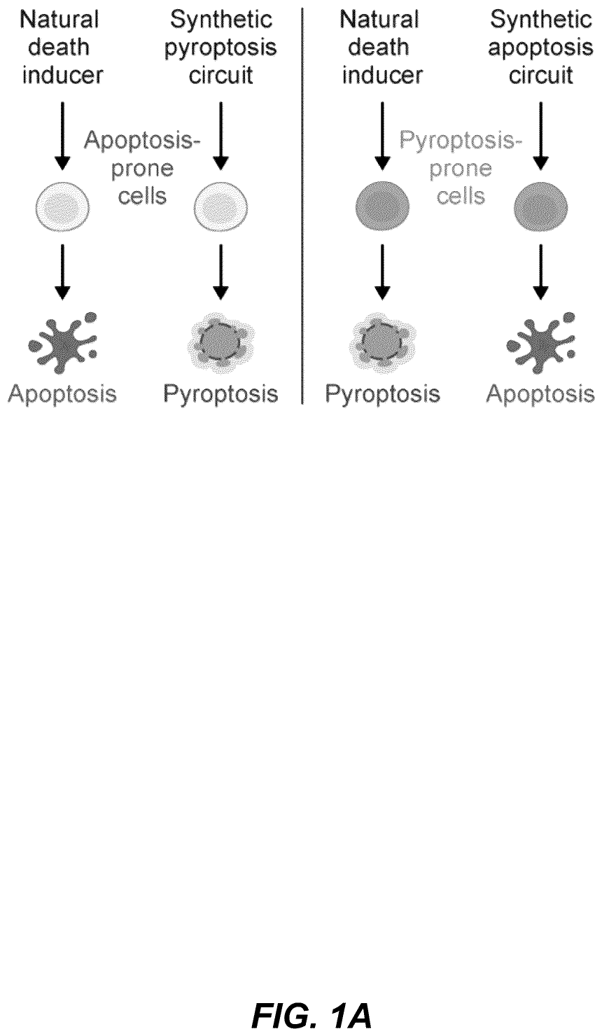

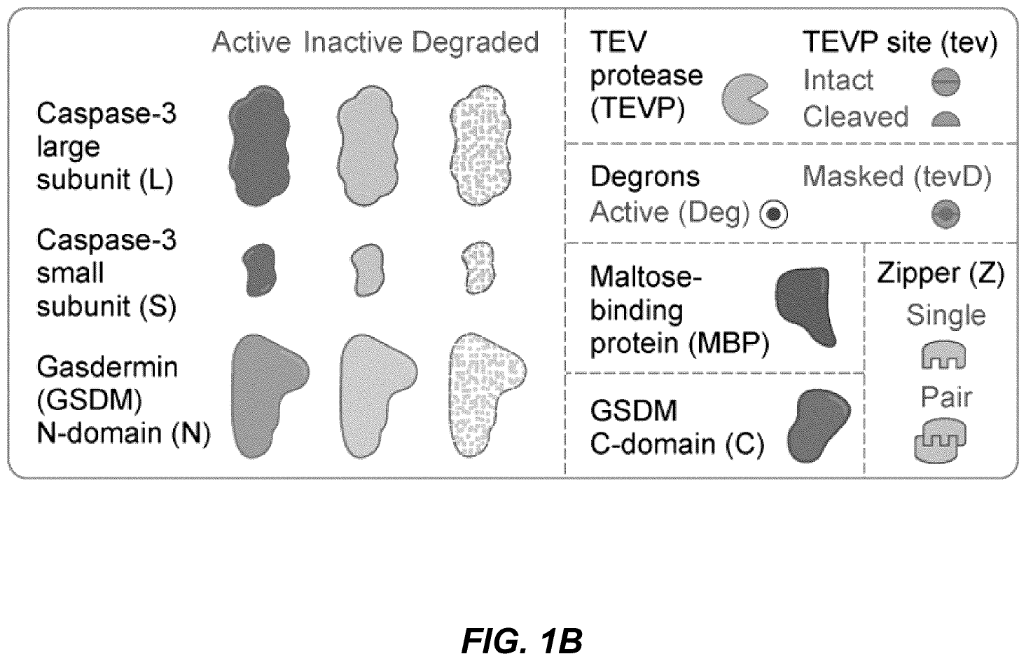

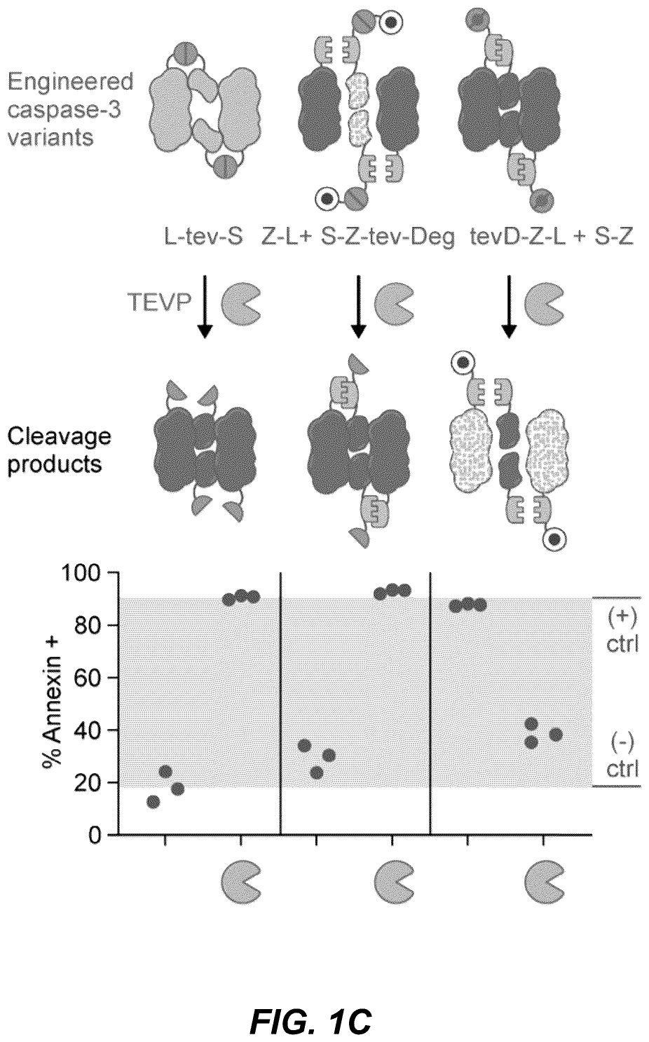

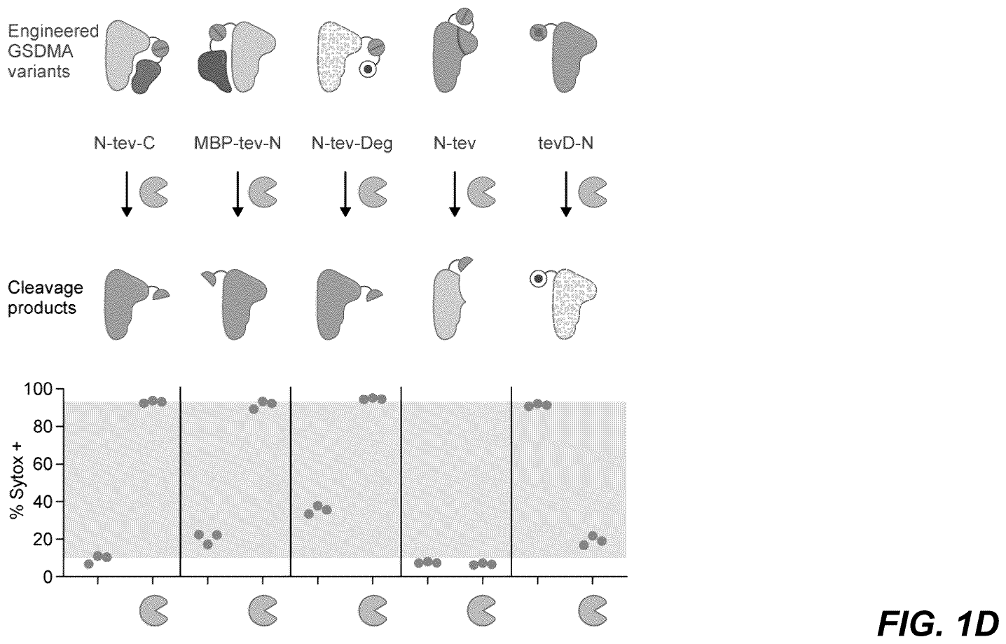

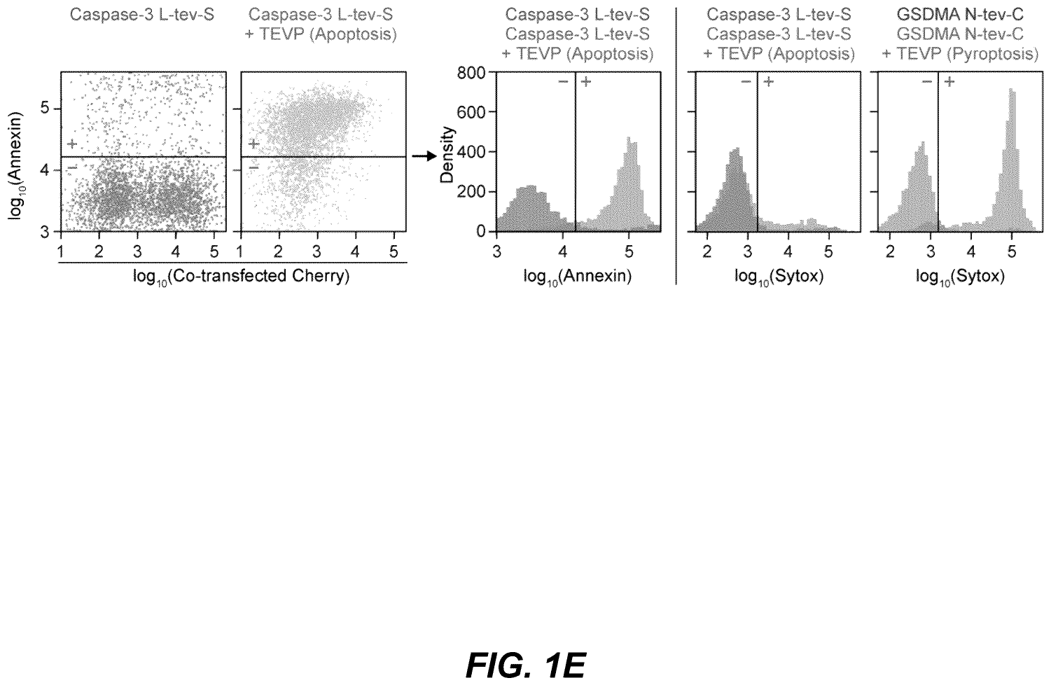

To design synpoptosis circuits capable of steering the mode of cell death (FIG. 1A), inspiration was drawn from naturally occurring cell death programs. Caspases, as proteases, contribute to apoptosis by activating the caspase-activated DNase and the XKR8 phospholipid scramblase, among other pro-apoptotic factors. Caspases can also cleave GSDMs for pore formation in pyroptosis. Additionally, in synthetic biology, proteases represent a “common currency”, as they can be engineered to perform various signal-processing tasks. Therefore, proteolysis is a promising mode of synthetic regulation of cell death (FIG. 1B-FIG. 1D).

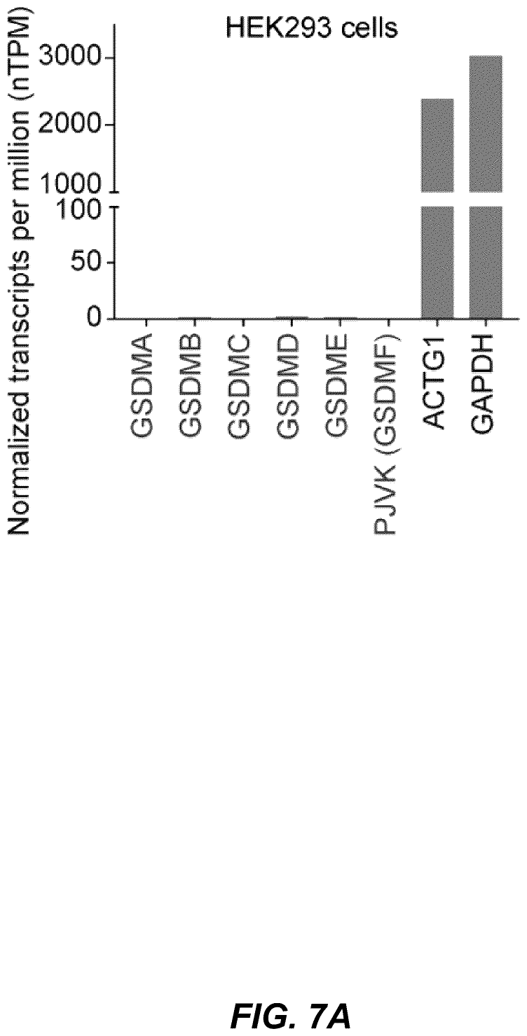

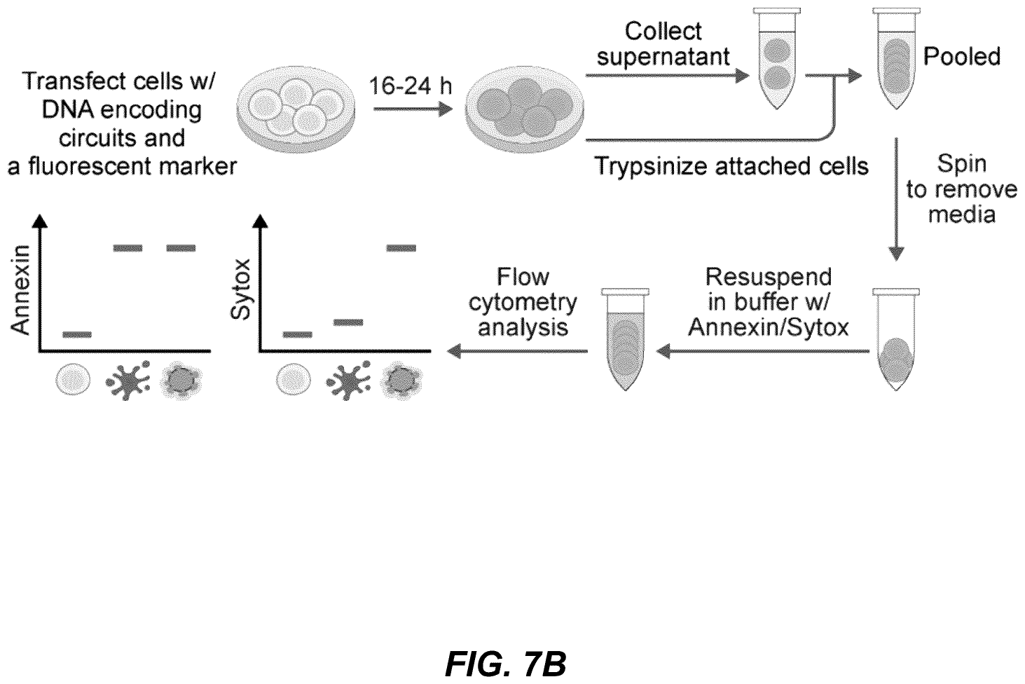

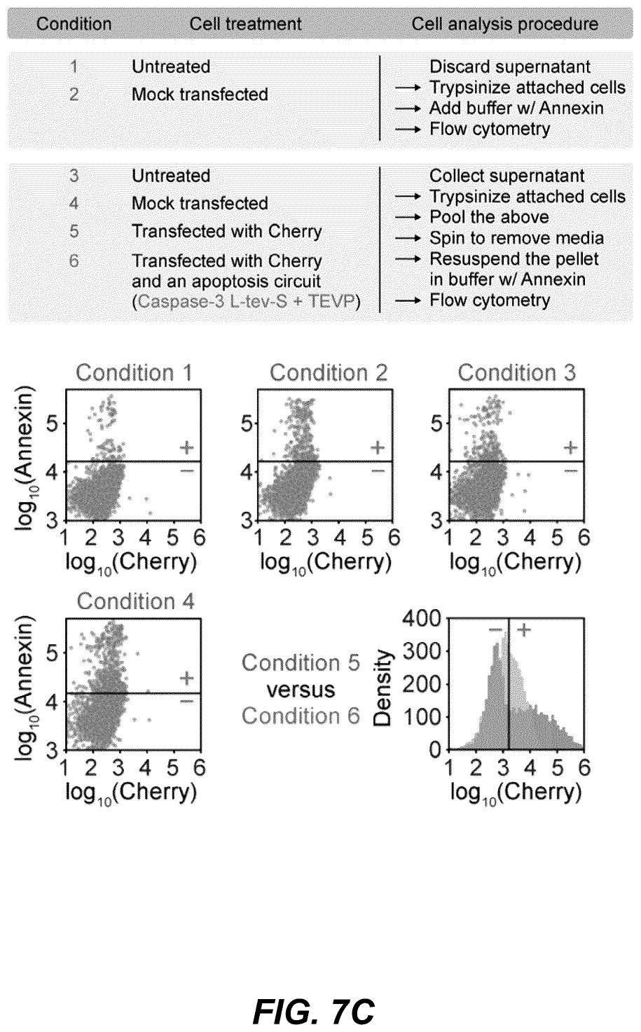

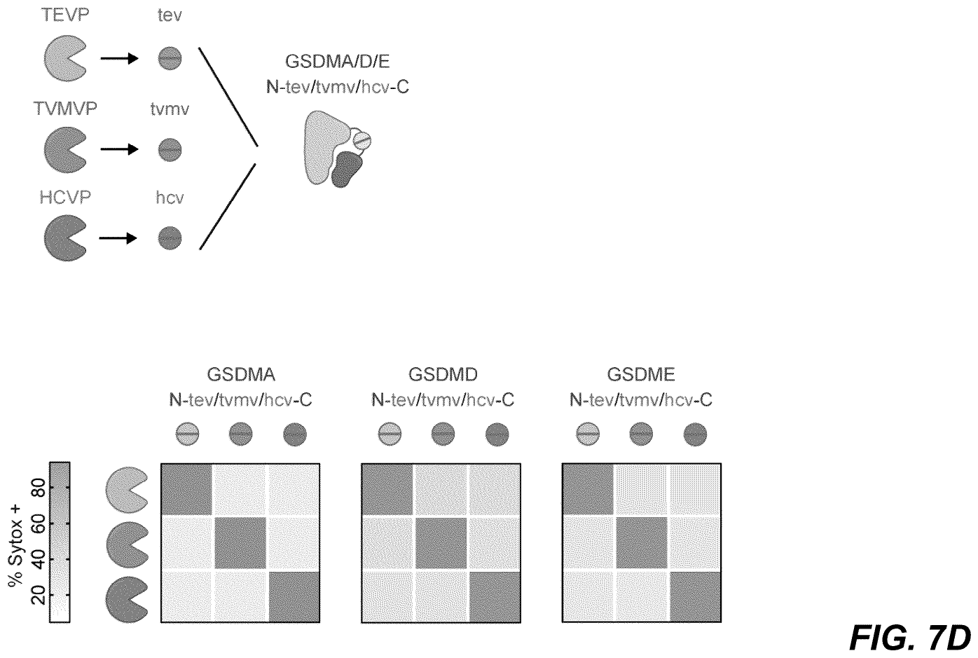

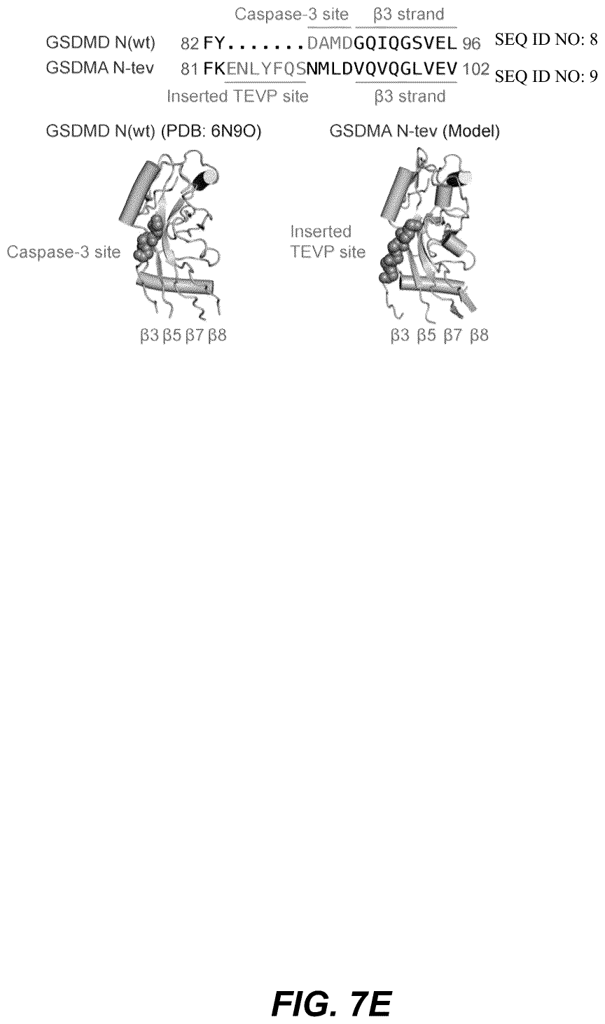

In most experiments, human embryonic kidney 293 (HEK) cells were used because they express low levels of endogenous GSDMs, providing a clear background for synthetically induced cell death (FIG. 7A). HEK cells were transiently co-transfected with DNA encoding the circuits, often along with Cherry, a fluorescent protein marker, and cells were analyzed by flow cytometry 16 to 24 hours later. To quantify cell death, the cells were strained with Annexin, Sytox, or both. Annexin stains apoptotic cells by binding to phosphatidylserine exposed on the outer leaflet, and pyroptotic cells by binding to phosphatidylserine in the inner leaflet after membrane permeabilization. In contrast, Sytox, a membrane-impermeable dye, primarily stains pyroptotic cells. Therefore, apoptotic cells are typically Annexin-high and Sytox-low, while pyroptotic cells are Annexin-high and Sytox-high (FIG. 7B). For simplicity, in experiments involving only a single death mode, a single relevant dye was often used. To focus on cells influenced by the circuits, the fraction of Annexin- or Sytox-positive cells was calculated after gating on the fluorescent co-transfection marker (FIG. 7C).

Previous work showed that the tobacco etch virus protease (TEVP) can activate a modified caspase-3 whose natural cleavage site between its large and small subunits was replaced by a TEVP cleavage site. Using Annexin staining, the ability of the modified caspase-3 to kill HEK cells when co-transfected with TEVP was verified (FIG. 1C, first module). Further, the cells only modestly took up Sytox, suggesting apoptosis (FIG. 1E).

To enable more complex functions for control of apoptosis downstream, additional caspase-3 variants that can be modulated by TEVP cleavage were engineered. First, the large and small subunits of caspase-3 were fused to heterodimerizing leucine zippers to produce a constitutively active non-covalent complex. Then, a TEVP-removable dihydrofolate reductase degron was appended to the small subunit. This degron is constitutively active, substantially reducing caspase-3 activity in the absence of TEVP. When TEVP was expressed, proteolytic removal of the degron efficiently activated apoptosis (FIG. 1C, second module). To enable deactivation of apoptosis through proteolysis, a TEVP site-caged N-degron was fused to the large subunit of caspase-3. The caged N-degron is inactive because its N-terminal destabilizing residue, a tyrosine, is preceded by the TEVP site sequence. Upon TEVP cleavage, the destabilizing residue is exposed, activating the N-degron. As such, TEVP cleavage repressed apoptosis (FIG. 1C, third module).

Next, proteolytic control of pyroptosis was engineered. The cleavage sites of TEVP, tobacco vein mottling virus protease (TVMVP), and hepatitis C virus protease (HCVP) were inserted into a linker region between the N-terminal (pore-forming) and C-terminal (auto-inhibitory) domains of three mammalian GSDMs. Each engineered GSDM effectively triggered pyroptosis in HEK cells in the presence of the cognate protease, as shown by Sytox staining (FIG. 1D, first module, FIG. 1E, FIG. 7D). GSDMA was focused on in subsequent experiments because it is thought to be orthogonal to endogenous host pathways.

As with caspase-3, additional GSDM variants were designed that allow positive and negative control over pyroptosis. GSDM structures showed that the N-termini of GSDMs need to be accessible to lipids for pore formation. Therefore, the activity of the GSDMA N-terminal domain was caged by blocking its N-terminus with a bulky maltose-binding protein. Insertion of a TEVP cleavage site allowed proteolytic removal of the bulky tag to restore pore-forming activity (FIG. 1D, second module). In a complementary approach, we fused a constitutively active degron to the GSDMA N-terminal domain to lower its activity. In this configuration, removal of the degron by TEVP also induced pyroptosis (FIG. 1D, third module). To proteolytically switch off GSDM activity, a natural mechanism was first mimicked in which the GSDMD N-terminal domain is inactivated through cleavage by caspase-3. However, inserting the TEVP cleavage site at an equivalent location within GSDMA abolished its activity (FIG. 1D, fourth module). As an alternative strategy, the GSDMA N-terminal domain was fused to a TEVP site-caged N-degron. This design allowed protease-mediated suppression of pyroptosis (FIG. 1D, fifth module). It is noted that cells also naturally use loop architectures to downregulate GSDM activity. These loops are at present challenging to construct synthetically, but can also provide synpoptosis circuit designs that fine-tune the penetrance of cell death.

Together, these results demonstrate that synthetic regulated proteolysis circuit modules can bidirectionally control apoptosis and pyroptosis.

### Synpoptosis Circuits Lead to Canonical Features of Cell Death

Cell death induced by synpoptosis circuits exhibited Annexin and Sytox staining patterns indicative of apoptosis and pyroptosis. To further characterize circuit-induced cell death, described herein are additional canonical features associated with naturally occurring cell death.

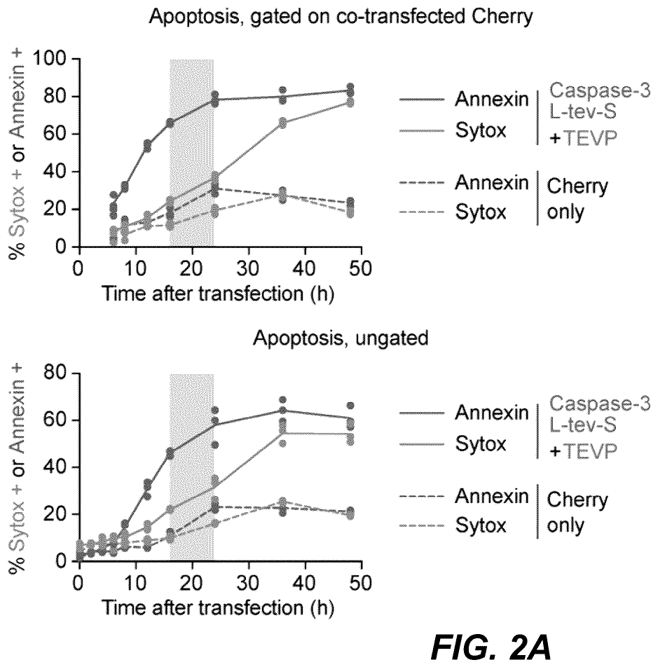

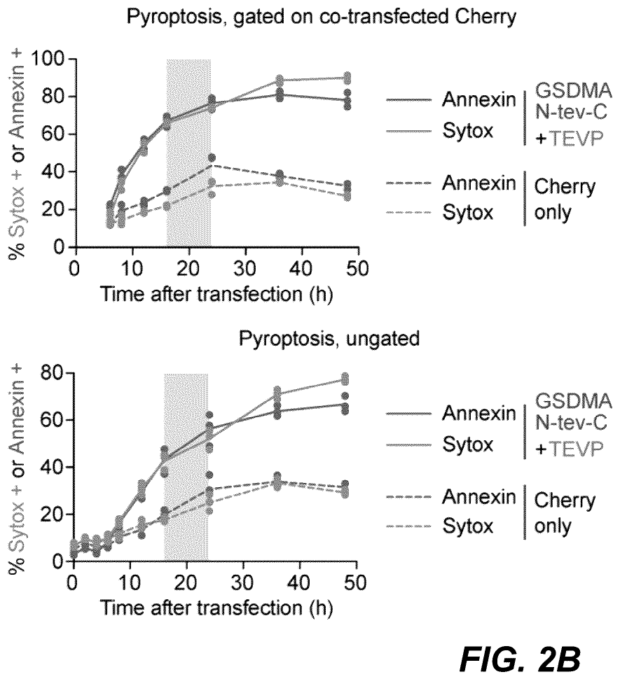

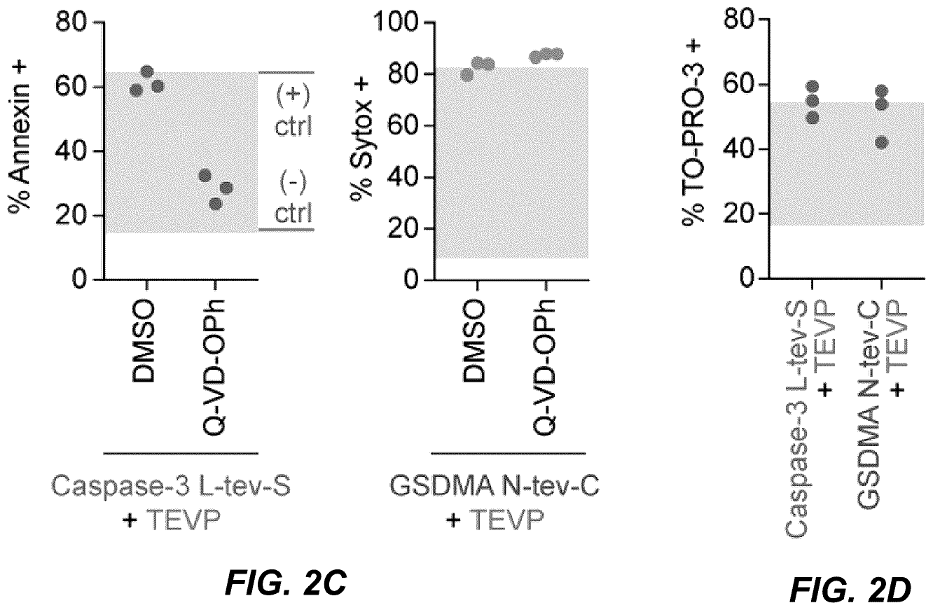

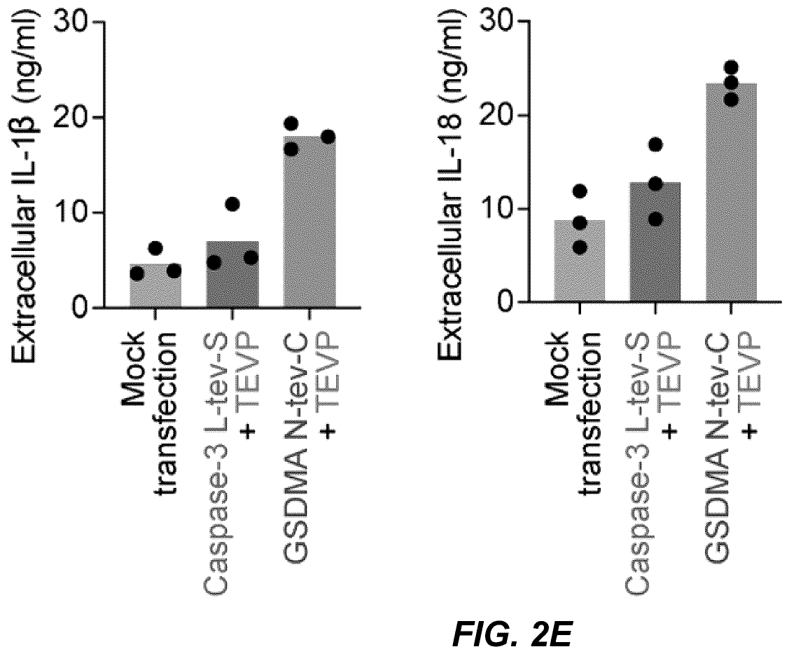

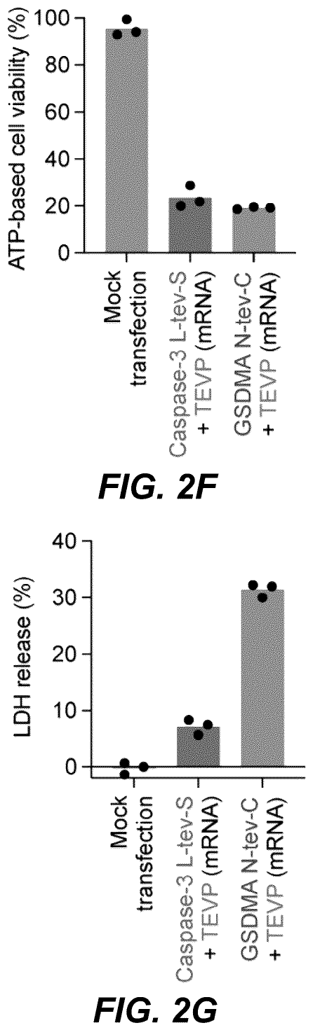

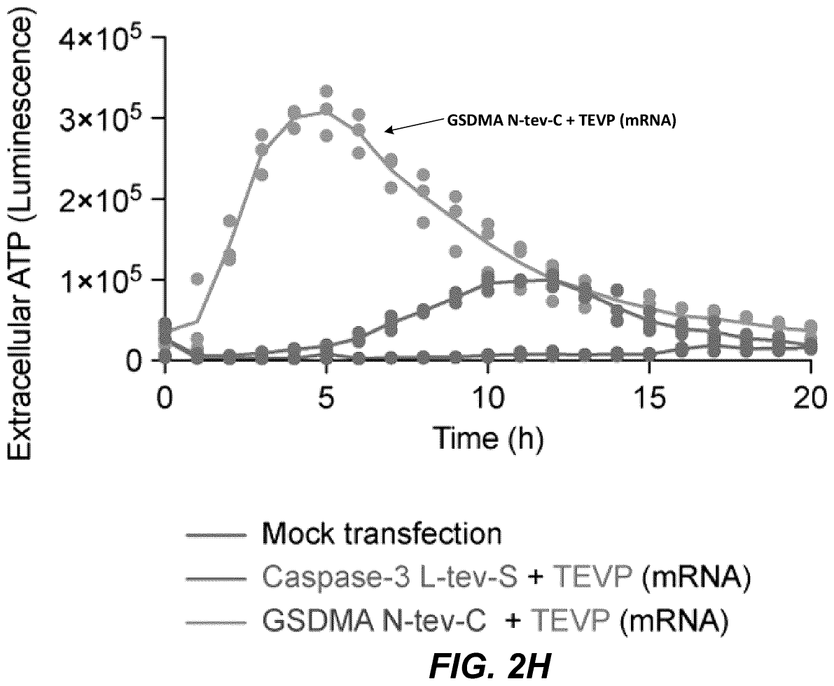

Drugs and death ligands typically induce cell death asynchronously within a cell population. To assess whether synpoptosis is similarly asynchronous, HEK cells were transfected with TEVP-activated caspase-3 and GSDMA circuits, and then cells were double-stained with Annexin and Sytox for flow cytometry analysis over a time span. Both synthetic apoptosis and pyroptosis exhibited asynchrony (FIG. 2A-FIG. 2B). In the case of apoptosis, Annexin-positivity preceded Sytox-positivity, indicating a sequence of apoptosis followed by eventual cell lysis (FIG. 2A). In contrast, the pyroptotic cells displayed a simultaneous increase in Annexin and Sytox signals (FIG. 2B). Similar dynamic patterns were observed without gating on the co-transfected Cherry, demonstrating robust circuit effects on a population level (FIG. 2A-FIG. 2B). Because the apoptotic cells eventually progress to cell lysis, flow cytometry analysis was confined to a window between 16 and 24 hours post-transfection to distinguish between apoptosis and pyroptosis (FIG. 7B).

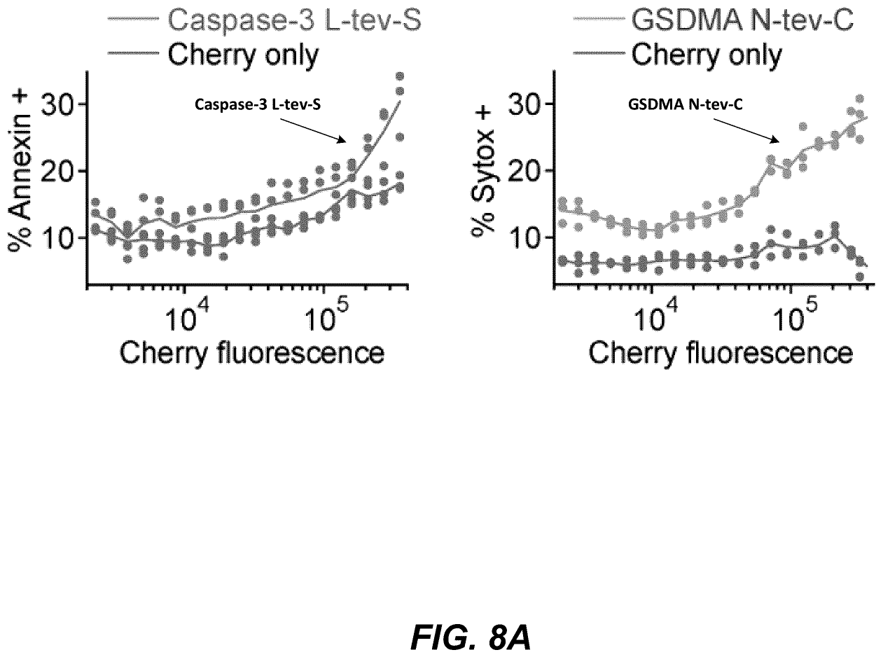

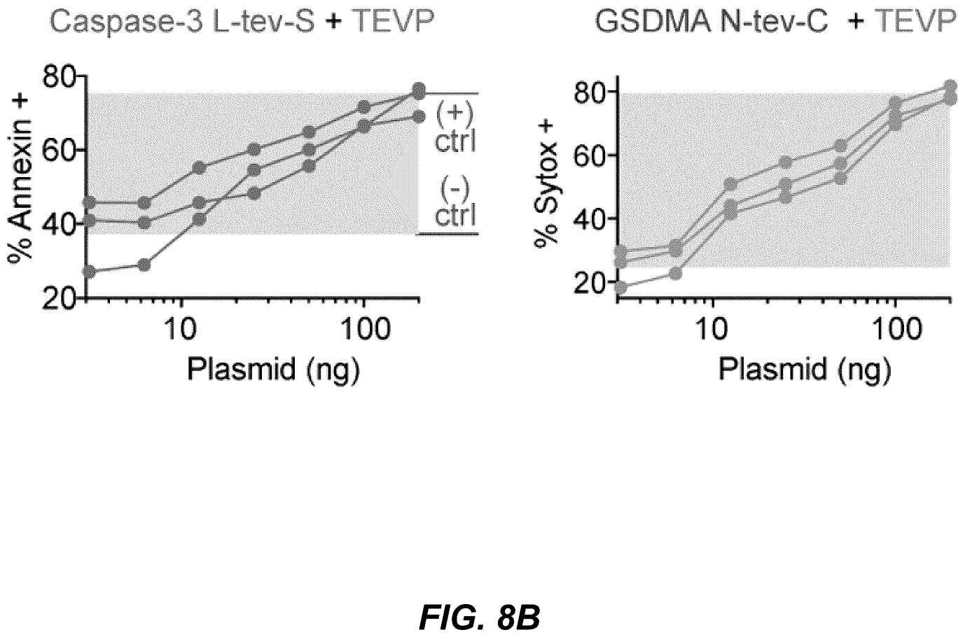

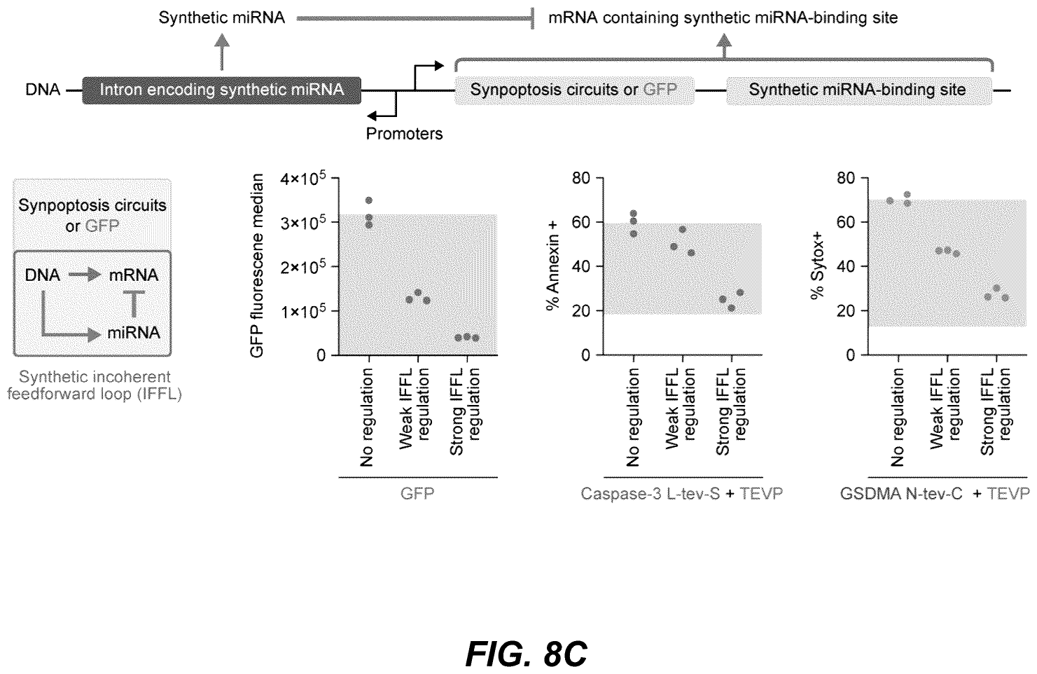

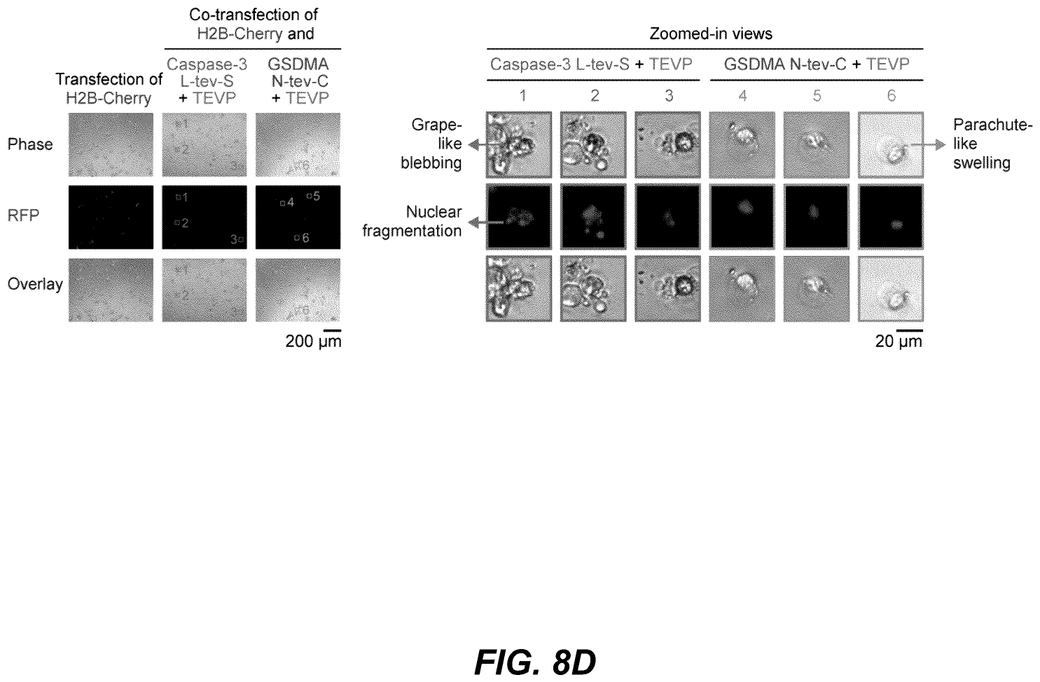

Next, considering that naturally occurring cell death is influenced by the concentration of executioner molecules, it was investigated whether synpoptosis is concentration-sensitive. Indeed, engineered auto-inhibited caspase-3 and GSDMA, when expressed at high levels indicated by co-transfected Cherry, induced noticeable cell death even without the activating TEVP (FIG. 8A). Further, titrating the amount of plasmid DNA encoding the synpoptosis circuits enabled DNA dose-dependent control over killing fractions (FIG. 8B). At a constant amount of plasmid DNA, tuning could achieved by adjusting mRNA dosage (FIG. 8C) through a synthetic miRNA-based incoherent feedforward loop (IFFL) motif, which allows DNA dosage-independent control of protein expression.

Then, because naturally occurring cell death can be modulated by small-molecule compounds, the effect of Q-VD-OPh, a caspase inhibitor, on synpoptosis circuits was investigated (FIG. 2C). In line with expectations, Q-VD-OPh markedly attenuated apoptosis induced by the caspase-3 circuit, but did not affect pyroptosis mediated by the GSDMA circuit. It was also asked whether cells treated with synpoptosis circuits stain positive for TO-PRO-3, a dye that enters apoptotic cells through pannexin channels and pyroptotic cells through permeabilized membranes. As anticipated, cells treated with either circuit showed positive staining for TO-PRO-3 (FIG. 2D).

A physiologically important feature of pyroptosis is the release of pro-inflammatory cytokines, notably the interleukin (IL)-1 family including IL-1β and IL-18. For demonstration purposes, a HEK cell line that stably expresses IL-1β and IL-18 was generated. An obvious increase in supernatant levels of IL-1β and IL-18 was observed in cells treated with the GSDMA circuit, compared to cells treated with the caspase-3 circuit or the mock transfection control (FIG. 2E).

Furthermore, the morphological characteristics of cells treated with synpoptosis circuits was examined (FIG. 8D). Cell shapes were analyzed by phase-contrast microscopy. Mock-transfected cells displayed a mostly flat and extended appearance. In contrast, cells transfected with the caspase-3 circuit showed extensive grape-like membrane blebbing and cell shrinkage, characteristic of apoptosis. On the other hand, cells transfected with the GSDMA circuit showed cell rounding and parachute-like membrane swelling, indicative of pyroptosis. Additionally, the nuclear localized H2B-Cherry marker allowed us to visualize nuclear fragmentation in cells treated with the caspase-3 circuit.

Lastly, mRNA represents a rapidly growing modality for transient therapeutic protein expression. To determine whether the mRNA versions of the synpoptosis circuits could generate the expected killing effects, mRNA was generated by in vitro transcription and used to transfect cells, and then three indicators of cell death were assessed. A luciferase-based assay to quantify ATP levels in the cell culture, an indicator of viable cells, revealed that mRNA-encoded synpoptosis circuits metabolically inactivated cells after killing (FIG. 2F). The GSDMA circuit resulted in substantial release of lactate dehydrogenase (LDH), a pyroptotic signature (FIG. 2G), as well as adenosine triphosphate (ATP), a small-molecule DAMP (FIG. 2H).

Together, these results demonstrate that synpoptosis circuits induce canonical features of cell death programs and function expectedly whether delivered as DNA or mRNA.

### Synpoptosis Circuits Direct the Mode of Cell Death

Instead of leaving the choice of death mode up to cells, synpoptosis circuits described herein were used to actively direct cell death. Specifically, the aim was to drive pyroptosis in apoptosis-prone cells, promote apoptosis in pyroptosis-prone cells, and trigger either death program in cells capable of both apoptosis and pyroptosis. Because these experiments involve mixed programs, the cells with Sytox and Annexin were double-stained to distinguish between apoptosis and pyroptosis. Given that Annexin labels both apoptotic and pyroptotic cells, it was as a proxy for total cell death.

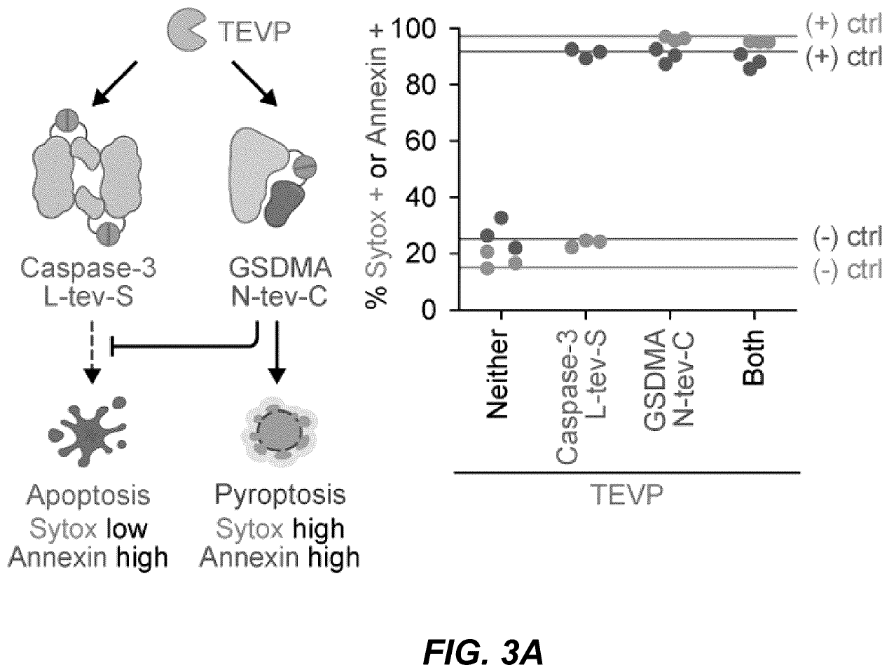

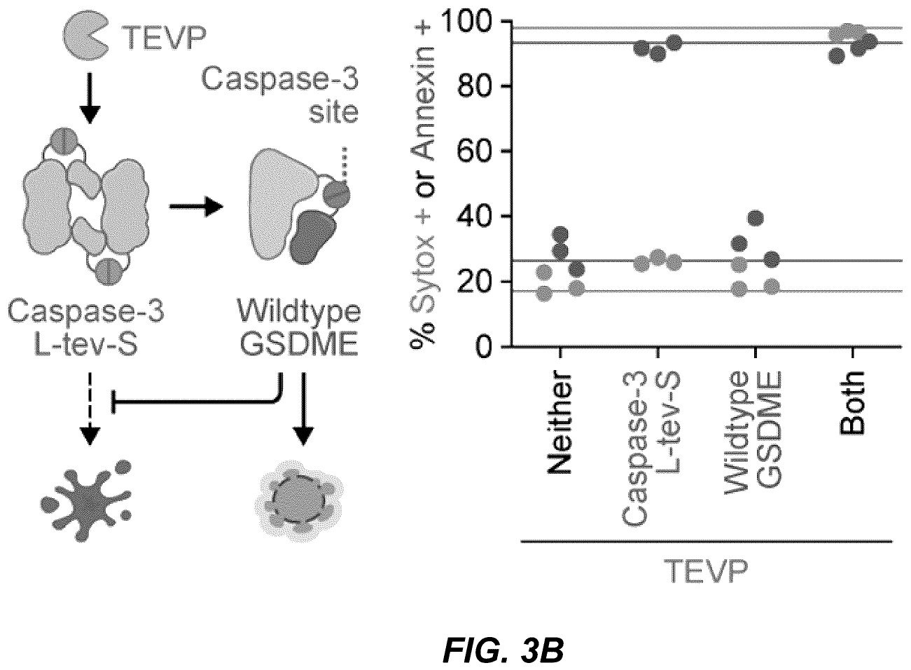

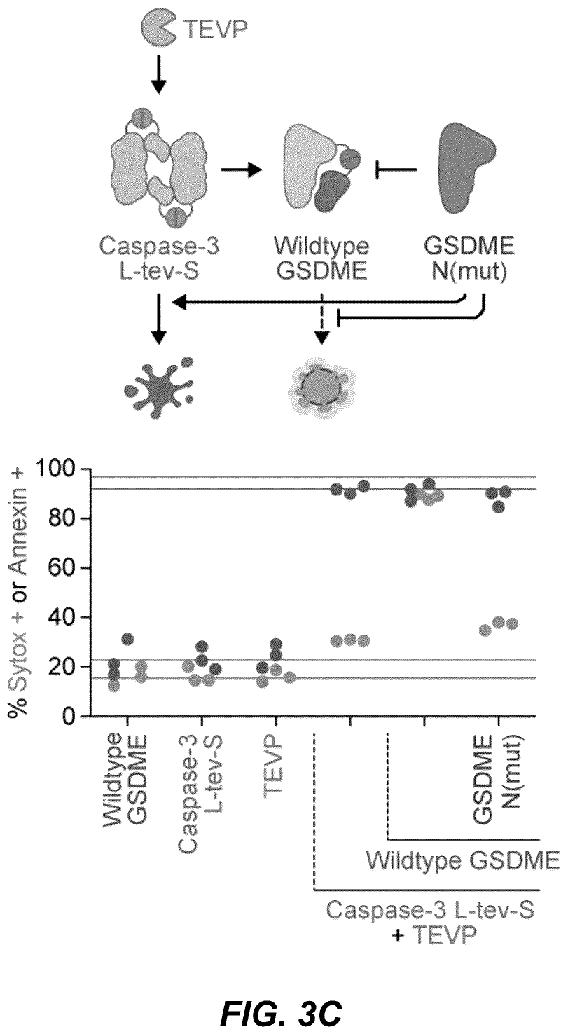

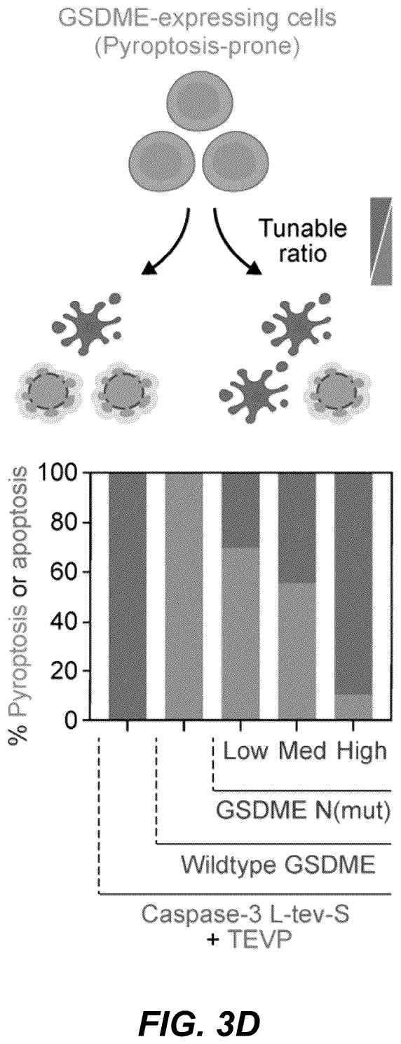

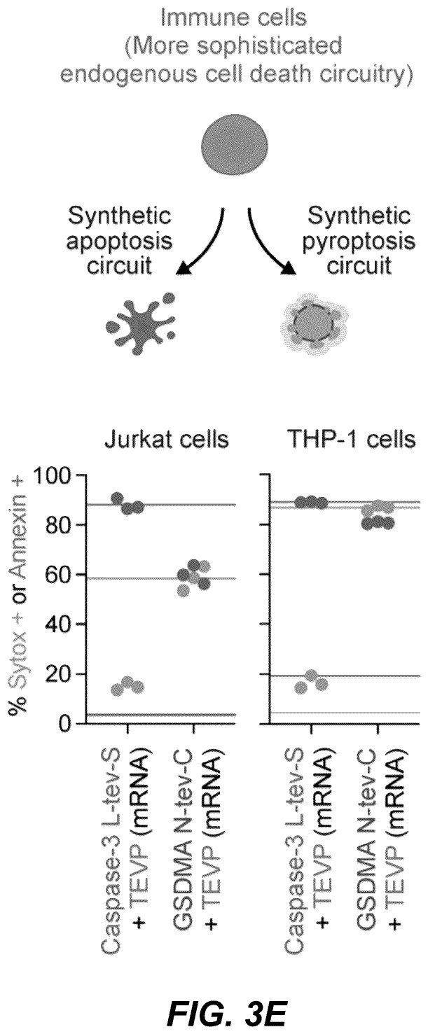

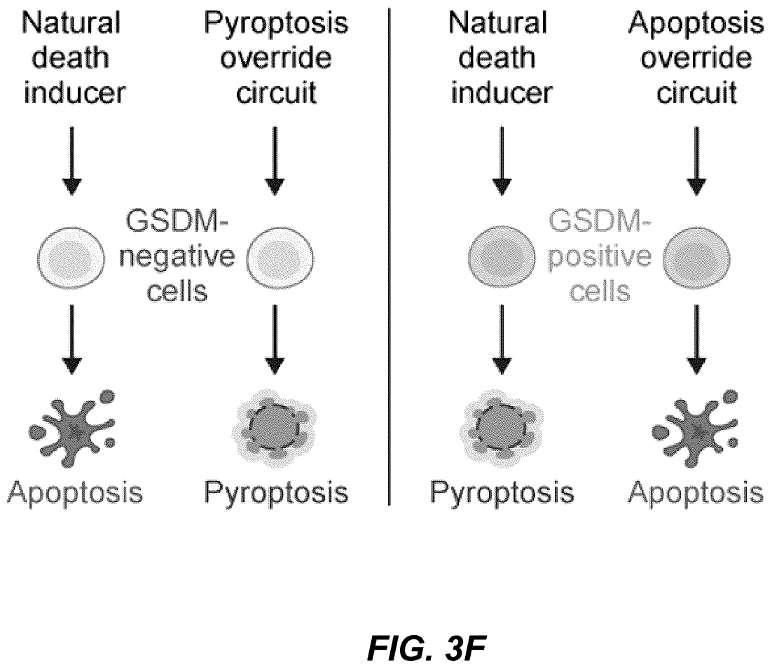

To drive pyroptosis in apoptosis-prone cells, a synthetic system that mimics a GSDM-negative cell context was established, with TEVP activating caspase-3 to induce apoptosis. In this background, ectopically expressing TEVP-activatable GSDMA converted apoptosis to pyroptosis (FIG. 3A), suggesting that GSDMA dominated over caspase-3. While this dominance provided a straightforward approach to drive pyroptosis in GSDM-negative cells, it could be exploited by cells seeking to evade apoptosis. Indeed, cells that express GSDME, a natural substrate for caspase-3, undergo pyroptosis downstream of caspase-3 activation. This effect can be recapitulated by ectopically expressing wildtype GSDME, which alone did not induce cell death, but caused pyroptosis in response to TEVP-mediated caspase-3 activation (FIG. 3B).

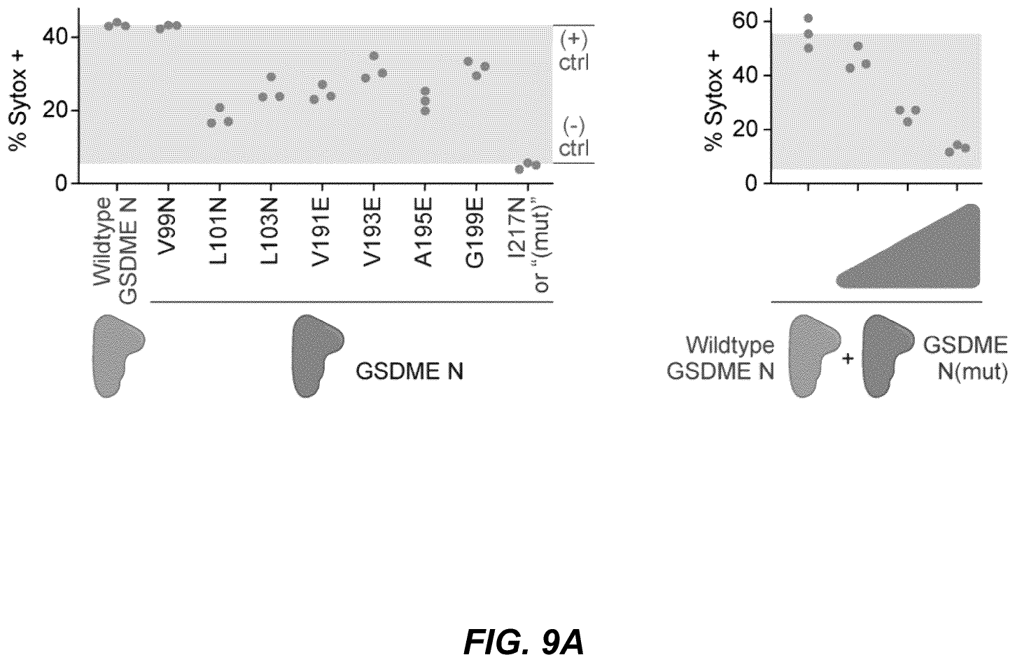

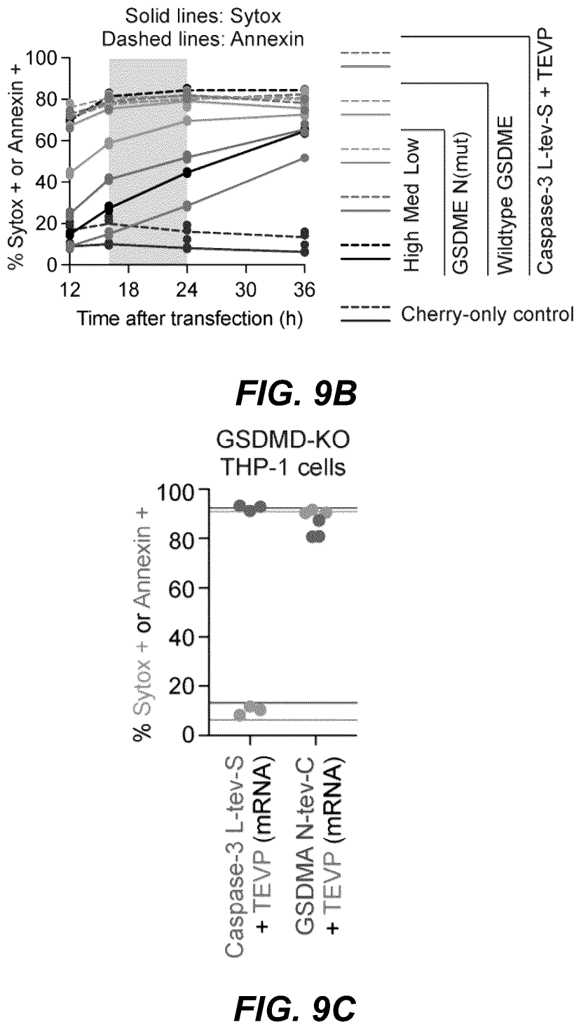

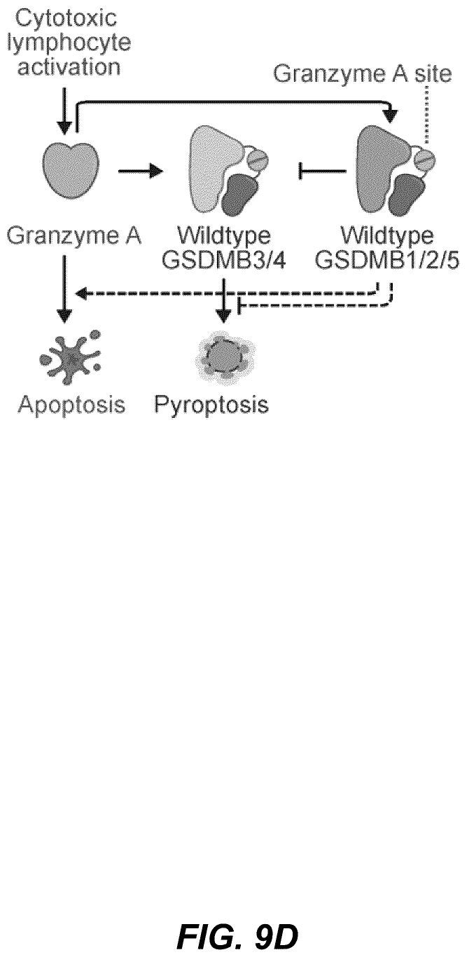

To promote apoptosis in cells expressing GSDME, a protein-level GSDME inhibitor was searched for. Recent studies revealed that GSDMB has alternatively spliced variants, with the non-pyroptotic variants inhibiting the pyroptotic ones. Motivated by this trans inhibition mechanism, a panel of GSDME N-terminal domain mutants were tested, including some associated with cancer, and I217N was identified as defective in inducing pyroptosis and capable of inhibiting the wildtype counterpart (FIG. 9A). Then, this mutant was co-transfected along with wildtype GSDME, TEVP, and TEVP-activatable caspase-3. The mutant expectedly suppressed pyroptosis, as evidenced by reduced Sytox uptake, while permitting caspase-3-induced apoptosis, as read out by high Annexin levels (FIG. 3C).

The results above demonstrate that synpoptosis circuits can guide cells towards apoptosis or pyroptosis irrespective of their intrinsic preferences. However, to leverage the pro-inflammatory benefits of pyroptosis without triggering excessive inflammation, or conversely, the immunosuppressive benefits of apoptosis without curbing beneficial inflammation, it would be ideal to be able to adjust the relative levels between the two modes of cell death. By titrating down the GSDME inhibitor, pyroptosis could be attenuated to various degrees while still allowing activated caspase-3 to induce apoptosis FIG. 9B). Further quantification using the Annexin signal as a measure of total cell death and the Sytox signal as an indicator of pyroptosis revealed the tunable ratio between the two death programs (FIG. 3D).

HEK cells are pyroptosis-incompetent without ectopically introduced GSDMs. To assess the efficacy of synpoptosis circuits in cells with more sophisticated endogenous death circuitry, two widely used immune cell lines, Jurkat and THP-1 were selected. Jurkat and THP-1 cells were transfected with mRNA encoding TEVP-activated caspase-3 and GSDMA circuits and found that the cells died through the anticipated programs (FIG. 3E). Further, to validate the orthogonality of synpoptosis circuits to endogenous death circuitry, the circuits were tested in GSDMD-knockout (KO) THP-1 cells. As expected, the circuits killed the GSDMD-KO THP-1 cells similarly to the wildtype cells (FIG. 9C).

Together, these results demonstrate that synpoptosis circuits can direct the mode of cell death in various cell contexts and tune the ratios between apoptosis and pyroptosis.

### Synpoptosis Circuits Perform Combinatorial Computation

While engineered executioners enable control of cell death modes, taking advantage of upstream inputs would allow synpoptosis circuits to target specific cells. Perhaps for similar reasons, natural cell death pathways respond to logical combinations of inputs. For instance, either caspase-1 or caspase-11 activates GSDMD, functioning as an OR-like gate. Both GSDMD cleavage and its lipidation are necessary for pyroptosis, forming an AND-like gate.

The activation and repression synpoptosis modules developed above can be combined to achieve such combinatorial logic gating functions. Three biologically relevant gates were focused on: triggering cell death in the presence of Inputs 1 AND 2; in the presence of Input 1 OR 2; and in the presence of Input 1 AND the absence of Input 2. While the first and third gates increase specificity by killing cells with the two inputs in the right combinations, the second gate broadens specificity and may mitigate antigen escape.

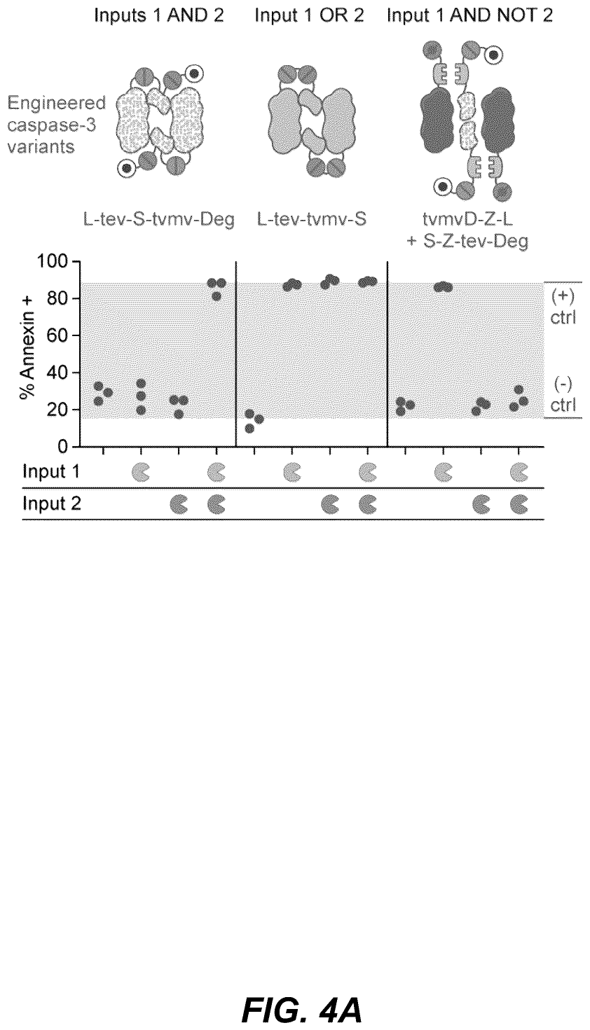

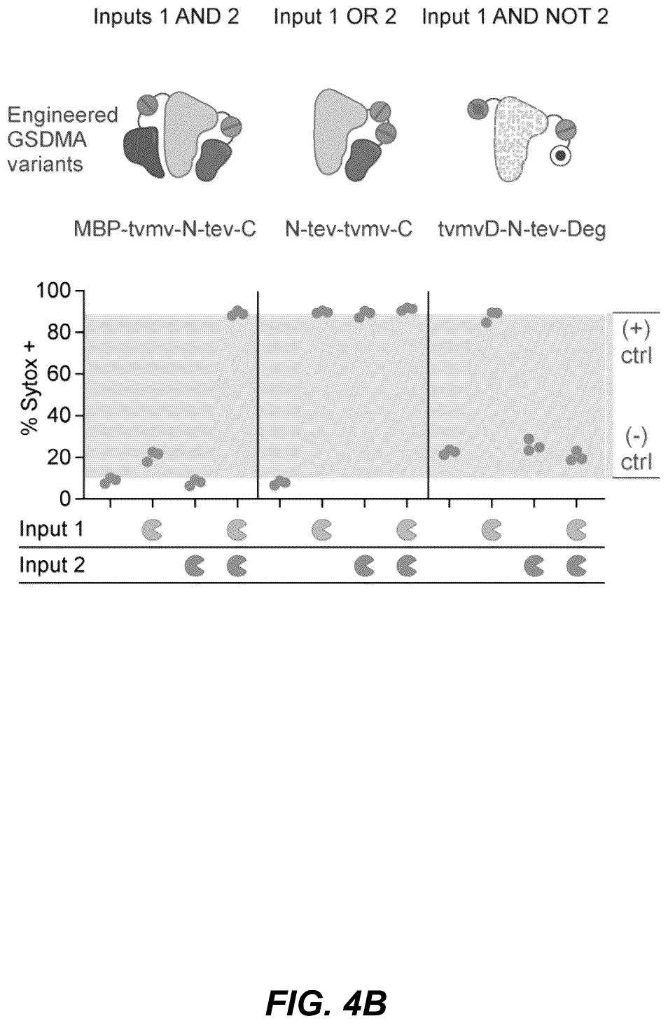

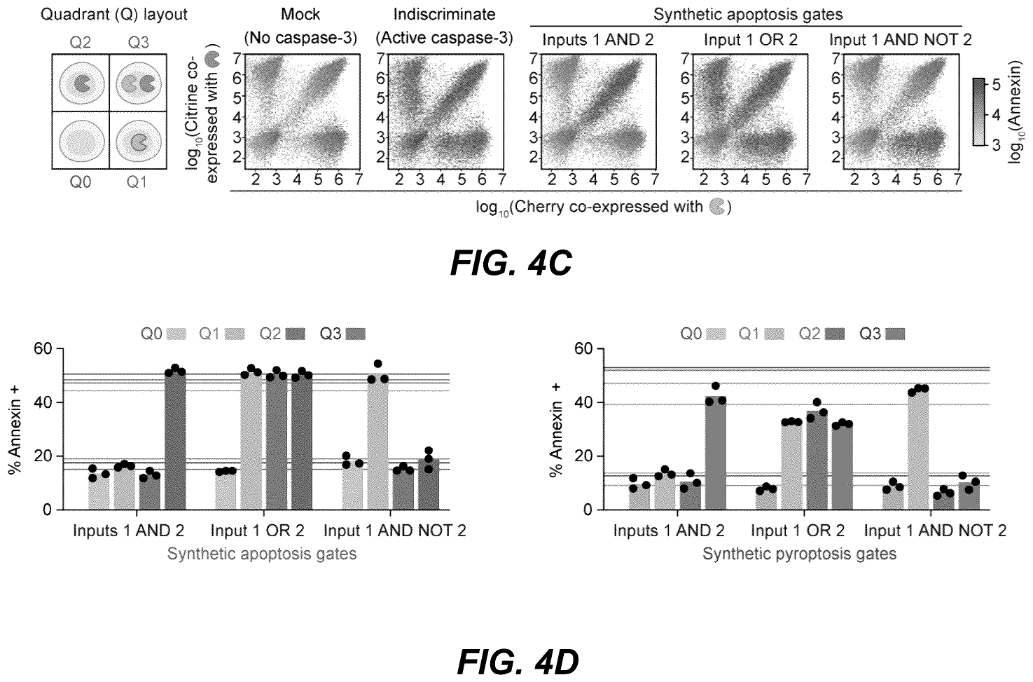

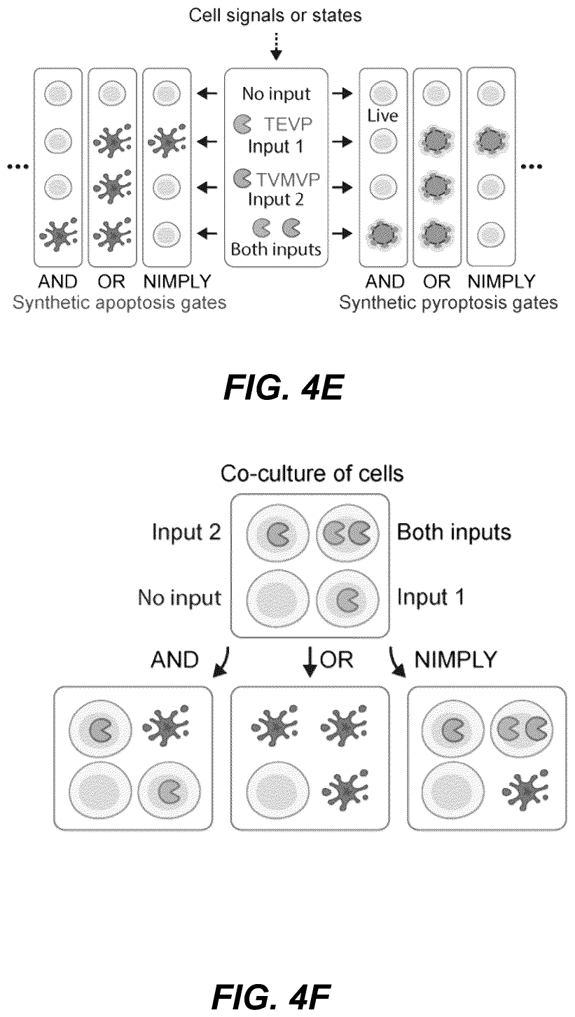

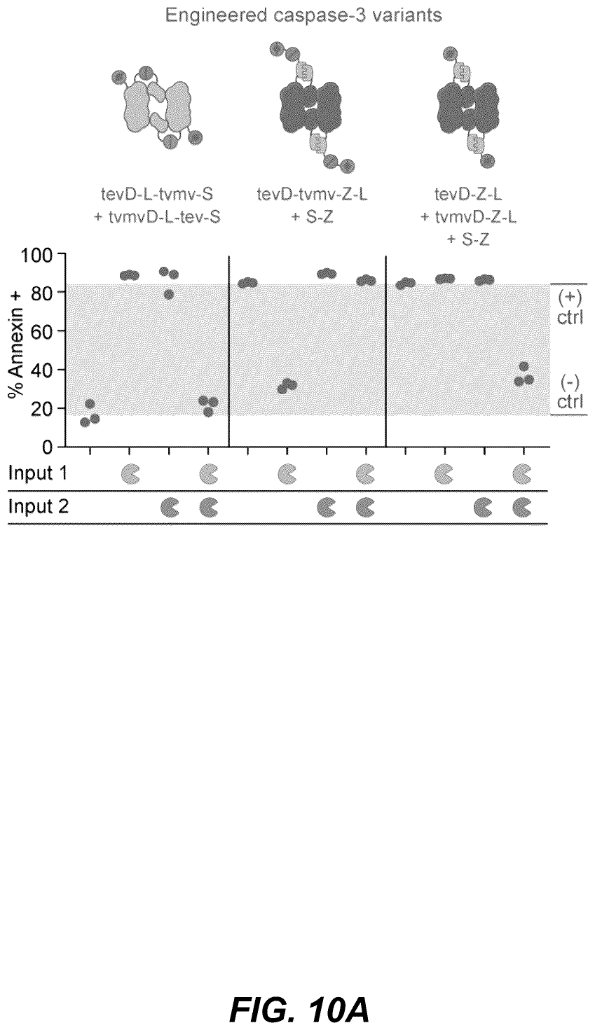

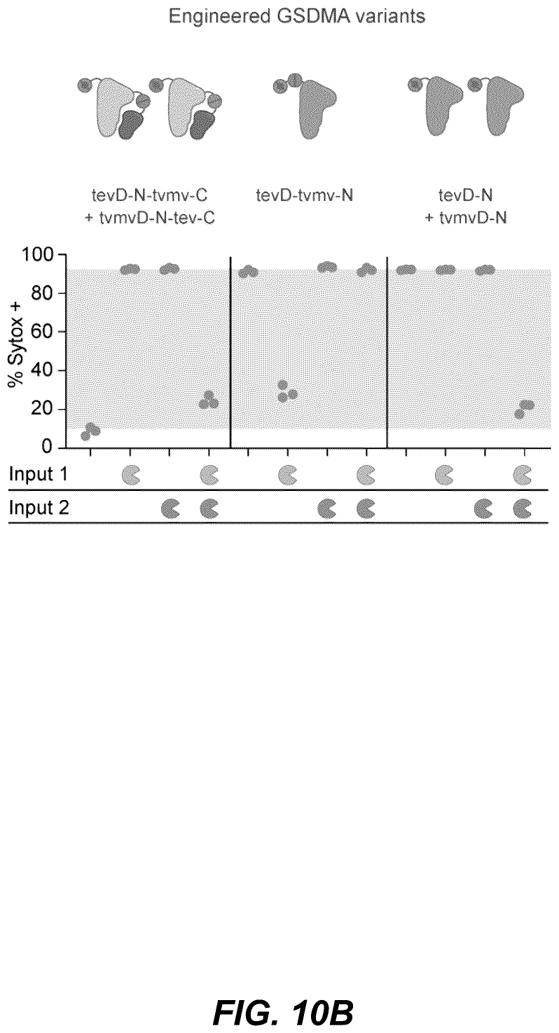

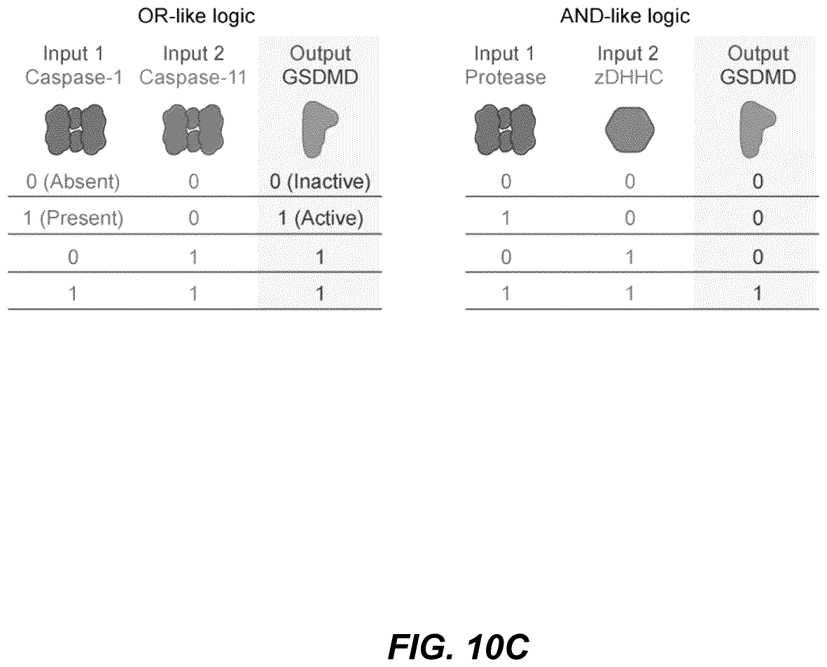

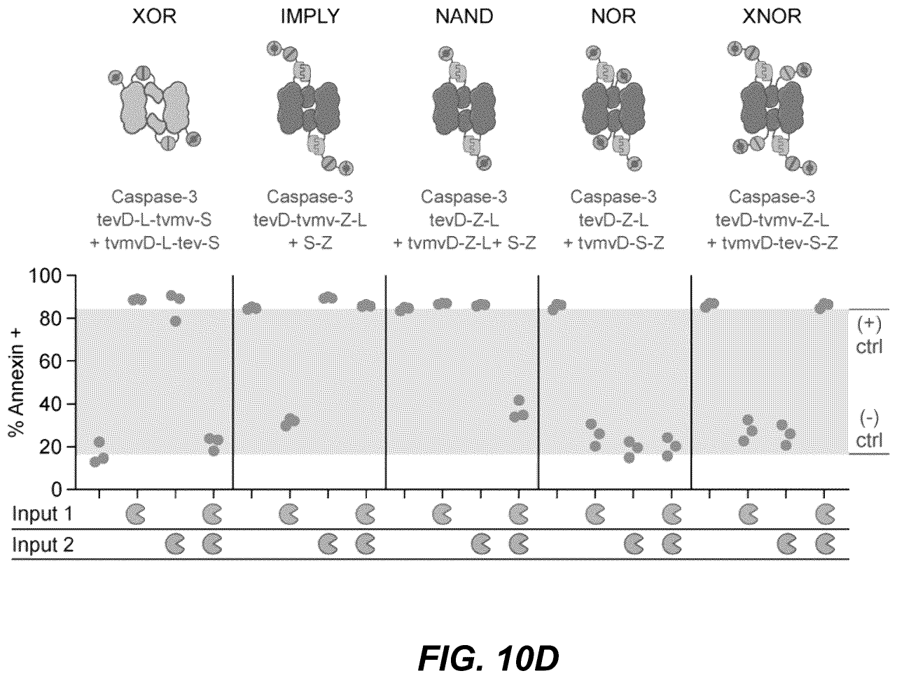

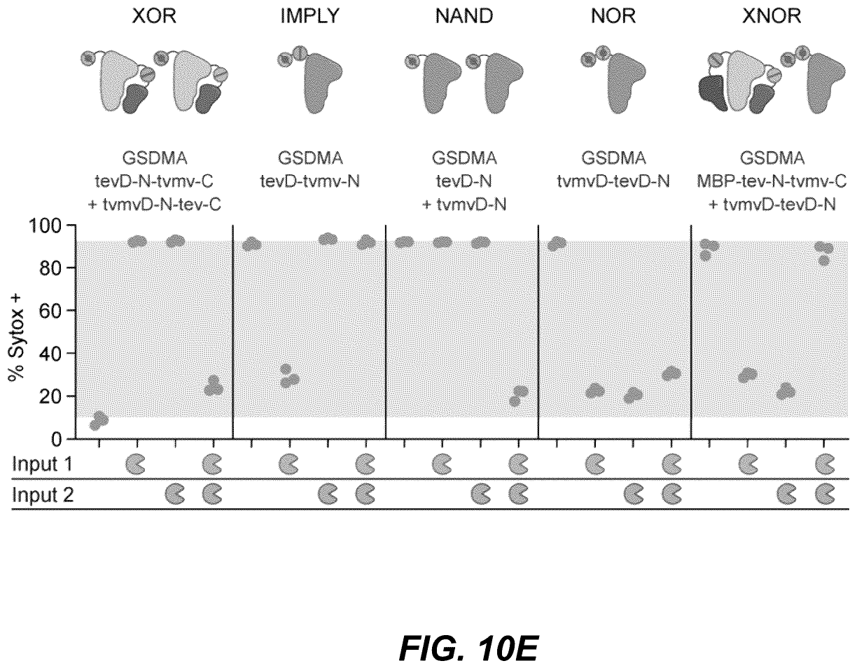

To trigger apoptosis in the presence of Inputs 1 (TEVP) AND 2 (TVMVP), TEVP-activatable caspase-3 was fused to a TVMVP-removable degron (FIG. 4A, Inputs 1 AND 2). In this design, Input 1 releases the linker constraint between the large and small subunits of the engineered caspase-3, while Input 2 protects the caspase-3 from degradation. By contrast, to trigger apoptosis in the presence of Input 1 OR 2, we placed tandem TEVP and TVMVP cleavage sites at the inter-subunit linker of caspase-3 (FIG. 4A, Input 1 OR 2). In this configuration, either protease is sufficient to activate the engineered caspase-3. Similar principles can be used to trigger apoptosis in the presence of Input 1 AND the absence of Input 2, by adding a TEVP-removable degron to the small subunit and a TVMVP-activatable N-degron to the large subunit (FIG. 4A, Input 1 AND NOT 2). By extension, using different configurations of degrons and cleavage sites, synthetic apoptosis executioners were constructed that perform more binary logic operations (FIG. 10A). With some modifications, the gate designs were transferable to pyroptosis programs (FIG. 4B and FIG. 10B).

As a proof of concept for combinatorial targeting, the synpoptosis gates were tested in a co-culture of engineered HEK cells that stably express Input 1, Input 2, or both, and wildtype cells that express neither. Input 1 with Cherry and Input 2 with Citrine were co-expressed, such that the two fluorescent protein profiles represented input patterns (FIG. 4C, Quadrant layout). The co-culture of the four cell lines were then transfected with the synthetic apoptosis gates to analyze their killing specificity. Transfections of no caspase-3 (FIG. 4C, Mock) and active caspase-3 (FIG. 4C, Indiscriminate) were used to establish background and ceiling apoptosis levels, respectively. Transfection of the AND-gated caspase-3 triggered apoptosis predominantly in double-positive cells, suggesting specific killing of cells exhibiting both inputs (FIG. 4C-FIG. 4D, Inputs 1 AND 2). By contrast, the OR-gated caspase-3 caused apoptosis in single-positive and double-positive cells, sparing only double-negative cells (FIG. 4C-FIG. 4D, Input 1 OR 2). The caspase-3 variant designed to activate in the presence of Input 1 and the absence of Input 2 restricted apoptosis to the corresponding cells, as expected (FIG. 4C-FIG. 4D, Input 1 AND NOT 2). These results are qualitatively consistent with experiments performed in a non-co-culture context (FIG. 4A). Similar killing specificities were observed when the synthetic pyroptosis gates were transfected into the co-culture (FIG. 4D).

Together, these results demonstrate that synpoptosis circuits can respond to combinatorial protease inputs and eliminate specific cells among mixed populations through logic gating.

### Synpoptosis Circuits Selectively Eliminate Target Cells

A major motivation for engineering input-responsive synpoptosis circuits is to achieve target cell selectivity. Tissues generally contain heterogeneous mixtures of healthy and harmful cells. To protect healthy cells, synpoptosis circuits were engineered that selectively eliminate harmful cells by conditional circuit activation.

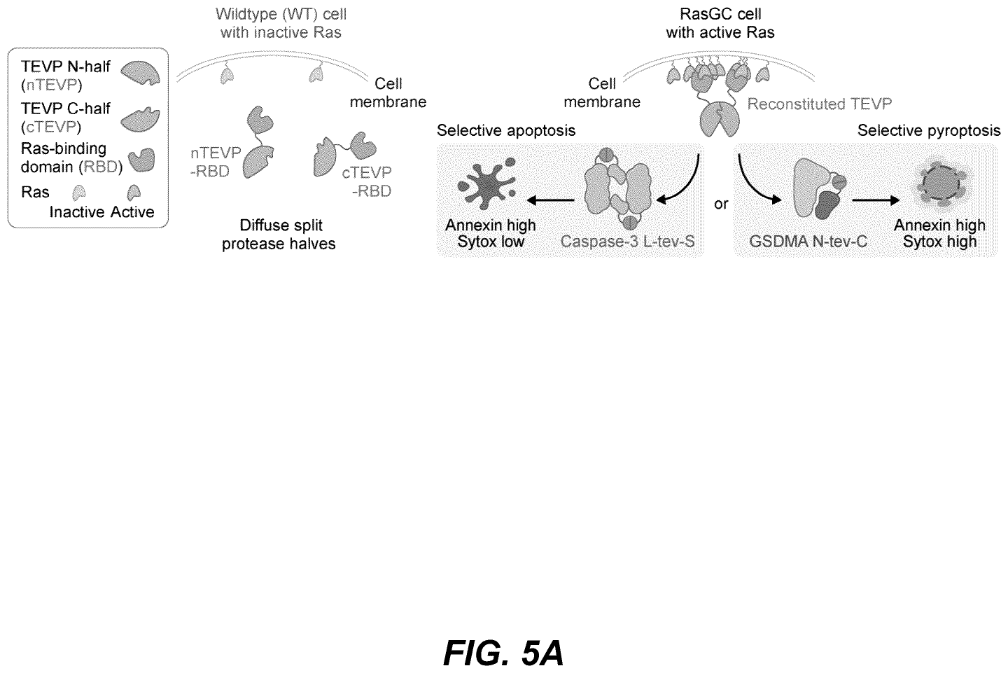

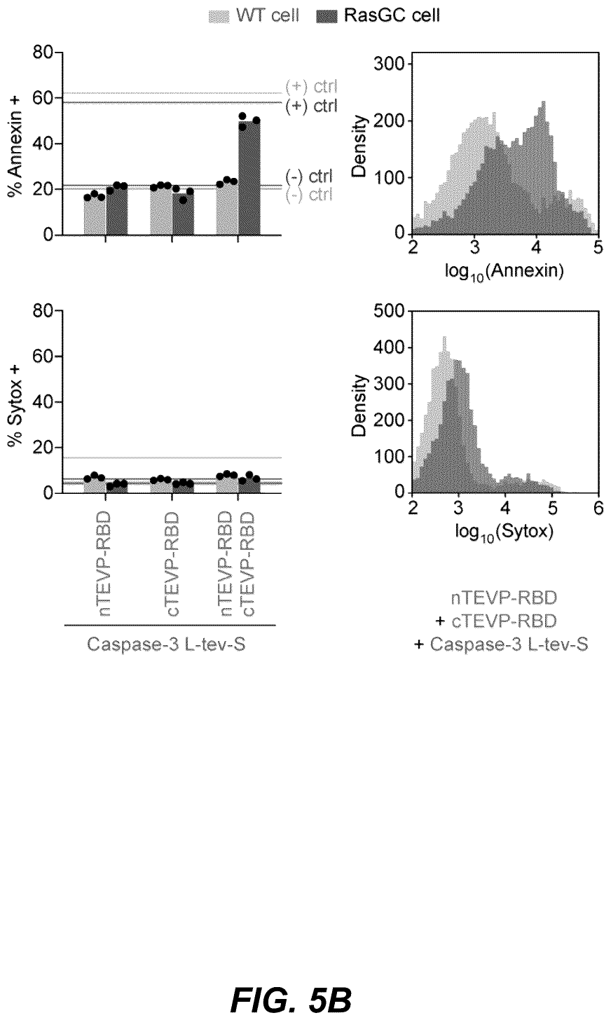

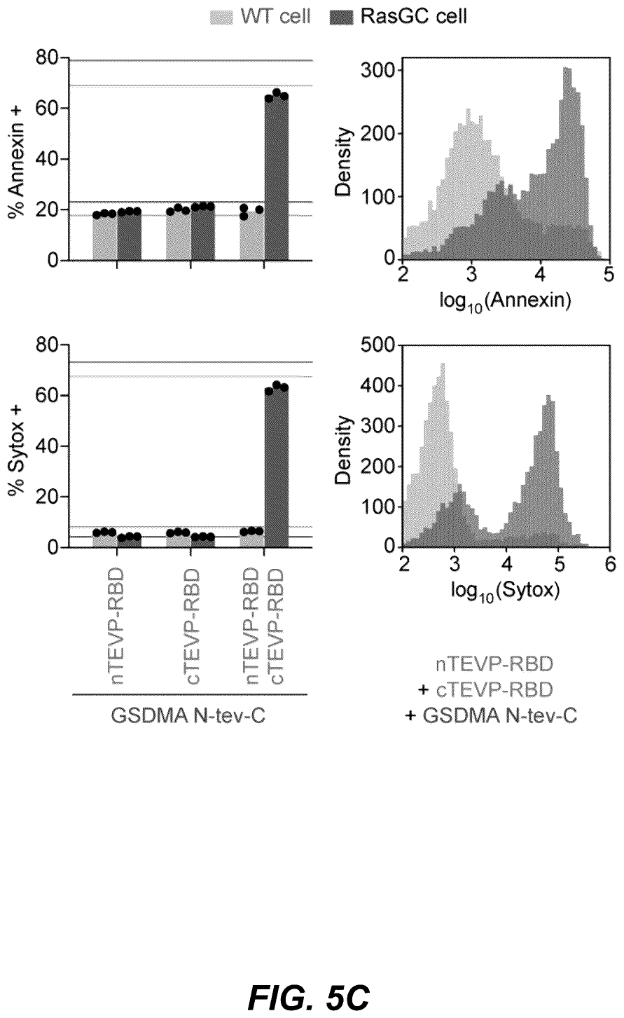

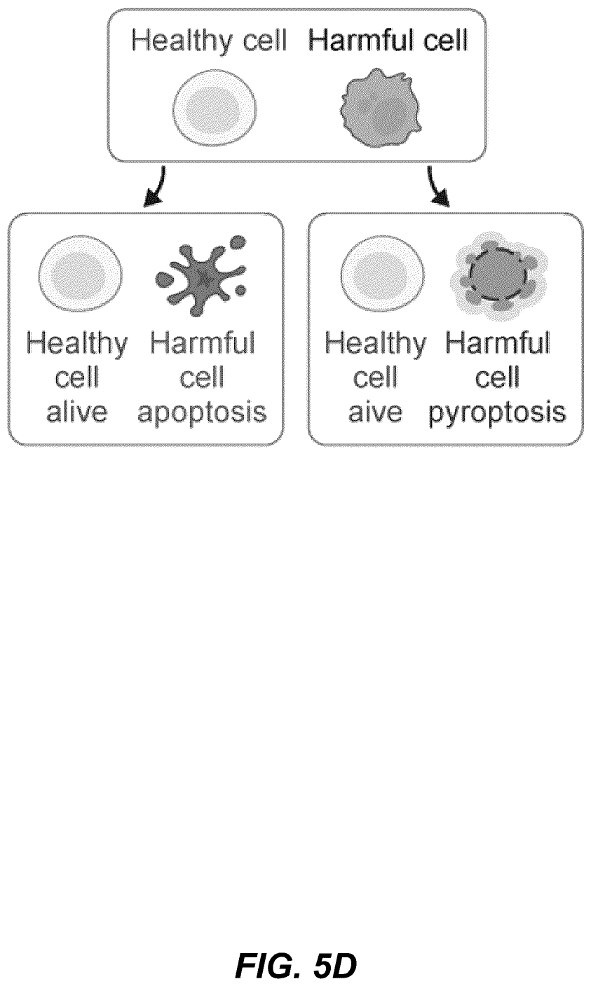

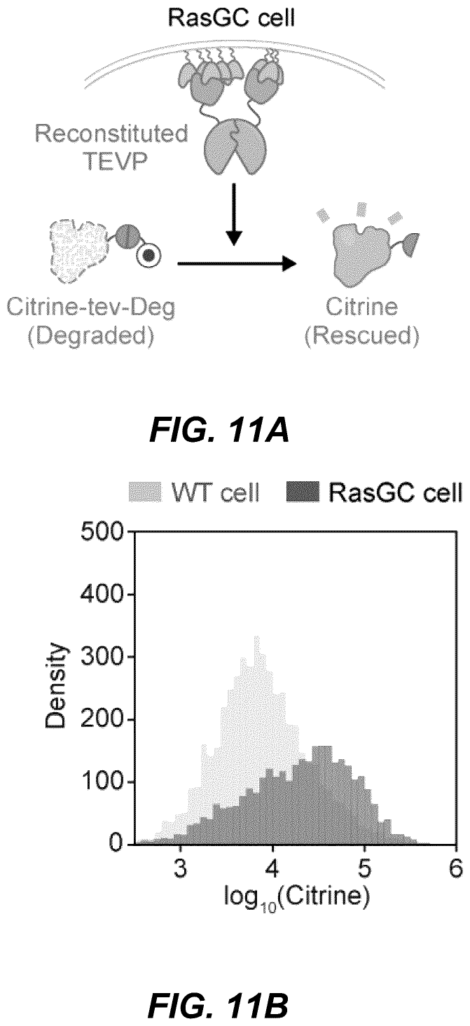

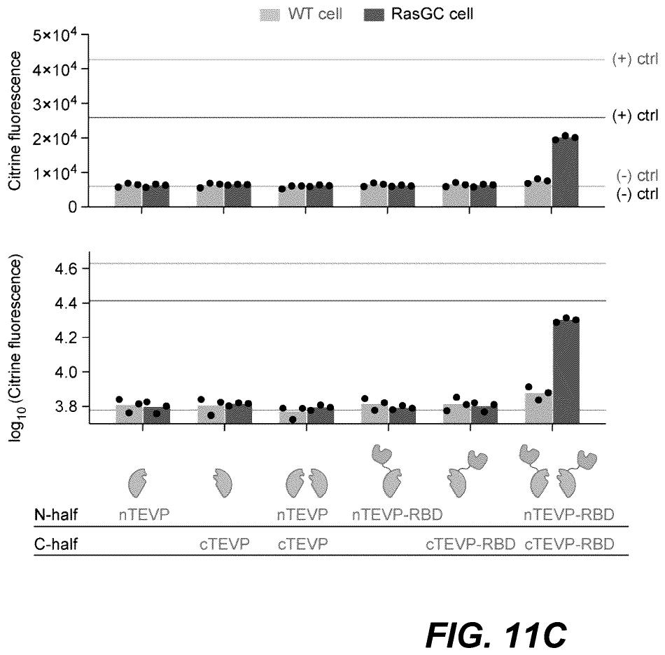

As a demonstration for target cell selectivity, a synthetic sensor of Ras oncogene activity was incorporated into synpoptosis circuits. The sensor consists of the inactive N-terminal and C-terminal halves of a split TEVP, with each half fused to a Ras-binding domain. Active Ras clusters at the membrane and binds the modified TEVP halves, reconstituting TEVP by proximity (FIG. 5A). To evaluate the effectiveness of the Ras sensor, it was tested in HEK cells stably expressing the constitutively active H-Ras G12V mutant and a Cerulean fluorescent marker (hereafter, “RasGC cells”), along with a destabilized Citrine reporter that can be rescued by TEVP cleavage (FIG. 11A). As a control, a similar Citrine reporter cell line without the active Ras mutant (hereafter, “wildtype (WT) cells”) was analyzed. When the sensor was transiently transfected into the two cell lines, Citrine fluorescence increased in the RasGC cells more than the WT cells (FIG. 11B-FIG. 11C), suggesting that the sensor can classify Ras activity states.

Next, the sensor was integrated into synpoptosis circuits for selective killing of RasGC cells. A circuit consisting of the sensor and TEVP-activatable caspase-3 was co-transfected into RasGC and WT cells (FIG. 5B). According to Annexin staining, the RasGC cells underwent apoptosis to a degree similar to the positive control, a constitutively active apoptosis circuit containing full-length TEVP and TEVP-activatable caspase-3. The RasGC cells remained Sytox-low, indicating apoptosis rather than pyroptosis. Likewise, in synthetic pyroptosis experiments using TEVP-activatable GSDMA as the executioner instead of caspase-3, pyroptosis primarily occurred in the RasGC cells, shown by double positivity in Annexin and Sytox staining (FIG. 5C). In both apoptosis and pyroptosis experiments, WT cells maintained low levels of Annexin and Sytox staining, suggesting selective killing of RasGC cells.

Together, these experiments demonstrate the ability of synpoptosis circuits to selectively kill target cells and spare non-target ones by interfacing with an endogenous intracellular signal.

### Synpoptosis Circuits Support Intercellular Operations

Described below is cell-based delivery of synpoptosis circuits described herein. In this paradigm, engineered sender cells release virus-like particles (VLPs) containing cargo that can be internalized by non-engineered receiver cells. This approach can enable the development of a synthetic killer cell that secretes VLPs expressing synpoptosis circuits. The circuits can then trigger user-selectable death programs in receiver cells, providing control over the mode of cell death.

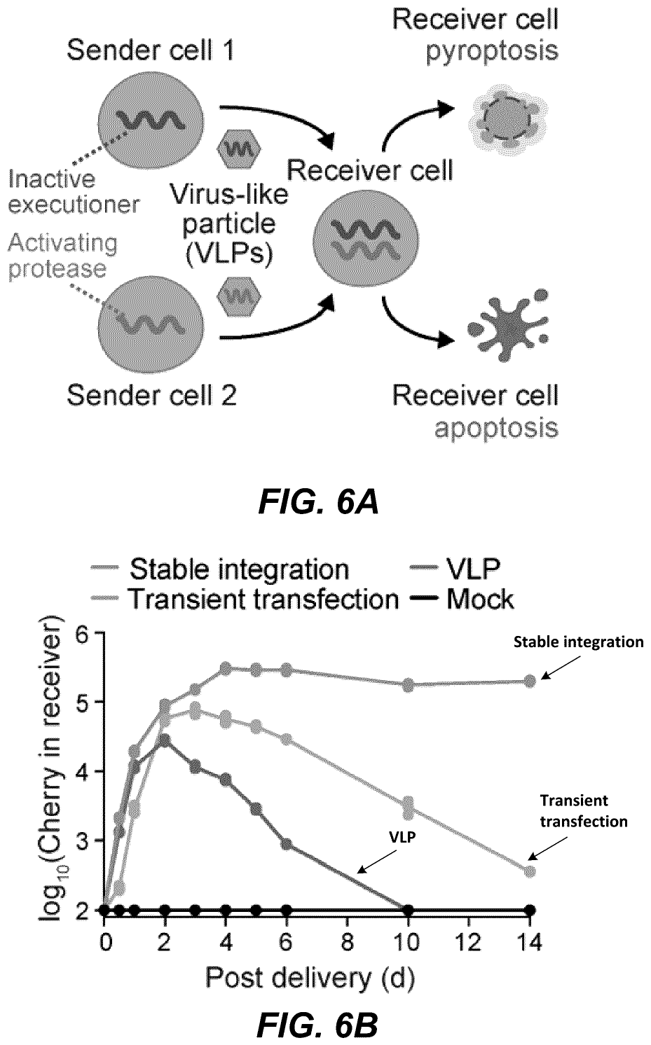

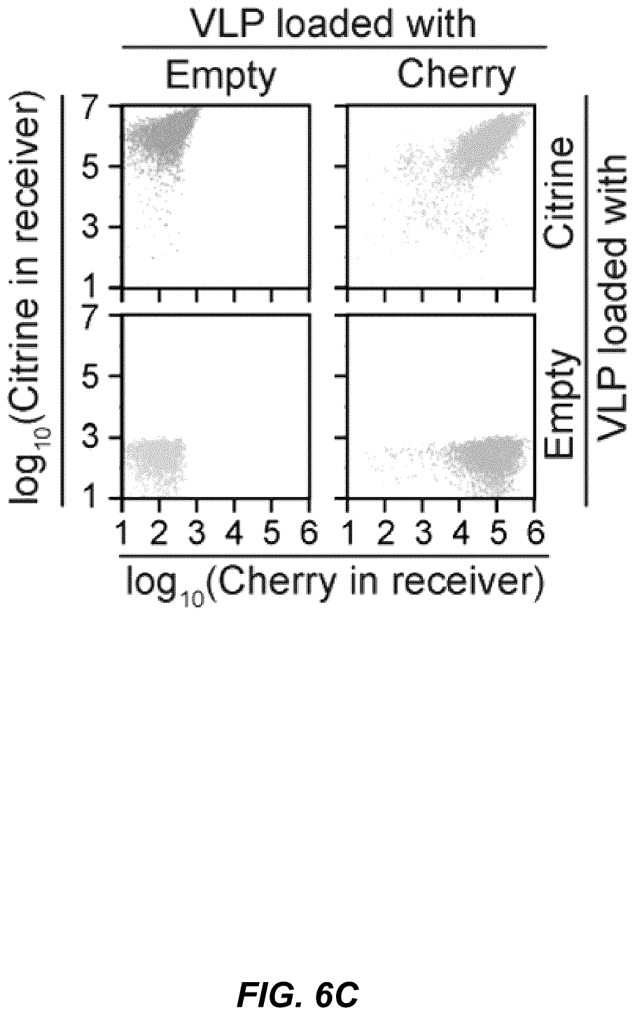

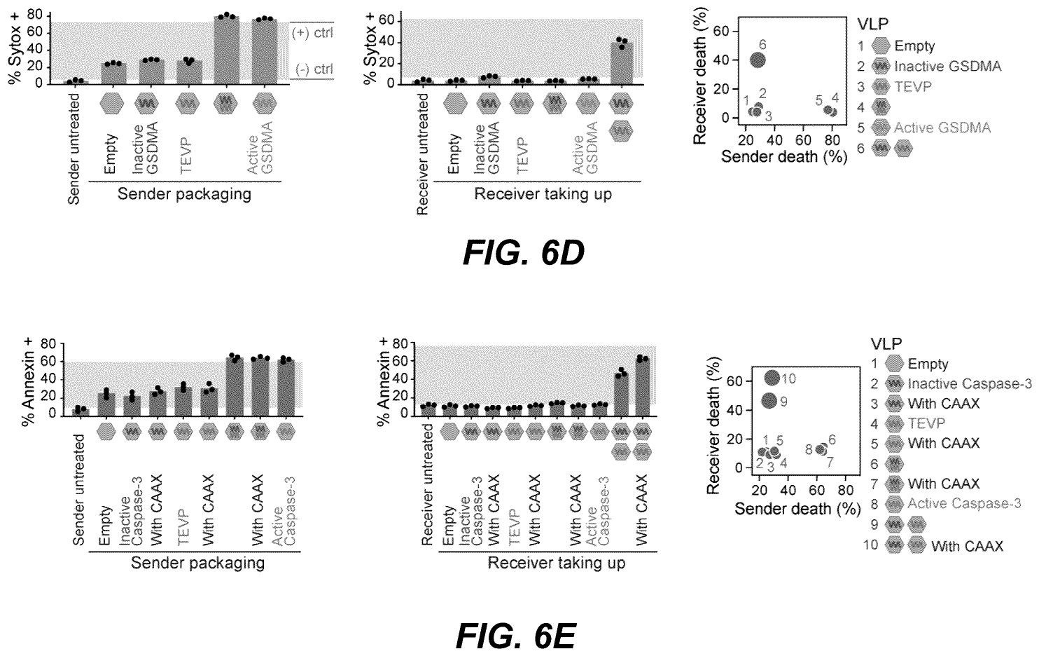

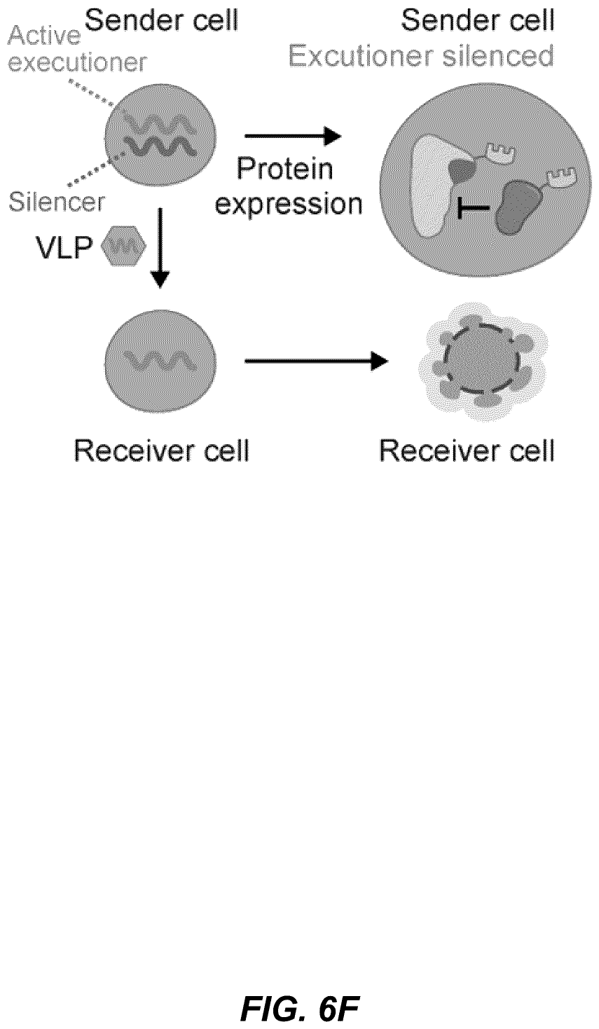

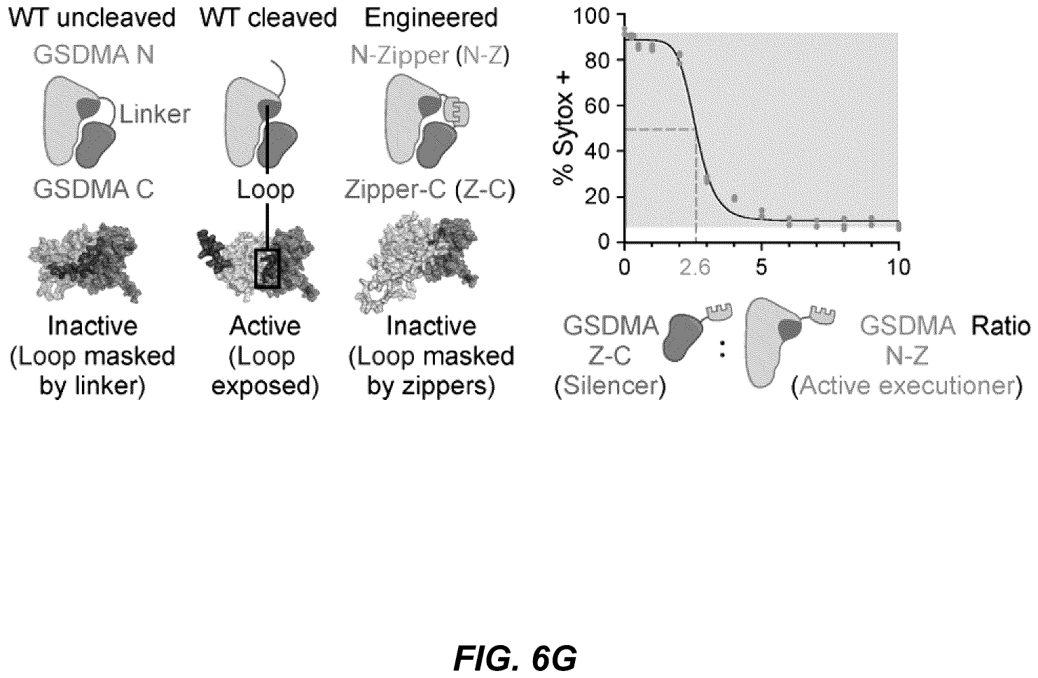

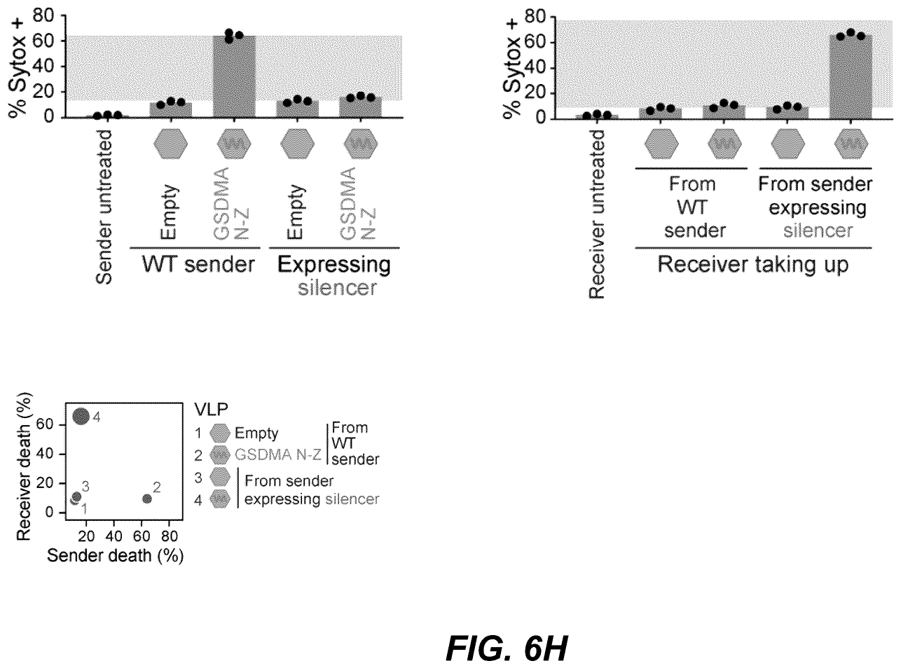

To deliver synpoptosis circuits with VLPs (FIG. 6A), it was first tested whether VLPs could enable high expression of nucleic acid-encoded model protein cargoes, Cherry and Citrine. As a model VLP, integration-deficient lentiviruses were used. HEK cells were used as senders to generate VLPs expressing Cherry. Supernatant containing the secreted VLPs led to transient expression of Cherry in receiver HEK cells (FIG. 6B). Furthermore, separate VLPs encoding Cherry and Citrine can be co-delivered to the same receiver cells without cross-interference (FIG. 6C).

It was next asked whether synpoptosis circuits can be transmitted intercellularly using VLPs. The key challenge is achieving high expression of the circuits in receivers without triggering cell death in senders. A simple way to avoid sender cell death is through an intersectional strategy, splitting a circuit into two components packaged by two separate senders, which, termed herein a split-sender system (FIG. 6A). As expected, when a pyroptosis circuit was split into TEVP and TEVP-activatable GSDMA, neither circuit component alone triggered substantial sender cell death. On the receiver side, HEK cells that took up separately packaged VLPs expressing both circuit components underwent pyroptosis, whereas cells that received VLPs loaded with only one cargo or empty VLPs survived (FIG. 6D). The same split-sender system also induced apoptosis (FIG. 6E). To boost apoptosis efficiency, local proximity of the circuit components was enforced by appending a CAAX membrane-localization sequence to each.

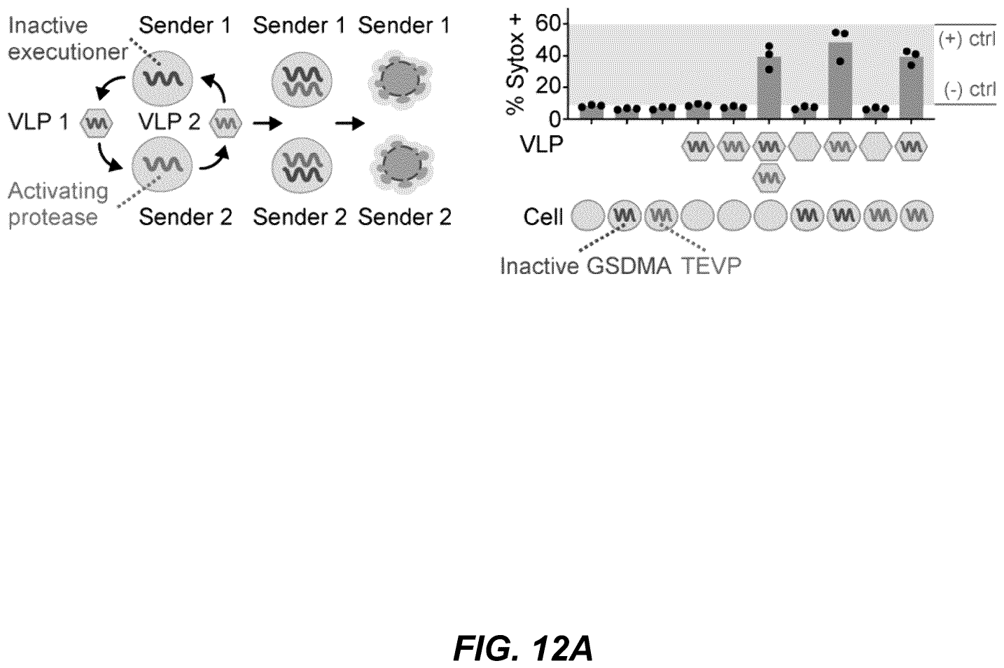

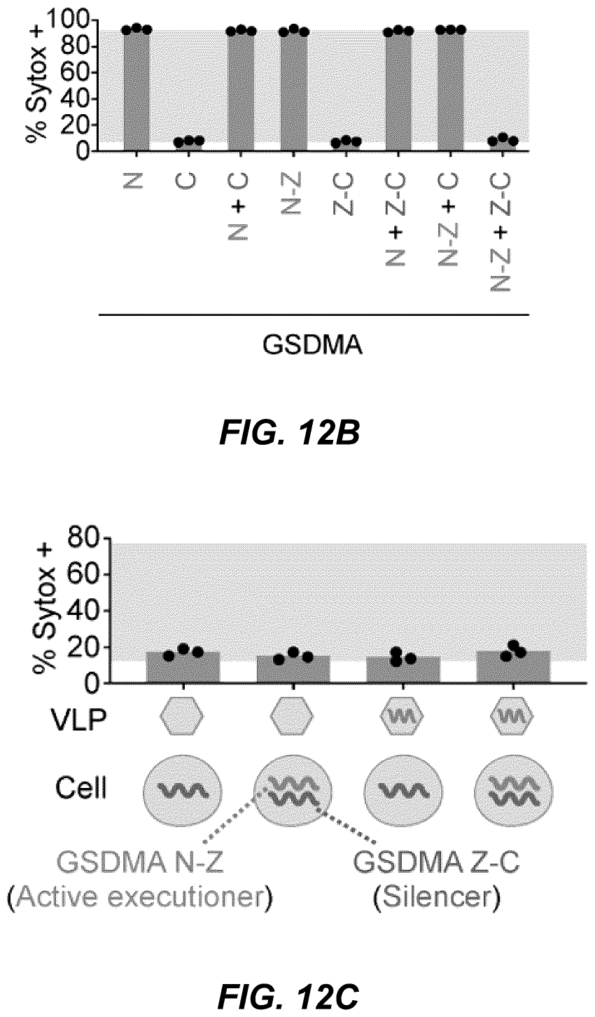

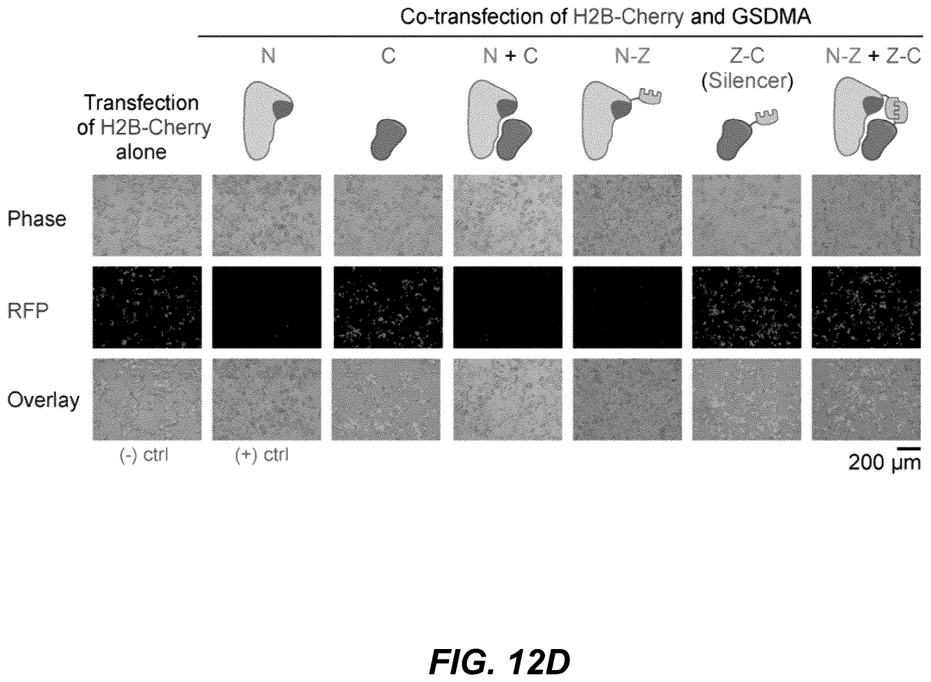

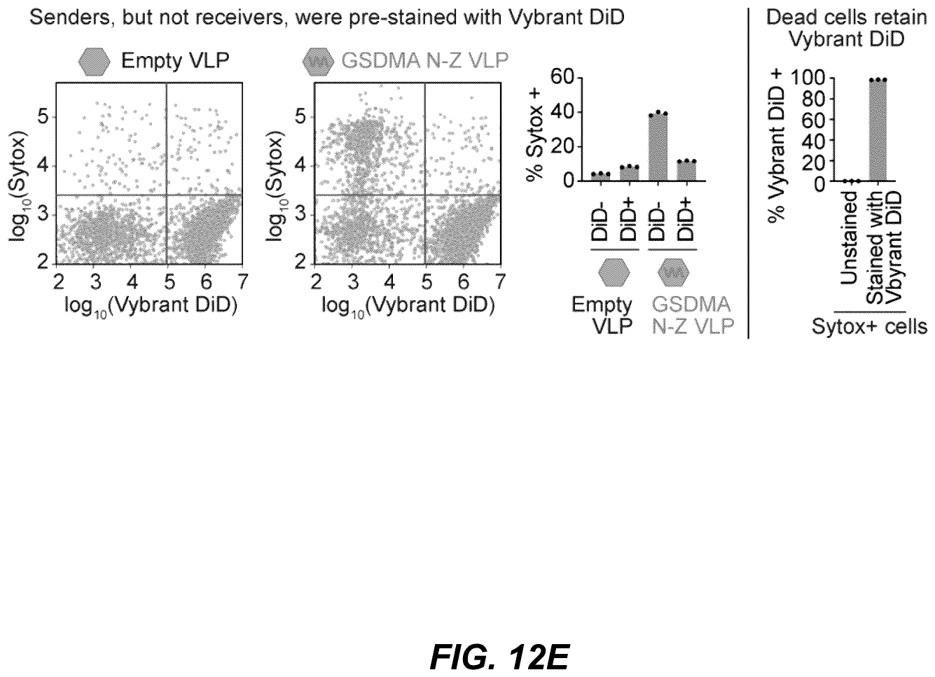

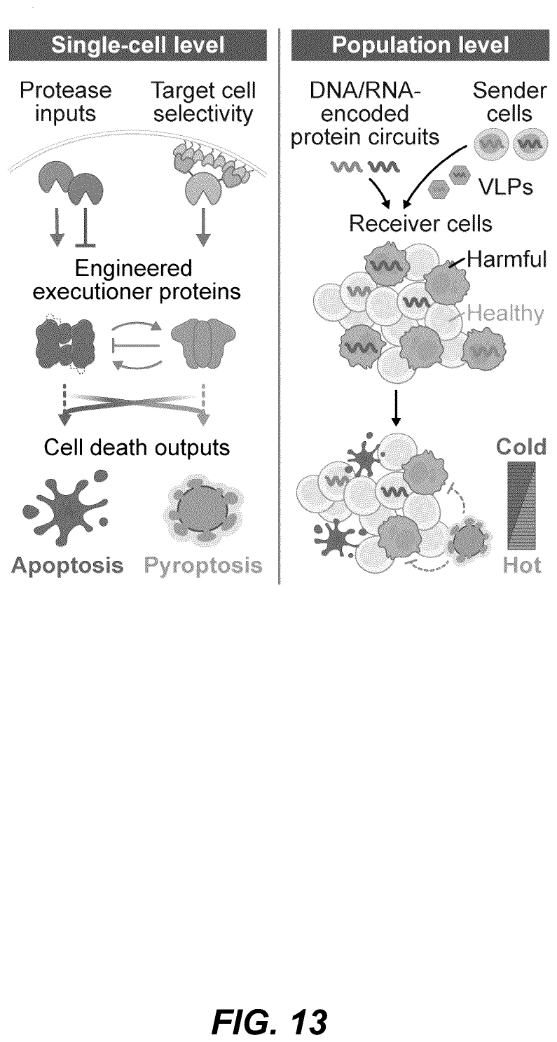

The split-sender system described above demonstrates the capability of intercellularly transmitted synpoptosis circuits. However, it requires two sender populations that are toxic to each other, as VLPs produced by one population complete the death circuit in the other (FIG. 12A). Engineering a compact single-sender system necessitates blocking the activity of synpoptosis circuits in sender cells without interfering with their activity in receivers (FIG. 6F). Small-molecule inhibitors exist for caspases and GSDMD, but it is difficult to restrict their activity to sender cells. An alternative strategy is to attach conditional degrons that are active only in sender cells to the circuit components. However, degrons incorporated in GSDMA and caspase-3 could not fully inhibit cell death caused by either protein (FIG. 1C-FIG. 1D).

To overcome these challenges, a GSDMA N-terminal domain variant was engineered, whose activity in senders can be caged by a separately expressed C-terminal domain variant (FIG. 6G). We exploited the fact that in wildtype uncleaved GSDMA, the C-terminal domain folds back onto the N-terminal domain to sterically repress its pyroptotic activity by masking a loop that mediates lipid binding. Upon cleavage of the inter-domain linker, inhibition of GSDMA N-terminal domain is relieved because the cleaved linker no longer sterically masks the loop. Inspired by these natural interactions, the N- and C-terminal domains were fused to heterodimerizing leucine zippers, which should form an artificial linker-like motif that masks the lipid-binding loop like the natural uncleaved linker. Indeed, the zipper-attached C-terminal domain (silencer) markedly suppressed pyroptosis induced by the zipper-attached N-terminal domain (active executioner) (FIG. 12B-FIG. 12D). As expected, the silencer did not inhibit pyroptosis induced by wildtype GSDMA N-terminal domain (FIG. 12B). Finally, sender cell death was specifically suppressed by expressing the silencer in senders. The silencer-expressing senders did not undergo appreciable rates of pyroptosis when internally producing, or treated with externally produced, active executioner VLPs (FIG. 6H and FIG. 12C). By contrast, non-engineered receivers taking up the VLPs underwent pyroptosis, whether the senders and receivers were separately cultured (FIG. 6H) or co-cultured (FIG. 12E).

Together, these results demonstrate that synpoptosis circuits provide a foundation for engineering synthetic killer cells that eliminate other cells without killing themselves.

Mammalian systems trigger specific cell death programs depending on cell state and desired immunological outcome. Similarly, many therapeutic challenges can be addressed if one could kill the right cells in the right way. For example, synpoptosis circuits may be delivered to tumors to conditionally induce cancer cell death, in a manner that safely amplifies anti-tumor immunity. The circuits can also be used to eliminate cells playing other harmful roles, such as senescent cells, fibrotic cells, self-targeting immune cells, and infected host cells, and could do so with appropriate immune stimulation.

The utility of cell killing has been long recognized. Previous efforts mainly used death ligands, chemogenetics, such as inducible expression and chemically induced dimerization of executioners, and, more recently, optogenetics, which allows for spatiotemporal flexibility and higher-order assembly of executioners. While these methods generally enable cell death induction, they face several challenges: they have limited ability to direct the mode of cell death in various cell contexts; they largely cannot respond to combinatorial inputs and endogenous intracellular signals; and, they do not support cell-cell transmission, which is crucial for engineering synthetic killer cells that eliminate other cells without harming themselves.

To address these challenges, there are provided herein engineered synpoptosis circuits using several design principles. First, proteolytic removal of inhibitory domains activates executioners (FIG. 1A-FIG. 2H). This principle is also used by nature, as both caspases and GSDMs are cleavage-activated proteins. Second, and conversely, cleavage-activated degrons allow proteases to suppress executioners (FIG. 1A-FIG. 1E). This suppression is useful in situations where inputs from healthy cells should deactivate cell death. Third, the two mechanisms above can be combined to allow for combinatorial control, which enables targeted death induction in specific cells (FIG. 4A-FIG. 4D). Fourth, mutant executioners can inhibit the activity of their wildtype counterpart (FIG. 3A-FIG. 3E).

In natural contexts, mammals use mixed cell death programs to modulate immunity. By contrast, traditional methods of inducing cell death either lead to only a single death mode, or passively delegate the choice of death mode to the target cell. This lack of active control poses a problem because the target cell often selects an undesirable death mode. Synpoptosis circuits offer active control by directing cell death independently of endogenous death programs and by adjusting the ratio between apoptosis and pyroptosis (FIG. 3A-FIG. 3E).

Endogenous intracellular signals can interact with engineered proteases, a common currency in synthetic protein circuits. Engineered executioners can in turn interface with such protease inputs, alone or in combination (FIG. 4A-FIG. 5C). As a proof of principle for target cell-selective killing, a Ras sensor was incorporated as an input to synpoptosis circuits (FIG. 5A-FIG. 5C). In some embodiments, synpoptosis circuits can be activated using combinations of natural pathway activities and inputs from synthetic receptors like synNotch. The synpoptosis circuits provided herein cam serve fulfill unmet needs in various synthetic biology applications.

From a translational standpoint, delivery challenges have limited the advancement of synthetic circuits. A potential paradigm to improve delivery is the engineering of sender cells that navigate the body, home to disease sites, and selectively transmit synthetic circuits into receiver cells. Demonstrated herein are the abilities to rationally program synpoptosis circuits, deliver them from one cell to another, and have them trigger customizable death programs in receiver cells (FIG. 6A-FIG. 6H). In some embodiments, synthetic killer cells can additionally provide the ability to evade exhaustion, as they would not be affected by immunosuppression that limits natural cytotoxic lymphocytes. Synpoptosis circuits can fulfill an unmet need to facilitate programmable control of cell death in various contexts, including cancer, senescence, fibrosis, autoimmunity, and infection.

### Experimental Model and Study Participant Details

### Tissue Culture and Cell Lines

All cells were cultured under standard conditions, on tissue culture grade plastic plates and dishes (Thermo Fisher) or flasks (CELLTREAT), uncoated or coated with poly-D-lysine (Thermo Fisher), at 37° C. with 5% CO₂ in humidified incubators (CellXpert C170i, Eppendorf). All cells were handled in sterile safety cabinets (SterilGARD III Advance or SterilGARD e3, Baker), and counted using the Countess 3 automated cell counter (Thermo Fisher). Dead cells were calculated using trypan blue (Invitrogen). Human embryonic kidney (HEK) cells (HEK293, T-REx-293, HEK293T, and HEK293FT variants), wildtype or engineered, were maintained in Dulbecco's Modified Eagle Media (Thermo Fisher) supplemented with 10% fetal bovine serum (Avantor), penicillin (1 unit/ml), streptomycin (1 μg/ml), glutamine (2 mM) (Thermo Fisher), sodium pyruvate (1 mM) (Thermo Fisher), and 1× Minimal Essential Media Non-Essential Amino Acids (Thermo Fisher). Before use, we passed the media through a 0.22 um vacuum filter (Falcon). Jurkat cells are maintained in RPMI 1640 with Glutamax and HEPES (Gibco), supplemented with 100× diluted pen/strep (Thermo Fisher) with 10% FBS and sodium pyruvate (100×). THP-1 cells (a gift from Mikhail Shapiro's lab at Caltech) and GSDMD-knockout THP-1 cells (a gift from Hao Wu's lab at Harvard) were maintained in RPMI 1640 with Glutamax and HEPES (Gibco), supplemented with 100× diluted pen/strep (Thermo Fisher) and 10% FBS. For routine passage or seeding, we lifted HEK cells from plates using 0.05% Trypsin-EDTA (Thermo Fisher). Jurkat and THP-1 cells were resuspended by vigorous pipetting and did not require trypsinization. HEK cells were maintained at a confluence between 10% and 90% by estimation. Jurkat and THP-1 cells were maintained at a density between 200,000 cells/ml and 2 million cells/ml. All cells were used before they reached 30 passages and then discarded. The volume of media was 100 ul to 200 ul per well on a 96-well plate, 500 ul to 1 ml per well on a 24-well plate, 1 ml-2 ml per well on a 12-well plate, 2-3 ml per well on a 6-well plate, and 10 ml-12 ml on a 10-cm dish. Regarding sex, HEK cells are female, Jurkat cells are male, and THP-1 cells are male. We verified the cells to be free of mycoplasma by polymerase chain reaction (PCR) using the MycoStrip kit (InvivoGen).

### Method Details

### Study Design

The objective of this study was to engineer protein-level synthetic synpoptosis circuits that enable programmable control of cell death in mammalian cells. Molecular biology and cell biology experiments were performed and designed to investigate the capabilities of synpoptosis circuits. Results shown represent three independent replicates, as indicated in figure legends. This study was not blinded.

### Plasmid Construction

We used the NEBuilder HiFi DNA Assembly Master Mix (New England BioLabs) to assemble gene inserts, synthesized (Integrated DNA Technologies) or amplified from previous plasmids in the lab, into restriction digested in-house cloning vectors. The insert sequences for expression in human cells were designed using a codon optimization tool (Integrated DNA Technologies). To generate mutant plasmids, we performed Gibson assembly of synthesized mutant genes or used the Q5 Site-Directed Mutagenesis Kit (New England BioLabs). For transfection, the plasmids were purified manually using the QIAprep Spin Miniprep Kit (Qiagen) or by the Qiacube machine (Qiagen). To verify their sequences before use, we sent the plasmids to Primordium or Laragen.

mRNA Production

Genes of interest were cloned into a DNA backbone containing a T7 promoter followed by an AG dinucleotide. The DNA templates were linearized and a 3′ 120-base pair (bp) polyA tract was added using PCR. The PCR product was run on an agarose gel and cleaned using the Zymoclean Gel DNA Recovery Kit (Zymo). In vitro transcribed mRNA was then synthesized using the HiScribe T7 High Yield RNA Synthesis kit (NEB). Reactions consisted of a 10× Reaction Buffer, 5 mM each of ATP, GTP, CTP, and N1-Methyl-Pseudouridine-5′-Triphosphate (TriLink), 4 mM CleanCap AG (TriLink), 500 ng of linearized DNA template, 2 μL of T7 RNA polymerase mix, and water for a final volume of 20 μL. Reactions were incubated for 2 hours at 37° C. After incubation, 5 μL of DNAse I buffer, 1 μL of DNAse I and 25 μL of water were added to the reactions. Reactions were then incubated for 15 minutes at 37° C. Finally, mRNA was purified using the RNA Clean and Concentrator-5 kit (Zymo).

### Transient Transfection

On the day before transfection, HEK cells were seeded at a density of 0.3-0.6 million cells per well on a 12-well plate, 0.1-0.3 million cells per well on a 24-well plate, or 15,000-50,000 cells per well on a 96-well plate. The cells were allowed to attach to the plate overnight, for at least 8 hours. On the day of transfection, we treated the cells with purified DNA plasmids encoding circuit components. In addition to plasmids encoding circuit components, we often included a plasmid encoding a fluorescent co-transfection marker, at roughly 50 ng DNA per well on a 24-well plate. Given the different number of molecular components in each synthetic circuit and their various relative ratios, the total amount of DNA ranged from 50 to 1 μg per well on a 24-well plate, and was scaled by 0.5 or 4 when other 12-well or 96-well plates were used, respectively. On the same plate, we often equalized the total amount of DNA across all transfected wells, using a filler plasmid encoding the neomycin resistance gene. To transfect cells with plasmid DNA, we used three types of transfection reagents—Lipofectamine 2000 (Thermo Fisher), Lipofectamine 3000 (Thermo Fisher), and FuGENE HD (Promega), according to manufacturers' instructions. Briefly, for a typical transfection experiment on a 24-well plate, we mixed plasmids and the transfection reagent, around 200 ng of DNA per 1 μl of Lipofectamine 2000, in 25 μl of Opti-MEM reduced serum medium (Thermo Fisher), incubated the mixture for 5-10 min at room temperature, and then added another 25 μl of Opti-MEM before transferring the mixture dropwise to culture wells. We also performed some transfection experiments using the Lipofectamine 3000 kit (Thermo Fisher), at a ratio of 2,000 ng of DNA, 2 ul of 4 ul of P3000, and 3 μl of Lipofectamine 3000. We similarly diluted the mixture in Opti-MEM before adding it to cells. For transfection with FuGENE, we used a ratio of 4 μl FuGENE:1 μg of DNA, and incubated the DNA-FuGENE mixture at room temperature for 5 min before transfection. To enable protein expression from plasmids with a doxycycline-inducible promoter, doxycycline was added to the cell culture at a final concentration of 100 ng/mL. In experiments involving Q-VD-OPh, the compound was added to the cell culture at a final concentration of 30 μM, immediately after the cells received the transfection mixture. To transfect cells with in vitro transcribed mRNA, Lipofectamine MessengerMax (Thermo Fisher) was used. Briefly, for transfection of one well in a 96-well plate, 0.1 μL of Lipofectamine MessengerMAX was diluted in 1.25 μL Opti-MEM and incubated for 10 minutes at room temperature. mRNA was then added to Opti-MEM to the appropriate conditions at the following amounts: 12.5 ng of fluorescent co-transfection marker, 56.25 ng of protease, and 56.25 ng of engineered executioner. Diluted mRNA was then added to diluted MessengerMAX reagent and incubated for 5 minutes at room temperature before addition to Jurkat or THP-1 cells in RPMI media.

### Cell Line Engineering

For experiments that required stable transgene expression, stable cell lines were engineered using lentiviruses followed by antibiotic selection, or using the PiggyBac transposase system. To generate lentiviruses, HEK293 cells were plated (maintained in geneticin at 500 μg/mL) at a density of 0.6 million cells per well on a 12-well plate, or to around 90% confluence by estimation in a T25 flask. The next day, the cells were co-transfected using Lipofectamine 2000 with three plasmids, pLVX-M-PURO (Addgene plasmid #125839) containing the gene of interest, the TEVP-activatable Citrine reporter for instance, psPAX2 (Addgene plasmid #12260), and pCMV-VSV-G (Addgene plasmid #8454). We kept the mass ratio of the three plasmids at 9:9:2 (450 ng:450 ng:100 ng). In some experiments involving cells stably expressing two or more transgenes, the lentiviruses were generated using multiple pLVX-M-PURO plasmids carrying different genes of interest. An alternative ratio of pLVX-M-PURO:psPAX2:pCMV-VSV-G was 2:2:1 (2.4 μg:2.4 μg:1.2 μg), which was used to generate lentiviruses for stable integration of IL-1β and IL-18 genes. 48 hours post transfection, supernatants were collected containing the lentiviruses and used them to transduce HEK cells, 0.3 million per well, on a 24-well plate. We then waited 48 hours to allow integration and transgene expression in treated cells. Afterwards, the transduced cells were transferred to a 6-well plate and added puromycin (1 μg/ml, Sigma-Aldrich) to select polyclonal cells with successfully integrated transgenes over a period of two weeks, during which the media was refreshed every two days. To generate control cells, empty lentiviruses were used. The RasGC line, or HEK293 cells stably expressing human H-Ras G12V mutant along with a Cerulean fluorescence marker, was generated using the PiggyBac method. The mutant Ras and Cerulean marker were cloned into a PiggyBac DNA vector connected by a 2A peptide. Expression of the mutant Ras and Cerulean marker was driven by a dox-inducible CMV promoter. Briefly, 50 ng of the PiggyBac transposase and 200 ng of the cargo-carrying DNA vector were co-transfected into HEK293 cells seeded in a 24-well plate. One week later, the cells were sorted based on Cerulean fluorescence to obtain the final RasGC line.

### Intercellular Transmission

As a model VLP that demonstrates intercellular transmission of synpoptosis circuits, integrase-deficient lentiviruses were used. We built the VLP packaging plasmid by introducing the inactivating D64V mutation into the integrase gene within the standard lentiviral vector psPAX2 (hereafter, modified psPAX2, or M-psPAX2). For experiments that involved circuit senders and receivers, sender HEK cells were seeded on a 12-well plate at a density of 0.6 million cells per well (day 1). On the following day (day 2), using Lipofectamine 3000 (Thermo Fisher), we transfected the sender cells with 450 ng of pLVX-M-PURO plasmid containing the cargo, 450 ng of M-psPAX2, and 100 ng of pCMV-VSV-G. To generate VLPs encoding two cargoes, we performed similar transfections using two cargo-containing plasmids, each at 225 ng. At 8 hours post transfection, we removed the media containing residual Lipofectamine particles, washed the cells gently with fresh media, and replaced the media. One day after transfection of sender cells (day 3), we typically seeded receiver HEK cells on a 24-well plate at a density of around 0.3 million cells per well or on a 96-well plate at around 75,000 cells per well. We also tried other receiver densities, in which case we needed to dilute the cells if they approached confluency during the course of the experiment. Two days after transfection of sender cells (day 4), we collected the sender supernatant, centrifuged it at 3,000 g for 5 min to remove cell debris, and passed it through a cellulose acetate filter with 0.45 μm pore size (VWR). We transferred the VLP-containing conditioned media, without concentrating them, to receiver cells. One day after media transfer (day 5), the media was refreshed. Finally, two days after media transfer (day 6), we collected receiver cells for further analysis. In co-culture experiments, sender HEK cells were pre-stained with Vybrant DiD (Invitrogen) by incubation at 37° C. for 15 min and washed three times to remove the unbound stain, according to manufacturer's instructions. Then, the senders were transiently transfected with DNA encoding the silencer, VLP-packaging components, and the active executioner cargo (GSDMA N-Z, or empty cargo as control). 8 hours post transfection, the supernatant was discarded and the transfected senders were washed twice with DPBS. Then, the senders were lifted by trypsinization and mixed with receivers, which were not stained, at a roughly 1:1 cell number ratio in a 24-well plate. After about 40 hours, we collected the co-cultured cells, stained them with Sytox, and analyzed cell death by flow cytometry.

### Cell Staining and Flow Cytometry

For experiments involving DNA transfection, the cells were collected from culture wells between 16 and 24 hours post transfection, a selected time window during which substantial cell death occurs while apoptotic cells remain largely unlysed, allowing Sytox to reliably distinguish between apoptotic and pyroptotic cells. To collect all cells, we first transferred the supernatant containing floating cells to a 1.5-ml Eppendorf tube or a 15-ml Falcon tube. Then, we gently washed the adherent cells with Dulbecco's phosphate-buffered saline (DPBS, Gibco) and trypsinized them using 0.05% Trypsin-EDTA (ThermoFisher) for 5 min at room temperature. Afterwards, we pooled the trypsinized cells with the supernatant and pelleted the cells in a tabletop centrifuge (Eppendorf 5424 or 5804R) at 200 g for 5 min. We then carefully removed the supernatant and resuspended the pellet in 1.5 ml of the Hank's Balanced Salt Solution (HBSS, Gibco) containing 2.5 mg/ml bovine serum albumin (BSA) and 2.5 mM calcium chloride. For Sytox staining, we used one drop of the SYTOX Green Ready Flow reagent (R37168, Invitrogen). For Annexin staining, we used one drop of Annexin Ready Flow reagents, conjugated with either Pacific Blue (R37177) or Alexa Fluor 488 (R37174). For staining with both dyes, one drop of each was added. For TO-PRO-3 staining, an indicator of apoptosis or membrane damage during pyroptosis, we used TO-PRO-3 (Invitrogen T3605) at a final concentration of 1 μM. Afterwards, the cells were incubated on ice in the dark for 15 min. For experiments involving lentiviruses or VLPs, we collected cells for staining around 2 days post treatment with lentiviruses or VLPs. For experiments involving mRNA transfection, cells were collected for staining between 8 and 12 hours post transfection. To analyze cells by flow cytometry, we filtered the collected and stained cells through a 40-μm cell strainer (Falcon) and then transferred them to a U-bottom 96-well plate on top of a cold block. The volume in each well was 150 to 250 μl. We then subjected the cells to analysis by the CytoFLEX S flow cytometer (Beckman Coulter), with forward and side scatters set at 185 and 115 (other similar settings were also used). Mainly four channels recorded cellular fluorescence of interest in the study, the FITC channel (for Citrine, Sytox Green, and Annexin-Alexa Fluor 488; excitation 488 nm, emission 525/40 nm; gain set at 1), the ECD channel (for Cherry; excitation 561 nm, emission 610/20 nm; gain set at around 50-100), the PB450 channel (for Annexin-Pacific Blue; excitation 405 nm, emission 450/45 nm; gain set at around 50-100), and the APC channel (for Vybrant DiD; excitation 638 nm, emission 660/20; gain set at around 100). For experiments involving multiple colors, we collected single-color controls to set the compensation matrix, using built-in functions in the FlowJo software (version 10.4, BD Biosciences). Gray windows or horizontal lines that mark y-axis ranges in the figures were established by negative and positive transient transfection controls, established by mock transfection (lipofectamine only, transfection of Cherry only, or transfection of an inactive circuit containing a mutant executioner, such as caspase-3-C163A or GSDMA-E14K-L184D mutant, and the TEVP-C151A mutant) and transfection of a constitutively active killer (protease-activated circuit or an active executioner), respectively. In transfection experiments involving incoherent feedforward loop (IFFL)-regulated GFP expression, cells were left unstained, and the gray window indicates negative and positive controls of the GFP signal, set by mock transfection and transfection of GFP without IFFL regulation, respectively. In experiments involving a fluorescent co-transfection marker, such as Cherry, we often gated on the co-transfection marker to analyze the effects of synpoptosis circuits on transfected cells. Some cells were Annexin-positive and Cherry-negative. These cells were of several possible origins and excluded from analysis.

### Cell Imaging

Live and dead cells were examined using the EVOS FL Auto cell imaging system (Life Technologies) approximately 16-24 hours post DNA transient transfection. The three EVOS light cubes correspond to cyan fluorescence protein (excitation 445/45 nm, emission 510/42 nm), yellow fluorescent protein (excitation 500/24 nm, emission 542/27 nm), and red fluorescent protein (excitation 531/40 nm, emission 593/40 nm). For magnification, we typically used the EVOS AMG 10× or 20× objective. The exposure was usually set at around 200 milliseconds and the gain at around 10 decibels. Images were captured as tiff files for quantification purposes. Images within a figure panel were presented using the same brightness and color scales.

### Cell Viability and Cytotoxicity Experiments

For ATP-based viability experiments, 15,000 HEK293 cells were seeded to each well of an opaque 96-well plate and incubated overnight under standard culture conditions. After 24 hours, the cells were transfected with 100 ng in vitro transcribed mRNA, previously prepared using the Lipofectamine MessengerMax transfection kit as per the manufacturer's protocol (0.3 μL Lipofectamine per 100 ng mRNA, in 12.5 μL total volume of mRNA-Lipofectamine transfection mix). Cells were incubated for another 8 hours before their total ATP levels were measured using the CellTiter-Glo 2.0 cell viability assay (Promega). Both cells and CellTiter-Glo 2.0 reagent were equilibrated to room temperature before use. 100 μL of the assay reagent was added to 100 μL of sample medium per well. After mixing by orbital shaking (1 mm shaking radius, 300 cycles per minute, 2 min), the plate was incubated for 10 min at RT and luminescence was subsequently recorded using a GloMax Discover microplate reader (Promega) without a filter and with a 0.7-second integration time. ATP-based cell viability was calculated by dividing the luminescence of the compound treated by the luminescence of untreated sample wells. For LDH release experiments, the same number of cells were seeded into a standard 96-well plate, and after 24 hours, transfected with the same amounts of mRNA using the same transfection procedures. Wells used to determine the levels of spontaneously released LDH were transfected with Opti-MEM only. At 8 hours post transfection, the Invitrogen's CyQUANT LDH cytotoxicity assay kit (Invitrogen) was used to measure LDH release per well. The reaction reagent was prepared by adding ddH2O (11.4 mL) and assay buffer stock solution (0.6 mL) to the substrate mix. To determine the maximum LDH activity, 10× lysis buffer (10 μL) was added to corresponding wells serving as maximum LDH activity controls. After 45 min incubation under standard culture conditions, 50 μL of supernatant sample medium per well was transferred to a black-walled clear bottom 96-well plate. After adding 50 μL reaction mixture reagent per well, the plate was mixed and incubated at room temperature for 30 min. Formazan production, proportional to LDH amounts, was stopped by supplementing 50 μL stop solution per well. Absorbance at 490 nm and 680 nm was then measured using a BioTek Cytation 5 cell imaging multimodal reader (Agilent). LDH release was calculated by subtracting the spontaneous from circuit-treated LDH activity and subsequent division by the difference between maximum and spontaneous LDH activity. For ATP release experiments, the same number of cells were seeded into an opaque 96-well plate and transfected the same way with mRNA after 24 hours. ATP release was measured using the RealTime-Glo Extracellular ATP assay (Promega). Briefly, 4× RealTime-Glo Extracellular ATP assay reagent was prepared by adding supplemented, pre-warmed DMEM culture medium (Gibco) to RealTime-Glo Extracellular ATP assay substrate. 50 μL of the assay reagent was added to 150 μL sample medium per well, and the plate was mixed by orbital shaking (2 mm shaking radius, 300 cycles per minute, 45 s). Immediately after adding the assay reagent, luminescence was recorded every hour over 20 hours using a GloMax Discover microplate reader (Promega) without a filter. The integration time was 0.7 seconds, and the incubation temperature was 37° C.

### Cytokine Measurements

Concentrations of IL-1β and IL-18 in the cell culture supernatant were measured using enzyme-linked immunosorbent assays (ELISA). HEK293 cells stably expressing IL-1β and IL-18 were seeded into 24-well plates at 150,000 cells per well and then transiently transfected with lipofectamine only (mock transfection control) or 300 ng of DNA-encoded TEVP-activated apoptosis or pyroptosis circuits. Between 16 and 24 hours post-transfection, supernatants were collected into 1.7-ml tubes and spun at 15,000 g for 5 min using a tabletop centrifuge (Eppendorf). Clarified supernatants were transferred to clean 1.5-ml tubes, and then diluted 100- to 1000-fold before ELISA using SimpleStep IL-1β and IL-18 ELISA kits (Abcam). Briefly, 50 μl of diluted samples and 50 μl of the antibody cocktail solution, which contained both capture and detection antibodies, were added to each well of the microplate strips. The strips were incubated for around 1 hour at room temperature on a shaker. Each well was washed with 350 μl of wash buffer three times before 100 μl of development solution containing TMB (tetramethylbenzidine), a substrate for horseradish peroxidase (HRP), was added. The strips were covered in foil and shaken for 10 min at room temperature. 100 μl of stop solution was added to each well to quench the reaction. Absorbance at 450 nm was recorded using a Cytation 3 plate reader (BioTek). Diluted IL-1β and IL-18 concentrations were calculated based on standard curves established using lyophilized purified proteins dissolved to known concentrations. Undiluted concentrations were then obtained by multiplying the diluted concentrations by the corresponding dilution factor.

### Protein Structure Analysis

To evaluate molecular constraints on the design of synpoptosis circuit components, several structures from the protein data bank (PDB) database were analyzed, which can be located by the following accession numbers. These structures include cryo-electron microscopy (cryo-EM) structures of GSDM pores (PDB 6CB8, 6VFE, 8GTN, 8ET2, and 8SL0), as well as X-ray crystal structures of GSDMA (GSDMA3 variant) (PDB 5B5R), GSDMD (PDB 6N9O), bGSDM (PDB 7N51), caspase-3 (PDB 1I3O), TEVP (PDB 1Q31), and TVMVP (PDB 3MMG). To examine molecules whose structures are not available, such as engineered GSDMA containing a TEVP cleavage site or leucine zippers, we generated models using AlphaFold2 (ColabFold v1.5.2). For molecular visualization, we used PyMOL software (2.5.2) (Schrodinger).

### Quantification and Statistical Analysis

Sample sizes and analytical measures are indicated in the figure legends and method details. To generate data visualizations, we used ImageJ, Excel (Microsoft), the built-in layout editor in FlowJo (version 10.4, BD Biosciences), GraphPad Prism (version 9, Dotmatics), and Matplotlib (Python). Sigmoidal curve was fitted using the four parameter logistic (4PL) nonlinear regression method in GraphPad Prism. For plots that required coding, we used the Jupyter Notebook (jupyter.org) with the assistance of ChatGPT (version 4, OpenAI). To reduce visual overload, each flow cytometry scatter plot shows 5,000 randomly sampled data points, and each flow cytometry histogram shows the fluorescence distribution of 5,000 randomly sampled data points divided into 50 bins on the x-scale. In plots that quantify fractional killing, the upper and lower limits of signal quantification were defined by positive and negative controls, respectively, as indicated in method details. In transfection experiments involving a fluorescent co-transfection marker, we often gated on the co-transfection marker by flow cytometry to identify transfected cells before quantifying killing fractions. This approach allowed us to attribute cell death to synpoptosis circuits.

### Example 2

### Modifications of Synpoptosis Circuits

Provided in this example are further modifications and improvements to the synpoptosis circuits of the disclosure.

### Strategy to Fine-Tune Activation Threshold of Cell Death Executioner Proteins

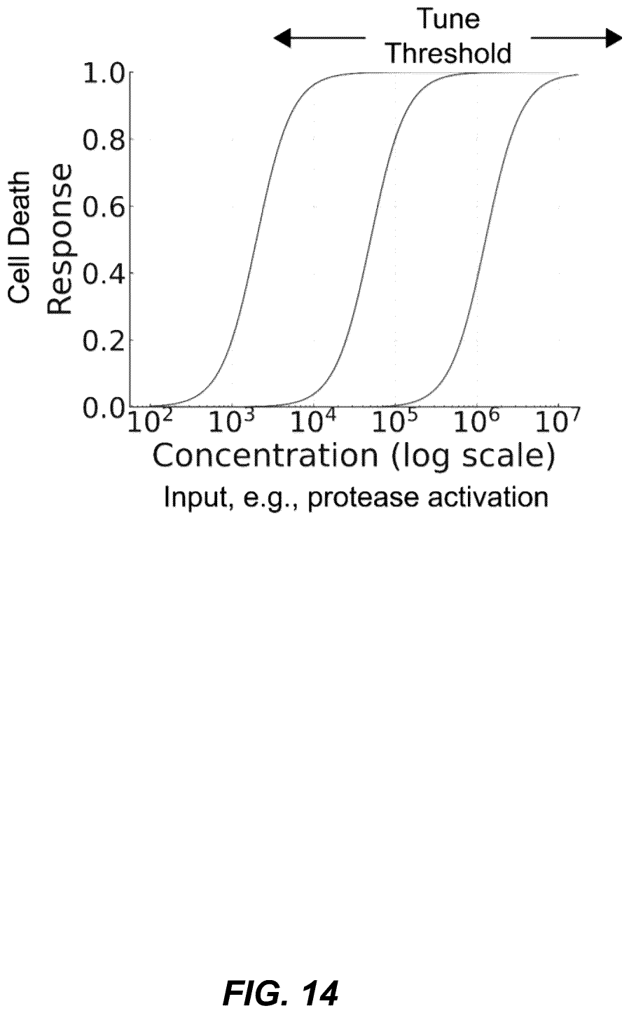

Cell death executioner proteins can be activated by protease-catalyzed cleavage of cognate protease cleavage sites. Once the amount of cleaved cell death executioner proteins reaches a critical threshold, the corresponding cell will undergo cell death. For many applications, the input protease activities that should trigger or not trigger cell death are set, e.g., in the context of a cancer sensor, by the endogenous expression levels of the target. For instance, physiological Ras expression levels define sensor activation in Ras-driven cancer cells. To prevent the elimination of off-target cells (e.g., cells with non-oncogenic Ras signaling) while achieving complete killing of on-target cells (e.g., cells with oncogenic Ras signaling), one needs to tune the input protease activity threshold that triggers cell death. Here, we developed a strategy for this, enabling fine tuning cell death executioner activation thresholds (FIG. 14).

The cleavage efficiency of proteases is known to depend on corresponding accessible cleavage sites. Mutations to native protease cleavage sites can slow (i) the recruitment of substrates to the protease active site, (ii) catalytic reaction steps, or (iii) the release of the substrate from the protease. As described herein, this property to modulate cell death executioner activation thresholds. Specifically, we surveyed the following constructs with modified protease cleavage sites across a wide range of possible cell death executioner and input protease expression regimes:

Caspase 3 with ENLYFQS TEVP protease cleavage site (native protease cleavage site is ENLYFQG/S, SEQ ID NOs: 1-2), which serves as a control; Caspase 3 with ENLYFQY (SEQ ID NO: 3) TEVP protease cleavage site; Caspase 3 with ENLYFQQ (SEQ ID NO: 4) TEVP protease cleavage site; Caspase 3 with ENLFFQS (SEQ ID NO: 5) TEVP protease cleavage site; Caspase 3 with ENLFFQY (SEQ ID NO: 6) TEVP protease cleavage site; Caspase 3 with ENLFFQQ (SEQ ID NO: 7) TEVP protease cleavage site; Caspase 3 with both native TEVP and TVMVP cleavage sites separated by linker; Caspase 3 with native TEVP cleavage site fused to C-terminal TEVP-cleavable DHFR degron.

To evaluate how the mutated protease cleavage sites affect Caspase 3 activation, we polytransfected (i) input proteases (P3/P4-zipper reconstituted TEVP as a positive control, split TEVP without reconstitution domain as a negative control, or NeoR to evaluate caspase baseline activity; all with IRES-BFP to enable expression tracking) and (ii) output caspase (caspase+co-transfected GFP for expression tracking) in HEK293FT cells. Annexin staining as described above in Example 1 was used to quantify cell death.

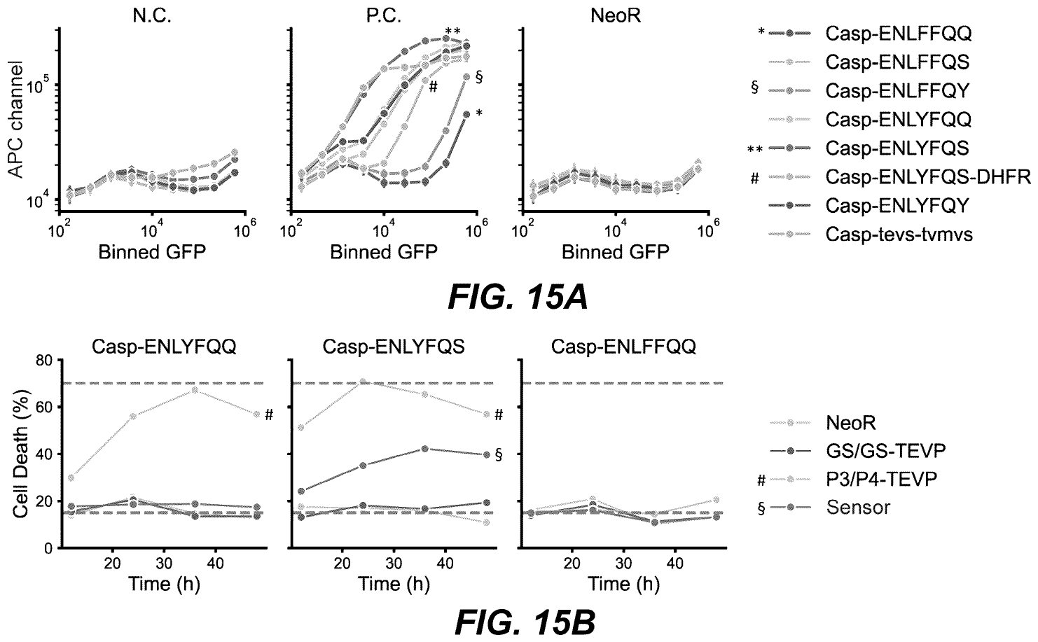

It was found that at high caspase expression and in the presence of some non-reconstituted split protease, caspases with native cleavage sites or both TEVP and TVMVP cleavage sites show leaky activation. Further, the data show that mutation of the caspase cleavage site enables robust and fine-grained tuning of caspase activation thresholds. “ENLFFQQ” (SEQ ID NO: 7) requires the highest input/output expression for activation, followed by “ENLFFQY” (SEQ ID NO: 6), “ENLYFQS (SEQ ID NO: 2)+TEVP-cleavable DHFR”, “ENLFFQS” (SEQ ID NO: 5)˜“ENLYFQY” (SEQ ID NO: 3)˜“ENLYFQQ” (SEQ ID NO: 4), “ENLYFQS” (SEQ ID NO: 2), “both TEVP and TVMVP cleavage site” in decreasing order (FIG. 15A).

Moreover, it was evaluated if some of these mutations affect the maximal caspase activation accessible and the time dependency between caspase activation and cell death by co-transfecting (i) input proteases (P3/P4-zipper reconstituted TEVP as a positive control, split TEVP without reconstitution domain as a negative control, or NeoR to evaluate caspase baseline activity; all with IRES-BFP to enable expression tracking) and (ii) output caspase (“ENLYFQS” (SEQ ID NO: 2)/“ENLYFQQ” (SEQ ID NO: 4)/“ENLFFQQ” (SEQ ID NO: 7)+co-transfected GFP for expression tracking) in HEK293FT cells and measuring cell death every 24 h over the course of 4 days. While the “ENLFFQQ” (SEQ ID NO: 7) reduces the maximum activation to a degree where the complete killing of the cell population is not possible anymore, “ENLYFQQ” (SEQ ID NO: 4) and “ENLYFQS” (SEQ ID NO: 2) lead to a comparable maximal fraction of dead cells, showing that the tunability of the caspase does not come at the cost of reduced maximal activation (FIG. 15B).

Thus, as described herein, alternation of protease cut sites enables fine-tuning of caspase activation thresholds without affecting maximal cell death. These principles can translate to all the other synthetic cell death executioner proteins described herein. Further, this modulation scheme is not specific to TEVP. TEVP can be switched out for any other protease as long as cleavage sites are updated correspondingly.

### Localization of Cell Death Executioners to Cellular Compartments Enables Signal Amplification and Attenuation.

A key advantage of the engineered cell death execution circuits of this disclosure, is their flexibility in responding to diverse endogenous cell inputs. For instance, cell death execution can be conditional on Ras, beta-catenin, or p53 signaling. Natural signaling often occurs at specific cellular sites, e.g., the cellular membrane, liquid condensates, or the nucleus. Thus, actively localizing cell death execution proteins to different cellular compartments may tune the characteristics of the resulting apoptosis and pyroptosis processes.

### Co-Localization or Separation of Input and Output Modules Enables Signal Processing.

For example, the Ras pathway predominantly signals on the cell membrane. To boost the detection sensitivity of a Ras sensor (See, e.g., US patent application publication US20230220011, the content of which is hereby incorporated by reference in its entirety), one can direct a protease-activatable apoptosis execution protein (Caspase 3) to the cellular membrane. In addition, compartment localization of cell death proteins may have opposite, attenuating effects on cell death. For example, a cytoplasmic Ras sensor can equally detect membrane and endoplasmic reticulum (ER) localized Ras signaling. However, if the goal is to condition cell death only on membrane Ras signaling, then a non-discriminatory, cytoplasmic Casp3 could induce excessive cell death. On the other hand, a membrane-localized Casp3 would reduce the amount of detected ER Ras signaling and consequently lower the amount of undesired cell death.

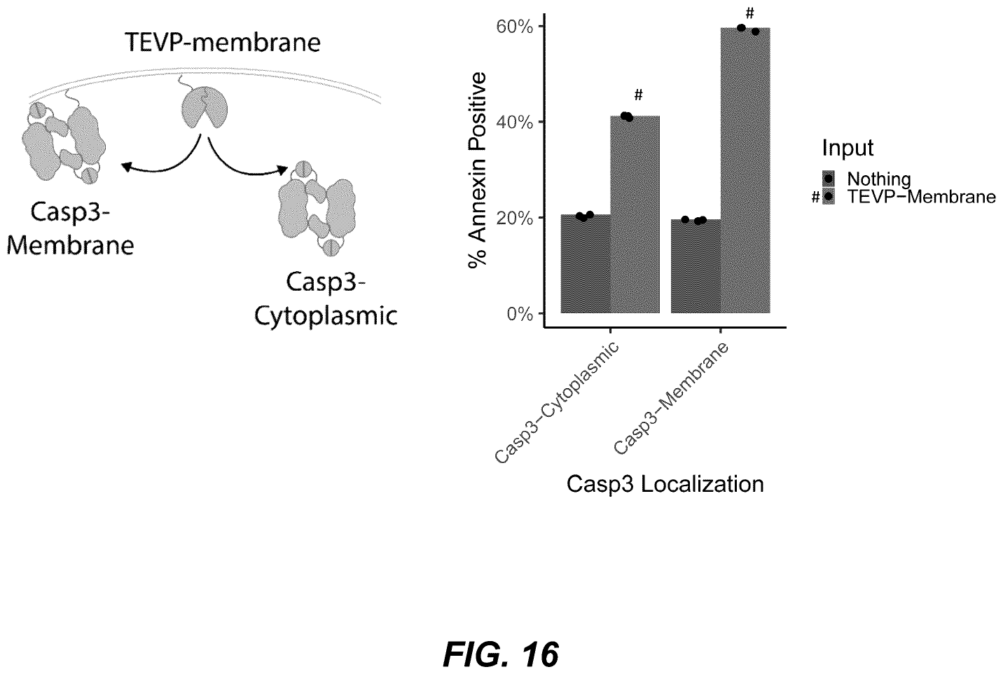

HEK293 cells were polytransfected with (i) plasmids encoding cytoplasmic or membrane-bound (CAAX) TEVP-activatable Caspase 3 and (ii) either a membrane-bound TEVP (CAAX) or a filler, empty plasmid (“nothing”). As described above in Example 1, cell death was quantified via Annexin V staining and flow cytometry. In the presence of only the filler plasmid, both Casp3 conditions induce a low level of background cell killing. In the presence of membrane TEVP, cytoplasmic Casp3 results in ˜40% cell death. On the other hand, co-localization of membrane TEVP and Casp3 results in almost double the amount of cell killing. (FIG. 16).

### Re-Localization of Output Modules Tunes Output Signal Strength

Independently of target and sensor localization, localization of cell death execution proteins in and of itself can enable tuning of the sensitivity and specificity of cell death execution. For example, Gasdermin (GSDM) family proteins are primarily cytoplasmic but form membrane pores after activation. As such, a synthetic protein design where Gasdermins are directly membrane localized can directly influence the characteristics of the pyroptosis process. Specifically, membrane localization of GSDMA maintains its ability to be cleaved and activated by a constitutive TEV protease. On the other hand, in the absence of TEV protease activation, membrane-localized GSDMA can self-activate at high expression levels. This can be beneficial in contexts where some baseline inflammation is desirable (e.g., in immunologically cold tumor microenvironments). Nuclear export and import signals bias the protein localization of attached proteins to the cytoplasm or nucleus, respectively. As many GSDM functions require membrane localization, nuclear localization of these proteins will inactivate a large portion of these proteins. This mechanism can serve as a signal attenuation mechanism. Additionally, this attenuation can either be constitutive, biasing the proteins to shuttle into the nucleus over cytoplasmic, or conditional on proteolytic cleavage, such that the nuclear localization signal is conditionally removed.

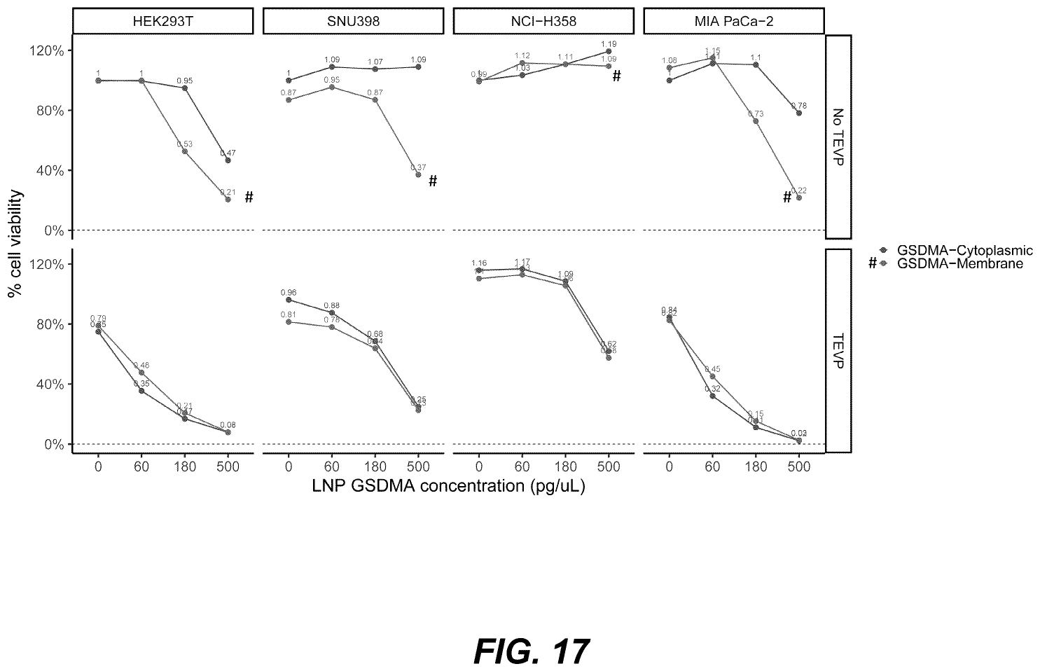

Four cancer cell lines were transfected with different circuit components that were in vitro transcribed as mRNAs and encapsulated/delivered using lipid nanoparticles (LNPs). Across all cell types and with increasing concentrations of the GSDM proteins, both cytoplasmic and membrane-bound GSDM proteins are activatable by TEVP, yielding comparable maximal killing. On the other hand, GSDM proteins showed different amounts of self-activation in the absence of TEVP when they are either membrane-localized or cytoplasmic (FIG. 17).

Altogether, the described design strategies enable the amplification or attenuation of both on-target or off-target signals in the context of Synpoptosis circuits.

### Rational Protein Design Tunes Cell Death Protein On- and Off-Target Killing.

The TEVP activatable Caspase 3 designs described herein can induce TEVP-independent cell killing at high concentrations. As described below, rational engineering of the original Caspase 3 designs can tune the on- and off-target killing efficiency.

In the TEVP-activatable Caspase 3 design described in Example 1, the large and small subunits of Caspase 3 are fused via a TEVP cleavage site, locking the large and small subunits in an inactive conformation. Subsequently, cleavage by TEVP releases this inhibition and activates the caspase. In some cases, input-independent and dependent caspase-induced cell death needs to be strengthened or reduced for specific therapeutic applications, thus, we further engineered the Caspase 3 designs from Example 1 above to reduce TEVP-independent cell killing.

Two constructs that exemplify the types of engineering that can be performed to change Caspase 3 are described below.

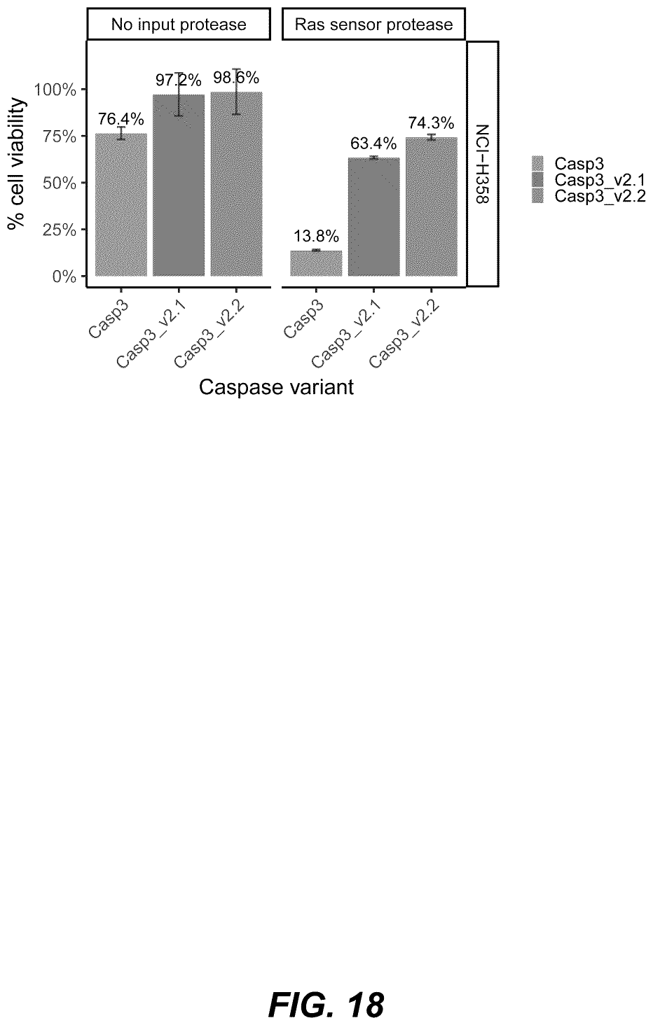

The first variant is termed Caspase3_v2.1 (nHalo-tevs-Casp3te-tevs-cHalo-CAAX): We fused a spit halo protein to opposite ends of the Caspase 3 protein. Due to the reconstitution of N- and C-halo protein halves, the entire synthetic caspase is stabilized, making TEVP-independent Casp3 activation less likely (FIG. 18). This stabilization is conditional, as it can be removed by TEVP.

The second variant is termed Caspase3_v2.2 (nCasp3-tevs-nHalo-tevs-cHalo-tevs-cCasp3-CAAX): We fused the same split Halo protein in the middle of the Casp3 protein to further introduce strain around the cleavage site. Similarly, background Casp3 activation is reduced (FIG. 18).

In this experiment, we tested the wild-type TEVP activatable Casp3 protein against the engineered Casp3 variants (v2.1 and v2.2) as described above. Each Casp3 variant was tested without an input protease and with a Ras sensor protease. Without an input protease, the original Casp3 variant induced a non-negligible cell death (>20%). With the v2.1 and v2.2 additions, this background cell death was reduced to statistically non-significant amounts.

In both examples, rational engineering from first principles allowed for engineering of changed Caspase 3 components with different on- and off-target activities. This showcases the generality of using rational engineering to tune the quantitative characteristics of the output modules.

Split Caspases can Sense Cell States and Conditionally Trigger Cell Death without Requiring Additional Sensor Modules

Synpoptosis actuator modules described above in Example 1 require proteases for their activation. Most input proteases currently available are of non-human origin and can provoke immune reactions when expressed in humans. Thus, there is a need for alternative synpoptosis actuator activation strategies that do not depend on non-human proteases or proteases in general.

While split proteases can be used as endogenous signaling sensors, it remains unclear if a similar strategy can be applied to use split Caspases to sense endogenous pathways. In the split protease design, when fused to domains that bind to a target protein, such split proteases conditionally reconstitute only if the cognate target is present. Here, we apply the same concept of target-conditional enzyme reconstitution to caspases instead of proteases. Caspase 3 contains two domains that need to pair in a particular conformation for the enzyme to induce cell death. Naturally, both domains are expressed as a single protein but fused in a fashion that prevents cell death. To engineer Caspase 3 as a single sensor-response module, the enzyme was split into two domains at the site where Capase 3 naturally gets cleaved upon activation. Then, the corresponding Caspase 3 halves were fused to binders, enabling input sensing.

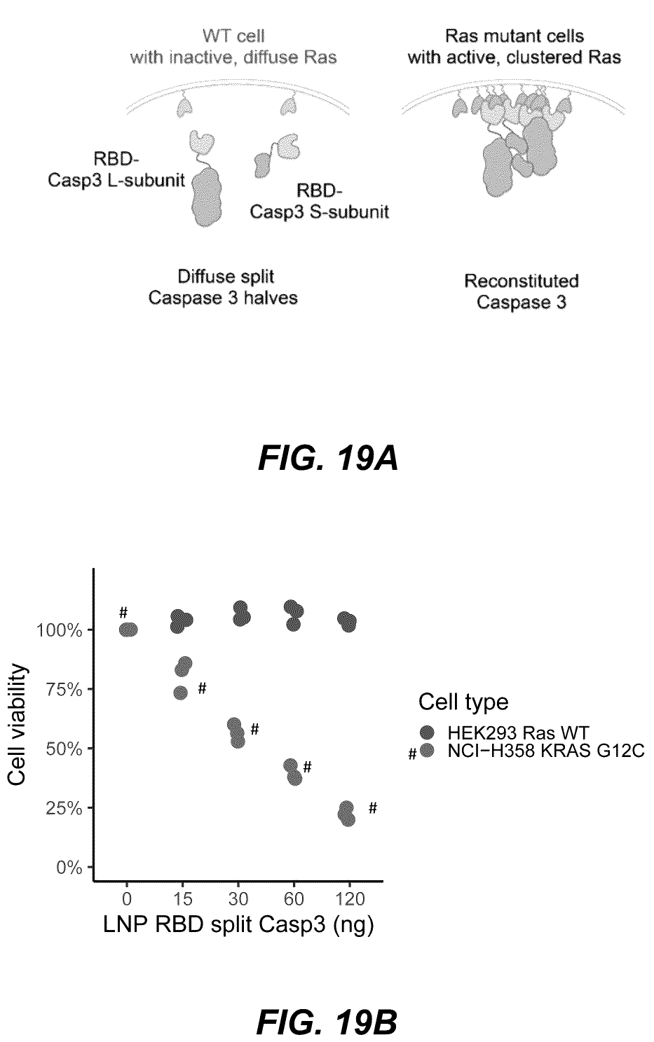

The complementary Caspase 3 halves were fused to binders selectively targeting the mutant Ras. We in vitro transcribed mRNAs encoding this split Caspase 3 Ras binding domain (RBD) designs and encapsulated them in lipid nanoparticles. We added increasing amounts of this lipid nanoparticle (0-120 ng) in an off-target HEK293 wild-type Ras cell line and an on-target NCI-H358 KRAS G12C mutant cell line and measured cell viability by CellTiterGlo. Cell viability was normalized to the 0 ng condition. It was found that cell death increased as a function of increasing amounts of the RBD-split Casp3 construct in the Ras mutant cell line H358 but not in the HEK293. See, e.g., FIG. 19A-FIG. 19B.

Taken together, these results show that a split Caspase 3 enzyme, or more generally, split cell death executioner proteins, can sense endogenous inputs while preserving high sensitivity and specificity.

TABLE 4
EXEMPLARY SEQUENCES OF THE DISCLOSURE

| | | SEQ |
|---|---|---|
| | | ID |
| Name/Description | Sequence | NO: |
| TEVP cleavage | ENLYFQG | 1 |
| site | | |
| TEVP cleavage | ENLYFQS | 2 |
| site | | |
| Modified | ENLYFQY | 3 |
| cleavage site | | |
| Modified | ENLYFQQ | 4 |
| cleavage site | | |
| Modified | ENLFFQS | 5 |
| cleavage site | | |
| Modified | ENLFFQY | 6 |
| cleavage site | | |
| Modified | ENLFFQQ | 7 |
| cleavage site | | |
| Gasdermin TEV | FYDAMDGQIQGSVEL | 8 |
| insertion site | | |

wild type

| | | |
|---|---|---|
| Gasdermin with | FKENLYFQSNMLDVQVQGLVEV | 9 |
| TEV insertion | | |
| Caspase-3-L-tev- | MGSENTENSVDSKSIKNLEPKIIHGSESMDSGISLDNSYKMDYPEMGL | 10 |
| S | CIIINNKNFHKSTGMTSRSGTDVDAANLRETFRNLKYEVRNKNDLTRE | |
| | EIVELMRDVSKEDHSKRSSFVCVLLSHGEEGIIFGTNGPVDLKKITNF | |
| | FRGDRCRSLTGKPKLFIIQACRGTELDCGIETENLYFQSGVDDDMACH | |
| | KIPVEADFLYAYSTAPGYYSWRNSKDGSWFIQSLCAMLKQYADKLEFM | |
| | HILTRVNRKVATEFESFSFDATFHAKKQIPCIVSMLTKELYFYH | |
| Caspase-3-Z-L | MGSEQLEKKLQALEKKLAQLEWKNQALEKKLAQENTENSVDSKSIKNL | 11 |
| | EPKIIHGSESMDSGISLDNSYKMDYPEMGLCIIINNKNFHKSTGMTSR | |
| | SGTDVDAANLRETFRNLKYEVRNKNDLTREEIVELMRDVSKEDHSKRS | |
| | SFVCVLLSHGEEGIIFGTNGPVDLKKITNFFRGDRCRSLTGKPKLFII | |
| | QACRGTELDCGIETD | |
| Caspase-3-S-Z- | MGSSGVDDDMACHKIPVEADFLYAYSTAPGYYSWRNSKDGSWFIQSLC | 12 |
| tev-Deg | AMLKQYADKLEFMHILTRVNRKVATEFESFSFDATFHAKKQIPCIVSM | |
| | LTKELYFYHGGSGALKKELQANKKELAQLKWELQALKKELAQGGSGLE | |
| | NLYFQSGSGSISLIAALAVDYVIGMENAMPWNLPADLAWFKRNTLNKP | |
| | VIMGRHTWESIGRPLPGRKNIILSSQPSTDDRVTWVKSVDEAIAACGD | |
| | VPEIMVIGGGRVIEQFLPKAQKLYLTHIDAEVEGDTHFPDYEPDDWES | |
| | VFSEFHDADAQNSHSYCFEILERR | |
| Caspase-3-tevD- | MGSENLYFQYHKSGAWKLPVSLVKEQLEKKLQALEKKLAQLEWKNQAL | 13 |
| Z-L | EKKLAQENTENSVDSKSIKNLEPKIIHGSESMDSGISLDNSYKMDYPE | |
| | MGLCIIINNKNFHKSTGMTSRSGTDVDAANLRETFRNLKYEVRNKNDL | |
| | TREEIVELMRDVSKEDHSKRSSFVCVLLSHGEEGIIFGTNGPVDLKKI | |
| | TNFFRGDRCRSLTGKPKLFIIQACRGTELDCGIETD | |
| Caspase-3-S-Z | MGSSGVDDDMACHKIPVEADFLYAYSTAPGYYSWRNSKDGSWFIQSLC | 14 |
| | AMLKQYADKLEFMHILTRVNRKVATEFESFSFDATFHAKKQIPCIVSM | |
| | LTKELYFYHGGSGALKKELQANKKELAQLKWELQALKKELAQ | |
| TEVP | MGSGESLFKGPRDYNPISSTICHLTNESDGHTTSLYGIGFGPFIITNK | 15 |
| | HLFRRNNGTLLVQSLHGVFKVKNTTTLQQHLIDGRDMIIIRMPKDFPP | |
| | FPQKLKFREPQREERICLVTTNFQTKSMSSMVSDTSCTFPSSDGIFWK | |
| | HWIQTKDGQCGSPLVSTRDGFIVGIHSASNFTNTNNYFTSVPKNFMEL | |
| | LTNQEAQQWVSGWRLNADSVLWGGHKVFMVKPEEPFQPVKEATQLMNS | |
| GSDMA-N-tev-C | MGSPVFEDVTRALVRELNPRGDLTPLDSLIDFKHFRPFCLVLRKRKST | 16 |
| | LFWGARYVRTDYTLLDLLEPGSSPSDLTDSGNFSFKNMLDVQVQGLVE | |
| | VPKTVKVKGTAGLSQSSTLEVQTLSVAPSALENLKKERKLSADHSFLN | |
| | EMRYHEKNLYVVMEAVEAKQEVTVEQTGNANAIFSLPSLALLGLQGSL | |
| | NNNKAVTIPKGCVLAYRVRLLRVFLFNLWDIPYICNDSMQTFPKIRRV | |
| | PCSAFISPTQMISEEPEEEKLIGEENLYFQSMHEDFKTLKEEVQRETQ | |
| | EVEKLSPVGRSSLLTSLSHLLGKKKELQDLEQKLEGALDKGQKVTLEA | |
| | LPKDVLLSKDAMDAILYFLGALTELTEEQLKILVKSLEKKILPVQLKL | |
| | VESTLEQNFLQDKEGVFPLQPDLLSSLGEEELTLTEALVGLSGLEVQR | |
| | SGPQYAWDPDTRHNLCALYAGLSLLHLLSRKSNALTYCALS | |
| GSDMA-MBP- | MGSMKIEEGKLVIWINGDKGYNGLAEVGKKFEKDTGIKVTVEHPDKLE | 17 |
| tev-N | EKFPQVAATGDGPDIIFWAHDRFGGYAQSGLLAEITPDKAFQDKLYPF | |
| | TWDAVRYNGKLIAYPIAVEALSLIYNKDLLPNPPKTWEEIPALDKELK | |
| | AKGKSALMENLQEPYFTWPLIAADGGYAFKYENGKYDIKDVGVDNAGA | |
| | KAGLTFLVDLIKNKHMNADTDYSIAEAAFNKGETAMTINGPWAWSNID | |
| | TSKVNYGVTVLPTFKGQPSKPFVGVLSAGINAASPNKELAKEFLENYL | |
| | LTDEGLEAVNKDKPLGAVALKSYEEELAKDPRIAATMENAQKGEIMPN | |
| | IPQMSAFWYAVRTAVINAASGRQTVDEALKDAQTGSGSSENLYFQSGG | |
| | GGSPVFEDVTRALVRELNPRGDLTPLDSLIDFKHFRPFCLVLRKRKST | |
| | LFWGARYVRTDYTLLDLLEPGSSPSDLTDSGNFSFKNMLDVQVQGLVE | |
| | VPKTVKVKGTAGLSQSSTLEVQTLSVAPSALENLKKERKLSADHSFLN | |
| | EMRYHEKNLYVVMEAVEAKQEVTVEQTGNANAIFSLPSLALLGLQGSL | |
| | NNNKAVTIPKGCVLAYRVRLLRVFLFNLWDIPYICNDSMQTFPKIRRV | |
| | PCSAFISPTQMISEEPEEEKLIGE | |
| GSDMA-N-tev- | MGSPVFEDVTRALVRELNPRGDLTPLDSLIDFKHFRPFCLVLRKRKST | 18 |
| Deg | LFWGARYVRTDYTLLDLLEPGSSPSDLTDSGNFSFKNMLDVQVQGLVE | |
| | VPKTVKVKGTAGLSQSSTLEVQTLSVAPSALENLKKERKLSADHSFLN | |
| | EMRYHEKNLYVVMEAVEAKQEVTVEQTGNANAIFSLPSLALLGLQGSL | |
| | NNNKAVTIPKGCVLAYRVRLLRVFLFNLWDIPYICNDSMQTFPKIRRV | |
| | PCSAFISPTQMISEEPEEEKLIGEGGSGLENLYFQSGSGSISLIAALA | |
| | VDYVIGMENAMPWNLPADLAWFKRNTLNKPVIMGRHTWESIGRPLPGR | |
| | KNIILSSQPSTDDRVTWVKSVDEAIAACGDVPEIMVIGGGRVIEQFLP | |
| | KAQKLYLTHIDAEVEGDTHFPDYEPDDWESVFSEFHDADAQNSHSYCF | |
| | EILERR | |
| GSDMA-N-tev | MGSPVFEDVTRALVRELNPRGDLTPLDSLIDFKHFRPFCLVLRKRKST | 19 |
| | LFWGARYVRTDYTLLDLLEPGSSPSDLTDSGNFSFKENLYFQSNMLDV | |
| | QVQGLVEVPKTVKVKGTAGLSQSSTLEVQTLSVAPSALENLKKERKLS | |
| | ADHSFLNEMRYHEKNLYVVMEAVEAKQEVTVEQTGNANAIFSLPSLAL | |
| | LGLQGSLNNNKAVTIPKGCVLAYRVRLLRVFLFNLWDIPYICNDSMQT | |
| | FPKIRRVPCSAFISPTQMISEEPEEEKLIGE | |
| GSDMA-tevD-N | MGSENLYFQRHKSGAWKLPVSLVKPVFEDVTRALVRELNPRGDLTPLD | 20 |
| | SLIDFKHFRPFCLVLRKRKSTLFWGARYVRTDYTLLDLLEPGSSPSDL | |
| | TDSGNFSFKNMLDVQVQGLVEVPKTVKVKGTAGLSQSSTLEVQTLSVA | |
| | PSALENLKKERKLSADHSFLNEMRYHEKNLYVVMEAVEAKQEVTVEQT | |
| | GNANAIFSLPSLALLGLQGSLNNNKAVTIPKGCVLAYRVRLLRVFLFN | |
| | LWDIPYICNDSMQTFPKIRRVPCSAFISPTQMISEEPEEEKLIGE | |
| Wildtype | MGFAKATRNFLREVDADGDLIAVSNLNDSDKLQLLSLVTKKKRFWCWQ | 21 |
| GSDME | RPKYQFLSLTLGDVLIEDQFPSPVVVESDFVKYEGKFANHVSGTLETA | |
| | LGKVKLNLGGSSRVESQSSFGTLRKQEVDLQQLIRDSAERTINLRNPV | |
| | LQQVLEGRNEVLCVLTQKITTMQKCVISEHMQVEEKCGGIVGIQTKTV | |
| | QVSATEDGNVTKDSNVVLEIPAATTIAYGVIELYVKLDGQFEFCLLRG | |
| | KQGGFENKKRIDSVYLDPLVFREFAFIDMPDAAHGISSQDGPLSVLKQ | |
| | ATLLLERNFHPFAELPEPQQTALSDIFQAVLFDDELLMVLEPVCDDLV | |
| | SGLSPTVAVLGELKPRQQQDLVAFLQLVGCSLQGGCPGPEDAGSKQLF | |
| | MTAYFLVSALAEMPDSAAALLGTCCKLQIIPTLCHLLRALSDDGVSDL | |
| | EDPTLTPLKDTERFGIVQRLFASADISLERLKSSVKAVILKDSKVFPL | |
| | LLCITLNGLCALGREHS | |
| Mutant GSDME- | MGFAKATRNFLREVDADGDLIAVSNLNDSDKLQLLSLVTKKKRFWCWQ | 22 |
| N | RPKYQFLSLTLGDVLIEDQFPSPVVVESDFVKYEGKFANHVSGTLETA | |
| | LGKVKLNLGGSSRVESQSSFGTLRKQEVDLQQLIRDSAERTINLRNPV | |
| | LQQVLEGRNEVLCVLTQKITTMQKCVISEHMQVEEKCGGIVGIQTKTV | |
| | QVSATEDGNVTKDSNVVLEIPAATTNAYGVIELYVKLDGQFEFCLLRG | |
| | KQGGFENKKRIDSVYLDPLVFREFAFIDMPD | |
| Caspase-3-L-tev- | MGSENTENSVDSKSIKNLEPKIIHGSESMDSGISLDNSYKMDYPEMGL | 23 |
| S-tvmv-Deg | CIIINNKNFHKSTGMTSRSGTDVDAANLRETFRNLKYEVRNKNDLTRE | |
| | EIVELMRDVSKEDHSKRSSFVCVLLSHGEEGIIFGTNGPVDLKKITNF | |
| | FRGDRCRSLTGKPKLFIIQACRGTELDCGIETENLYFQSGVDDDMACH | |
| | KIPVEADFLYAYSTAPGYYSWRNSKDGSWFIQSLCAMLKQYADKLEFM | |
| | HILTRVNRKVATEFESFSFDATFHAKKQIPCIVSMLTKELYFYHGGSG | |
| | LETVRFQSGSGSISLIAALAVDYVIGMENAMPWNLPADLAWFKRNTLN | |
| | KPVIMGRHTWESIGRPLPGRKNIILSSQPSTDDRVTWVKSVDEAIAAC | |
| | GDVPEIMVIGGGRVIEQFLPKAQKLYLTHIDAEVEGDTHFPDYEPDDW | |
| | ESVFSEFHDADAQNSHSYCFEILERR | |
| Caspase-3-L-tev- | MGSENTENSVDSKSIKNLEPKIIHGSESMDSGISLDNSYKMDYPEMGL | 24 |
| tvmv-S | CIIINNKNFHKSTGMTSRSGTDVDAANLRETFRNLKYEVRNKNDLTRE | |
| | EIVELMRDVSKEDHSKRSSFVCVLLSHGEEGIIFGTNGPVDLKKITNF | |
| | FRGDRCRSLTGKPKLFIIQACRGTELDCGIETENLYFQSETVRFQSGV | |
| | DDDMACHKIPVEADFLYAYSTAPGYYSWRNSKDGSWFIQSLCAMLKQY | |
| | ADKLEFMHILTRVNRKVATEFESFSFDATFHAKKQIPCIVSMLTKELY | |
| | FYH | |
| Caspase-3- | MGSETVRFQYHKSGAWKLPVSLVKEQLEKKLQALEKKLAQLEWKNQAL | 25 |
| tvmvD-Z-L | EKKLAQENTENSVDSKSIKNLEPKIIHGSESMDSGISLDNSYKMDYPE | |
| | MGLCIIINNKNFHKSTGMTSRSGTDVDAANLRETFRNLKYEVRNKNDL | |
| | TREEIVELMRDVSKEDHSKRSSFVCVLLSHGEEGIIFGTNGPVDLKKI | |
| | TNFFRGDRCRSLTGKPKLFIIQACRGTELDCGIETD | |
| Caspase-3-S-Z- | MGSSGVDDDMACHKIPVEADFLYAYSTAPGYYSWRNSKDGSWFIQSLC | 26 |
| tev-Deg | AMLKQYADKLEFMHILTRVNRKVATEFESFSFDATFHAKKQIPCIVSM | |
| | LTKELYFYHGGSGALKKELQANKKELAQLKWELQALKKELAQGGSGLE | |
| | NLYFQSGSGSISLIAALAVDYVIGMENAMPWNLPADLAWFKRNTLNKP | |
| | VIMGRHTWESIGRPLPGRKNIILSSQPSTDDRVTWVKSVDEAIAACGD | |
| | VPEIMVIGGGRVIEQFLPKAQKLYLTHIDAEVEGDTHFPDYEPDDWES | |
| | VFSEFHDADAQNSHSYCFEILERR | |
| TVMVP | MGSSKALLKGVRDFNPISACVCLLENSSDGHSERLFGIGFGPYIIANQ | 27 |
| | HLFRRNNGELTIKTMHGEFKVKNSTQLQMKPVEGRDIIVIKMAKDFPP | |
| | FPQKLKFRQPTIKDRVCMVSTNFQQKSVSSLVSESSHIVHKEDTSFWQ | |
| | HWITTKDGQCGSPLVSIIDGNILGIHSLTHTTNGSNYFVEFPEKFVAT | |
| | YLDAADGWCKNWKFNADKISWGSFTLVEDAPEDDFMAKKTVAAIMDS | |
| GSDMA-MBP- | MGSMKIEEGKLVIWINGDKGYNGLAEVGKKFEKDTGIKVTVEHPDKLE | 28 |
| tvmv-N-tev-C | EKFPQVAATGDGPDIIFWAHDRFGGYAQSGLLAEITPDKAFQDKLYPF | |
| | TWDAVRYNGKLIAYPIAVEALSLIYNKDLLPNPPKTWEEIPALDKELK | |
| | AKGKSALMENLQEPYFTWPLIAADGGYAFKYENGKYDIKDVGVDNAGA | |
| | KAGLTFLVDLIKNKHMNADTDYSIAEAAFNKGETAMTINGPWAWSNID | |
| | TSKVNYGVTVLPTFKGQPSKPFVGVLSAGINAASPNKELAKEFLENYL | |
| | LTDEGLEAVNKDKPLGAVALKSYEEELAKDPRIAATMENAQKGEIMPN | |
| | IPQMSAFWYAVRTAVINAASGRQTVDEALKDAQTGSGSSETVRFQSGG | |
| | GGSPVFEDVTRALVRELNPRGDLTPLDSLIDFKHFRPFCLVLRKRKST | |
| | LFWGARYVRTDYTLLDLLEPGSSPSDLTDSGNFSFKNMLDVQVQGLVE | |
| | VPKTVKVKGTAGLSQSSTLEVQTLSVAPSALENLKKERKLSADHSFLN | |
| | EMRYHEKNLYVVMEAVEAKQEVTVEQTGNANAIFSLPSLALLGLQGSL | |
| | NNNKAVTIPKGCVLAYRVRLLRVFLFNLWDIPYICNDSMQTFPKIRRV | |
| | PCSAFISPTQMISEEPEEEKLIGEENLYFQSMHEDFKTLKEEVQRETQ | |
| | EVEKLSPVGRSSLLTSLSHLLGKKKELQDLEQKLEGALDKGQKVTLEA | |
| | LPKDVLLSKDAMDAILYFLGALTELTEEQLKILVKSLEKKILPVQLKL | |
| | VESTLEQNFLQDKEGVFPLQPDLLSSLGEEELTLTEALVGLSGLEVQR | |
| | SGPQYAWDPDTRHNLCALYAGLSLLHLLSRKSNALTYCALS | |
| GSDMA-N-tev- | MGPVFEDVTRALVRELNPRGDLTPLDSLIDFKHFRPFCLVLRKRKSTL | 29 |
| tvmv-C | FWGARYVRTDYTLLDLLEPGSSPSDLTDSGNFSFKNMLDVQVQGLVEV | |
| | PKTVKVKGTAGLSQSSTLEVQTLSVAPSALENLKKERKLSADHSFLNE | |
| | MRYHEKNLYVVMEAVEAKQEVTVEQTGNANAIFSLPSLALLGLQGSLN | |
| | NNKAVTIPKGCVLAYRVRLLRVFLFNLWDIPYICNDSMQTFPKIRRVP | |
| | CSAFISPTQMISEEPEEEKLIGEENLYFQSETVRFQSMHEDFKTLKEE | |
| | VQRETQEVEKLSPVGRSSLLTSLSHLLGKKKELQDLEQKLEGALDKGQ | |
| | KVTLEALPKDVLLSKDAMDAILYFLGALTELTEEQLKILVKSLEKKIL | |
| | PVQLKLVESTLEQNFLQDKEGVFPLQPDLLSSLGEEELTLTEALVGLS | |
| | GLEVQRSGPQYAWDPDTRHNLCALYAGLSLLHLLSRKSNALTYCALS | |
| GSDMA-tvmvD- | MGSETVRFQRHKSGAWKLPVSLVKPVFEDVTRALVRELNPRGDLTPLD | 30 |
| N-tev-Deg | SLIDFKHFRPFCLVLRKRKSTLFWGARYVRTDYTLLDLLEPGSSPSDL | |
| | TDSGNFSFKNMLDVQVQGLVEVPKTVKVKGTAGLSQSSTLEVQTLSVA | |
| | PSALENLKKERKLSADHSFLNEMRYHEKNLYVVMEAVEAKQEVTVEQT | |
| | GNANAIFSLPSLALLGLQGSLNNNKAVTIPKGCVLAYRVRLLRVFLFN | |
| | LWDIPYICNDSMQTFPKIRRVPCSAFISPTQMISEEPEEEKLIGEGGS | |
| | GLENLYFQSGSGSISLIAALAVDYVIGMENAMPWNLPADLAWFKRNTL | |
| | NKPVIMGRHTWESIGRPLPGRKNIILSSQPSTDDRVTWVKSVDEAIAA | |
| | CGDVPEIMVIGGGRVIEQFLPKAQKLYLTHIDAEVEGDTHFPDYEPDD | |
| | WESVFSEFHDADAQNSHSYCFEILERR | |
| Caspase-3-tevD- | MGSENLYFQYHKSGAWKLPVSLVKENTENSVDSKSIKNLEPKIIHGSE | 31 |
| L-tvmv-S | SMDSGISLDNSYKMDYPEMGLCIIINNKNFHKSTGMTSRSGTDVDAAN | |
| | LRETFRNLKYEVRNKNDLTREEIVELMRDVSKEDHSKRSSFVCVLLSH | |
| | GEEGIIFGTNGPVDLKKITNFFRGDRCRSLTGKPKLFIIQACRGTELD | |
| | CGIETETVRFQSGVDDDMACHKIPVEADFLYAYSTAPGYYSWRNSKDG | |
| | SWFIQSLCAMLKQYADKLEFMHILTRVNRKVATEFESFSFDATFHAKK | |
| | QIPCIVSMLTKELYFYH | |
| Caspase-3- | MGSETVRFQYHKSGAWKLPVSLVKENTENSVDSKSIKNLEPKIIHGSE | 32 |
| tvmvD-L-tev-S | SMDSGISLDNSYKMDYPEMGLCIIINNKNFHKSTGMTSRSGTDVDAAN | |
| | LRETFRNLKYEVRNKNDLTREEIVELMRDVSKEDHSKRSSFVCVLLSH | |
| | GEEGIIFGTNGPVDLKKITNFFRGDRCRSLTGKPKLFIIQACRGTELD | |
| | CGIETENLYFQSGVDDDMACHKIPVEADFLYAYSTAPGYYSWRNSKDG | |
| | SWFIQSLCAMLKQYADKLEFMHILTRVNRKVATEFESFSFDATFHAKK | |
| | QIPCIVSMLTKELYFYH | |
| Caspase-3-tevD- | MGSENLYFQYHKSGAWKLPVSLVKETVRFQSEQLEKKLQALEKKLAQL | 33 |
| tvmv-Z-L | EWKNQALEKKLAQENTENSVDSKSIKNLEPKIIHGSESMDSGISLDNS | |
| | YKMDYPEMGLCIIINNKNFHKSTGMTSRSGTDVDAANLRETFRNLKYE | |
| | VRNKNDLTREEIVELMRDVSKEDHSKRSSFVCVLLSHGEEGIIFGTNG | |
| | PVDLKKITNFFRGDRCRSLTGKPKLFIIQACRGTELDCGIETD | |
| Caspase-3-S-Z | MGSSGVDDDMACHKIPVEADFLYAYSTAPGYYSWRNSKDGSWFIQSLC | 34 |
| | AMLKQYADKLEFMHILTRVNRKVATEFESFSFDATFHAKKQIPCIVSM | |
| | LTKELYFYHGGSGALKKELQANKKELAQLKWELQALKKELAQ | |
| Caspase-3-tevD- | MGSENLYFQYHKSGAWKLPVSLVKEQLEKKLQALEKKLAQLEWKNQAL | 35 |
| Z-L | EKKLAQENTENSVDSKSIKNLEPKIIHGSESMDSGISLDNSYKMDYPE | |
| | MGLCIIINNKNFHKSTGMTSRSGTDVDAANLRETFRNLKYEVRNKNDL | |
| | TREEIVELMRDVSKEDHSKRSSFVCVLLSHGEEGIIFGTNGPVDLKKI | |
| | TNFFRGDRCRSLTGKPKLFIIQACRGTELDCGIETD | |
| Caspase-3- | MGSETVRFQYHKSGAWKLPVSLVKEQLEKKLQALEKKLAQLEWKNQAL | 36 |
| tvmvD-Z-L | EKKLAQENTENSVDSKSIKNLEPKIIHGSESMDSGISLDNSYKMDYPE | |
| | MGLCIIINNKNFHKSTGMTSRSGTDVDAANLRETFRNLKYEVRNKNDL | |
| | TREEIVELMRDVSKEDHSKRSSFVCVLLSHGEEGIIFGTNGPVDLKKI | |
| | TNFFRGDRCRSLTGKPKLFIIQACRGTELDCGIETD | |
| GSDMA-tevD-N- | MGSENLYFQRHKSGAWKLPVSLVKPVFEDVTRALVRELNPRGDLTPLD | 37 |
| tvmv-C | SLIDFKHFRPFCLVLRKRKSTLFWGARYVRTDYTLLDLLEPGSSPSDL | |
| | TDSGNFSFKNMLDVQVQGLVEVPKTVKVKGTAGLSQSSTLEVQTLSVA | |
| | PSALENLKKERKLSADHSFLNEMRYHEKNLYVVMEAVEAKQEVTVEQT | |
| | GNANAIFSLPSLALLGLQGSLNNNKAVTIPKGCVLAYRVRLLRVFLFN | |
| | LWDIPYICNDSMQTFPKIRRVPCSAFISPTQMISEEPEEEKLIGEETV | |
| | RFQSMHEDFKTLKEEVQRETQEVEKLSPVGRSSLLTSLSHLLGKKKEL | |
| | QDLEQKLEGALDKGQKVTLEALPKDVLLSKDAMDAILYFLGALTELTE | |
| | EQLKILVKSLEKKILPVQLKLVESTLEQNFLQDKEGVFPLQPDLLSSL | |
| | GEEELTLTEALVGLSGLEVQRSGPQYAWDPDTRHNLCALYAGLSLLHL | |
| | LSRKSNALTYCALS | |
| GSDMA-tvmvD- | MGSETVRFQRHKSGAWKLPVSLVKPVFEDVTRALVRELNPRGDLTPLD | 38 |
| N-tev-C | SLIDFKHFRPFCLVLRKRKSTLFWGARYVRTDYTLLDLLEPGSSPSDL | |
| | TDSGNFSFKNMLDVQVQGLVEVPKTVKVKGTAGLSQSSTLEVQTLSVA | |
| | PSALENLKKERKLSADHSFLNEMRYHEKNLYVVMEAVEAKQEVTVEQT | |
| | GNANAIFSLPSLALLGLQGSLNNNKAVTIPKGCVLAYRVRLLRVFLFN | |
| | LWDIPYICNDSMQTFPKIRRVPCSAFISPTQMISEEPEEEKLIGEENL | |
| | YFQSMHEDFKTLKEEVQRETQEVEKLSPVGRSSLLTSLSHLLGKKKEL | |
| | QDLEQKLEGALDKGQKVTLEALPKDVLLSKDAMDAILYFLGALTELTE | |
| | EQLKILVKSLEKKILPVQLKLVESTLEQNFLQDKEGVFPLQPDLLSSL | |
| | GEEELTLTEALVGLSGLEVQRSGPQYAWDPDTRHNLCALYAGLSLLHL | |
| | LSRKSNALTYCALS | |
| GSDMA-tevD- | MGSENLYFQRHKSGAWKLPVSLVKETVRFQSPVFEDVTRALVRELNPR | 39 |
| tvmv-N | GDLTPLDSLIDFKHFRPFCLVLRKRKSTLFWGARYVRTDYTLLDLLEP | |
| | GSSPSDLTDSGNFSFKNMLDVQVQGLVEVPKTVKVKGTAGLSQSSTLE | |
| | VQTLSVAPSALENLKKERKLSADHSFLNEMRYHEKNLYVVMEAVEAKQ | |
| | EVTVEQTGNANAIFSLPSLALLGLQGSLNNNKAVTIPKGCVLAYRVRL | |
| | LRVFLFNLWDIPYICNDSMQTFPKIRRVPCSAFISPTQMISEEPEEEK | |
| | LIGE | |
| GSDMA-tevD-N | MGSENLYFQRHKSGAWKLPVSLVKPVFEDVTRALVRELNPRGDLTPLD | 40 |
| | SLIDFKHFRPFCLVLRKRKSTLFWGARYVRTDYTLLDLLEPGSSPSDL | |
| | TDSGNFSFKNMLDVQVQGLVEVPKTVKVKGTAGLSQSSTLEVQTLSVA | |
| | PSALENLKKERKLSADHSFLNEMRYHEKNLYVVMEAVEAKQEVTVEQT | |
| | GNANAIFSLPSLALLGLQGSLNNNKAVTIPKGCVLAYRVRLLRVFLFN | |
| | LWDIPYICNDSMQTFPKIRRVPCSAFISPTQMISEEPEEEKLIGE | |
| GSDMA-tvmvD- | MGSETVRFQRHKSGAWKLPVSLVKPVFEDVTRALVRELNPRGDLTPLD | 41 |
| N | SLIDFKHFRPFCLVLRKRKSTLFWGARYVRTDYTLLDLLEPGSSPSDL | |
| | TDSGNFSFKNMLDVQVQGLVEVPKTVKVKGTAGLSQSSTLEVQTLSVA | |
| | PSALENLKKERKLSADHSFLNEMRYHEKNLYVVMEAVEAKQEVTVEQT | |
| | GNANAIFSLPSLALLGLQGSLNNNKAVTIPKGCVLAYRVRLLRVFLFN | |
| | LWDIPYICNDSMQTFPKIRRVPCSAFISPTQMISEEPEEEKLIGE | |
| GSDMA-N-Z | MGSPVFEDVTRALVRELNPRGDLTPLDSLIDFKHFRPFCLVLRKRKST | 42 |
| | LFWGARYVRTDYTLLDLLEPGSSPSDLTDSGNFSFKNMLDVQVQGLVE | |
| | VPKTVKVKGTAGLSQSSTLEVQTLSVAPSALENLKKERKLSADHSFLN | |
| | EMRYHEKNLYVVMEAVEAKQEVTVEQTGNANAIFSLPSLALLGLQGSL | |
| | NNNKAVTIPKGCVLAYRVRLLRVFLFNLWDIPYICNDSMQTFPKIRRV | |
| | PCSAFISPTQMISEEPEEEKLIGESGGTQALEKKLAQLEWKLQALEKK | |
| | NAQLEKKLASSGSG | |
| GSDMA-Z-C | MGSEQLEKKLQALEKKLAQLEWKNQALEKKLAQGGSGSSMHEDFKTLK | 43 |
| | EEVQRETQEVEKLSPVGRSSLLTSLSHLLGKKKELQDLEQKLEGALDK | |
| | GQKVTLEALPKDVLLSKDAMDAILYFLGALTELTEEQLKILVKSLEKK | |
| | ILPVQLKLVESTLEQNFLQDKEGVFPLQPDLLSSLGEEELTLTEALVG | |
| | LSGLEVQRSGPQYAWDPDTRHNLCALYAGLSLLHLLSRKSNALTYCAL | |
| | S | |
| Casp3_v2.1 | MEIGTGFPFDPHYVEVLGERMHYVDVGPRDGTPVLFLHGNPTSSYVWR | 44 |
| | NIIPHVAPGSENLYFQSGSENTENSVDSKSIKNLEPKIIHGSESMDSG | |
| | ISLDNSYKMDYPEMGLCIIINNKNFHKSTGMTSRSGTDVDAANLRETF | |
| | RNLKYEVRNKNDLTREEIVELMRDVSKEDHSKRSSFVCVLLSHGEEGI | |
| | IFGTNGPVDLKKITNFFRGDRCRSLTGKPKLFIIQACRGTELDCGIET | |
| | ENLYFQSGVDDDMACHKIPVEADFLYAYSTAPGYYSWRNSKDGSWFIQ | |
| | SLCAMLKQYADKLEFMHILTRVNRKVATEFESFSFDATFHAKKQIPCI | |
| | VSMLTKELYFYHSTGSENLYFQSGSTHRCIAPDLIGMGKSDKPDLGYF | |
| | FDDHVRFMDAFIEALGLEEVVLVIHDWGSALGFHWAKRNPERVKGIAF | |
| | MEFIRPIPTWDEWPEFARETFQAFRTTDVGRKLIIDQNVFIEGTLPMG | |
| | VVRPLTEVEMDHYREPFLNPVDREPLWRFPNELPIAGEPANIVALVEE | |
| | YMDWLHQSPVPKLLFWGTPGVLIPPAEAARLAKSLPNCKAVDIGPGLN | |
| | LLQEDNPDLIGSEIARWLSTLEISGGSSGGGGRSKLNPPDESGPGCMS | |
| | CKCVLS | |
| Casp3_v2.2 | MENTENSVDSKSIKNLEPKIIHGSESMDSGISLDNSYKMDYPEMGLCI | 45 |
| | IINNKNFHKSTGMTSRSGTDVDAANLRETFRNLKYEVRNKNDLTREEI | |
| | VELMRDVSKEDHSKRSSFVCVLLSHGEEGIIFGTNGPVDLKKITNFFR | |
| | GDRCRSLTGKPKLFIIQACRGTELDCGIETENLYFQSEIGTGFPFDPH | |
| | YVEVLGERMHYVDVGPRDGTPVLFLHGNPTSSYVWRNIIPHVAPENLY | |
| | FQSTHRCIAPDLIGMGKSDKPDLGYFFDDHVRFMDAFIEALGLEEVVL | |
| | VIHDWGSALGFHWAKRNPERVKGIAFMEFIRPIPTWDEWPEFARETFQ | |
| | AFRTTDVGRKLIIDQNVFIEGTLPMGVVRPLTEVEMDHYREPFLNPVD | |
| | REPLWRFPNELPIAGEPANIVALVEEYMDWLHQSPVPKLLFWGTPGVL | |
| | IPPAEAARLAKSLPNCKAVDIGPGLNLLQEDNPDLIGSEIARWLSTLE | |
| | ISENLYFQSGVDDDMACHKIPVEADFLYAYSTAPGYYSWRNSKDGSWF | |
| | IQSLCAMLKQYADKLEFMHILTRVNRKVATEFESFSFDATFHAKKQIP | |
| | CIVSMLTKELYFYHSTGGSSGGGGRSKLNPPDESGPGCMSCKCVLS | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A synthetic protein circuit comprising:
a first apoptosis polypeptide comprising a large subunit of an apoptotic effector protein and a small subunit of an apoptotic effector protein separated by a first heterologous protease cleavage site,
wherein two first apoptosis polypeptides are capable of associating with each other to constitute a first apoptotic protein complex in a first apoptotic protein complex inactive state,
wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state,
wherein the first apoptotic protein complex in the first apoptotic protein complex active state is capable of inducing apoptosis in a cell, and
optionally the synthetic protein circuit further comprises a first input polypeptide comprising the first heterologous protease.

2. A synthetic protein circuit comprising:
a first apoptosis polypeptide comprising a large subunit of an apoptotic effector protein and a first partner domain; and
a second apoptosis polypeptide comprising a small subunit of an apoptotic effector protein, a second partner domain capable of binding the first partner domain, a first heterologous protease cleavage site, and a first degron,
wherein a first heterologous protease in a first heterologous protease active state is capable of cutting the first heterologous protease cleavage site of the second apoptosis polypeptide and thereby releasing the first degron, and wherein the first heterologous protease cleavage site of the second apoptosis polypeptide being cut changes the second apoptosis polypeptide from a second apoptosis polypeptide destabilized state to a second apoptosis polypeptide stabilized state,
wherein the first apoptosis polypeptide and the second apoptosis polypeptide in the second apoptosis polypeptide stabilized state are capable of associating via binding of the first partner domain and the second partner domain to form a subunit, and
wherein two subunits are capable of associating with each other to constitute a first apoptotic protein complex capable of being in a first apoptotic protein complex active state,
wherein the first apoptotic protein complex in the first apoptotic protein complex active state is capable of inducing apoptosis in a cell, and
optionally the synthetic protein circuit further comprises a first input polypeptide comprising the first heterologous protease.

3. A synthetic protein circuit comprising:
a first apoptosis polypeptide comprising a large subunit of an apoptotic effector protein, a first partner domain, a first degron, and a first heterologous protease cleavage site; and
a second apoptosis polypeptide comprising a small subunit of an apoptotic effector protein and a second partner domain capable of binding the first partner domain,
wherein the first apoptosis polypeptide and the second apoptosis polypeptide are capable of associating via binding of the first partner domain and the second partner domain to form a subunit,
wherein two subunits are capable of associating with each other to constitute a first apoptotic protein complex capable of being in a first apoptotic protein complex active state, wherein the first apoptotic protein complex in the first apoptotic protein complex active state is capable of inducing apoptosis in a cell,
wherein a first heterologous protease in a first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state, and
optionally the synthetic protein circuit further comprises a first input polypeptide comprising the first heterologous protease.

4. The synthetic protein circuit of any one of claims 1-3, wherein:
the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof;
the first heterologous protease comprises or is derived from one or more of a prokaryotic protease, bacterial protease, archaeal protease, eukaryotic protease, fungal protease, plant protease, mammalian protease, and human protease, optionally the first heterologous protease is engineered, optionally wherein the first heterologous protease comprises tobacco etch virus (TEV) protease, tobacco vein mottling virus (TVMV) protease, hepatitis C virus protease (HCVP), derivatives thereof, or any combination thereof, optionally the first heterologous protease cleavage site comprises the sequence of SEQ ID NO: 1 or SEQ ID NO: 2;
the first heterologous protease cleavage site is natural or engineered, optionally the cleavage sequence of a human apoptotic effector protease, optionally a caspase cleavage sequence, optionally wherein the first heterologous protease cleavage site is configured to modulate an activation threshold of the synthetic protein circuit, optionally wherein said configuring comprises introducing one or more amino acid insertions, substitutions, or deletions relative to a wild type first heterologous protease cleavage site, further optionally the first heterologous protease cleavage site comprises the sequence of any one of SEQ ID NOs: 3-7;
the first apoptotic protein complex in the first apoptotic protein complex active state is capable of being inhibited by a small molecule inhibitor of apoptosis, optionally, the small molecule inhibitor comprises Quinoline-Val-Asp-Difluorophenoxymethylketone (Q-VD-OPh), carbobenzoxy-valyl-alanyl-aspartyl-[O-methyl]-fluoromethylketone (Z-VAD-FMK), and/or emricasan; and/or
the first apoptosis polypeptide and/or the second apoptosis polypeptide comprise one or more third partner domains and one or more fourth partner domains capable of binding the third partner domain, wherein binding of the third partner domain and the fourth partner domain is capable of inhibiting change of the first apoptotic protein complex from the first apoptotic protein complex inactive state to the first apoptotic protein complex active state in the absence of the first heterologous protease in the first heterologous protease active state, optionally wherein said third partner domains and fourth partner domains are capable of multimerization.

5. A synthetic protein circuit comprising:
a first pyroptosis polypeptide comprising a pyroptosis effector domain and a first inhibitory domain separated by a first heterologous protease cleavage site, wherein the first inhibitory domain is capable of inhibiting activity of the pyroptosis effector domain, thereby the first pyroptosis polypeptide is in a first pyroptosis polypeptide inactive state,
wherein a first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state,
wherein the first pyroptosis polypeptide in the first pyroptosis polypeptide active state is capable of inducing pyroptosis in a cell, and
optionally the synthetic protein circuit further comprises a first input polypeptide comprising the first heterologous protease.

6. The synthetic protein circuit of claim 5, wherein the first inhibitory domain comprises a protein domain configured to reduce or abrogate the activity of the pyroptosis effector domain, optionally a bulky domain, further optionally the first inhibitory domain comprises an auto-inhibitory domain of a gasdermin family of pore-forming proteins or maltose-binding protein.

7. A synthetic protein circuit comprising:
a first pyroptosis polypeptide comprising a pyroptosis effector domain and a first degron separated by a first heterologous protease cleavage site,
wherein a first heterologous protease in a first heterologous protease active state is capable of cutting the first heterologous protease cleavage site of the first pyroptosis polypeptide and thereby releasing the first degron, and wherein the first heterologous protease cleavage site of the first pyroptosis polypeptide being cut changes the first pyroptosis polypeptide from a first pyroptosis polypeptide destabilized state to a first pyroptosis polypeptide stabilized state,
wherein the first pyroptosis polypeptide in the first pyroptosis polypeptide stabilized state is capable of being in a first pyroptosis polypeptide active state,
wherein the first pyroptosis polypeptide in the first pyroptosis polypeptide active state is capable of inducing pyroptosis in a cell,
optionally the synthetic protein circuit further comprises a first input polypeptide comprising the first heterologous protease.

8. A synthetic protein circuit comprising:
a first pyroptosis polypeptide comprising a pyroptosis effector domain, a first degron, and a first heterologous protease cleavage site, wherein the first pyroptosis polypeptide is capable of being in a first pyroptosis polypeptide active state, wherein the first pyroptosis polypeptide in the first pyroptosis polypeptide active state is capable of inducing pyroptosis in a cell, and
wherein a first heterologous protease in a first heterologous protease active state is capable of cutting the first heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state, and
optionally the synthetic protein circuit further comprises a first input polypeptide comprising the first heterologous protease.

9. The synthetic protein circuit of any one of claims 5-8, wherein:
the pyroptosis effector domain comprises an N-terminal domain of a gasdermin (GSDM) protein;
the GSDM protein is from the gasdermin (GSDM) family, including GSDMA, GSDMB, GSDMC, GSDMD, GSDME, or DFNB59;
the first heterologous protease comprises or is derived from one or more of a prokaryotic protease, bacterial protease, archaeal protease, eukaryotic protease, fungal protease, plant protease, mammalian protease, and human protease, optionally the first heterologous protease is engineered, optionally wherein the first heterologous protease comprises tobacco etch virus (TEV) protease, tobacco vein mottling virus (TVMV) protease, hepatitis C virus protease (HCVP), derivatives thereof, or any combination thereof, optionally the first heterologous protease cleavage site comprises the sequence of SEQ ID NO: 1 or SEQ ID NO: 2;
the first heterologous protease cleavage site is natural or engineered, optionally the cleavage sequence of a human apoptotic effector protease, optionally a caspase cleavage sequence, optionally wherein the first heterologous protease cleavage site is configured to modulate an activation threshold of the synthetic protein circuit, optionally wherein said configuring comprises introducing one or more amino acid insertions, substitutions, or deletions relative to a wild type first heterologous protease cleavage site, further optionally the first heterologous protease cleavage site comprises the sequence of any one of SEQ ID NOs: 3-7;
the first pyroptosis polypeptide comprises one or more third partner domains and one or more fourth partner domains capable of binding the third partner domain, wherein binding of the third partner domain and the fourth partner domain is capable of inhibiting change of the first pyroptosis polypeptide from the first pyroptosis polypeptide inactive state to the first pyroptosis polypeptide active state in the absence of the first heterologous protease in the first heterologous protease active state, optionally wherein said third partner domains and fourth partner domains are capable of multimerization; and/or
inducing pyroptosis in the cell causes the cell to induce key signatures of pyroptosis: chromatin condensation and DNA fragmentation, pore formation, cell swelling, and osmotic lysis, followed by release of one or more inflammatory cytokines, optionally the one or more inflammatory cytokines comprise IL-18, IL-1β, IL-6, IL-8, interferon gamma (IFN-γ), and/or tumor necrosis factor-alpha (TNF-α).

10. A synthetic protein circuit comprising:
one or more apoptosis polypeptides; and/or
one or more pyroptosis polypeptides; and/or
one or more input polypeptides.

11. A synthetic protein circuit comprising: (i) one or more apoptosis polypeptides or one or more pyroptosis polypeptides and (ii) one or more input polypeptides, configured to form one or more logic gates selected from the group comprising an OR logic gate, AND logic gate, NOR logic gate, NAND logic gate, IMPLY logic gate, NIMPLY logic gate, XOR logic gate, and an XNOR logic gate.

12. The synthetic protein circuit of claim 11, comprising:
(i) a first apoptosis polypeptide comprising a large subunit of an apoptotic effector protein and a small subunit of an apoptotic effector protein separated by a first heterologous protease cleavage site, a second heterologous protease cleavage site, and a first degron, wherein the first apoptosis polypeptide is capable of being in a first apoptosis polypeptide destabilized state;
(ii) a first input polypeptide comprising a first heterologous protease; and/or
(iii) a second input polypeptide comprising a second heterologous protease,
wherein the second heterologous protease in a second heterologous protease active state is capable of cutting the second heterologous protease cleavage site of the first apoptosis polypeptide,
wherein the second heterologous protease cleavage site of the first apoptosis polypeptide being cut changes the first apoptosis polypeptide from the first apoptosis polypeptide destabilized state to a first apoptosis polypeptide stabilized state; wherein two of the first apoptosis polypeptides in the first apoptosis polypeptide stabilized state are capable of forming a first apoptotic protein complex in a first apoptotic protein complex inactive state, and
wherein the first heterologous protease in a first heterologous protease active state is capable of cutting the first heterologous protease cleavage site of the first apoptosis polypeptide,
wherein the first heterologous protease cleavage site of the first apoptosis polypeptide being cut changes the first apoptotic protein complex from the first apoptotic protein complex inactive state to a first apoptotic protein complex active state, and
wherein the first apoptotic protein complex in the first apoptotic protein complex active state is capable of inducing apoptosis in a cell.

13. The synthetic protein circuit of claim 11, comprising:
(i) a first apoptosis polypeptide comprising a large subunit of an apoptotic effector protein and a small subunit of an apoptotic effector protein separated by a first heterologous protease cleavage site and a second heterologous protease cleavage site, wherein two of the first apoptosis polypeptides are capable of forming a first apoptotic protein complex in a first apoptotic protein complex inactive state;
(ii) a first input polypeptide comprising a first heterologous protease; and/or
(iii) a second input polypeptide comprising a second heterologous protease, wherein:
(I) the first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state;
(II) the second heterologous protease in a second heterologous protease active state is capable of cutting the second heterologous protease cleavage site of the first apoptosis polypeptide, and wherein the second heterologous protease cleavage site of the first apoptosis polypeptide being cut changes the first apoptotic protein complex from the first apoptotic protein complex inactive state to the first apoptotic protein complex active state; or
(III) the first heterologous protease in the first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to the first apoptotic protein complex active state, and/or
the second heterologous protease in the second heterologous protease active state is capable of cutting the second heterologous protease cleavage site of the first apoptosis polypeptide, and wherein the second heterologous protease cleavage site of the first apoptosis polypeptide being cut changes the first apoptotic protein complex from the first apoptotic protein complex inactive state to the first apoptotic protein complex active state,
wherein the first apoptotic protein complex in the first apoptotic protein complex active state is capable of inducing apoptosis in a cell.

14. The synthetic apoptosis protein circuit of claim 11, comprising:
(i) a first apoptosis polypeptide comprising a large subunit of an apoptotic effector protein, a first partner domain, a first degron, and a first heterologous protease cleavage site;
(ii) a second apoptosis polypeptide comprising a small subunit of an apoptotic effector protein, a second partner domain capable of binding the first partner domain, a second heterologous protease cleavage site, and a second degron,
(iii) a first input polypeptide comprising a first heterologous protease; and/or
(iv) a second input polypeptide comprising a second heterologous protease, wherein:
(I) the first heterologous protease in a first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state;
(II) the second heterologous protease in a second heterologous protease active state is capable of cutting the second heterologous protease cleavage site of the second apoptosis polypeptide and thereby releasing the second degron, and wherein the second heterologous protease cleavage site of the second apoptosis polypeptide being cut changes the second apoptosis polypeptide from a second apoptosis polypeptide destabilized state to a second apoptosis polypeptide stabilized state,
wherein the first apoptosis polypeptide and the second apoptosis polypeptide in the second apoptosis polypeptide stabilized state are capable of associating via binding of the first partner domain and the second partner domain to form a subunit, and
wherein two subunits are capable of associating with each other to constitute a first apoptotic protein complex capable of being in a first apoptotic protein complex active state,
wherein the first apoptotic protein complex in the first apoptotic protein complex active state is capable of inducing apoptosis in a cell; or
(III) wherein the first heterologous protease in the first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from the first apoptosis polypeptide stabilized state to the first apoptosis polypeptide destabilized state, and
the second heterologous protease in the second heterologous protease active state is capable of cutting the second heterologous protease cleavage site of the second apoptosis polypeptide and thereby releasing the second degron, and wherein the second heterologous protease cleavage site of the second apoptosis polypeptide being cut changes the second apoptosis polypeptide from the second apoptosis polypeptide destabilized state to the second apoptosis polypeptide stabilized state.

15. The synthetic protein circuit of claim 11, comprising:
(i) a first apoptosis polypeptide comprising a large subunit of an apoptotic effector protein and a small subunit of an apoptotic effector protein separated by a first heterologous protease cleavage site, a second heterologous protease cleavage site, and a first degron;
(ii) a second apoptosis polypeptide comprising the large subunit of the apoptotic effector protein and the small subunit of the apoptotic effector protein separated by the second heterologous protease cleavage site, the first heterologous protease cleavage site, and a second degron,
wherein two of the first apoptosis polypeptides are capable forming a first apoptotic protein complex in a first apoptotic protein complex inactive state, two of the second apoptosis polypeptides are capable forming a second apoptotic protein complex in a second apoptotic protein complex inactive state, and/or one of the first apoptosis polypeptide and one of the second apoptosis polypeptide are capable forming a third apoptotic protein complex in a third apoptotic protein complex inactive state;
(iii) a first input polypeptide comprising a first heterologous protease; and/or
(iv) a second input polypeptide comprising a second heterologous protease, wherein:
(I) the first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, and
the first heterologous protease in the first heterologous protease active state is capable of cutting the first heterologous protease cleavage site of the second apoptosis polypeptide to expose the second degron, and wherein the second degron of the second apoptosis polypeptide being exposed changes the second apoptosis polypeptide from a second apoptosis polypeptide stabilized state to a second apoptosis polypeptide destabilized state,
wherein the first apoptotic protein complex in the first apoptotic protein complex active state is capable of inducing apoptosis in a cell;
(II) wherein the second heterologous protease in a second heterologous protease active state is capable of cutting the second heterologous protease cleavage site of the second apoptosis polypeptide, and wherein the second heterologous protease cleavage site of the second apoptosis polypeptide being cut changes the second apoptotic protein complex from the second apoptotic protein complex inactive state to a second apoptotic protein complex active state, and
wherein the second heterologous protease in the second heterologous protease active state is capable of cutting the second heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state,
wherein the second apoptotic protein complex in the second apoptotic protein complex active state is capable of inducing apoptosis in the cell; or
(III) wherein the first heterologous protease in the first heterologous protease active state is capable of cutting the first heterologous protease cleavage site of the second apoptosis polypeptide to expose the first degron, and wherein the first degron of the second apoptosis polypeptide being exposed changes the second apoptosis polypeptide from the second apoptosis polypeptide stabilized state to the second apoptosis polypeptide destabilized state; and
wherein the second heterologous protease in the second heterologous protease active state is capable of cutting the second heterologous protease cleavage site of the first apoptosis polypeptide to expose the first degron, and wherein the first degron of the first apoptosis polypeptide being exposed changes the first apoptosis polypeptide from the first apoptosis polypeptide stabilized state to the first apoptosis polypeptide destabilized state.

16. The synthetic protein circuit of claim 11, comprising:
(i) a first apoptosis polypeptide comprising a large subunit of an apoptotic effector protein, and a first partner domain and a first degron separated by a first heterologous protease cleavage site, and a second heterologous protease cleavage site;
(ii) a second apoptosis polypeptide comprising a small subunit of an apoptotic effector protein and a second partner domain capable of binding the first partner domain, wherein
the first apoptosis polypeptide and the second apoptosis polypeptide are capable of associating via binding of the first partner domain and the second partner domain to form a subunit; wherein two subunits are capable of associating with each other to constitute a first apoptotic protein complex capable of being in a first apoptotic protein complex active state,
wherein the first apoptotic protein complex in the first apoptotic protein complex active state is capable of inducing apoptosis in a cell;
(iii) a first input polypeptide comprising a first heterologous protease; and/or
(iv) a second input polypeptide comprising a second heterologous protease, wherein:
(I) the first heterologous protease in a first heterologous protease active state is capable of cutting the first heterologous protease cleavage site of the first apoptosis polypeptide, thereby releasing the first degron;
(II) the second heterologous protease in a second heterologous protease active state is capable of cutting the second heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state.

17. The synthetic protein circuit of claim 11, comprising:
(i) a first apoptosis polypeptide comprising a large subunit of an apoptotic effector protein, a first partner domain, a first degron, and a first heterologous protease cleavage site;
(ii) a second apoptosis polypeptide comprising the large subunit of the apoptotic effector protein, the first partner domain, a second degron, and a second heterologous protease cleavage site;
(iii) a third apoptosis polypeptide comprising a small subunit of an apoptotic effector protein and a second partner domain capable of binding the first partner domain, wherein
the first apoptosis polypeptide and the third apoptosis polypeptide are capable of associating via binding of the first partner domain and the second partner domain to form a first subunit, and/or the second apoptosis polypeptide and the third apoptosis polypeptide are capable of associating via binding of the first partner domain and the second partner domain to form a second subunit,
wherein two first subunits, two second subunits, and/or one first subunit and one second subunit are capable of associating with each other to constitute a first apoptotic protein complex capable of being in a first apoptotic protein complex active state, wherein the first apoptotic protein complex in the first apoptotic protein complex active state is capable of inducing apoptosis in a cell;
(iv) a first input polypeptide comprising a first heterologous protease; and/or
(v) a second input polypeptide comprising a second heterologous protease, wherein:
(I) the first heterologous protease in a first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from a first apoptosis polypeptide stabilized state to a first apoptosis polypeptide destabilized state;
(II) the second heterologous protease in a second heterologous protease active state is capable of cutting the second heterologous protease cleavage site of the second apoptosis polypeptide to expose the second degron, and wherein the second degron of the second apoptosis polypeptide being exposed changes the second apoptosis polypeptide from a second apoptosis polypeptide stabilized state to a second apoptosis polypeptide destabilized state; or
(III) the first heterologous protease in the first heterologous protease active state is capable of cutting the first heterologous protease cleavage site of the first apoptosis polypeptide to expose the first degron, and wherein the first degron of the first apoptosis polypeptide being exposed changes the first apoptosis polypeptide from the first apoptosis polypeptide stabilized state to the first apoptosis polypeptide destabilized state, and
the second heterologous protease in the second heterologous protease active state is capable of cutting the second heterologous protease cleavage site of the second apoptosis polypeptide to expose the second degron, and wherein the second degron of the second apoptosis polypeptide being exposed changes the second apoptosis polypeptide from the second apoptosis polypeptide stabilized state to the second apoptosis polypeptide destabilized state.

18. The synthetic protein circuit of any one of claims 1-17, wherein:
the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof;
the first and/or second heterologous protease comprises or is derived from one or more of a prokaryotic protease, bacterial protease, archaeal protease, eukaryotic protease, fungal protease, plant protease, mammalian protease, and human protease, optionally the first and/or second heterologous protease is engineered, optionally wherein the first and/or second heterologous protease comprises tobacco etch virus (TEV) protease, tobacco vein mottling virus (TVMV) protease, hepatitis C virus protease (HCVP), derivatives thereof, or any combination thereof, optionally the first and/or second heterologous protease cleavage site comprises the sequence of SEQ ID NO: 1 or SEQ ID NO: 2;
the first and/or second heterologous protease cleavage site is natural or engineered, optionally the cleavage sequence of a human apoptotic effector protease, optionally a caspase cleavage sequence, optionally wherein the first and/or the second heterologous protease cleavage site is configured to modulate an activation threshold of the synthetic protein circuit, optionally wherein said configuring comprises introducing one or more amino acid insertions, substitutions, or deletions relative to a wild type first and/or second heterologous protease cleavage site, further optionally the first and/or second heterologous protease cleavage site comprises the sequence of any one of SEQ ID NOs: 3-7;
the first and second heterologous protease are different from each other;
the first, second, and/or third apoptotic protein complex in the first, second, and/or third apoptotic protein complex active state is capable of being inhibited by a small molecule inhibitor of apoptosis, optionally, the small molecule inhibitor comprises Quinoline-Val-Asp-Difluorophenoxymethylketone (Q-VD-OPh), carbobenzoxy-valyl-alanyl-aspartyl-[O-methyl]-fluoromethylketone (Z-VAD-FMK), and/or emricasan; and/or
the first apoptosis polypeptide, the second apoptosis polypeptide and/or the third apoptosis polypeptide comprises one or more third partner domains and one or more fourth partner domains capable of binding the third partner domain, wherein binding of the third partner domain and the fourth partner domain is capable of inhibiting change of the first, second, and/or third apoptotic protein complex in the first, second, and/or third apoptotic protein complex inactive state to the first, second, and/or third apoptotic protein complex active state in the absence of the first heterologous protease in the first heterologous protease active state and/or the second heterologous protease in the second heterologous protease in the second heterologous protease active state, optionally wherein said third partner domains and fourth partner domains are capable of multimerization.

19. The synthetic protein circuit of claim 11, comprising:
(i) a first pyroptosis polypeptide comprising a pyroptosis effector domain, a first inhibitory domain, and a second inhibitory domain, wherein the pyroptosis effector domain and the first inhibitory domain are separated by a first heterologous protease cleavage site, and the pyroptosis effector domain and the second inhibitory domain are separated by a second heterologous cleavage site,
wherein the first and/or second inhibitory domain is capable of inhibiting activity of the pyroptosis effector domain, thereby the first pyroptosis polypeptide is in a first pyroptosis polypeptide inactive state;
(ii) a first input polypeptide comprising a first heterologous protease; and/or
(iii) a second input polypeptide comprising a second heterologous protease, wherein:
(I) the first heterologous protease in a first heterologous protease active state is capable of cutting the first heterologous protease cleavage site of the first pyroptosis polypeptide, and wherein the first heterologous protease cleavage site of the first pyroptosis polypeptide being cut releases the first inhibitory domain from the first pyroptosis polypeptide, thereby the second inhibitory domain is capable of inhibiting the activity of the pyroptosis effector domain, thereby the first pyroptosis polypeptide is in the first pyroptosis polypeptide inactive state;
(II) the second heterologous protease in a second heterologous protease active state is capable of cutting the second heterologous protease cleavage site of the first pyroptosis polypeptide, and wherein the second heterologous protease cleavage site of the first pyroptosis polypeptide being cut releases the second inhibitory domain from the first pyroptosis polypeptide, thereby the first inhibitory domain is capable of inhibiting the activity of the pyroptosis effector domain, thereby the first pyroptosis polypeptide is in the first pyroptosis polypeptide inactive state; or
(III) wherein the first heterologous protease in the first heterologous protease active state is capable of cutting the first heterologous protease cleavage site of the first pyroptosis polypeptide, and wherein the first heterologous protease cleavage site of the first pyroptosis polypeptide being cut releases the first inhibitory domain from the first pyroptosis polypeptide, and
the second heterologous protease in the second heterologous protease active state is capable of cutting the second heterologous protease cleavage site of the first pyroptosis polypeptide, and wherein the second heterologous protease cleavage site of the first pyroptosis polypeptide being cut releases the second inhibitory domain from the first pyroptosis polypeptide,
wherein the first heterologous protease cleavage site of the first pyroptosis polypeptide being cut and the second heterologous protease cleavage site of the first pyroptosis polypeptide being cut changes the first pyroptosis polypeptide from the first pyroptosis polypeptide inactive state to a first pyroptosis polypeptide active state, and
wherein the first pyroptosis polypeptide in the first pyroptosis polypeptide active state is capable of inducing pyroptosis in a cell.

20. The synthetic protein circuit of claim 19, wherein the first and/or second inhibitory domain comprises a protein domain configured to reduce or abrogate the activity of the pyroptosis effector domain, optionally a bulky domain, further optionally the first and/or second inhibitory domain comprises an auto-inhibitory domain of a gasdermin family of pore-forming proteins or maltose-binding protein.

21. The synthetic protein circuit of claim 11, comprising:
(i) a first pyroptosis polypeptide comprising a pyroptosis effector domain and a first inhibitory domain separated by a first heterologous protease cleavage site and a second heterologous protease cleavage site, wherein the first inhibitory domain is capable of inhibiting activity of the pyroptosis effector domain, thereby the first pyroptosis polypeptide is in a first pyroptosis polypeptide inactive state;
(ii) a first input polypeptide comprising a first heterologous protease; and/or
(iii) a second input polypeptide comprising a second heterologous protease, wherein:
(I) the first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state,
wherein the first pyroptosis polypeptide in the first pyroptosis polypeptide active state is capable of inducing pyroptosis in a cell;
(II) the second heterologous protease in a second heterologous protease active state is capable of cutting the second heterologous protease cleavage site of the first pyroptosis polypeptide, and wherein the second heterologous protease cleavage site of the first pyroptosis polypeptide being cut changes the first pyroptosis polypeptide from the first pyroptosis polypeptide inactive state to a first pyroptosis polypeptide active state,
wherein the first pyroptosis polypeptide in the first pyroptosis polypeptide active state is capable of inducing pyroptosis in a cell; or
(III) the first heterologous protease in the first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state, and/or
the second heterologous protease in the second heterologous protease active state is capable of cutting the second heterologous protease cleavage site of the first pyroptosis polypeptide, and wherein the second heterologous protease cleavage site of the first pyroptosis polypeptide being cut changes the first pyroptosis polypeptide from the first pyroptosis polypeptide inactive state to a first pyroptosis polypeptide active state,
wherein the first pyroptosis polypeptide in the first pyroptosis polypeptide active state is capable of inducing pyroptosis in the cell.

22. The synthetic protein circuit of claim 21, wherein the first inhibitory domain comprises a protein domain configured to reduce or abrogate the activity of the pyroptosis effector domain, optionally a bulky domain, further optionally the first inhibitory domain comprises an auto-inhibitory domain of a gasdermin family of pore-forming proteins or maltose-binding protein.

23. The synthetic protein circuit of claim 11, comprising:
(i) a first pyroptosis polypeptide comprising a pyroptosis effector domain and a first degron separated by a first heterologous protease cleavage site, a second degron, and a second heterologous protease cleavage site, wherein the first pyroptosis polypeptide is capable of being in a first pyroptosis polypeptide destabilized state;
(ii) a first input polypeptide comprising a first heterologous protease; and/or
(iii) a second input polypeptide comprising a second heterologous protease, wherein:
(I) the first heterologous protease in a first heterologous protease active state is capable of cutting the first heterologous protease cleavage site of the first pyroptosis polypeptide and thereby releasing the first degron, and wherein the first heterologous protease cleavage site of the first pyroptosis polypeptide being cut changes the first pyroptosis polypeptide from the first pyroptosis polypeptide destabilized state to a first pyroptosis polypeptide stabilized state,
wherein the first pyroptosis polypeptide in the first pyroptosis polypeptide stabilized state is capable of being in a first pyroptosis polypeptide active state,
wherein the first pyroptosis polypeptide in the first pyroptosis polypeptide active state is capable of inducing pyroptosis in a cell; or
(II) the first heterologous protease in the first heterologous protease active state is capable of cutting the first heterologous protease cleavage site of the first pyroptosis polypeptide and thereby releasing the first degron, and wherein the first heterologous protease cleavage site of the first pyroptosis polypeptide being cut changes the first pyroptosis polypeptide from the first pyroptosis polypeptide destabilized state to the first pyroptosis polypeptide stabilized state, and
the second heterologous protease in the second heterologous protease active state is capable of cutting the second heterologous protease cleavage site of the first pyroptosis polypeptide in the first pyroptosis polypeptide destabilized state to expose the second degron, and wherein the second degron of the first pyroptosis polypeptide in the first pyroptosis polypeptide stabilized state being exposed changes the first pyroptosis polypeptide from the first pyroptosis polypeptide stabilized state to the first pyroptosis polypeptide destabilized state.

24. The synthetic protein circuit of claim 11, comprising:
(i) a first pyroptosis polypeptide comprising a pyroptosis effector domain and a first inhibitory domain separated by a first heterologous protease cleavage site, a second heterologous cleavage site, and a first degron, wherein the first inhibitory domain is capable of inhibiting activity of the pyroptosis effector domain of the first pyroptosis polypeptide, thereby the first pyroptosis polypeptide is in a first pyroptosis polypeptide inactive state;
(ii) a second pyroptosis polypeptide comprising a pyroptosis effector domain and a second inhibitory domain separated by the second heterologous protease cleavage site, the first heterologous cleavage site, and a second degron, wherein the second inhibitory domain is capable of inhibiting activity of the pyroptosis effector domain of the second pyroptosis polypeptide, thereby the second pyroptosis polypeptide is in a second pyroptosis polypeptide inactive state;
(iii) a first input polypeptide comprising a first heterologous protease; and/or
(iv) a second input polypeptide comprising a second heterologous protease, wherein:
(I) the first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state,
wherein the first pyroptosis polypeptide in the first pyroptosis polypeptide active state is capable of inducing pyroptosis in a cell, and
wherein the first heterologous protease in the first heterologous protease active state is capable of cutting the first heterologous protease cleavage site of the second pyroptosis polypeptide to expose the second degron, and wherein the second degron of the second pyroptosis polypeptide being exposed changes the second pyroptosis polypeptide from a second pyroptosis polypeptide stabilized state to a second pyroptosis polypeptide destabilized state;
(II) the second heterologous protease in a second heterologous protease active state is capable of cutting the second heterologous protease cleavage site of the second pyroptosis polypeptide, and wherein the second heterologous protease cleavage site of the second pyroptosis polypeptide being cut changes the second pyroptosis polypeptide from the second pyroptosis polypeptide inactive state to a second pyroptosis polypeptide active state,
wherein the second pyroptosis polypeptide in the second pyroptosis polypeptide active state is capable of inducing pyroptosis in the cell, and
wherein the second heterologous protease in the second heterologous protease active state is capable of cutting the second heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state; or
(III) the first heterologous protease in the first heterologous protease active state is capable of cutting the first heterologous protease cleavage site of the second pyroptosis polypeptide to expose the second degron, and wherein the second degron of the second pyroptosis polypeptide being exposed changes the second pyroptosis polypeptide from the second pyroptosis polypeptide stabilized state to the second pyroptosis polypeptide destabilized state, and
wherein the second heterologous protease in the second heterologous protease active state is capable of cutting the second heterologous protease cleavage site of the first pyroptosis polypeptide to expose the first degron, and wherein the first degron of the first pyroptosis polypeptide being exposed changes the first pyroptosis polypeptide from the first pyroptosis polypeptide stabilized state to the first pyroptosis polypeptide destabilized state.

25. The synthetic protein circuit of claim 24, wherein the first and/or second inhibitory domain comprises a protein domain configured to reduce or abrogate the activity of the pyroptosis effector domain, optionally a bulky domain, further optionally the first and/or second inhibitory domain comprises an auto-inhibitory domain of a gasdermin family of pore-forming proteins or maltose-binding protein.

26. The synthetic protein circuit of claim 11, comprising:
(i) a first pyroptosis polypeptide comprising a pyroptosis effector domain and a first degron separated by a first heterologous protease cleavage site, and a second heterologous protease cleavage site, wherein the first pyroptosis polypeptide is capable of being in a first pyroptosis polypeptide active state, wherein the first pyroptosis polypeptide in the first pyroptosis polypeptide active state is capable of inducing pyroptosis in a cell;
(ii) a first input polypeptide comprising a first heterologous protease; and/or
(iii) a second input polypeptide comprising a second heterologous protease, wherein:
(I) the first heterologous protease in a first heterologous protease active state is capable of cutting the first heterologous protease cleavage site of the first pyroptosis polypeptide and thereby releasing the first degron from the first pyroptosis polypeptide; or
(II) the second heterologous protease in a second heterologous protease active state is capable of cutting the second heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state.

27. The synthetic protein circuit of claim 11, comprising:
(i) a first pyroptosis polypeptide comprising a pyroptosis effector domain, a first degron, and a first heterologous protease cleavage site, wherein the first pyroptosis polypeptide is capable of being in a first pyroptosis polypeptide active state, wherein the first pyroptosis polypeptide in the first pyroptosis polypeptide active state is capable of inducing pyroptosis in a cell;
(ii) a second pyroptosis polypeptide comprising a pyroptosis effector domain, a second degron, and a second heterologous protease cleavage site, wherein the second pyroptosis polypeptide is capable of being in a second pyroptosis polypeptide active state, wherein the second pyroptosis polypeptide in the second pyroptosis polypeptide active state is capable of inducing pyroptosis in the cell;
(iii) a first input polypeptide comprising a first heterologous protease; and/or
(iv) a second input polypeptide comprising a second heterologous protease, wherein:
(I) the first heterologous protease in a first heterologous protease active state is capable of cutting the first heterologous protease cleavage site of the first pyroptosis polypeptide to expose the first degron, and wherein the first degron of the first pyroptosis polypeptide being exposed changes the first pyroptosis polypeptide from a first pyroptosis polypeptide stabilized state to a first pyroptosis polypeptide destabilized state;
(II) the second heterologous protease in a second heterologous protease active state is capable of cutting the second heterologous protease cleavage site of the second pyroptosis polypeptide to expose the second degron, and wherein the second degron of the second pyroptosis polypeptide being exposed changes the second pyroptosis polypeptide from a second pyroptosis polypeptide stabilized state to a second pyroptosis polypeptide destabilized state; or
(III) the first heterologous protease in the first heterologous protease active state is capable of cutting the first heterologous protease cleavage site of the first pyroptosis polypeptide to expose the first degron, and wherein the first degron of the first pyroptosis polypeptide being exposed changes the first pyroptosis polypeptide from the first pyroptosis polypeptide stabilized state to the first pyroptosis polypeptide destabilized state, and
the second heterologous protease in the second heterologous protease active state is capable of cutting the second heterologous protease cleavage site of the second pyroptosis polypeptide to expose the second degron, and wherein the second degron of the second pyroptosis polypeptide being exposed changes the second pyroptosis polypeptide from the second pyroptosis polypeptide stabilized state to the second pyroptosis polypeptide destabilized state.

28. The synthetic protein circuit of any one of claims 1-27, wherein:
the pyroptosis effector domain comprises an N-terminal domain of a gasdermin (GSDM) protein;
the GSDM protein is from the gasdermin (GSDM) family, including GSDMA, GSDMB, GSDMC, GSDMD, GSDME, or DFNB59;
the first and/or second heterologous protease comprises or is derived from one or more of a prokaryotic protease, bacterial protease, archaeal protease, eukaryotic protease, fungal protease, plant protease, mammalian protease, and human protease, optionally the first and/or second heterologous protease is engineered, optionally wherein the first and/or second heterologous protease comprises tobacco etch virus (TEV) protease, tobacco vein mottling virus (TVMV) protease, hepatitis C virus protease (HCVP), derivatives thereof, or any combination thereof, optionally the first and/or second heterologous protease cleavage site comprises the sequence of SEQ ID NO: 1 or SEQ ID NO: 2;
the first and/or second heterologous protease cleavage site is natural or engineered, optionally the cleavage sequence of a human apoptotic effector protease, optionally a caspase cleavage sequence, optionally wherein the first and/or second heterologous protease cleavage site is configured to modulate an activation threshold of the synthetic protein circuit, optionally wherein said configuring comprises introducing one or more amino acid insertions, substitutions, or deletions relative to a wild type first and/or second heterologous protease cleavage site, further optionally the first and/or second heterologous protease cleavage site comprises the sequence of any one of SEQ ID NOs: 3-7;
the first pyroptosis polypeptide and/or the second pyroptosis polypeptide comprise one or more third partner domains and one or more fourth partner domains capable of binding the third partner domain, wherein binding of the third partner domain and the fourth partner domain is capable of, inhibiting change of the first and/or second pyroptosis polypeptide from the first and/or second pyroptosis polypeptide inactive state to the first and/or second pyroptosis polypeptide active state, in the absence of the first and/or second heterologous protease in the first and/or second heterologous protease active state, optionally wherein said third partner domains and fourth partner domains are capable of multimerization; and/or
inducing pyroptosis in the cell causes the cell to release one or more inflammatory cytokines, optionally the one or more inflammatory cytokines comprise IL-18, IL-1β, IL-6, IL-8, interferon gamma (IFN-γ), and/or tumor necrosis factor-alpha (TNF-α).

29. The synthetic protein circuit of any one of claims 1-28, wherein:
(i) the first, second, and/or third apoptosis polypeptide;
(ii) the first and/or second pyroptosis polypeptide; and/or
(iii) the first and/or second input polypeptide,
are configured to be in a first localized state, optionally
the first localized state comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the first, second, and/or third apoptosis polypeptide, the first and/or second pyroptosis polypeptide, and/or the first and/or second input polypeptide is: (i) tethered to an intracellular organelle and/or membrane; or (ii) fused to a polypeptide that recruits to said localized state.

30. The synthetic protein circuit of claim 29, wherein:
(i) the first, second, and/or third apoptosis polypeptide;
(ii) the first and/or second pyroptosis polypeptide; and/or
(iii) the first and/or second input polypeptide,
are configured to be in second localized state(s), wherein the first localized state and the second localized state(s) are different, optionally
the second localized state(s) comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the first, second, and/or third apoptosis polypeptide, the first and/or second pyroptosis polypeptide, and/or the first and/or second input polypeptide is: (i) tethered to an intracellular organelle and/or membrane; or (ii) fused to a polypeptide that recruits to said localized state.

31. The synthetic protein circuit of any one of claims 29-30, wherein:
(i) the first, second, and/or third apoptosis polypeptide;
(ii) the first and/or second pyroptosis polypeptide; and/or
(iii) the first and/or second input polypeptide comprise a first localization signal, optionally the first localization signal is adjacent to a third degron and/or a third heterologous cleavage site.

32. The synthetic protein circuit of any one of claims 29-31, wherein:
(i) the first, second, and/or third apoptosis polypeptide;
(ii) the first and/or second pyroptosis polypeptide; and/or
(iii) the first and/or second input polypeptide comprise second localization signal(s), optionally the second localization signal is adjacent to a third degron and/or a third heterologous protease cleavage site.

33. The synthetic protein circuit of any one of claims 29-32, wherein the presence of the third degron and/or wherein the third heterologous cleavage site being cut changes:
(i) the first, second, and/or third apoptosis polypeptide from a non-localized state or the first localized state to the second localized state(s) or to a non-localized state;
(ii) the first and/or second pyroptosis polypeptide from a non-localized state or the first localized state to the second localized state(s) or to a non-localized state; and/or
(iii) the first and/or second input polypeptide from a non-localized state or the first localized state to the second localized state(s) or to a non-localized state.

34. The synthetic protein circuit of any one of claims 29-33, wherein:
the synthetic protein circuit further comprises a third input polypeptide comprising a third heterologous protease capable of cutting the third heterologous protease cleavage site, optionally the third heterologous protease comprises or is derived from one or more of a prokaryotic protease, bacterial protease, archaeal protease, eukaryotic protease, fungal protease, plant protease, mammalian protease, and human protease, optionally the third heterologous protease is engineered, optionally wherein the third heterologous protease comprises tobacco etch virus (TEV) protease, tobacco vein mottling virus (TVMV) protease, hepatitis C virus protease (HCVP), derivatives thereof, or any combination thereof, further optionally the third heterologous protease cleavage site comprises the sequence of SEQ ID NO: 1 or SEQ ID NO: 2; and/or
the third heterologous protease cleavage site is natural or engineered, optionally the cleavage sequence of a human apoptotic effector protease, optionally a caspase cleavage sequence, optionally wherein the third heterologous protease cleavage site is configured to modulate an activation threshold of the synthetic protein circuit, optionally wherein said configuring comprises introducing one or more amino acid insertions, substitutions, or deletions relative to a wild type third heterologous protease cleavage site, further optionally the third heterologous protease cleavage site comprises the sequence of any one of SEQ ID NOs: 3-7.

35. The synthetic protein circuit of any one of claims 29-34, wherein:
(a) the first, second, and/or third apoptosis polypeptide in the first localized state;
(b) the first, second, and/or third apoptosis polypeptide in the second localized state(s);
(c) the first and/or second pyroptosis in the first localized state;
(d) the first and/or second pyroptosis polypeptide in the second localized state(s);
(e) the first and/or second input polypeptide in the first localized state; and/or
(f) the first and/or second input polypeptide in the second localized state(s), is capable of modulating an activation threshold and/or sensitivity of the synthetic protein circuit.

36. The synthetic protein circuit of any one of claims 1-35, wherein the synthetic protein circuit is present in a cell, wherein the cell is:
a cell of a subject, optionally a subject suffering from a disease or disorder, optionally the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof;
a cell derived from a donor; and/or
an in vivo cell, an ex vivo cell, an in vitro cell or an in situ cell.

37. A synthetic protein circuit comprising:
one or more first, second, or third apoptosis polypeptides;
one or more first or second pyroptosis polypeptides;
one or more input polypeptides;
one or more pyroptosis effector proteins; and/or
one or more mutant pyroptosis effector proteins,
wherein the synthetic protein circuit is capable of inducing in a cell:
apoptosis via a first apoptotic protein complex in first apoptotic protein complex active state; and/or
pyroptosis via a first and/or second pyroptosis polypeptide in a first and/or second pyroptosis polypeptide active state or a pyroptosis effector protein in a pyroptosis effector protein active state.

38. A synthetic protein circuit comprising:
(i) a first apoptosis polypeptide comprising a large subunit of an apoptotic effector protein and a small subunit of an apoptotic effector protein separated by a first heterologous protease cleavage site,
wherein two of the first apoptosis polypeptides are capable of forming a first apoptotic protein complex in a first apoptotic protein complex inactive state, and
wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic complex inactive state to a first apoptotic protein complex active state,
wherein the first apoptotic protein complex in the first apoptotic protein complex active state is capable of inducing apoptosis in a cell; and
(ii) a first pyroptosis polypeptide comprising a pyroptosis effector domain and a first inhibitory domain separated by the first heterologous protease cleavage site, wherein the first inhibitory domain is capable of inhibiting activity of the pyroptosis effector domain, thereby the first pyroptosis polypeptide is capable of being in a first pyroptosis polypeptide inactive state,
wherein the first heterologous protease in the first heterologous protease active state is capable of cutting the first heterologous protease cleavage site of the first of the first pyroptosis polypeptide, wherein the first pyroptosis polypeptide being cut changes the first pyroptosis polypeptide from the first pyroptosis polypeptide inactive state to a first pyroptosis polypeptide active state,
and wherein the first pyroptosis polypeptide in the first pyroptosis polypeptide active state is capable of:
(a) inducing pyroptosis in the cell; and
(b) inhibiting the induction of apoptosis in the cell by the first apoptotic protein complex in the first apoptotic protein complex active state, optionally said inhibition is at least 10%,
optionally the synthetic protein circuit further comprises a first input polypeptide comprising the first heterologous protease.

39. The synthetic protein circuit of claim 38, wherein:
the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof;
the pyroptosis effector domain comprises an N-terminal domain of a gasdermin (GSDM) protein;
the GSDM protein is GSDMA, GSDMB, GSDMC, GSDMD, GSDME, or DFNB59;
the first heterologous protease comprises or is derived from one or more of a prokaryotic protease, bacterial protease, archaeal protease, eukaryotic protease, fungal protease, plant protease, mammalian protease, and human protease, optionally the first heterologous protease is engineered, optionally wherein the first heterologous protease comprises tobacco etch virus (TEV) protease, tobacco vein mottling virus (TVMV) protease, hepatitis C virus protease (HCVP), derivatives thereof, or any combination thereof, optionally the first heterologous protease cleavage site comprises the sequence of SEQ ID NO: 1 or SEQ ID NO: 2;
the first heterologous protease cleavage site is natural or engineered, optionally the cleavage sequence of a human apoptotic effector protease, optionally a caspase cleavage sequence, optionally wherein the first heterologous protease cleavage site is configured to modulate an activation threshold of the synthetic protein circuit, optionally wherein said configuring comprises introducing one or more amino acid insertions, substitutions, or deletions relative to a wild type first heterologous protease cleavage site, further optionally the first heterologous protease cleavage site comprises the sequence of any one of SEQ ID NOs: 3-7;
the first apoptosis polypeptide comprises one or more third partner domains and one or more fourth partner domains capable of binding the third partner domain, wherein binding of the third partner domain and the fourth partner domain is capable of inhibiting change of the first apoptotic protein complex from the first apoptotic protein complex inactive state to the first apoptotic protein complex active state in the absence of the first heterologous protease in the first heterologous protease active state, optionally wherein said third partner domains and fourth partner domains are capable of multimerization;
the first pyroptosis polypeptide comprises one or more third partner domains and one or more fourth partner domains capable of binding the third partner domain, wherein binding of the third partner domain and the fourth partner domain is capable of inhibiting change of the first pyroptosis polypeptide from the first pyroptosis polypeptide inactive state to the first pyroptosis polypeptide active state in the absence of the first heterologous protease in the first heterologous protease active state, optionally wherein said third partner domains and fourth partner domains are capable of multimerization; and/or
the first inhibitory domain comprises a protein domain configured to reduce or abrogate the activity of the pyroptosis effector domain, optionally a bulky domain, further optionally the first inhibitory domain comprises an auto-inhibitory domain of a gasdermin family of pore-forming proteins or maltose-binding protein.

40. A synthetic protein circuit comprising:
a first apoptosis polypeptide comprising a large subunit of an apoptotic effector protein and a small subunit of an apoptotic effector protein, separated by a first heterologous protease cleavage site,
wherein two of the first apoptosis polypeptides are capable of forming a first apoptotic protein complex in a first apoptotic protein complex inactive state,
wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptosis protease complex inactive state to a first apoptotic protein complex active state,
wherein the first apoptotic protein complex in the first apoptotic protein complex active state is capable of:
(a) inducing apoptosis in a cell expressing a pyroptosis effector protein; and/or
(b) cutting the pyroptosis effector protein, wherein the pyroptosis effector protein being cut changes the pyroptosis effector protein from a pyroptosis effector protein inactive state to a pyroptosis effector protein active state,
wherein the pyroptosis effector protein in the pyroptosis effector protein active state is capable of:
(c) inducing pyroptosis in the cell; and
(d) inhibiting the induction of apoptosis in the cell by the first apoptotic protein complex in the first apoptotic protein complex active state, and
optionally the synthetic protein circuit further comprises a first input polypeptide comprising the first heterologous protease.

41. A synthetic protein circuit comprising:
(i) a first apoptosis polypeptide comprising a large subunit of an apoptotic effector protein and a small subunit of an apoptotic effector protein, separated by a first heterologous protease cleavage site,
wherein two of the first apoptosis polypeptides are capable of forming a first apoptotic protein complex in a first apoptotic protein complex inactive state,
wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state,
wherein the first apoptotic protein complex in the first apoptotic protein complex active state is capable of:
(a) inducing apoptosis in a cell expressing a pyroptosis effector protein; and
(b) cutting the pyroptosis effector protein,
wherein the pyroptosis effector protein being cut changes the pyroptosis effector protein from a pyroptosis effector protein inactive state to a pyroptosis effector protein active state, wherein the pyroptosis effector protein in the pyroptosis effector protein active state is capable of:
(c) inducing pyroptosis in the cell; and
(d) inhibiting the induction of apoptosis in the cell by the apoptotic protein complex in the first apoptotic protein complex active state; and
(ii) a first pyroptosis polypeptide comprising a mutant pyroptosis effector domain comprising a mutation, capable of inhibiting the pyroptosis effector protein in the pyroptosis effector protein active state, thereby changing the pyroptosis effector protein from the pyroptosis effector protein active state to a pyroptosis effector protein inactive state;
thereby the first apoptotic protein complex in the first apoptotic protein complex active state is capable of inducing apoptosis in the cell,
optionally the synthetic protein circuit comprises a first input polypeptide comprising the first heterologous protease.

42. The synthetic protein circuit of any one of claims 40-41, wherein:
the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof;
the first apoptosis polypeptide comprises one or more third partner domains and one or more fourth partner domains capable of binding the third partner domain, wherein binding of the third partner domain and the fourth partner domain is capable of inhibiting change of the first apoptotic protein complex from the first apoptotic protein complex inactive state to the first apoptotic protein complex active state, in the absence of the first heterologous protease in the first heterologous protease active state, optionally wherein said third partner domains and fourth partner domains are capable of multimerization;
the pyroptosis effector protein comprises a gasdermin (GSDM) protein;
the GSDM protein is GSDMA, GSDMB, GSDMC, GSDMD, GSDME, or DFNB59;
the first heterologous protease comprises or is derived from one or more of a prokaryotic protease, bacterial protease, archaeal protease, eukaryotic protease, fungal protease, plant protease, mammalian protease, and human protease, optionally the first heterologous protease is engineered, optionally wherein the first heterologous protease comprises tobacco etch virus (TEV) protease, tobacco vein mottling virus (TVMV) protease, hepatitis C virus protease (HCVP), derivatives thereof, or any combination thereof, optionally the first heterologous protease cleavage site comprises the sequence of SEQ ID NO: 1 or SEQ ID NO: 2;
the first heterologous protease cleavage site is natural or engineered, optionally the cleavage sequence of a human apoptotic effector protease, optionally a caspase cleavage sequence, optionally wherein the first heterologous protease cleavage site is configured to modulate an activation threshold of the synthetic protein circuit, optionally wherein said configuring comprises introducing one or more amino acid insertions, substitutions, or deletions relative to a wild type first heterologous protease cleavage site, further optionally the first heterologous protease cleavage site comprises the sequence of any one of SEQ ID NOs: 3-7;
the mutant pyroptosis effector domain is derived from an N-terminal domain of a gasdermin (GSDM) protein, optionally the GSDM protein is GSDMA, GSDMB, GSDMC, GSDMD, GSDME, or DFNB59;
the one or more mutations comprises a V99N, L101N, L103N, V193E, A195E, G199E, and/or I217N mutation in GSDME; and/or
the mutation is an I217N mutation in GSDME.

43. The synthetic protein circuit of any one of claims 38-42, wherein inducing pyroptosis in the cell causes the cell to release one or more inflammatory cytokines, optionally the one or more inflammatory cytokines comprise IL-18, IL-1β, IL-6, IL-8, interferon gamma (IFN-γ), and/or tumor necrosis factor-alpha (TNF-α).

44. The synthetic protein circuit of any one of claims 38-43, wherein inhibition of the pyroptosis effector protein by the first pyroptosis polypeptide is dose-dependent, thereby the induction of apoptosis in the cell is dose-dependent.

45. The synthetic protein circuit of claim 44, wherein a concentration of the first pyroptosis peptide is at least two-folder higher than a concentration of the pyroptosis effector protein in the cell, thereby the inhibition of the pyroptosis effector protein by the first pyroptosis polypeptide is increased relative to a cell wherein the concentration of the first pyroptosis polypeptide is not at least two-fold higher than the concentration of the pyroptosis effector protein.

46. The synthetic protein circuit of any one of claims 38-45, wherein:
(i) the first apoptosis polypeptide;
(ii) the first pyroptosis polypeptide, and/or
(iii) the first input polypeptide,
are configured to be in a first localized state, optionally
the first localized state comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the first apoptosis polypeptide, the first pyroptosis polypeptide, and/or the first input polypeptide is: (i) tethered to an intracellular organelle and/or membrane; or (ii) fused to a polypeptide that recruits to said localized state.

47. The synthetic protein circuit of claim 46, wherein:
(i) the first apoptosis polypeptide;
(ii) the first pyroptosis polypeptide, and/or
(iii) the first input polypeptide,
are configured to be in second localized state(s), wherein the first localized state and the second localized state(s) are different, optionally
the second localized state(s) comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the first apoptosis polypeptide, the first pyroptosis polypeptide, and/or the first input polypeptide is: (i) tethered to an intracellular organelle and/or membrane; or (ii) fused to a polypeptide that recruits to said localized state.

48. The synthetic protein circuit of any one of claims 46-47, wherein:
(i) the first apoptosis polypeptide;
(ii) the first pyroptosis polypeptide, and/or
(iii) the first input polypeptide comprise a first localization signal, optionally the first localization signal is adjacent to a third degron and/or a third heterologous cleavage site.

49. The synthetic protein circuit of any one of claims 46-48, wherein:
(i) the first apoptosis polypeptide;
(ii) the first pyroptosis polypeptide, and/or
(iii) the first input polypeptide comprise second localization signal(s), optionally the second localization signal is adjacent to a third degron and/or a third heterologous protease cleavage site.

50. The synthetic protein circuit of any one of claims 46-49, wherein the presence of the third degron and/or wherein the third heterologous cleavage site being cut changes:
(i) the first apoptosis polypeptide from a non-localized state or the first localized state to the second localized state(s) or a non-localized state;
(ii) the first pyroptosis polypeptide from a non-localized state or the first localized state to the second localized state(s) or a non-localized state; and/or
(iii) the first input polypeptide from a non-localized state or the first localized state to the second localized state(s) or a non-localized state.

51. The synthetic protein circuit of any one of claims 46-50, wherein the synthetic protein circuit further comprises a third input polypeptide comprising a third heterologous protease capable of cutting the third heterologous protease cleavage site, optionally wherein the third heterologous protease comprises or is derived from one or more of a prokaryotic protease, bacterial protease, archaeal protease, eukaryotic protease, fungal protease, plant protease, mammalian protease, and human protease, optionally the third heterologous protease is engineered, optionally wherein the third heterologous protease comprises tobacco etch virus (TEV) protease, tobacco vein mottling virus (TVMV) protease, hepatitis C virus protease (HCVP), derivatives thereof, or any combination thereof, further optionally the third heterologous protease cleavage site comprises the sequence of SEQ ID NO: 1 or SEQ ID NO: 2.

52. The synthetic protein circuit of claim 51, wherein the third heterologous protease cleavage site is natural or engineered, optionally the cleavage sequence of a human apoptotic effector protease, optionally a caspase cleavage sequence, optionally wherein the third heterologous protease cleavage site is configured to modulate an activation threshold of the synthetic protein circuit, optionally wherein said configuring comprises introducing one or more amino acid insertions, substitutions, or deletions relative to a wild type third heterologous protease cleavage site, further optionally the third heterologous protease cleavage site comprises the sequence of any one of SEQ ID NOs: 3-7.

53. The synthetic protein circuit of any one of claims 46-52, wherein:
(a) the first apoptosis polypeptide in the first localized state;
(b) the first apoptosis polypeptide in the second localized state(s);
(c) the first pyroptosis in the first localized state;
(d) the first pyroptosis polypeptide in the second localized state(s);
(e) the first input polypeptide in the first localized state; and/or
(f) the first second input polypeptide in the second localized state(s),
is capable of modulating an activation threshold and/or sensitivity of the synthetic protein circuit.

54. The synthetic protein circuit of any one of claims 38-53, wherein the synthetic protein circuit is present in a cell, wherein the cell is:
a cell of a subject, optionally a subject suffering from a disease or disorder, optionally the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof;
a cell derived from a donor; and/or
an in vivo cell, an ex vivo cell, an in vitro cell or an in situ cell.

55. A synthetic protein circuit comprising:
a first polypeptide comprising a first signal transducer binding domain and a first part of a first cell death executioner, wherein the first signal transducer binding domain is capable of binding a first signal transducer to form a first signal transducer-bound polypeptide;
a second polypeptide comprising a second signal transducer binding domain and a second part of the first cell death executioner, wherein the second signal transducer binding domain is capable of binding a second signal transducer to form a second signal transducer-bound polypeptide, and wherein the first part of the first cell death executioner and the second part of the first cell death executioner are capable of associating with each other to constitute a first cell death executioner capable of being in a first cell death executioner active state when the first signal transducer and the second signal transducer are in close proximity at an association location; and
wherein the first cell death executioner in the first cell death executioner active state is capable of inducing apoptosis or pyroptosis in a cell.

56. The synthetic protein circuit of claim 55, wherein:
the first signal transducer binding domain of the first polypeptide and the second signal transducer binding domain of the second polypeptide are identical;
the first transducer and the second transducer are identical and/or are the same protein;
the first cell death executioner comprises an apoptosis effector protein or a pyroptosis effector protein;
the first part of the first cell death executioner comprises a large subunit of the apoptotic effector protein and/or wherein the second part of second first cell death executioner comprises a small subunit of the apoptotic effector protein;
the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof;
the pyroptosis effector protein comprises a gasdermin (GSDM) protein; and/or
the GSDM protein is GSDMA, GSDMB, GSDMC, GSDMD, GSDME, or DFNB59.

57. The synthetic protein circuit of any one of claims 55-56, wherein:
the first signal transducer, the second signal transducer, or both, are capable of being localized at the association location;
the first signal transducer when in a first signal transducer active state, the second signal transducer when in a second signal transducer active state, or both, are capable of being localized at the association location;
the first signal transducer when in a first inactive state, the second signal transducer when in a second inactive state, or both, are capable of being localized at the association location;
the first signal transducer binding domain of the first polypeptide is capable of binding to the first signal transducer, wherein the second signal transducer binding domain of the second polypeptide is capable of binding to the second signal transducer, or both;
the first signal transducer binding domain of the first polypeptide is capable of binding to the first signal transducer in a first signal transducer active state, wherein the second signal transducer binding domain of the second polypeptide is capable of binding to the second signal transducer in a second signal transducer active state, or both;
the first signal transducer binding domain of the first polypeptide is capable of binding to the first signal transducer in a first inactive state, wherein the second signal transducer binding domain of the second polypeptide is capable of binding to the second signal transducer in a second inactive state, or both;
the signal transducer binding domain of the first polypeptide is capable of binding the first signal transducer to form the first signal transducer-bound polypeptide at the association location, wherein the signal transducer binding domain of the first polypeptide is capable of binding the first signal transducer to form the first signal transducer-bound polypeptide at the association location, or both;
the signal transducer binding domain of the first polypeptide is capable of binding the first signal transducer to form the first signal transducer-bound polypeptide at a first cellular location other than the association location, wherein the signal transducer binding domain of the first polypeptide is capable of binding the first signal transducer to form the first signal transducer-bound polypeptide at a second cellular location other than the association location, or both;
the first cellular location, the second cellular location, or both comprise one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof; and/or
the association location comprises one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof.

58. The synthetic protein circuit of any one of claims 55-57, wherein:
a first concentration of the first signal transducer-bound polypeptide is at least two-fold higher at the association location as compared to a first cellular location other than the association location when the first signal transducer is in a first signal transducer active state, and/or wherein a second concentration of the second signal transducer-bound polypeptide is at least two-fold higher at the association location as compared to a second cellular location other than the association location when the second signal transducer is in a second signal transducer active state;
a first concentration of the first cell death executioner in the first cell death executioner active state is at least two-fold higher at the association location as compared to a cellular location other than the association location when the first signal transducer is in a first signal transducer active state and/or when the second signal transducer is in a second signal transducer active state;
the first part of the first cell death executioner and the second part of the first cell death executioner have the weak association affinity when the first signal transducer is in a first signal transducer inactive state and/or the second signal transducer is in a second signal transducer inactive state;
the first part of the first cell death executioner and the second part of the first cell death executioner are incapable of associating to form the first cell death executioner in the first cell death executioner active state when the first signal transducer is in a first signal transducer inactive state and/or the second signal transducer is in a second signal transducer inactive state;
a first concentration of the first signal transducer-bound polypeptide and a second concentration of the second signal transducer-bound polypeptide at the association location are insufficient for the first part of the first cell death executioner and the second part of the first cell death executioner to form an active first cell death executioner when the first signal transducer is in a first signal transducer inactive state and/or the second signal transducer is in a second signal transducer inactive state;
a first concentration of the first signal transducer-bound polypeptide at the association location is comparable to a first cellular location other than the association location when the first signal transducer is in a first signal transducer inactive state, and/or wherein a second concentration of the second signal transducer-bound polypeptide at the association location is comparable to a second cellular location other than the association location when the second signal transducer is in a second signal transducer inactive state;
the first part of the first cell death executioner and the second part of the first cell death executioner are capable of associating with each other to form the first cell death executioner in the first cell death executioner active state at a threshold first polypeptide concentration and a threshold second polypeptide concentration at the association location; and/or
the threshold first polypeptide concentration and the threshold second polypeptide concentration at the association location are reached at a threshold signal transducer activation level of the signal transducer.

59. The synthetic protein circuit of any one of claims 55-58, wherein:
the first signal transducer binding domain and/or the second signal transducer binding domain are identical;
the first signal transducer binding domain and/or the second signal transducer binding domain are different;
the first signal transducer binding domain and/or the second signal transducer binding domain each is capable of binding molecules of the first signal transducer and/or the second signal transducer;
the first signal transducer and/or the second signal transducer belong to a signal transduction pathway;
the first signal transducer binding domain and/or the second signal transducer binding domain comprise a RAS binding domain (RBD) and/or RAS association domain (RAD); optionally the RAS binding domain comprises or is derived from a RAS interacting protein, optionally selected from the group comprising AGO2, APBB1IP, APPL1, ARAF, ARL1, ARL2, ARRB1, ARRB2, BAIAP2, BCL2, BCL2L1, BRAF, BRAP, BSG, CALM1, CALM3, CALML3, CALML4, CALML5, CALML6, CNKSR1, CNKSR2, CSK, DAB2IP, EGFR, ERBIN, FGA, FGB, FGG, FN1, GRB2, HK1, IFNGR1, IL6, IQGAP1, ITGA2B, ITGB3, KSR1, KSR2, LGALS3, LYN, LZTR1, MAP2K1, MAP2K2, MAPK1, MAPK14, MAPK3, MAPKAP1, MARK2, MARK3, MBP, MSI2, MTOR, NCBP2AS2, NF1, NIBAN2, PDE4DIP, PDE6D, PDPK1, PEBP1, PIK3CA, PIK3CB, PIK3CD, PIK3R1, PIK3R2, PIP5K1A, PLCE1, PPIA, PRKCZ, PTGS2, RAF1, RALB, RALGDS, RAP1A, RAP1B, RAP1GDS1, RASA1, RASA2, RASA3, RASA4, RASAL1, RASAL2, RASAL3, RASSF1, RASSF2, RASSF5, RGL1, RGL3, RIN1, SHOC2, SOS1, SOS2, SPRED1, SPRED2, SPRED3, SRC, SYNGAP1, TIAM1, TLN1, VCL, VWF, YWHAB, or any combination thereof;
the first signal transducer binding domain and/or the second signal transducer binding domain comprises a lipid binding domain;
the lipid binding domain comprises a Pleckstrin homology (PH) domain; and/or
the first signal transducer binding domain and/or the second signal transducer binding domain comprises an antibody, an antibody fragment, a binding domain derived from a natural protein, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

60. The synthetic protein circuit of any one of claims 55-59, wherein:
the first signal transducer is capable of binding the first signal transducer binding domain and/or the second signal transducer is capable of binding the second signal transducer binding domain following a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof;
the first signal transducer, the second signal transducer, or both are endogenous proteins;
the first signal transducer, the second signal transducer, or both comprise AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STAT3, STAT5, STAT6, or any combination thereof;
the first signal transducer and/or the second signal transducer are capable of regulating cell survival, cell growth, cell proliferation, cell adhesion, cell migration, cell metabolism, cell morphology, cell differentiation, apoptosis, or any combination thereof;
the first signal transducer, the second signal transducer, or both comprise a RAS protein;
the RAS protein is KRAS, NRAS, HRAS, or any combination thereof;
the first signal transducer, the second signal transducer, or both are exogenous proteins;
the synthetic protein circuit comprises the first signal transducer, the second signal transducer, or both;
the first signal transducer, the second signal transducer, or both comprise a lipid;
the lipid comprises a phospholipid; and/or
the phospholipid is phosphatidylinositol 3-phosphate.

61. The synthetic protein circuit of any one of claims 55-60, wherein:
the synthetic protein circuit is capable of detecting an activity of the first signal transducer and an activity of the second signal transducer;
an activity of the first cell death executioner correlates with an activity of the first signal transducer and/or an activity of the second signal transducer;
the synthetic protein circuit is capable of detecting activities of the first signal transducer and activities of the second signal transducer over a period of time;
activities of the first cell death executioner correlate with activities of the first signal transducer and activities of the second signal transducer over a period of time;
the synthetic protein circuit is capable of detecting an aberrant signaling;
aberrant signaling involves an active signal transducer;
the aberrant signaling involves an overactive signal transducer;
the aberrant signaling involves a constitutively active signal transducer over a period of time;
the synthetic protein circuit is capable of detecting an activity of a signal transducer activator and/or an activity of a signal transducer repressor;
the aberrant signaling involves an active signal transducer repressor and an active signal transducer;
the aberrant signaling involves an inactive signal transducer activator and an active signal transducer;
the aberrant signaling involves an inactive signal transducer;
the aberrant signaling involves an underactive signal transducer;
the aberrant signaling involves a constitutively inactive signal transducer over a period of time;
the aberrant signaling involves an inactive signal transducer repressor and an inactive signal transducer;
the aberrant signaling involves an active signal transducer activator and an inactive signal transducer;
the aberrant signaling involves an active signal transducer, and wherein the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof;
the synthetic protein circuit is capable of directly or indirectly inducing cell death in the presence of the aberrant signaling;
the first cell death executioner is capable of directly or indirectly inducing cell death in the presence of aberrant signaling;
the synthetic protein circuit is capable of directly or indirectly inducing cell death when a first level of activation of the first signal transducer is above a first signal transducer activation threshold and/or a second level of activation of the second signal transducer is below a second signal transducer activation threshold;
the effector protein is capable of directly or indirectly inducing cell death when a first level of activation of the first signal transducer is above a first signal transducer activation threshold and/or a second level of activation of the second signal transducer is below a second signal transducer activation threshold;
the synthetic protein circuit is present in a cell; and/or
the cell is:
a cell of a subject, optionally a subject suffering from a disease or disorder, optionally the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof;
a cell derived from a donor; and/or
an in vivo cell, an ex vivo cell, an in vitro cell or an in situ cell.

62. A nucleic acid composition, comprising:
one or more polynucleotides encoding the synthetic protein circuit of any one of claims 1-61, optionally the one or more polynucleotides comprise:
one or more first polynucleotides encoding a first apoptosis polypeptide, a first pyroptosis polypeptide, or a first polypeptide;
one or more second polynucleotides encoding a second apoptosis polypeptide, a second pyroptosis polypeptide, or a second polypeptide; and/or
one or more third polynucleotides encoding a third apoptosis polypeptide,
optionally, the nucleic acid composition comprises one or more polynucleotides encoding a first and/or second input polypeptide.

63. The nucleic acid composition of claim 62, wherein:
at least two of the one or more polynucleotides are operably linked to a tandem gene expression element;
the one or more polynucleotides comprise:
a 5′UTR and/or a 3′UTR;
a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or
a transcript stabilization element, optionally the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof;
the one or more polynucleotides are operably connected to a promoter selected from the group comprising:
a minimal promoter, optionally TATA, miniCMV, and/or miniPromo;
a tissue-specific promoter and/or a lineage-specific promoter; and/or
a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof;
the nucleic acid composition is configured to enhance stability, durability, and/or expression level,
optionally a 5′ untranslated region (UTR), a 3′ UTR, and/or a 5′ cap;
optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or
optionally a modified nucleotide in place of one or more uridines,
optionally the modified nucleoside is selected from pseudouridine (ψ), N 1-methyl-pseudouridine (m 1ψ), and 5-methyl-uridine (m5U);
the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition;
the nucleic acid composition is, comprises, or further comprises, one or more vectors,
optionally at least one of the one or more vectors is a viral vector, a plasmid, a transposable element, a naked DNA vector, a lipid nanoparticle (LNP), a bacterial cell, a bacteriophage, or any combination thereof,
optionally the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and
optionally the transposable element is piggybac transposon or sleeping beauty transposon;
the one or more polynucleotides are comprised in the one or more vectors,
optionally the one or more polynucleotides are comprised in the same vector and/or different vectors,
optionally the one or more polynucleotides are situated on the same nucleic acid and/or different nucleic acids;
the nucleic acid composition comprises circular mRNA, circular DNA, self-amplifying RNA, self-amplifying RNA, and/or mRNA;
the nucleic acid composition is configured to achieve relative levels of the first apoptotic polypeptide, the second apoptotic polypeptide, the third apoptotic polypeptide, the first input polypeptide, the second input polypeptide, the first polypeptide and/or the second polypeptide desired by a user;
the nucleic acid composition is configured to achieve relative levels of the first pyroptotic polypeptide, the second pyroptotic polypeptide, the first input polypeptide, the second input polypeptide, the first polypeptide and/or the second polypeptide desired by a user;
the expression of one or more of the first apoptotic polypeptide, the second apoptotic polypeptide, the third apoptotic polypeptide, first pyroptotic polypeptide, the second pyroptotic polypeptide, the first input polypeptide, and/or the second polypeptide is configured to be dosage invariant and/or robust to tissue tropism and stochastic expression; and/or
the induction of apoptosis or the induction of pyroptosis can be tuned by adjusting the relative levels of the first apoptotic polypeptide, the second apoptotic polypeptide, the third apoptotic polypeptide, first pyroptotic polypeptide, the second pyroptotic polypeptide, the first input polypeptide, and/or the second polypeptide.

64. A composition, comprising:
a first population of sender cells comprising:
(i) one or more first polynucleotide(s) encoding a first apoptosis polypeptide comprising a large subunit of an apoptotic effector protein and a small subunit of an apoptotic effector protein separated by a first heterologous protease cleavage site,
wherein two of the first apoptosis polypeptides are capable of forming a first apoptotic protein complex in a first apoptotic protein complex inactive state,
wherein a first heterologous protease in a first heterologous protease active state is capable of cutting the first heterologous protease cleavage site of the first apoptosis polypeptide, and wherein the first heterologous protease cleavage site of the first apoptosis polypeptide being cut changes the first apoptotic protein complex from the first apoptotic protein complex inactive state to a first apoptotic protein complex active state, and
wherein the first apoptotic protein complex in the first apoptotic protein complex active state is capable of inducing apoptosis in a cell; and
(ii) a first vector; and
a second population of sender cells comprising:
(i) one or more second polynucleotide(s) encoding a first input polypeptide; and
(ii) a second vector.

65. The composition of claim 64, wherein the first vector of the first population of sender cells and the second vector of the second population of sender cells are capable of delivering the one or more first polynucleotide(s) and the one or more second polynucleotide(s) to receiver cells.

66. The composition of claim 65, wherein the first apoptosis polypeptide and the first input polypeptide are expressed in the receiver cells, thereby the first apoptotic protein complex in the first apoptotic protein complex active state is capable of inducing apoptosis in the receiver cells.

67. The composition of any one of claims 64-66, wherein:
the apoptotic effector protein comprises caspase-1, caspase-2, caspase-3, caspase-4, caspase-5, caspase-6, caspase-7, caspase-8, caspase-9, caspase-10, caspase-14 or any variant, portion, or derivative thereof;
the first heterologous protease comprises or is derived from one or more of a prokaryotic protease, bacterial protease, archaeal protease, eukaryotic protease, fungal protease, plant protease, mammalian protease, and human protease, optionally the first heterologous protease is engineered, optionally wherein the first heterologous protease comprises tobacco etch virus (TEV) protease, tobacco vein mottling virus (TVMV) protease, hepatitis C virus protease (HCVP), derivatives thereof, or any combination thereof, optionally the first heterologous protease cleavage site comprises the sequence of SEQ ID NO: 1 or SEQ ID NO: 2;
the first heterologous protease cleavage site is natural or engineered, optionally the cleavage sequence of a human apoptotic effector protease, optionally a caspase cleavage sequence, optionally wherein the first heterologous protease cleavage site is configured to modulate an activation threshold of the synthetic protein circuit, optionally wherein said configuring comprises introducing one or more amino acid insertions, substitutions, or deletions relative to a wild type first heterologous protease cleavage site, further optionally the first heterologous protease cleavage site comprises the sequence of any one of SEQ ID NOs: 3-7; and/or
the first apoptosis polypeptide, the first input polypeptide, or both comprise a membrane-localization domain, optionally the membrane-localization domain comprises a CAAX domain.

68. A composition, comprising:
a first population of sender cells comprising:
(i) one or more first polynucleotide(s) encoding a first pyroptosis polypeptide comprising a pyroptosis effector domain and a first inhibitory domain separated by a first heterologous protease cleavage site, wherein the first inhibitory domain is capable of inhibiting activity of the pyroptosis effector domain, thereby the first pyroptosis polypeptide is in a first pyroptosis polypeptide inactive state,
wherein a first heterologous protease in a first heterologous protease active state is capable of cutting the first heterologous protease cleavage site of the first pyroptosis polypeptide, and wherein the first heterologous protease cleavage site of the first pyroptosis polypeptide being cut changes the first pyroptosis polypeptide from the first pyroptosis polypeptide inactive state to a first pyroptosis polypeptide active state,
wherein the first pyroptosis polypeptide in the first pyroptosis polypeptide active state is capable of inducing pyroptosis in a cell, and
(ii) a first vector; and
a second population of sender cells comprising:
(i) one or more second polynucleotide(s) encoding a first input polypeptide comprising the first heterologous protease; and
(ii) a second vector.

69. The composition of claim 68, wherein:
the first vector of the first population of sender cells and the second vector of the second population of sender cells are capable of delivering the one or more first polynucleotide(s) and the one or more second polynucleotide(s) to receiver cells;
the first pyroptosis polypeptide and the first input polypeptide are expressed in the receiver cells, thereby the first pyroptosis polypeptide in the first pyroptosis polypeptide active state is capable of inducing pyroptosis in the receiver cells;
the pyroptosis effector domain comprises an N-terminal domain of a gasdermin (GSDM) protein;
the GSDM protein is GSDMA, GSDMB, GSDMC, GSDMD, GSDME, or DFNB59;
the first heterologous protease comprises or is derived from one or more of a prokaryotic protease, bacterial protease, archaeal protease, eukaryotic protease, fungal protease, plant protease, mammalian protease, and human protease, optionally the first heterologous protease is engineered, optionally wherein the first heterologous protease comprises tobacco etch virus (TEV) protease, tobacco vein mottling virus (TVMV) protease, hepatitis C virus protease (HCVP), derivatives thereof, or any combination thereof, optionally the first heterologous protease cleavage site comprises the sequence of SEQ ID NO: 1 or SEQ ID NO: 2;
the first heterologous protease cleavage site is natural or engineered, wherein the first heterologous protease cleavage site is configured to modulate an activation threshold of the synthetic protein circuit, optionally wherein said configuring comprises introducing one or more amino acid insertions, substitutions, or deletions relative to a wild type first heterologous protease cleavage site, further optionally the first heterologous protease cleavage site comprises the sequence of any one of SEQ ID NOs: 3-7; and/or
the first inhibitory domain comprises a protein domain configured to reduce or abrogate the activity of the pyroptosis effector domain, optionally a bulky domain, further optionally the first inhibitory domain comprises an auto-inhibitory domain of a gasdermin family of pore-forming proteins or maltose-binding protein.

70. The composition of any one of claims 68-69, wherein the first apoptosis polypeptide or the first pyroptosis polypeptide comprise one or more third partner domains and one or more fourth partner domains capable of binding the third partner domain,
wherein binding of the third partner domain and the fourth partner domain of the first apoptosis polypeptide is capable of inhibiting change of the first apoptotic protein complex from the first apoptotic protein complex inactive state to the first apoptotic protein complex active state in the absence of the first heterologous protease in the first heterologous protease active state, or
wherein binding of the third partner domain and the fourth partner domain of the first pyroptosis polypeptide is capable of inhibiting change of the first pyroptosis polypeptide from the first pyroptosis polypeptide inactive state to the first pyroptosis polypeptide active state in the absence of the first heterologous protease in the first heterologous protease active state,
optionally wherein said third partner domains and fourth partner domains are capable of multimerization.

71. A composition, comprising:
a first population of sender cells comprising:
(i) one or more first polynucleotide(s) encoding a first pyroptosis polypeptide comprising a pyroptosis effector domain and a first partner domain; and
(ii) a first vector,
wherein the first population of sender cells express a silencer polypeptide comprising a first inhibitory domain and a second partner domain capable of binding the first partner domain, and
wherein the inhibitor domain of the silencer polypeptide is capable of inhibiting the first pyroptosis polypeptide when the first pyroptosis polypeptide associates with the silencer polypeptide via binding of the first partner domain and the second partner domain, thereby the first pyroptosis polypeptide is in a first pyroptosis polypeptide inactive state.

72. The composition of claim 71, wherein:
the first vector is capable of delivering the one or more first polynucleotide(s) to receiver cells;
the first pyroptosis polypeptide is expressed in the receiver cells, wherein the first pyroptosis polypeptide is capable of being in a first pyroptosis polypeptide active state, wherein the first pyroptosis polypeptide in the first pyroptosis polypeptide active state is capable of inducing pyroptosis in the receiver cells;
the receiver cells do not express the silencer polypeptide;
the first inhibitory domain comprises a protein domain configured to reduce or abrogate the activity of the pyroptosis effector domain, optionally a bulky domain, further optionally the first inhibitory domain comprises an auto-inhibitory domain of a gasdermin family of pore-forming proteins or maltose-binding protein;
the pyroptosis effector domain comprises an N-terminal domain of a gasdermin (GSDM) protein; and/or
the GSDM protein is GSDMA, GSDMB, GSDMC, GSDMD, GSDME, or DFNB59.

73. The composition of any one of claims 68-72, wherein inducing pyroptosis in a receiver cell causes the receiver cell to release one or more inflammatory cytokines, optionally the one or more inflammatory cytokines comprise IL-18, IL-1β, IL-6, IL-8, interferon gamma (IFN-γ), and/or tumor necrosis factor-alpha (TNF-α).

74. The composition of any one of claims 68-73, wherein:
(i) the first apoptosis polypeptide;
(ii) the first pyroptosis polypeptide, and/or
(iii) the first input polypeptide,
are configured to be in a first localized state, optionally
the first localized state comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the first apoptosis polypeptide, the first pyroptosis polypeptide, and/or the first input polypeptide is: (i) tethered to an intracellular organelle and/or membrane; or (ii) fused to a polypeptide that recruits to said localized state.

75. The composition of any one of claims 64-74, wherein:
(i) the first apoptosis polypeptide;
(ii) the first pyroptosis polypeptide, and/or
(iii) the first input polypeptide,
are configured to be in second localized state(s), wherein the first localized state and the second localized state(s) are different, optionally:
the second localized state(s) comprises a state created by phase separation, a state defined by the proximity to a given protein, and/or localization to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the first apoptosis polypeptide, the first pyroptosis polypeptide, and/or the first input polypeptide is: (i) tethered to an intracellular organelle and/or membrane; or (ii) fused to a polypeptide that recruits to said localized state.

76. The synthetic protein circuit of any one of claims 64-75, wherein:
(i) the first apoptosis polypeptide;
(ii) the first pyroptosis polypeptide, and/or
(iii) the first input polypeptide comprise a first localization signal, optionally the first localization signal is adjacent to a third degron and/or a third heterologous cleavage site.

77. The composition of any one of claims 64-76, wherein:
(i) the first apoptosis polypeptide;
(ii) the first pyroptosis polypeptide, and/or
(iii) the first input polypeptide comprise second localization signal(s), optionally the second localization signal is adjacent to a third degron and/or a third heterologous protease cleavage site.

78. The composition of any one of claims 64-77, wherein the presence of the third degron and/or wherein the third heterologous cleavage site being cut changes:
(i) the first apoptosis polypeptide from a non-localized state or the first localized state to the second localized state(s) or a non-localized state;
(ii) the first pyroptosis polypeptide from a non-localized state or the first localized state to the second localized state(s) or a non-localized state; and/or
(iii) the first input polypeptide from a non-localized state or the first localized state to the second localized state(s) or a non-localized state.

79. The composition of any one of claims 64-78, wherein:
the synthetic protein circuit further comprises a third input polypeptide comprising a third heterologous protease capable of cutting the third heterologous protease cleavage site, optionally wherein the third heterologous protease comprises or is derived from one or more of a prokaryotic protease, bacterial protease, archaeal protease, eukaryotic protease, fungal protease, plant protease, mammalian protease, and human protease, optionally the third heterologous protease is engineered, optionally wherein the third heterologous protease comprises tobacco etch virus (TEV) protease, tobacco vein mottling virus (TVMV) protease, hepatitis C virus protease (HCVP), derivatives thereof, or any combination thereof, further optionally the third heterologous protease cleavage site comprises the sequence of SEQ ID NO: 1 or SEQ ID NO: 2; and/or
wherein the third heterologous protease cleavage site is natural or engineered, optionally the cleavage sequence of a human apoptotic effector protease, optionally a caspase cleavage sequence, optionally wherein the third heterologous protease cleavage site is configured to modulate an activation threshold of the synthetic protein circuit, optionally wherein said configuring comprises introducing one or more amino acid insertions, substitutions, or deletions relative to a wild type third heterologous protease cleavage site, further optionally the third heterologous protease cleavage site comprises the sequence of any one of SEQ ID NOs: 3-7.

80. The composition of any one of claims 64-79, wherein:
(a) the first apoptosis polypeptide in the first localized state;
(b) the first apoptosis polypeptide in the second localized state(s);
(c) the first pyroptosis in the first localized state;
(d) the first pyroptosis polypeptide in the second localized state(s);
(e) the first input polypeptide in the first localized state; and/or
(f) the first second input polypeptide in the second localized state(s),
is capable of modulating an activation threshold and/or sensitivity of the synthetic protein circuit.

81. The composition of any one of claims 64-80, wherein the first and/or second vector is a viral vector, a plasmid, a naked DNA vector, a naked RNA vector, a lipid nanoparticle, or any combination thereof, optionally the viral vector is an AAV vector, a lentivirus vector, a retrovirus vector, an integration-deficient lentivirus (IDLV) vector, further optionally the AAV vector comprises single-stranded AAV (ssAAV) vector or a self-complementary AAV (scAAV) vector.

82. The composition of any one of claims 64-81, wherein the receiver cell or the sender cell is:
a cell of a subject, optionally a subject suffering from a disease or disorder, optionally the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof;
a cell derived from a donor; and/or
an in vivo cell, an ex vivo cell, or an in situ cell.

83. The composition of any one of claims 64-82, wherein the first and/or the second vector is capable of delivering the one or more first polynucleotides and/or the one or more second polynucleotides to one or more tissues of a subject, optionally:
the receiver cells are situated within one or more tissues of a subject; and/or
the one or more tissues comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof.

84. The composition of any one of claims 64-83, wherein the receiver cell comprises a unique cell type and/or a unique cell state.

85. The composition of claim 84,
wherein the unique cell type and/or the unique cell state comprises a unique gene expression pattern, optionally the unique cell type and/or unique cell state comprises (i) one or more mutations of a protein, (ii) structural variants and/or copy-number alternations of one or more protein-coding genes, (iii) epigenetic signature(s), and/or (iv) a unique anatomic location, further optionally the unique cell type and/or the unique cell state comprises anatomically locally unique gene expression;
wherein the unique cell type and/or the unique cell state is caused by hereditable, environmental, and/or idiopathic factors;
wherein the unique cell type and/or the cell in the unique cell state (i) causes and/or aggravates a disease or disorder and/or (ii) is associated with the pathology of a disease or disorder; and/or
wherein the unique cell state comprises a senescent cell state induced by a tumor microenvironment, optionally the senescent cell state induced by a tumor microenvironment comprises expression of CD57, IKRLG1, TIGIT, p21, p53, phospho-p53, DEC1, PPP1A, γH2AX, 53BPI, Rad17, ATR, ATM, MDC1, TIF, IL-6, IL-8, CXCR2, IGF2, IGFBP3, IGFBP5, IGFBP7, STC1, GDF15, SERPIN, ICAM-1, DEP1, B2MG, NOTCH3, DcR2, or any combination thereof.

86. The composition of any one of claims 84-85, wherein the unique cell state and/or unique cell type is characterized by one or more of:
aberrant signaling of one or more signal transducer(s);
cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment;
acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression; and
nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell sternness, survival, and invasion.

87. The composition of any one of claims 84-86, wherein the unique cell state comprises:
a physiological state, optionally a cell cycle state, a differentiation state, a development state a metabolic state, or a combination thereof; and/or
a pathological state, optionally a disease state, a human disease state, a diabetic state, an immune disorder state, a neurodegenerative disorder state, an oncogenic state, or a combination thereof.

88. The composition of any one of claims 64-87, wherein the receiver cell is characterized by aberrant signaling of one or more signal transducers, and wherein the aberrant signaling involves:
an overactive signal transducer;
a constitutively active signal transducer over a period of time;
an active signal transducer repressor and an active signal transducer;
an inactive signal transducer activator and an active signal transducer;
an inactive signal transducer;
an underactive signal transducer;
a constitutively inactive signal transducer over a period of time;
an inactive signal transducer repressor and an inactive signal transducer; and/or
an active signal transducer activator and an inactive signal transducer.

89. The composition of claim 88, wherein the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof, optionally the signal transduscer(s) is AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCγ, NF-kB, FAK, CREB, αIIIβ3, FcεRI, BAD, p70S6K, STAT1, STAT2, STAT3, STAT5, STAT6, or any combination thereof.

90. A method of selectively killing a target cell comprising:
expressing the synthetic protein circuit of any one of claims 1-61 or the nucleic acid composition of any one of claims 62-63 in the target cell,
wherein the synthetic protein circuit is configured to be responsive to a unique cell type and/or unique cell state of the target cell, optionally wherein the first and/or second heterologous protease is configured to be in the first and/or second heterologous protease active state in response to the unique cell type and/or unique cell state of the target cell.

91. The method of claim 90, wherein:
the unique cell type and/or the unique cell state comprises a unique gene expression pattern, optionally the unique cell type and/or unique cell state comprises: (i) one or more mutations of a protein, (ii) structural variants and/or copy-number alternations of one or more protein-coding genes, (iii) epigenetic signature(s), and/or (iv) a unique anatomic location, further optionally the unique cell type and/or the unique cell state comprises anatomically locally unique gene expression;
the unique cell type and/or the unique cell state is caused by hereditable, environmental, and/or idiopathic factors;
the unique cell type and/or the cell in the unique cell state (i) causes and/or aggravates a disease or disorder and/or (ii) is associated with the pathology of a disease or disorder;
the unique cell state comprises a senescent cell state induced by a tumor microenvironment, optionally the senescent cell state induced by a tumor microenvironment comprises expression of CD57, IKRLG1, TIGIT, p21, p53, phospho-p53, DEC1, PPP1A, γH2AX, 53BPL Rad17, ATR, ATM, MDC1, TIF, IL-6, IL-8, CXCR2, IGF2, IGFBP3, IGFBP5, IGFBP7, STC1, GDF15, SERPIN, ICAM-1, DEP1, B2MG, NOTCH3, DcR2, or any combination thereof;
the unique cell state and/or unique cell type is characterized by aberrant signaling of one or more signal transducer(s);
the unique cell state comprises: a physiological state, optionally a cell cycle state, a differentiation state, a development state a metabolic state, or a combination thereof; and/or a pathological state, optionally a disease state, a human disease state, a diabetic state, an immune disorder state, a neurodegenerative disorder state, an oncogenic state, or a combination thereof;
the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment;
the unique cell state and/or unique cell type is characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression;
the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion;
the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer; and/or
wherein the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof, optionally the signal transducer(s) is AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCγ, NF-kB, FAK, CREB, αIIIβ3, FcεRI, BAD, p70S6K, STAT1, STAT2, STAT3, STAT5, STAT6.

92. The method of any one of claims 90-91, wherein configuring the first and/or the second heterologous protease to be in the first and/or the second heterologous protease active state in response to the unique cell type and/or the unique cell state of the target cell comprises:
expressing a second synthetic protein circuit in the target cell, wherein the second synthetic protein circuit comprises:
a first polypeptide comprising a first signal transducer binding domain and a first part of a first protease domain of the first or second heterologous protease, wherein the first signal transducer binding domain is capable of binding a first signal transducer to form a first signal transducer-bound polypeptide;
a second polypeptide comprising a second signal transducer binding domain and a second part of the first protease domain of the first or second heterologous protease, wherein the second signal transducer binding domain is capable of binding a second signal transducer to form a second signal transducer-bound polypeptide, wherein the first part of the first protease domain and the second part of the first protease domain have weak association affinity, and wherein the first part of the first protease domain and the second part of the first protease domain are capable of associating with each other to constitute the first or second heterologous protease capable in a first or second heterologous protease active state capable of cutting:
(i) the first, second, or third apoptosis polypeptide at the first or second heterologous protease cleavage site when the first signal transducer and the second signal transducer are in close proximity at an association location; or
(ii) the first or second pyroptosis polypeptide at the first or second heterologous protease cleavage site when the first signal transducer and the second signal transducer are in close proximity at an association location.

93. The method of any one of claims 90-92, wherein:
the first signal transducer binding domain of the first polypeptide and the second signal transducer binding domain of the second polypeptide are identical;
the first signal transducer and the second signal transducer are identical and/or are the same protein;
the first signal transducer, the second signal transducer, or both, are capable of being localized at the association location;
the first signal transducer when in a first signal transducer active state, the second signal transducer when in a second signal transducer active state, or both, are capable of being localized at the association location;
the first signal transducer when in a first inactive state, the second signal transducer when in a second inactive state, or both, are capable of being localized at the association location;
the first signal transducer binding domain of the first polypeptide is capable of binding to the first signal transducer, wherein the second signal transducer binding domain of the second polypeptide is capable of binding to the second signal transducer, or both;
the first signal transducer binding domain of the first polypeptide is capable of binding to the first signal transducer in a first signal transducer active state, wherein the second signal transducer binding domain of the second polypeptide is capable of binding to the second signal transducer in a second signal transducer active state, or both;
the first signal transducer binding domain of the first polypeptide is capable of binding to the first signal transducer in a first inactive state, wherein the second signal transducer binding domain of the second polypeptide is capable of binding to the second signal transducer in a second inactive state, or both;
the first signal transducer binding domain of the first polypeptide is capable of binding the first signal transducer to form the first signal transducer-bound polypeptide at the association location, wherein the signal transducer binding domain of the first polypeptide is capable of binding the first signal transducer to form the first signal transducer-bound polypeptide at the association location, or both;
the signal transducer binding domain of the first polypeptide is capable of binding the first signal transducer to form the first signal transducer-bound polypeptide at a first cellular location other than the association location, wherein the signal transducer binding domain of the first polypeptide is capable of binding the first signal transducer to form the first signal transducer-bound polypeptide at a second cellular location other than the association location, or both;
the first cellular location, the second cellular location, or both comprise one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof;
the association location comprises one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof;
the first signal transducer binding domain and the second signal transducer binding domain are different;
the first signal transducer binding domain and/or the second signal transducer binding domain each is capable of binding molecules of the first signal transducer and/or the second signal transducer;
the first signal transducer and/or the second signal transducer belong to a signal transduction pathway;
the first signal transducer binding domain and/or the second signal transducer binding domain comprise a RAS binding domain (RBD) and/or RAS association domain (RAD), optionally the RAS binding domain comprises or is derived from a RAS interacting protein, optionally selected from the group comprising AGO2, APBB1IP, APPL1, ARAF, ARL1, ARL2, ARRB1, ARRB2, BAIAP2, BCL2, BCL2L1, BRAF, BRAP, BSG, CALM1, CALM3, CALML3, CALML4, CALML5, CALML6, CNKSR1, CNKSR2, CSK, DAB2IP, EGFR, ERBIN, FGA, FGB, FGG, FN1, GRB2, HK1, IFNGR1, IL6, IQGAP1, ITGA2B, ITGB3, KSR1, KSR2, LGALS3, LYN, LZTR1, MAP2K1, MAP2K2, MAPK1, MAPK14, MAPK3, MAPKAP1, MARK2, MARK3, MBP, MSI2, MTOR, NCBP2AS2, NF1, NIBAN2, PDE4DIP, PDE6D, PDPK1, PEBP1, PIK3CA, PIK3CB, PIK3CD, PIK3R1, PIK3R2, PIP5K1A, PLCE1, PPIA, PRKCZ, PTGS2, RAF1, RALB, RALGDS, RAP1A, RAP1B, RAP1GDS1, RASA1, RASA2, RASA3, RASA4, RASAL1, RASAL2, RASAL3, RASSF1, RASSF2, RASSF5, RGL1, RGL3, RIN1, SHOC2, SOS1, SOS2, SPRED1, SPRED2, SPRED3, SRC, SYNGAP1, TIAM1, TLN1, VCL, VWF, YWHAB, or any combination thereof;
the first signal transducer binding domain and/or the second signal transducer binding domain comprises a lipid binding domain;
the lipid binding domain comprises a Pleckstrin homology (PH) domain;
the first signal transducer binding domain and/or the second signal transducer binding domain comprises an antibody, an antibody fragment, a binding domain derived from a natural protein, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof;
the first signal transducer is capable of binding the first signal transducer binding domain and/or the second signal transducer is capable of binding the second signal transducer binding domain following a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof;
the first signal transducer, the second signal transducer, or both are endogenous proteins;
the first signal transducer, the second signal transducer, or both comprise AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCγ, NF-kB, FAK, CREB, αIIIβ3, FcεRI, BAD, p70S6K, STAT1, STAT2, STAT3, STAT5, STAT6, or any combination thereof;
the first signal transducer and/or the second signal transducer are capable of regulating cell survival, cell growth, cell proliferation, cell adhesion, cell migration, cell metabolism, cell morphology, cell differentiation, apoptosis, or any combination thereof;
the first signal transducer, the second signal transducer, or both comprise a RAS protein, a CTNNB1 protein, or a TP53 protein;
the RAS protein is KRAS, NHAS, HRAS, or any combination thereof, optionally the RAS protein comprises a G12 mutation, G13 mutation, a Q61 mutation, and/or an A146 mutation, optionally the G12 mutation is selected from the group comprising G12A, G12C, G12D, G12R, G12S, G12V, and any combination thereof, optionally the G13 mutation is selected from the group comprising G13C, G13D, G13dup, G13R, G13S, G13V, and any combination thereof, optionally the Q61 mutation is selected from the group comprising Q61H, Q61K, Q61L, Q61R, Q61E, Q61P, Q61*, and any combination thereof, optionally the A146 mutation is selected from the group comprising A146P, A146T, A146V, and any combination thereof;
the signature detected by the input polypeptide(s) is correlated with any other cellular signature, protein state, cell type, and/or cell state capable of being read out as a biomarker;
the signal transducer is CTNNB1, and wherein the cell state is defined by CTNNB1 mutation(s) and/or localization and/or concentration and/or protein turnover and/or multimerization and/or PTM(s);
the signal transducer is TP53, and wherein the cell state is defined by TP53 mutation(s) and/or elevated TP53 concentration and/or altered TP53 oligomerization/multimerization state and/or TP53 localization pattern and/or PTM(s) and/or turnover;
the signal transducer(s) are associated with disease, optionally cancer;
the first signal transducer, the second signal transducer, or both are exogenous proteins;
the second synthetic protein circuit comprises the first signal transducer, the second signal transducer, or both; and/or
the first signal transducer, the second signal transducer, or both comprise a lipid, optionally the lipid comprises a phospholipid, further optionally the phospholipid is phosphatidylinositol 3-phosphate.

94. A method of treating or preventing a disease or disorder in a subject in need thereof, comprising:
expressing the synthetic protein circuit of any one of claims 1-61 or the first synthetic protein circuit and/or second synthetic protein circuit of any one of claims 90-93, in a cell of the subject.

95. A method of treating or preventing a disease or disorder in a subject in need thereof, comprising:
administering to the subject an effective amount of the nucleic acid composition of any one of claims 62-63 or the composition of any one of claims 64-89, thereby treating or preventing the disease or disorder in the subject.

96. The method of claim 95, wherein administering comprises:
(i) isolating one or more cells from the subject;
(ii) contacting said one or more cells with the nucleic acid composition of any one of claims 62-63, thereby generating engineered cells, optionally the contacting comprises transfection; and
(iii) administering the one or more engineered cells into a subject after the contacting step.

97. The method of any one of claims 90-96, wherein the disease or disorder is a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof, optionally a solid tumor.

98. The synthetic protein circuit, nucleic acid composition, or method of any one of claims 1-97, further comprising a supplementary protein circuit comprising:
a first polypeptide comprising an optional first supplementary domain and a first part of a first protease domain of a supplementary heterologous protease;
a second polypeptide comprising a second supplementary domain and a second part of the first protease domain of the supplementary protease,
wherein the first part of the first protease domain and the second part of the first protease domain have weak association affinity, and wherein the first part of the first protease domain and the second part of the first protease domain are capable of associating with each other to constitute the supplementary heterologous protease,
optionally the first and/or second supplementary domain is a signal transducer binding domain,
optionally the supplementary heterologous protease in a supplementary heterologous protease active state is capable of cutting (i) the first, second, or third apoptosis polypeptide at the first or second heterologous protease cleavage site, and/or (ii) the first or second pyroptosis polypeptide at the first or second heterologous protease cleavage site, further optionally when a first signal transducer and a second signal transducer are in close proximity at an association location,
optionally the supplementary heterologous protease is the first or second heterologous protease.

99. The synthetic protein circuit, nucleic acid composition, or method of any one of claims 1-98, wherein the induction of apoptosis and/or pyroptosis is dependent on the dose of one or more synthetic protein circuit components, optionally cell death is triggered when a threshold amount of one or more of the following is reached: (i) the first, second, and/or third apoptotic protein complex in the first, second, and/or third apoptotic protein complex active state; (ii) the first and/or second pyroptosis polypeptide in a first and/or second pyroptosis polypeptide active state; and/or (iii) a pyroptosis effector protein in a pyroptosis effector protein active state.


SEQUENCE LISTING (45 sequences)

SEQ ID NO: 1 - protein, 7 aa, synthetic construct
ENLYFQG

SEQ ID NO: 2 - protein, 7 aa, synthetic construct
ENLYFQS

SEQ ID NO: 3 - protein, 7 aa, synthetic construct
ENLYFQY

SEQ ID NO: 4 - protein, 7 aa, synthetic construct
ENLYFQQ

SEQ ID NO: 5 - protein, 7 aa, synthetic construct
ENLFFQS

SEQ ID NO: 6 - protein, 7 aa, synthetic construct
ENLFFQY

SEQ ID NO: 7 - protein, 7 aa, synthetic construct
ENLFFQQ

SEQ ID NO: 8 - protein, 15 aa, synthetic construct
FYDAMDGQIQGSVEL

SEQ ID NO: 9 - protein, 22 aa, synthetic construct
FKENLYFQSNMLDVQVQGLVEV

SEQ ID NO: 10 - protein, 284 aa, synthetic construct
MGSENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 11 - protein, 207 aa, synthetic construct
MGSEQLEKKLQALEKKLAQLEWKNQALEKKLAQENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETD

SEQ ID NO: 12 - protein, 312 aa, synthetic construct
MGSSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHGGSGALKKELQANKKELAQLKWELQALKKELAQGGSGLENLYFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 13 - protein, 228 aa, synthetic construct
MGSENLYFQYHKSGAWKLPVSLVKEQLEKKLQALEKKLAQLEWKNQALEKKLAQENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETD

SEQ ID NO: 14 - protein, 138 aa, synthetic construct
MGSSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHGGSGALKKELQANKKELAQLKWELQALKKELAQ

SEQ ID NO: 15 - protein, 240 aa, synthetic construct
MGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNS

SEQ ID NO: 16 - protein, 473 aa, synthetic construct
MGSPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEENLYFQSMHEDFKTLKEEVQRETQEVEKLSPVGRSSLLTSLSHLLGKKKELQDLEQKLEGALDKGQKVTLEALPKDVLLSKDAMDAILYFLGALTELTEEQLKILVKSLEKKILPVQLKLVESTLEQNFLQDKEGVFPLQPDLLSSLGEEELTLTEALVGLSGLEVQRSGPQYAWDPDTRHNLCALYAGLSLLHLLSRKSNALTYCALS

SEQ ID NO: 17 - protein, 648 aa, synthetic construct
MGSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGSGSSENLYFQSGGGGSPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGE

SEQ ID NO: 18 - protein, 438 aa, synthetic construct
MGSPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEGGSGLENLYFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 19 - protein, 271 aa, synthetic construct
MGSPVFEDVTRALVRELNPRGDLTPLDSLIDFKHFRPFCLVLRKRKSTLFWGARYVRTDYTLLDLLEPGSSPSDLTDSGNFSFKENLYFQSNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGE

SEQ ID NO: 20 - protein, 285 aa, synthetic construct
MGSENLYFQRHKSGAWKLPVSLVKPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGE

SEQ ID NO: 21 - protein, 497 aa, synthetic construct
MGFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPDAAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHS

SEQ ID NO: 22 - protein, 271 aa, synthetic construct
MGFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTNAYGVIELYVKLDGQFEFCLLRGKQGGFENKKRIDSVYLDPLVFREFAFIDMPD

SEQ ID NO: 23 - protein, 458 aa, synthetic construct
MGSENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHGGSGLETVRFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 24 - protein, 291 aa, synthetic construct
MGSENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSETVRFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 25 - protein, 228 aa, synthetic construct
MGSETVRFQYHKSGAWKLPVSLVKEQLEKKLQALEKKLAQLEWKNQALEKKLAQENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETD

SEQ ID NO: 26 - protein, 312 aa, synthetic construct; identical to SEQ ID NO: 12

SEQ ID NO: 27 - protein, 239 aa, synthetic construct
MGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMDS

SEQ ID NO: 28 - protein, 857 aa, synthetic construct
MGSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGSGSSETVRFQSGGGGSPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEENLYFQSMHEDFKTLKEEVQRETQEVEKLSPVGRSSLLTSLSHLLGKKKELQDLEQKLEGALDKGQKVTLEALPKDVLLSKDAMDAILYFLGALTELTEEQLKILVKSLEKKILPVQLKLVESTLEQNFLQDKEGVFPLQPDLLSSLGEEELTLTEALVGLSGLEVQRSGPQYAWDPDTRHNLCALYAGLSLLHLLSRKSNALTYCALS

SEQ ID NO: 29 - protein, 479 aa, synthetic construct
MGPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEENLYFQSETVRFQSMHEDFKTLKEEVQRETQEVEKLSPVGRSSLLTSLSHLLGKKKELQDLEQKLEGALDKGQKVTLEALPKDVLLSKDAMDAILYFLGALTELTEEQLKILVKSLEKKILPVQLKLVESTLEQNFLQDKEGVFPLQPDLLSSLGEEELTLTEALVGLSGLEVQRSGPQYAWDPDTRHNLCALYAGLSLLHLLSRKSNALTYCALS

SEQ ID NO: 30 - protein, 459 aa, synthetic construct
MGSETVRFQRHKSGAWKLPVSLVKPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEGGSGLENLYFQSGSGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 31 - protein, 305 aa, synthetic construct
MGSENLYFQYHKSGAWKLPVSLVKENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETETVRFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 32 - protein, 305 aa, synthetic construct
MGSETVRFQYHKSGAWKLPVSLVKENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 33 - protein, 235 aa, synthetic construct
MGSENLYFQYHKSGAWKLPVSLVKETVRFQSEQLEKKLQALEKKLAQLEWKNQALEKKLAQENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETD

SEQ ID NO: 34 - protein, 138 aa, synthetic construct; identical to SEQ ID NO: 14

SEQ ID NO: 35 - protein, 228 aa, synthetic construct; identical to SEQ ID NO: 13

SEQ ID NO: 36 - protein, 228 aa, synthetic construct; identical to SEQ ID NO: 25

SEQ ID NO: 37 - protein, 494 aa, synthetic construct
MGSENLYFQRHKSGAWKLPVSLVKPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEETVRFQSMHEDFKTLKEEVQRETQEVEKLSPVGRSSLLTSLSHLLGKKKELQDLEQKLEGALDKGQKVTLEALPKDVLLSKDAMDAILYFLGALTELTEEQLKILVKSLEKKILPVQLKLVESTLEQNFLQDKEGVFPLQPDLLSSLGEEELTLTEALVGLSGLEVQRSGPQYAWDPDTRHNLCALYAGLSLLHLLSRKSNALTYCALS

SEQ ID NO: 38 - protein, 494 aa, synthetic construct
MGSETVRFQRHKSGAWKLPVSLVKPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGEENLYFQSMHEDFKTLKEEVQRETQEVEKLSPVGRSSLLTSLSHLLGKKKELQDLEQKLEGALDKGQKVTLEALPKDVLLSKDAMDAILYFLGALTELTEEQLKILVKSLEKKILPVQLKLVESTLEQNFLQDKEGVFPLQPDLLSSLGEEELTLTEALVGLSGLEVQRSGPQYAWDPDTRHNLCALYAGLSLLHLLSRKSNALTYCALS

SEQ ID NO: 39 - protein, 292 aa, synthetic construct
MGSENLYFQRHKSGAWKLPVSLVKETVRFQSPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGE

SEQ ID NO: 40 - protein, 285 aa, synthetic construct; identical to SEQ ID NO: 20

SEQ ID NO: 41 - protein, 285 aa, synthetic construct
MGSETVRFQRHKSGAWKLPVSLVKPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGE

SEQ ID NO: 42 - protein, 302 aa, synthetic construct
MGSPVFEDVTRALVRELNPRGDLTPLDSLIDFKHFRPFCLVLRKRKSTLFWGARYVRTDYTLLDLLEPGSSPSDLTDSGNFSFKNMLDVQVQGLVEVPKTVKVKGTAGLSQSSTLEVQTLSVAPSALENLKKERKLSADHSFLNEMRYHEKNLYVVMEAVEAKQEVTVEQTGNANAIFSLPSLALLGLQGSLNNNKAVTIPKGCVLAYRVRLLRVFLFNLWDIPYICNDSMQTFPKIRRVPCSAFISPTQMISEEPEEEKLIGESGGTQALEKKLAQLEWKLQALEKKNAQLEKKLASSGSG

SEQ ID NO: 43 - protein, 241 aa, synthetic construct
MGSEQLEKKLQALEKKLAQLEWKNQALEKKLAQGGSGSSMHEDFKTLKEEVQRETQEVEKLSPVGRSSLLTSLSHLLGKKKELQDLEQKLEGALDKGQKVTLEALPKDVLLSKDAMDAILYFLGALTELTEEQLKILVKSLEKKILPVQLKLVESTLEQNFLQDKEGVFPLQPDLLSSLGEEELTLTEALVGLSGLEVQRSGPQYAWDPDTRHNLCALYAGLSLLHLLSRKSNALTYCALS

SEQ ID NO: 44 - protein, 630 aa, synthetic construct
MEIGTGFPFDPHYVEVLGERMHYVDVGPRDGTPVLFLHGNPTSSYVWRNIIPHVAPGSENLYFQSGSENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHSTGSENLYFQSGSTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSSGGGGRSKLNPPDESGPGCMSCKCVLS

SEQ ID NO: 45 - protein, 622 aa, synthetic construct
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSEIGTGFPFDPHYVEVLGERMHYVDVGPRDGTPVLFLHGNPTSSYVWRNIIPHVAPENLYFQSTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISENLYFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHSTGGSSGGGGRSKLNPPDESGPGCMSCKCVLS